(19) United States
(12) Patent Application Publication
(10) Pub. No.: US 2023/0192842 A1
(43) Pub. Date: Jun. 22, 2023

(54) CHIMERIC ANTIGEN RECEPTORS TARGETING CLAUDIN-3 AND METHODS FOR TREATING CANCER

(71) Applicant: GlaxoSmithKline Intellectual Property Development Limited, Brentford (GB)

(72) Inventor: Thomas Southgate, Stevenage (GB)

(73) Assignee: GlaxoSmithKline Intellectual Property Development Limited, Brentford (GB)

(21) Appl. No.: 17/950,667

(22) Filed: Sep. 22, 2022

Related U.S. Application Data
(63) Continuation of application No. PCT/IB2022/052443, filed on Mar. 17, 2022.
(60) Provisional application No. 63/163,217, filed on Mar. 19, 2021.

(51) Int. Cl. C07K 16/28; C07K 14/705; C07K 14/725; A61P 35/00; A61K 35/17; A61P 11/00; A61P 1/00

(52) U.S. Cl. CPC C07K 16/28; C07K 14/70517; C07K 14/70578; C07K 14/7051; A61P 35/00; A61K 35/17; A61P 11/00; A61P 1/00; C07K 2319/33; A61K 2039/505

(57) ABSTRACT

Chimeric antigen receptors (CARs) which include an antigen binding protein that binds to a discontinuous epitope on human claudin-3 comprising at least N38 and E153 of SEQ ID NO:13 are described. Also described herein includes polynucleotides encoding the antigen binding protein, the CARs, immune effector cells containing the CARs, pharmaceutical compositions containing the immune effector cells, and methods of treating cancer with the immune effector cells.

### CROSS REFERENCE TO RELATED APPLICATIONS

This application is a Continuation of International Application No. PCT/IB2022/052443 filed Mar. 17, 2022, which claims the benefit of U.S. Provisional Application No. 63/163,217 filed Mar. 19, 2021, the disclosures of which are incorporated herein in their entireties.

### SEQUENCE LISTING

The instant application contains a Sequence Listing which has been submitted electronically in XML format and is hereby incorporated by reference in its entirety. Said XML copy, created on Nov. 15, 2022, is named PU66983US01_SL.xml and is 48,850 bytes in size.

### FIELD OF THE INVENTION

The invention relates to chimeric antigen receptors (CARs) which comprise an antigen binding protein that binds at least one epitope of a cell junction protein, wherein said cell junction protein is located within a cell-cell junction and wherein said one or more epitopes of the cell junction protein is only accessible and/or available for binding by said CAR extracellular domain in cancer cells.

### BACKGROUND OF THE INVENTION

Adoptive T cell therapies are transformative medicines with curative potential for cancer patients. To engineer these potent autologous cell therapies for patients, peripheral blood is used to obtain T cells which are then genetically modified. Introducing a chimeric antigen receptor (CAR) to these cells enables them to specifically bind to an antigen of choice. These modified cells are multiplied ex vivo and reinfused into the patient with the objective of trafficking to and subsequently killing cancer cells expressing the matching antigens (Yeku et al., Am Soc Clin Oncol Educ Book. 2017; 37: 193-204; McBride et al., Wiley Interdiscip Rev Nanomed Nanobiotechnol. 2019; 11(5): e1557).

CARs are synthetic antigen receptors that redirect T cell specificity, function and persistence. CARS are composed of the antigen specific region of an antigen binding protein, such as a single-chain variable fragment (scFv), fused to the T cells activating domain, e.g., zeta chain of the CD3 complex, and a co-stimulatory domain, e.g., CD28 or 4-1BB. This configuration promotes antigen specific activation and enhances proliferation and antiapoptotic functions of human primary T cells.

CAR-T cells have demonstrated remarkable efficacy against a range of liquid tumour B-cell malignancies; with results of early clinical trials suggesting activity in multiple myeloma (Sadelain et al., Nature. 2017; 545(7655): 423-31; June et al., N Engl J Med. 2018; 379(1): 64-73; Brudno et al., Nat Rev Clin Oncol. 2017; 15(1): 31-46). The 2017 FDA approval of CD19 CAR-T's for lymphoma and leukaemia has reinvigorated focused efforts in developing CAR-T cells for solid tumours. However, CAR-T cell therapies have demonstrated limited efficacy in solid tumours to date.

To generate a safe and efficacious T cell therapy against solid cancers, the T cells must retain a high and efficacious killing potential throughout manufacturing, be capable of trafficking to the tumour, and overcome the immunosuppressive tumour microenvironment (TME). One of the major success factors is the selection of the antigen target. Typically, an antigen selected for targeting is expressed in sufficient amounts on the cancer cell surface, while normal tissue expression remains low to ensure a low cross-reactivity to healthy cells (both off- and on-target effect). CAR immunotherapy in solid tumours remains challenging, largely due to the lack of appropriate surface antigens whose expression is confined to malignant tissue. Off-tumour expression of the antigen target has potential to cause on-target toxicity with varying degrees of severity depending on the affected organ tissue (Watanabe et al., Front Immunol. 2018; 9: 2486; Park et al., Sci Rep. 2017; 7(1): 14366).

Accordingly, there is a need to identify new targets for CAR immunotherapy, particularly for generating T cell therapies against solid tumours and cancers.

### SUMMARY OF THE INVENTION

According to a first aspect, there is provided a chimeric antigen receptor (CAR) comprising:
a) an extracellular domain which comprises an isolated claudin-3 binding protein that binds to a discontinuous epitope on human claudin-3 comprising at least N38 and E153 of SEQ ID NO:13;
b) a transmembrane domain; and
c) one or more intracellular signalling domains.

In a further aspect, provided is a chimeric antigen receptor (CAR) comprising a polypeptide comprising:
a) an extracellular domain which comprises a claudin-3 binding domain comprising a heavy chain variable region (VH) comprising a heavy chain complementarity determining region 1 (CDRH1) sequence of SEQ ID NO: 1; a heavy chain complementarity determining region 2 (CDRH2) sequence of SEQ ID NO: 2; a heavy chain complementarity determining region 3 (CDRH3) sequence of SEQ ID NO: 3;
b) a transmembrane domain; and
c) one or more intracellular signalling domains.

In a further aspect, provided is an isolated claudin-3 binding protein that binds to a discontinuous epitope on human claudin-3 comprising at least N38 and E153 of SEQ ID NO:13.

In a further aspect, provided is a claudin-3 binding protein, comprising a heavy chain variable region (VH) comprising a heavy chain complementarity determining region 1 (CDRH1) sequence of SEQ ID NO: 1; a heavy chain complementarity determining region 2 (CDRH2) sequence of SEQ ID NO: 2; a heavy chain complementarity determining region 3 (CDRH3) sequence of SEQ ID NO: 3.

In a further aspect, there is provided a polypeptide comprising the amino acid sequence of a CAR or a claudin-3 binding protein disclosed herein.

According to other aspects, there is provided a polynucleotide comprising a sequence encoding a CAR or a claudin-3 binding protein disclosed herein, a vector comprising a polynucleotide sequence disclosed herein and a vector producer cell comprising a polynucleotide sequence and/or a vector disclosed herein.

In another aspect, there is provided an immune effector cell comprising a CAR, a polypeptide, a polynucleotide and/or a vector disclosed herein.

In another aspect, there is provided a pharmaceutical composition comprising an immune effector cell or a claudin-3 binding protein disclosed herein and a pharmaceutically acceptable excipient.

In another aspect, there is provided a method of generating an immune effector cell comprising a CAR disclosed herein, said method comprising introducing into an immune effector cell a polypeptide, polynucleotide and/or a vector disclosed herein.

In another aspect, there is provided a CAR, a claudin-3 binding protein, a polypeptide, a polynucleotide, a vector, an immune effector cell or a pharmaceutical composition disclosed herein for use in the treatment of cancer.

In another aspect, there is provided a method of treating cancer in a subject, said method comprising administering to the subject a therapeutically effective amount of a CAR or a claudin-3 binding protein, a polypeptide, a polynucleotide, a vector, an immune effector cell or a pharmaceutical composition disclosed herein.

In another aspect, there is provided a method of increasing cytotoxicity to cancer cells in a subject having cancer, said method comprising administering to the subject an effective amount of a CAR or a claudin-3 binding protein, a polypeptide, a polynucleotide, a vector, an immune effector cell or a pharmaceutical composition disclosed herein.

In another aspect, there is provided a method of decreasing the number of cancer cells in a subject having cancer, said method comprising administering to the subject an effective amount of a CAR or a claudin-3 binding protein, a polypeptide, a polynucleotide, a vector, an immune effector cell or a pharmaceutical composition disclosed herein.

In another aspect, there is provided use of a CAR or a claudin-3 binding protein, a polypeptide, a polynucleotide, a vector, an immune effector cell or a pharmaceutical composition disclosed herein in the manufacture of a medicament for treatment of cancer.

In another aspect, there is provided a CAR or a claudin-3 binding protein, a polypeptide, a polynucleotide, a vector, an immune effector cell or a pharmaceutical composition disclosed herein for use in therapy.

### DESCRIPTION OF THE DRAWINGS/FIGURES

FIG. 1: Schematic diagram showing the accessibility of claudin-3 for binding in cancer cells vs. healthy non-cancerous cells (“normal cells”). CLDN3 belongs to a large family of integral membrane proteins crucial for the formation of tight junctions (TJs) between epithelial cells. Disruption of the normal tissue architecture is a hallmark of cancer, and CLDN3 altered expression has been linked to the development of various epithelial cancers including those with high unmet need such as colorectal, breast, pancreatic and ovarian carcinomas (Singh, Sharma, and Dhawan 2010). As shown, CLDN3 is mis-localized outside of TJs in tumours but not in healthy tissues, a mechanism that turns CLDN3 into a CAR-T cell target for selective killing of tumour cells while sparing the normal cells where it is hidden in the tight junctions.

FIGS. 2A-2B: FIG. 2A: Enrichment of LNGFR+ CAR-T Batches. EasySep LNGFR positive selection was performed on CAR-T batches to produce 100% CAR-T cell populations for use in functional assays. FIG. 2B: Normalisation of CAR-T Batches to required frequency of LNGFR+ cells. CAR-T batches were normalised by the addition of untransduced T cells to achieve a transduction efficiency of 30% across all constructs.

FIGS. 3A-3H: FIG. 3A: Basal IFNγ cytokine secretion profile for untransduced and transduced CAR-T cells. Average and standard deviation of the concentration of IFNγ secretion in culture supernatants using CAR-T cells obtained from 6 different healthy donors (****=p<0.0001 calculated by Bonferroni ONEWAY ANOVA; UT=untransduced). FIG. 3B-3G: Basal exhaustion/activation phenotype (CD69⁺, TIM3⁺ and PD-1⁺) for untransduced and transduced CAR-T cells. Mean and standard deviation are shown (p values range from <0.02 to <0.0001 calculated by Bonferroni ONEWAY ANOVA; UT=untransduced). FIG. 3H: CAR specific pCD3ζ levels in transduced CAR-T cells (as assessed by PEGGY-SUE). Normalised pCD3ζ staining compared to negative control (anti-CD19 CAR).

FIGS. 4A-4F: FIG. 4A: Secretion of IFNγ, IL-2 and TNFa in response to Claudin proteins. Quantification of cytokine release from anti-claudin-3 or anti-CD19 CAR-T cells co-cultured with RKO KO cell lines overexpressing hCLDN3, hCLDN4, hCLDN5, hCLDN6, hCLDN8, hCLDN9, hCLDN17 or mCLDN3 (mean of n=3+95% Confidence Limit). FIGS. 4B and 4C: IFNγ secretion in response to Claudin proteins. Cytokine release from anti-claudin-3 CAR, anti-CD19 CAR (control) or untransduced T cells co-cultured with RKO KO overexpressing hCLDN3, hCLDN4, hCLDN6, hCLDN9 or mCLDN3. FIG. 4B: Concentration of IFNγ presented as the mean of 6 donors+95% Confidence Limit. FIG. 4C: The fold change of IFNγ secretion from anti-claudin-3 CAR-T cells compared to anti-CD19 control or untransduced T cells when cultured with cell lines expressing Claudin proteins. FIG. 4D-4F: Level of cytokines secreted in response to Claudin protein. The concentration of the cytokines (pg/mL) is overlaid on a heat map representing the fold change of each condition compared to RKO KO cultured with control T cells. The fold change is calculated within each experiment and donor and log transformed. This data is presented for three cytokines IFNγ (top panel), IL-2 (middle panel) and TNFα (bottom panel) with the specific donor indicated in the left column.

FIGS. 5A-5B: FIG. 5A: Images of anti-claudin-3 CAR-T cells co-cultured with RKO KO cells expressing Claudin proteins. These images were taken at day 4 for each cell line expressing: hCLDN3, hCLDN4, hCLDN6, hCLDN9 or mCLDN3. Left panels: The image is overlaid with the mask used to calculate the % confluency shown by yellow lines outlining the cell areas. Right panels: The image is overlaid with red fluorescence showing the intensity and localisation of the CYTOTOX red dye. FIG. 5B: Images were taken every 2 hours for 4 days and the % confluency of each well was calculated with the INCUCYTE software at each time point. For this experiment 6 donors were used in triplicate. The mean of the triplicate wells was calculated and each point in the graph above represents the mean of 6 donors±SEM.

FIG. 6: The cytotoxic response of CAR-T cells to Claudin family proteins. Anti-claudin-3 CAR (“906-009”), anti-CD19 CAR (“CD19”; control) and untransduced T cells (“UT”) were co-cultured with RKO KO cells expressing hCLDN3, hCLDN4, hCLDN6, hCLDN9 or mCLDN3 for 4 days. The absorbance of CYTOTOX red and consequent red fluorescence was analysed with a mask and the data was used to calculate the % Live Cells. The data from 1 representative donor is presented as the mean of 3 triplicate wells±SEM.

FIGS. 7A-7B: Surface molecule quantification using quantification beads. FIG. 7A: Average number of LNGFR molecules per T cell. T cells and Quantum Simply Cellular quantification beads were stained with anti-LNGFR PE and their mean fluorescence intensities (MFIs) were measured. The bead MFIs were used to create a standard curve that was used to interpolate LNGFR numbers per cell. n=3 for donor 12031 and 92024 and n=5 for donor D5 and the error bars determine the standard deviation. FIG. 7B: Quantification of hCLDN3 expression on RKO human colon cancer cells. RKO cells with endogenous hCLDN3 knocked out and then engineered to express hCLDN3 were sorted for low or high hCLDN3 expression. RKO cells and Quantum Simply Cellular quantification beads were stained with anti-hCLDN3-PE and their MFIs were measured. The bead MFIs were used to create a standard curve that was used to interpolate hCLDN3 numbers per cell. 4 replicates were measured and the error bars determine the standard deviation.

FIGS. 8A-8D: Example INCUCYTE and XCELLIGENCE killing assays. INCUCYTE raw data of LNGFR enriched anti-CD19 CAR-T cells (FIG. 8A) or anti-claudin-3 CAR-T cells (FIG. 8B) incubated with RKO-KO CLDN3 L14 for 90 hours is shown. An example killing curve is based on the raw data in FIGS. 8A-8B is shown in FIG. 8C. 3 replicates were measured and the error bars determine the standard deviation. FIG. 8D: XCELLIGENCE killing assay example. Normalised data is shown for unsorted anti-claudin-3 CAR-T cells or untransduced T cells co-cultured with the HT-29-LUC target cell line at a 1:1 ratio. The data is represented as the mean of n=3±standard deviation.

FIG. 9: INCUCYTE killing assay with varying numbers of RKO-KO hCLDN3-expressing target cells. RKO-KO and RKO-KO hCLDN3 polyclonal cells were mixed at varying ratios and cocultured with anti-CD19 control CAR-T cells or anti-claudin-3 CAR-T cells. The % of live cells over time was measured. 3 replicates were measured and the error bars represent the standard deviation.

FIGS. 10A-10D: A gradient of hCLDN3 expression showed a T cell dose response. RKO-KO cells were electroporated with a gradient of hCLDN3 mRNA and cocultured with CLDN3 and anti-CD19 control CAR-T cells produced from 3 donors. FIG. 10A: The expression of hCLDN3 assessed by flow cytometry related to the mass of hCLDN3 mRNA nucleofected. This data is presented as the Mean Fluorescence Intensity or the % of the target positive population. Pearson's R² was calculated for the correlation between mRNA mass and mean fluorescence intensity. FIG. 10B: After co-culture the anti-claudin-3 CAR-T cells were stained to identify CD69 expression. The % CD69 expression is shown as the mean of 3 donors+95% CI. FIGS. 10C-10D: The co-culture supernatant was used to quantify the concentration of cytokines secreted by T cells. FIG. 10C: IFNγ pg/ml presented as the mean of 3 donors+95% confidence interval. Ratio of IFNγ secretion from anti-claudin-3 vs anti-CD19 control CAR-T cells. FIG. 10D: Granzyme B pg/mL presented as the mean of 3 donors+95% confidence interval. Ratio of Granzyme B secretion from anti-claudin-3 vs anti-CD19 control CAR-T cells.

FIGS. 11A-11C: CLDN3 expression by flow cytometry and RT-qPCR and IFNγ secretion in response to various cell lines from different indications: colorectal (FIG. 11A), pancreatic cancer (FIG. 11B) and breast (FIG. 11C) cancer. hCLDN3 expression was measured at the protein level by flow cytometry (left) and at the mRNA level by RT-qPCR (middle). HT-29-LUC and RKO-KO cell lines were included in every experiment as a positive and negative control, respectively. IFNγ secretion in response to various cell lines from different indications was also assessed. T cells were incubated with target cells lines for 24 hours at a 1:1 ratio and IFNγ secretion was measured by MSD (right).

FIG. 12: Example killing images and raw data of selected cell lines. Shown are example images (top) and raw data (bottom) of two cell lines that showed complete killing (HT-29-LUC and MDA MB468), three cell lines that showed partial killing (HCC1954, HPAC and BxPC3) and one cell line that did not show any killing (COLO-320DM). Raw data is shown as the total of Cytotox Red per well. The raw data was used because data cannot be normalised between different cell lines making comparisons only possible with raw data.

FIG. 13: CAR expression determined by Protein L-Biotin (1^{st}) and anti-Biotin-PE (2^{nd}) staining. Transduction efficiencies were determined by LNGFR-PE staining. Here the CAR and LNGFR frequencies of the exemplary donor D5 are shown for day 7 after transduction with the named CAR variants.

FIG. 14: Results of a killing assay with CAR T cells (donor D5) are shown. Frequency of lysed target cells was calculated after measuring the luciferase activity in the co-culture of CAR-T cells with luciferase transduced T-47D cells (each data point represents the mean of technical replicates, n=2).

FIGS. 15A-15C: The concentration of secreted cytokines in response to anti-claudin-3 CAR-T cells having different scFv constructs was determined using the MACSPlex Cytokine 12 Kit (human). Supernatants of co-cultures with T cells and T-47D cells or of T cells alone were collected and analysed (undiluted). Concentrations of secreted IFNγ (FIG. 15A), IL-2 (FIG. 15B), and TNF-α (FIG. 15C) of T cell donor D5 are shown.

FIGS. 16A-16F: Results of a long term co-culture, of RKO-KO CLDN3 H1 (labelled as RKO-hCLD3) cells with CAR-T cells (donors G5 and H5) are shown with rounds 1-3 for Donor G5 shown in FIGS. 16A-16C and rounds 1-3 for Donor H5 shown in FIGS. 16C-16F. CAR-T cells were transferred onto of fresh RKO-KO CLDN3 H1 cells for a total of three rounds. The growth of the RKO-KO CLDN3 H1, expressing GFP was monitored with the INCUCYTE via green object confluence in percent and normalized to the starting value (hour 4). Conditions without replicates are marked with a star (*).

FIGS. 17A-17B: Exhaustion marker expression was determined by staining using anti-LAG3 (CD223)-VioBlue, anti-PD-1 (CD279)-PE-Vio770, and anti-TIM3 (CD366)-APC. LNGFR-positive T cells were evaluated for the expression of double (TIM3, PD-1; represented by filled circles) and triple (TIM3, PD-1, LAG3; represented by filled squares) positive exhaustion marker expression. Frequencies of double and triple positive CAR T cells for Donor H and Donor P are shown. Day 0 displays the frequencies before addition of target cells and day 1 after the first addition of target cells. On day 1, 2 and 3 fresh RKO-KO CLDN3 H1 cells were added. Results for each of Donor H and Donor P on Day 0 (FIG. 17A-17B), Day 1 (FIG. 17C-17D), Day 2 (FIG. 17E-17F), Day 3 (FIG. 17G-17H), and Day 6 (FIG. 17I-17J) are shown.

FIG. 18: Anti-claudin-3 CAR-T cells (906-009) exhibit enhanced Claudin-3-specific proliferative response compared with anti-claudin-3 CAR-T cells having other spacer or orientation variants (906-002, 906-004 and 906-007) following stimulation with Claudin-3 positive target cells.

FIGS. 19A-19B: Growth kinetics in NSG mice inoculated with HT-29 human colon adenocarcinoma cell line. Seven (7) days after inoculation, tumours were palpable and animals were dosed with PBS (no T cells), anti-CD19 (control CAR) or anti-claudin-3 CAR-T cells at a dose of 1×10⁷ cells. Day of Dosing is referred to as D0. FIG. 19A: Tumour volume results are presented as marginal means with 95% confidence intervals for each group at each measured timepoint. FIG. 19B: The difference between mean tumour volume in each group is shown with reference to the negative control anti-CD19 group. Larger negative values indicate that the anti-CD19 group has larger tumours than the comparator group. Stars are overlaid to indicate statistical significance: *p<0.05, **p<0.01, ***p<0.001.

FIG. 20: Percentage (%) of LNGFR-positive (+) CAR-T cells gated on the human CD3 cell population (CD45⁺, CD3⁺ LNGFR⁺) in the peripheral blood of CAR-T dosed NSG mice at Day 28 post dosing analysed via flow cytometry. Note that 6 mice of the PBS group (no T cells), 3 mice of the anti-CD19 CAR group and 8 mice of the anti-claudin-3 CAR-T cell group were still on study at day 28.

FIGS. 21A-21B: IFNγ release measured in blood serum of HT-29-tumour bearing NSG mice prior and Day 7 post dosing with PBS (no T cells), anti-CD19 (control CAR) or anti-claudin-3 CAR-T cells. N=6-8 mice per group, one data point represents the single measurement or the mean of technical control duplicates or triplicates for each mouse depending on blood volumes that could be collected. FIG. 21A: IFNγ release in pg/mL per group pre and post treatment. HD stands for highest density. FIG. 21B: Comparison of the IFNγ release between pre- and post-treatment between anti-claudin-3 CAR and anti-CD19 CAR control. Anti-claudin-3 CAR shows a 15-fold increase in IFNγ release when compared to anti-CD19 CAR group. The Bayesian posterior probability that this change is greater than a 1× change (i.e., the probability that IFNγ increase at all with treatment) is 98.2%. This indicates a strong probability of an increase in IFNγ following treatment with anti-claudin-3 CAR-T cells.

FIG. 22: Characterisation of colorectal patient derived xenograft (PDX) models by flow cytometry. The percentage (%) of EpCAM-CLDN3-double positive (+) cell populations detected after thawing in two separate experiments, one with models CR5052, CR5080, CR89 (panel A, left side) and another experiment with models CR5030, CR5087 (panel B, right side) is summarized. In both experiments CLDN3 positive (HT-29 Luc, referred to as HT-29) and negative control (RKO-KO) cell lines were included. Percentage of EpCAM-positive tumour cell population was ranging from 41 to 65% in the CR models. In the first experiment (panel A, left side) a range of CLDN3-expressing tumour cells from 34.6-55.4% was observed. In the second experiment (panel B, right side) percentage of CLDN3-expressing tumour cells was 26 to 38%. Furthermore, high CLDN3 expression was detected in the positive control (HT-29) and no CLDN3 was detected in the RKO-KO cells (negative control) as expected. The OV PDX model is not depicted as no population with EpCAM-CLDN3-double positive cells was present in the sample. Each experiment refers to one tumour sample per model. These experiments served to set up characterisation of the PDX sample with focus on target expression for future PDX in vitro assays with multiple biological replicates per model.

FIG. 23A-23F: Cytokine release of two co-culture experiments, one with models CR5030, CR5087, OV5287 (panel A, left side; FIGS. 23A, 23C, 23E) and another experiment with models CR5052, CR5080, CR89 (panel B, right side; FIGS. 23B, 23D, 23F). Both experiments were run with control cell lines HT-29 Luc (referred to as HT-29, positive control) and RKO-KO (negative control) at 50,000 cells per well for Panel A and 25,000 cells per well for Panel B and T cells alone at 50,000 cells per cell. CAR-T cells (effector) were added to the PDX cells (target) at a 1:1 target to effector ratio. Cytokines were elevated in all co-cultures with anti-claudin-3 CAR-T cells including the model OV5287 with low CLDN3 expression, but were not elevated in the CLDN3 negative control RKO-KO and T cells alone. Each experiment used one biological replicate (tumour) per model with one T cell donor in technical duplicates or triplicates depending on cell numbers available. No statistics were performed on these pilot experiment data.

FIG. 24: Available CD20 binding sites of anti-claudin-3 CAR-T cells (CD20_906_009) compared to B cells. Median fluorescence intensity of CD20⁺ anti-claudin-3 CAR-T Cells (CD20_906_009) and B cells are used to calculate the number of CD20 binding sites for each condition.

FIG. 25: Diagram outlining the experimental conditions of complement dependent cytotoxicity (CDC).

FIG. 26: Comparison of the proportion of cells deleted to CD20 expression across different CDC conditions. A mixed model was fixed to binomial proportions for proportion of CTV cells alive after 4 hours. Fixed effects of all combinations of Complement and Antibody and their interaction with CD20 expression. Random effects are then fit under a split-plot design, with Random intercepts for donor within random intercepts.

FIG. 27A-27E: CAR-T deletion by ADCC using CD20⁺ anti-claudin-3 CAR-T cells (CD20_906_009) and anti-claudin-3 CAR-T cells (906_009) with and without splice site optimisation (SO). Ratio of ‘proportion CTV’, between RTX:HI and RTX:RAB for different ADCC conditions. CAR T cells enriched on CAR expression by F(Ab)2.

FIG. 28: CD20⁺ anti-claudin-3 CAR-T cells (CD20_906_009) and control anti-claudin-3 CAR-T cells (906_009) alive at 20 hours of XCELLIGENCE cytotoxicity assay. A linear mixed effects model is fit to this data. % Alive is modelled as a response, and CAR is modelled as a fixed effect. As this is a split plot design, random effects are included for individual assay and Donor nested within assay. Linear contrasts are used to determine the difference in expected % alive between pairs of CARs, these are reported alongside p-values and 95% confidence intervals.

FIG. 29: XCELLIGENCE KT50 value of anti-claudin-3 CAR-T cells (906_009) and CD20⁺ anti-claudin-3 CAR-T cells (CD20_906_009). Fit linear model to KT50 with fixed effect of CAR (vector) and nested random effects of individual assay and donor. Use log 10 transform for KT50.

FIG. 30: Effect of splice site optimisation on the cells alive at 20 hours of XCELLIGENCE cytotoxicity assay. A linear mixed effects model is fit to this data. % Alive is modelled as a response, and CAR is modelled as a fixed effect. As this is a split plot design, random effects are included for individual assay and Donor nested within assay. Linear contrasts are used to determine the difference in expected % alive between pairs of CARs, these are reported alongside p-values and 95% confidence intervals.

FIG. 31: The effect of splice site optimisation on the XCELLIGENCE KT50 value. Fit linear model to KT50 with fixed effect of CAR (vector) and nested random effects of individual assay and donor. KT50 log 10 transformed.

FIG. 32A-32B: Effect of CD20 on Calcium flux in CAR-T cells. Calcium Flux in Untransduced, anti-claudin-3 CAR-T cells (906_009) and CD20⁺ anti-claudin-3 CAR-T cells (CD20_906_009) pre-treated with Thapsigargin (FIG. 32A) or DMSO (FIG. 32B) and subsequently stimulated with Ionomycin.

FIGS. 33A-33D: Plasma membrane protein array: Pre-screen study using untransduced cells, BCMA-CAR T cells and anti-claudin-3 CAR-T cells (906-009) from donor 90928. ZsGreen key spotting pattern for protein expression on HEK293 cells (FIG. 33A), untransduced T cells (FIG. 33B), BCMA CAR-T cells (FIG. 33C) and anti-claudin-3 CAR-T cells (906-009; FIG. 33D).

FIGS. 34A-34D: Plasma membrane protein array: confirmatory screen. Key to spotting pattern (FIG. 34A) in untransduced cells (FIG. 34B), BCMA CAR-T cells (FIG. 34C) and anti-claudin-3 CAR-T cells (906-009; FIG. 34D).

FIGS. 35A-35F: Secretion of IFNγ, IL-2 and TNF-α over time in NSG tumour-bearing mice after dosing with 902_007-LNGFR, SO-CD20-906_009 or CD20-CD19: Secreted levels of (FIGS. 35A-35B) IFNγ, (FIGS. 35C-35D) IL-2 and (FIGS. 35E-35F) TNF-α measured in blood serum of HT-29Luc tumour-bearing NSG mice prior to T cell dosing (baseline) or 3, 4, 5, 7 and 14 days post-T cell dosing with 902_007-LNGFR, SO-CD20-906_009 or CD20-CD19. Secreted levels are shown in pg/ml (y-axis). Each dot shows cytokine concentration at given time-point for a given mouse. Graphs (FIGS. 35A, 35C, 35E) show data as means and 95% confidence intervals for all timepoints. Graphs (FIGS. 35B, 35D, 35F) show marginal means and 95% confidence intervals from a linear mixed model are overlaid on the raw data for all timepoints except baseline.

FIG. 36: Secretion change over time for IFNγ, IL-10, IL-12p70, IL-13, IL-1β, IL-2, IL-4, IL-6, IL-8 and TNF-α in NSG tumour-bearing mice after dosing with 902_007-LNGFR, SO-CD20-906_009 or CD20-CD19: Heatmaps showing secretion change for: IFNγ, IL-10, IL-12p70, IL-13, IL-1β, IL-2, IL-4, IL-6, IL-8, TNF-α comparing each timepoint post-T cell dosing (3, 4, 5, 7 and 14 days) to the baseline (prior to T cell dosing) in HT-29Luc tumour-bearing NSG mice dosed with 902_007-LNGFR, SO-CD20-906_009 or CD20-CD19. Linear contrasts are used to calculate the secretion change at different time points post T cell dosing versus the Baseline level and presented here as fold changes.

FIG. 37: Tumour growth kinetics in NSG tumour-bearing mice dosed with 902_007-LNGFR, SO-CD20-906_009 or CD20-CD19 from the ‘day 14’ endpoint. Tumour growth kinetics in NSG tumour-bearing mice dosed with 902_007-LNGFR, SO-CD20-906_009 or CD20-CD19 from ‘day 14’ endpoint. Mice were inoculated with HT-29Luc cells on SD0 and were dosed with CAR T cells on SD23, when tumours reached ˜320 mm³. Mice from ‘day 14’ endpoint were culled on SD37; 14 days post-T cell dosing. Y-axis shows tumour volume (mm³) and x-axis shows study days for all calliper measurements. Two-way ANOVA followed by Bonferroni multiple comparisons was performed to compare all CAR T groups at all timepoints. Error bars indicate standard error of the mean. ns>0.05, **p<0.01, ****p<0.0001.

FIGS. 38A-38B: Tumour growth in NSG tumour-bearing mice dosed with 902_007-LNGFR, SO-CD20-906_009 or CD20-CD19 from the ‘day 4’ and ‘day 7’ endpoints. Tumour growth in NSG tumour-bearing mice dosed with 902_007-LNGFR, SO-CD20-906_009 or CD20-CD19 from (FIG. 38A) ‘day 4’ and (FIG. 38B) ‘day 7’ endpoints. Mice were inoculated with HT-29Luc cells on SD0 and were dosed with CAR T cells on SD23, when tumours reached ˜320 mm³. Mice from ‘day 4’ endpoint were culled on SD27; 4 days post-T cell dosing. Mice from ‘day 7’ endpoint were culled on SD30; 7 days post-T cell dosing. Y-axis shows tumour volume (mm³) and x-axis shows CAR T groups. One-way ANOVA followed by Tukey's multiple comparison test was performed to compare CAR T groups at the indicated endpoints. Error bars indicate standard error of the mean. ns>0.05. All comparisons were non-significant.

FIG. 39: Study Design. Schematic illustrates the study design. Briefly, female NSG mice were inoculated with HT-29Luc on study day (SD) 0. On SD23, mice were dosed with CAR T cells (when tumours reached ˜320 mm³). Blood samples were collected on SD5, SD26, SD27, SD28, SD30 and SD37. Tissues and tumours were collected on SD26, SD27, SD30 and SD37.

FIG. 40: Mouse model. NSG-SGM3 mice possess mouse macrophages and human cytokines are transgenically expressed. Human PBMCs and the human target cells (SO-CD20-906_009 T cells) are co-injected. These cells are generated from the same healthy donor. The Anti-CD20 mAb rituximab is injected to induce killing of SO-CD20-906_009 T cells in the peripheral blood and tissues. Control mice receive isotype or vehicle in presence of SO-CD20-906_009 T cells or rituximab in absence of SO-CD20-906_009 T cells.

FIG. 41: Study timeline. On day −1 (D-1) the cells were inoculated (1×10\hat{ }7 hPBMC, 1×10\hat{ }7 T cells with a SO-CD20-906_009 transduction efficiency of 38%). On Day 0 (D0), blood was collected from each mouse (pre-RTX blood as baseline) followed by RTX, Isotype or vehicle injection via the i.p. route. 24 and 72 hours post-mAb dosing, blood was collected again from each mouse. Day 7 and 8 post-mAb dosing, mice were humanely sacrificed and terminal blood and tissues were collected in a staggered approach to ensure high sample quality and feasibility.

FIG. 42A-42C: Characterisation of inoculates on day of injection. (A) Gating strategy for identifying SO-CD20-906_009 and PBMC sub-populations (B) PBMC composition in inoculates (C) Characterisation of CAR and CD20 expression on T cells in inoculates. Graph shows the average (n=3 replicates) percentage of CAR expressing T cells and CAR and CD20 co-expressing T cells. Error bars indicate standard deviation. F(ab′)2+ CD20+=SO-CD20-906_009. F(ab′)2=CAR.

FIG. 43A-43D: Characterisation and counting of SO-CD20-906_009 and hPBMCs in mouse terminal blood (Flow Cytometry). (A) Comparison of CD3+ (T cell) counts vs. f(ab′)2 counts in terminal blood 7 vs. 8 days post mAb/isotype treatment. (B) Total f(ab′)2 counts in mouse terminal blood (C) total CD3+ counts in mouse terminal blood. (D) proportion SO-CD20-906_009 of CD3+ in mouse terminal blood. (B,C,D) each dot represents a single mouse with marginal means and 95% confidence intervals. F(ab′)2=CAR, CD3+=T cells, mAb=monoclonal antibody (Rituximab), ISO=anti-RSV antibody. *=p.value≤0.05, **=p.value≤0.01, ***=p.value≤0.001.

FIG. 44: Comparison of CAR and CD20 expression on SO-CD20-906_009 pre and post-inoculation. Histograms showing CD20 and CAR (F(ab′)2) co-expression on SO-CD20-906_009 T cells in inoculates on day of injection (Day of inoculation) and from blood of mice on day of culling (day 7 or 8 post-mAb). F(ab′)2=CAR expression.

FIG. 45A-45B: SO-CD20-906_009 in blood is reduced in mAb-treated mice by 24 hrs post-mAb administration. Blood samples were collected from mice pre-mAb and at 24 hrs, 72 hrs and 7/8 days post mAb treatment (Terminal). HIV DNA copies were measured using ddPCR as a marker of the presence of SO-CD20-906_009 in mouse blood. (A) Pre-mAb, mice treated with SO-CD20-906_009 and no mAb ctrl, SO-CD20-906_009 and Isotype mAb ctrl and SO-CD20-906_009 and mAb had comparable expression of SO-CD20-906_009 in blood. 24 hrs post mAb administration, the mAb treated group had significantly reduced SO-CD20-906_009 compared to SO-CD20-906_009 and Isotype mAb group, which was sustained until the study terminal timepoint. Graph shows mean percentage change in HIV copies and 95% confidence intervals. ♦ shows mice treated with SO-CD20-906_009 and no mAb ctrl, ▪ shows mice treated with SO-CD20-906_009 and Isotype mAb ctrl and □ shows mice treated with SO-CD20-906_009 and mAb. Graphs show geometric means for each mouse and 95% confidence intervals. (B) Percentage change in HIV copies between SO-CD20-906_009 and mAb and SO-CD20-906_009 and Isotype mAb was calculated, which shows an 85.11% decrease in HIV copies after 24 hrs in the mAb treated group compared with the Isotype mAb treated group. At 72 hrs post mAb treatment and at the study terminal timepoint, there was a 70.44% and 61.56% decrease in HIV copies in SO-CD20-906_009 and mAb compared to SO-CD20-906_009 and Isotype mAb ctrl. Graph shows mean percentage change in HIV copies and 95% confidence intervals.

FIG. 46A-46B: SO-CD20-906_009 is reduced in the bone marrow, liver, lung and spleens of mAb-treated mice. At the terminal timepoint of the study (day 7/8 post mAb), bone marrow, liver, lung and spleen were collected and HIV DNA copies were measured as a marker of the presence of SO-CD20-906_009 in mouse tissues. (A) In bone marrow, Liver, Lung and Spleen, there was a significant decrease in HIV copies in the SO-CD20-906_009 and mAb group compared to the SO-CD20-906_009 and Isotype mAb group (p<0.0001 for all tissues). There was also a significantly lower number of HIV copies in the SO-CD20-906_009 and no mAb ctrl group compared to the SO-CD20-906_009 and Isotype mAb group. Graphs show geometric means for each mouse and 95% confidence intervals. ♦ shows mice treated with SO-CD20-906_009 and no mAb ctrl, ▪ shows mice treated with SO-CD20-906_009 and Isotype mAb ctrl and □ shows mice treated with SO-CD20-906_009 and mAb. (B) Percentage change in HIV copies between SO-CD20-906_009 and mAb and SO-CD20-906_009 and Isotype mAb ctrl was calculated. There were decreases in HIV copies of 95.75% in bone marrow, 88.05% in liver, 95.75% in lung and 98.66% in spleen. Graph shows mean percentage change in HIV copies and 95% confidence intervals.

FIG. 47A-47B: Expression of CLDN3 by a panel of NSCLC cell lines. (A) CLDN3 expression was measured by PCR and presented at 2^{-ΔCT}. (B) CLDN3 expression was measure by flow cytometry and the % CLDN3 positive population is presented. HT-29 and RKO KO were used as a positive and negative control, respectively. The cells were cultured over 6 weeks and three distinct experiments were performed. This data is presented as mean+standard error. Cell lines in orange were used for functional studies.

FIG. 48A-48B: Expression of CLDN3 by a panel of NSCLC and CRC cell lines for use in functional assays. (A) Relative CLDN3 expression was measured by qPCR and presented at 2^{-ΔCT}. This data is the mean+/−standard error (n=2 technical replicates). (B) hCLDN3 expression was measure by flow cytometry and presented as MFI of hCLDN3 normalised to the isotype control. The experiment was performed on the day that cells were plated for functional experiments. Data for both graphs is organised by low to high relative CLDN3.

FIG. 49A-49B: Quantification of activation factors from co-cultures of NSCLC cell lines with 906-009_LNGFR, CD19-LNGFR and UT. Five CRC cell lines of varying CLDN3 expression levels were used as controls. This represents activation factor levels 24 hours after the point of co-culture at a 1:1 CAR:Target ratio. Data is organised by relative CLDN3 (2^{-ΔCT}) expression on the day of target cell seeding and presented at mean+/−standard error mean. (n=3 donors) (A) IFNγ pg/mL (B) Granzyme B pg/mL.

FIG. 50A-50B: Modelling the relationship between T cell activation and relative CLDN3 mRNA expression (quantified by dCT aka 2^{-ΔCT}). (A) IFNγ (B) Granzyme B. The points on the graph represent activation factor secretion in co-cultures of cell lines (varying CLDN3 expression) with 906-009_LNGFR (three donors). 906-009_LNGFR (black), CD19_LNGFR (medium grey), UT (light grey).

FIG. 51: Images of target cell death in colon cancer cell lines. Images of cocultures of 906-009_LNGFR or CD19_LNGFR CAR-T cells with colon cancer cell lines. Images are shown for three donors. Images show Annexin V staining in blue, and the purple outline indicates the mask. Images are shown from the assay endpoint, to demonstrate target cell death in HT-29 and DLD1 cell lines in 906-009_LNGFR cocultures. RKO-KO did not show target cell death with 906-009_LNGFR.

FIG. 52: Images of target cell killing in NSCLC cell lines. Images of co-cultures of 906-009_LNGFR CAR-T cells or CD19_LNGFR CAR-T cells with a range of NSCLC cell lines. Images are shown for three donors. Images are shown from the assay endpoint, to demonstrate target cell killing.

FIG. 53A-53B: Images of co-cultures of 906-009_LNGFR or CD19_LNGFR CAR-T cells with CLDN3 low expression. Images are shown for three donors. Images are shown from the assay endpoint, and demonstrate partial cytotoxicity at this time point. Colo320DM showed partial target cell killing in donor PR19K133900 with 906-009_LNGFR only, which was not observed in donors PR19C133904 and PR19W133916. NCI-H1650 showed partial killing by donors PR19K133900 and PR19C133904 906-009_LNGFR co-cultures.

FIG. 54A-54B: Cell lines are ordered by expression of CLDN3 mRNA: RKO KO (CLDN3 protein KO), NCI-H1650, NCI-H2023, NCI-H1651 and DLD1. (A, C, E, G, I) Example plots of isotype stained cell lines. (B, D, F, H, J) Example plots of CLDN3 antibody stained plots. Gates were set based on the isotype stained controls.

FIG. 55A-55D: Effect of CLDN3 mutations on 906-mAb binding. (A) Gating strategy used to determine GFP-FITC and 906-mAb-PE positive populations. (B) Representative histogram overlay of GFP expression, in RKO KO target cells. (C) Graph showing fold-change in 906-mAb binding to mutant cell lines compared with WT, represented by median fluorescence intensity (MFI). (D) Graph showing change in % population of 906-mAb positive cells. (C and D n=2, 95% confidence intervals shown).

FIG. 56: Effect of CLDN3 mutations on activation of 906-009_LNGFR after co-culture with RKO KO target cells. IFNγ release following 24 h co-culture of 906-009_LNGFR with RKO KO target cells at a 1:1 target: transduced CAR T ratio. Data is expressed as % change IFNγ compared with WT, normalised to untransduced T cells (n=3). Data is mean of 3 T cell donors (n=3). 95% confidence intervals shown.

### DETAILED DESCRIPTION OF THE INVENTION

As used herein and in the claims, the singular forms “a”, “and” and “the” include plural reference unless the context clearly dictates otherwise. Thus, for example, reference to “a peptide chain” is a reference to one or more peptide chains and includes equivalents thereof known to those skilled in the art.

As used herein and in the claims, the term “comprising” encompasses “including” or “consisting” e.g., a composition “comprising” X may consist exclusively of X or may include additional elements, e.g., X+Y.

The term “consisting essentially of” limits the scope of the feature to the specified materials or steps and those that do not materially affect the basic characteristic(s) of the claimed feature. The term “consisting of” excludes the presence of any additional component(s).

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods disclosed herein.

Concentrations described herein are determined at ambient temperature and pressure. This may be, for example, the temperature and pressure at room temperature or in within a particular portion of a process stream. Preferably, concentrations are determined at a standard state of 25° C. and 1 bar of pressure.

The term “about” means a value within two standard deviations of the mean for any particular measured value.

### Overview

The recognition of neoepitopes or neoantigens—cancer-specific mutations or proteins expressed exclusively by cancer cells—has been important to the development of anti-cancer therapies. Such neoepitopes allow cancer cells to be distinguished from healthy, non-cancerous cells and allow anti-cancer agents and the patient's own immune system to be uniquely targeted while healthy, non-cancerous cells remain unaffected. A similar but less specific approach is to target tumour-associated self-antigens—proteins or other cellular components which are upregulated, or overexpressed, in cancer cells compared to in healthy, non-cancerous cells. However, the disadvantages with these approaches are that truly cancer-specific neoepitopes are rare and cannot be easily predicted, while targeting tumour-associated self-antigens can lead to off target effects due to their expression in healthy, non-cancerous cells.

In order to address these disadvantages, provided herein are chimeric antigen receptors (CARs) which bind at least one epitope of a cell junction protein, wherein said cell junction protein is located within a cell-cell junction and wherein said at least one epitope of the cell junction protein is only accessible for binding by said CAR extracellular domain in cancer cells. Such one or more epitopes are present or expressed on both cancer cells and healthy, non-cancerous cells and cells within organized tissues, while off target effects are reduced due to their inaccessibility and/or unavailability for binding in cells within organized tissue.

### Chimeric Antigen Receptors (CARs)

In various embodiments, genetically engineered receptors that redirect immune effector cells toward cancer cells expressing an epitope as described herein are provided. These genetically engineered receptors, referred to herein as chimeric antigen receptors (CARs), are molecules that combine antibody-based specificity for a desired antigen/epitope with a T cell receptor-activating intracellular domain to generate a chimeric protein that exhibits a specific cellular immune activity. The term “chimeric” describes being composed of parts of different proteins or DNAs from different origins.

In particular embodiments, there is provided a chimeric antigen receptor (CAR) comprising:
a) an extracellular domain which comprises a claudin-3 binding domain comprising a heavy chain variable region (VH) comprising a heavy chain complementarity determining region 1 (CDRH1) sequence of SEQ ID NO: 1; a heavy chain complementarity determining region 2 (CDRH2) sequence of SEQ ID NO: 2; a heavy chain complementarity determining region 3 (CDRH3) sequence of SEQ ID NO: 3;
b) a transmembrane domain; and
c) one or more intracellular signalling domains.

Engagement of the antigen binding domain of the CAR with the target antigen on the surface of a target cell results in clustering of the CAR and delivers an activation stimulus to the CAR-containing cell. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

### Extracellular Domain

In various embodiments, a CAR comprises an extracellular binding domain that comprises an antigen binding domain (e.g., a claudin-3 specific binding domain); a transmembrane domain; one or more co-stimulatory signalling domains; and one or more intracellular signalling domains.

The term “chimeric antigen receptor” (“CAR”) as used herein, refers to an engineered receptor comprising an extracellular antigen binding domain (usually derived from a monoclonal antibody, or fragment thereof, e.g., a VH domain in the form of a single-domain antibody (sdAb) or a VH domain and a VL domain in the form of a scFv), and optionally a spacer region, a transmembrane region, and one or more intracellular effector domains. In particular embodiments, the CAR further comprises a hinge region between the antigen binding domain and the intracellular signalling domain. The CAR may also comprise hinge domains or spacer domains between any of the extracellular binding domain, the transmembrane domain, the co-stimulatory domains and/or the intracellular signalling domains. CARS have also been referred to as chimeric T cell receptors or chimeric immunoreceptors (CIRs). CARs are genetically introduced into hematopoietic cells, such as T cells, to redirect T cell specificity for a desired cell-surface antigen, resulting in a CAR-T therapeutic. The term “spacer domain” as used herein, refers to an oligo- or polypeptide that functions to link the transmembrane domain to the extracellular antigen/target binding domain. This region may also be referred to as a “hinge domain” or “stalk domain”. The size of the spacer can be varied depending on the position of the target epitope in order to have optimal function upon CAR:target/antigen binding. In some instances, without wishing to be bound by any theories, optimal function may be achieved by maintaining a set distance (e.g., 14 nm) upon CAR:target/antigen binding.

In particular embodiments, CARs comprise an extracellular binding domain that comprises an antigen binding protein that specifically binds to an epitope which is present on multiple cells but only accessible and/or available for binding on a target cell, e.g., a cancer cell. As used herein, the terms “binding domain”, “antigen binding domain”, “extracellular domain”, “extracellular binding domain”, “antigen-specific binding domain” and “extracellular antigen specific binding domain” are used interchangeably and provide a CAR with the ability to specifically bind to the target antigen/epitope of interest. The binding domain may be derived from a natural, synthetic, semi-synthetic or recombinant source.

The term “antigen binding protein” as used herein refers to proteins, antibodies, antibody fragments (e.g., Fabs, scFv, etc.) and other antibody derived protein constructs, such as those comprising domain antibodies (dAbs) and sdAbs, which are capable of binding a target antigen.

The terms “antigen binding protein” and “epitope binding protein” are used interchangeably herein. This does not include the natural cognate ligand or receptor. In some embodiments, an antigen binding protein is capable of binding claudin-3 (also known as RVP1, HRVP1, C7orf1, CPE-R2, CPETR2), which can be referred to as a “claudin-3 binding protein” or “claudin-3 specific binding protein.” A “claudin-3 binding protein” refers to proteins, antibodies, antibody fragments (e.g., Fabs, scFv, etc.) and other antibody derived protein constructs, such as those comprising domains (e.g., dAbs, sdAbs, etc.) which are capable of binding claudin-3, preferably human claudin-3.

The term “antigen” as used herein refers to a structure of a macromolecule which is selectively recognized by an antigen binding protein. Antigens include but are not limited to protein (with or without polysaccharides) or protein composition comprising one or more T cell epitopes.

The term “epitope” as used herein refers to that portion of the antigen that makes contact with a particular binding domain of the antigen binding protein, also known as the paratope. An epitope may be linear or conformational/discontinuous. A conformational or discontinuous epitope comprises amino acid residues that are separated by other sequences, i.e., not in a continuous sequence in the antigen's primary sequence, and may be assembled by tertiary folding of the polypeptide chain. Although the residues may be from different regions of the polypeptide chain, they are in close proximity in the three-dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. Epitope mapping can be carried out using various techniques known to persons skilled in the art, including but are not limited to those described in publications such as Methods in Molecular Biology ‘Epitope Mapping Protocols’, Mike Schutkowski and Ulrich Reineke (volume 524, 2009) and Johan Rockberg and Johan Nilvebrant (volume 1785, 2018). Non-limiting exemplary methods include peptide-based approaches such as pepscan whereby a series of overlapping peptides are screened for binding using techniques such as ELISA or by in vitro display of large libraries of peptides or protein mutants, e.g., on phage. Detailed epitope information can be determined by structural techniques including, but is not limited to, X-ray crystallography, solution nuclear magnetic resonance (NMR) spectroscopy and cryogenic-electron microscopy (cryo-EM). Mutagenesis, such as alanine scanning, is another effective approach whereby loss of binding analysis is used for epitope mapping. Another method is hydrogen/deuterium exchange (HDX) combined with proteolysis and liquid-chromatography mass spectrometry (LC-MS) analysis to characterize discontinuous or conformational epitopes.

In particular embodiments, the extracellular binding domain of a CAR comprises an antibody or antigen binding domain thereof.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; dAb, sdAb, antigen binding antibody fragments, Fab, F(ab′)₂, Fv, disulphide linked Fv, scFv, disulphide-linked scFv, diabodies, TANDABS, etc. and modified versions of any of the foregoing (for a summary of alternative “antibody” formats see Holliger and Hudson 2005 Nature Biotechnology 23(9):1126-1136).

The terms, full, whole or intact antibody, used interchangeably herein, refer to a heterotetrameric glycoprotein with an approximate molecular weight of 150,000 daltons. An intact antibody is composed of two identical heavy chains (HCs) and two identical light chains (LCs) linked by covalent disulphide bonds. This H₂L₂ structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a κ or λ light chain. The majority of antibodies in the serum belong to the IgG class, and there are four isotypes of human IgG (IgG1, IgG2, IgG3 and IgG4), the sequences of which differ mainly in their hinge region.

Fully human antibodies can be obtained using a variety of methods, for example using yeast-based libraries or transgenic animals (e.g., mice) that are capable of producing repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B cell sorting techniques. Human antibodies produced using these techniques can then be characterised for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see e.g., U.S. Patent Application Publication Nos. 2005/0053973, 2005/0089932 and 2005/0164301) or an EGF domain.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2” and “CDRH3” used in the Examples follow the Kabat numbering convention. For further information, see Kabat et al., Sequences of Proteins of Immunological Interest, 4^{th} Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., 1989 Nature 342(6252):877-83. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 below to number the variable domain amino acid sequence. It should be noted that some of the CDR definitions may vary depending on the individual publication used.

TABLE 1

| | Kabat | Chothia | AbM | Contact | Minimum |
|---|---|---|---|---|---|
| | CDR | CDR | CDR | CDR | Binding Unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Exemplary claudin-3 binding proteins comprise any one or a combination of the following CDRs:
the CDRH1 of SEQ ID NO: 1;
the CDRH2 of SEQ ID NO: 2;
the CDRH3 of SEQ ID NO: 3;
the CDRL1 of SEQ ID NO: 4;
the CDRL2 of SEQ ID NO: 5; and/or
the CDRL3 of SEQ ID NO: 6,
   or
the CDRH1, CDRH2, CDRH3 from SEQ ID NO: 7; and/or
the CDRL1, CDRL2, CDRL3 from SEQ ID NO: 8,
   or
the CDRH1, CDRH2, CDRH3 from SEQ ID NO: 7; and
the CDRL1, CDRL2, CDRL3 from SEQ ID NO: 8.

CDRs may be modified by at least one amino acid substitution, deletion or addition, wherein the variant antigen binding protein substantially retains the biological characteristics of the unmodified protein.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 2 below.

TABLE 2

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the flanking residues that comprise the CDR as part of alternative definition(s) e.g. Kabat or Chothia, may be substituted with a conservative amino acid residue.

Such antigen binding proteins comprising variant CDRs as described above may be referred to herein as “functional CDR variants”.

In one embodiment, the claudin-3 binding protein comprises a heavy chain variable region (VH) comprising a heavy chain complementarity determining region 1 (CDRH1) sequence of SEQ ID NO: 1; a heavy chain complementarity determining region 2 (CDRH2) sequence of SEQ ID NO: 2; a heavy chain complementarity determining region 3 (CDRH3) sequence of SEQ ID NO: 3. In one embodiment, the claudin-3 binding protein further comprises a light chain variable region (VL) comprising a light chain complementarity determining region 1 (CDRL1) sequence of SEQ ID NO: 4; a light chain complementarity determining region 2 (CDRL2) sequence of SEQ ID NO: 5; a light chain complementarity determining region 3 (CDRL3) sequence of SEQ ID NO: 6. In particular embodiments, the extracellular domain of a CAR provided herein comprises the claudin-3 binding protein disclosed herein.

In one embodiment, claudin-3 binding proteins of the present disclosure show cross-reactivity between human claudin-3 and claudin-3 from another species, such as mouse claudin-3 and/or cynomolgus monkey claudin-3. In one embodiment, the claudin-3 binding proteins described herein specifically bind human, cynomolgus monkey, and murine claudin-3. This is particularly useful, since drug development typically requires testing of lead drug candidates in mouse systems before the drug is tested in humans. The provision of a drug that can bind human and mouse species allows one to test results in these systems and make side-by-side comparisons of data using the same drug. This avoids the complication of needing to find a drug that works against a mouse claudin-3 and a separate drug that works against human claudin-3, and also avoids the need to compare results in humans and mice using non-identical drugs. Cross reactivity between other species used in disease models such as dog or monkey, such as cynomolgus monkey, is also envisaged.

“Antigen binding domain” refers to a domain on an antigen binding protein that is capable of specifically binding to an antigen, this may be a single variable domain, or it may be paired VH/VL domains as can be found on a standard antibody. sdAbs or scFv domains can also provide antigen-binding sites. In one embodiment, the antigen binding protein is a claudin-3 binding protein. In one embodiment, the claudin-3 binding protein is an sdAb and comprises a heavy chain variable region (VH). In one embodiment, the claudin-3 binding protein is an scFv and comprises a heavy chain variable region (VH) and a light chain variable region (VL). In some embodiments, the VL is located at the N-terminus of the VH, or the VH is located at the N-terminus of the VL. In some embodiments, the VL and the VH are directly fused to each other via a peptide bond or linked to each other via a peptide linker

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” is a type of engineered Ab which contains a naturally occurring variable region (light and heavy chains) derived from a donor Ab in association with light and heavy chain constant regions derived from an acceptor Ab. Thus, in certain embodiments, a CAR contemplated herein comprises an antigen binding domain that is a chimeric antibody or antigen binding domain thereof.

In some embodiments, an antibody is a human antibody (such as a human monoclonal antibody) or fragment thereof that specifically binds to a human claudin-3 protein.

In one embodiment, a CAR comprises a “humanized” antibody or antibody binding fragment. A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see e.g., Queen, et al., Proc. Natl Acad Sci USA. 1989; 86(24): 10029-10032 and Hodgson, et al., Biotechnology, 1991; 9(5): 421-5). A suitable human acceptor antibody may be one selected from a conventional database, e.g., the KABAT database, Los Alamos database and Swiss Protein database, by homology to the nucleotide and amino acid sequences of the donor antibody. A human antibody characterised by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. Nonlimiting examples of ways to produce such humanized antibodies are detailed in EP-A-0239400 and EP-A-054951.

“Percent identity” between a query nucleic acid sequence and a subject nucleic acid sequence is the “Identities” value, expressed as a percentage, that is calculated using a suitable algorithm or software, such as BLASTN, FASTA, DNASTAR Lasergene, GeneDoc, Bioedit, EMBOSS needle or EMBOSS infoalign, over the entire length of the query sequence after a pair-wise global sequence alignment has been performed using a suitable algorithm or software, such as BLASTN, FASTA, ClustalW, MUSCLE, MAFFT, EMBOSS Needle, T-Coffee, and DNASTAR Lasergene. Importantly, a query nucleic acid sequence may be described by a nucleic acid sequence identified in one or more claims herein.

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50%, 60%, 70%, 75%, 80%, 85%, 90%, 95%, 96%, 97%, 98% or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The percent (%) identity may be determined across the entire length of the query sequence, including the CDRs. Alternatively, the percent identity may exclude one or more or all of the CDRs, for example all of the CDRs are 100% identical to the subject sequence and the percent identity variation is in the remaining portion of the query sequence, e.g., the framework sequence, so that the CDR sequences are fixed and intact.

It will be appreciated by a person skilled in the art that VH and/or VL domains disclosed herein may be incorporated, e.g., in the form of a sdAb or a scFv, into CAR-T therapeutics.

The disorganisation of tissue structure is a hallmark of cancer and the resulting disruption of cell-cell junctions can lead to epitopes being made accessible/available in cancerous tissue and cancer cells which would otherwise remain ‘hidden’ in organized or healthy tissue [Corsini et al. (2018) Oncotarget]. The alteration of cell-cell contacts also leads to the loss of cell polarity and to the exposure of a number of extracellular signals such as those from growth factors—in the absence of the apical-basal polarity, epithelial cells that receive growth signals not only in the apical domain tend to proliferate by an out-of-plane division promoted by the mis-orientation of the mitotic axis.

In some embodiments, a CAR comprises an extracellular domain comprising an antigen binding protein that binds at least one epitope of a cell junction protein (e.g., claudin-3) located within a cell-cell junction, wherein said at least one epitope of the cell junction protein (e.g., claudin-3) is only accessible for binding by said CAR extracellular domain when the cell junction protein is mislocalized outside of the cell-cell junction, and thereby exposed to the cell surface. A cell junction protein that is mislocalized outside of a cell-cell junction refers to aberrant localization of the cell junction protein such that the cell junction protein is not confined to the cell-cell junction, e.g., tight junction. As illustrative and non-limiting examples, a cell junction protein (e.g., claudin-3) is mislocalized outside of a cell-cell junction in cancer and exposed to the cell surface that could be recognized and bound by a CAR or an antibody disclosed herein. In contrast, a cell junction protein (e.g., claudin-3) located within tight junctions are properly confined to the tight junctions in healthy or noncancerous tissue, thereby prohibiting the access by a CAR or an antibody targeting the cell junction protein.

Thus, epitopes targeted by CARs described herein are found on cell junction proteins expressed on both healthy (non-cancerous) cells and on cancer cells. However, the epitope(s) of cell junction proteins bound by the antigen binding protein of the CAR extracellular domain is available and/or accessible for binding when said epitope(s) are mislocalized or presented outside of cell-cell junctions exposing cell junction proteins to the cell surface (e.g., cancer cells or cells in disorganized tissue). Alternatively, the epitope(s) bound by the antigen binding protein of the CAR extracellular domain is available and/or accessible for binding in cancer cells when the cell-cell junction is compromised (e.g., leaky) or disrupted. For example, in healthy, non-cancerous cells or cells within organized tissues, the one or more epitopes may be hidden as they are located within cell-cell junctions such that a CAR extracellular domain, antibody or antigen binding fragment is blocked from binding said epitope. Thus, in healthy, non-cancerous cells or in cell-cell junctions between healthy, non-cancerous cells or in cell-cell junctions located between cells within organized tissue the one or more epitopes is inaccessible/unavailable for binding by the CAR extracellular domain.

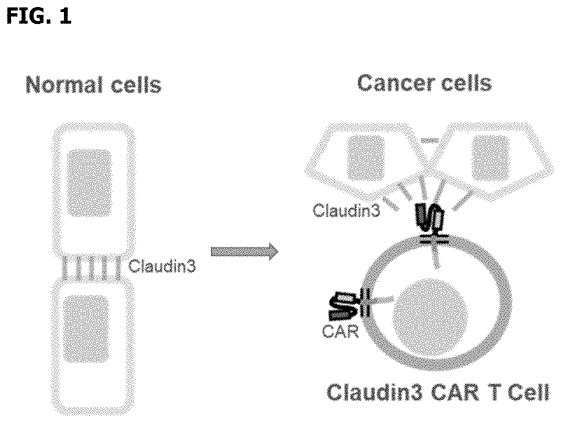

Therefore, without wishing to be bound by any particular theory, it is hypothesised that a CAR extracellular domain described herein binds one or more epitopes present in both healthy, non-cancerous cells and cancer cells but said epitope is only accessible and/or available for said binding in cancer cells or between cells in disorganized tissues (see FIG. 1). Such access and availability for binding by the CAR extracellular binding domain may be due to a conformational change in a cell junction protein resulting in the formation or exposure of the one or more epitopes to which the CAR extracellular domain binds, for example by unfolding a buried loop in the cell junction protein or by bringing together in a cancer cell amino acids which are not found in close proximity in healthy, non-cancerous cells or organized tissue. Alternatively, availability and/or access may be due to the cell junction protein not being in a complex with or engaged with a binding partner in cancer cells compared to in healthy, non-cancerous cells or organized tissue. It will be appreciated that cell junction proteins located within cell-cell junctions may comprise particularly attractive epitopes to target with CARs in this regard, with intact or uncompromised (e.g., undisrupted) cell-cell junctions in organized tissue preventing access by the CARS and rendering said epitopes inaccessible and/or unavailable for binding.

Thus, in certain embodiments, the one or more epitopes is present in a healthy, non-cancerous cell and is inaccessible for binding by the CAR extracellular domain and/or the one or more epitopes is located in a cell-cell junction and is inaccessible for binding by the CAR extracellular domain when said cell-cell junction is between healthy, non-cancerous cells. In further embodiments, the CAR extracellular domain is sterically hindered from binding the one or more epitopes in healthy, non-cancerous cells and/or is sterically hindered from binding the one or more epitopes located in a cell-cell junction between healthy, non-cancerous cells.

Thus, in one embodiment, a cell-cell junction is disrupted, such as disrupted between cells in disorganized tissue or between cancer cells compared to the cell-cell junctions present between cells in organized tissue, e.g., between healthy, non-cancerous cells. In a further embodiment, a cell-cell junction is compromised, such as compromised when between cells within disorganized tissue, between cancer cells, or between a cancer cell and a healthy, non-cancerous cell. It will therefore be appreciated that the terms “compromised” and “disrupted” may be used interchangeably herein and include wherein the cell-cell junction is physically disrupted, such as its structure is altered, and/or wherein the cell-cell junction is functionally compromised, e.g., has increased ‘leakiness’. In a yet further embodiment, a cell-cell junction is compromised and/or disrupted when proteins comprised within said junctions are mislocalized. In another embodiment, proteins comprised within a cell-cell junction are mislocalized when said junctions are compromised and/or disrupted.

Without wishing to be bound by any particular theory, it is hypothesised that cell-cell junctions which are structurally disrupted may cause (e.g., expose) epitopes which are usually ‘hidden’ in non-disrupted cell-cell junctions to become accessible/available for binding (e.g., to become sterically accessible/available for binding) and/or cell-cell junctions which are functionally compromised (e.g., having increased ‘leakiness’) may allow increased invasion/passage of lymphocytes through said cell-cell junctions, thus leading to the accessibility/availability of certain epitopes for binding, e.g., by an antigen binding protein, including an antigen binding protein incorporated into a CAR.

In one embodiment, the one or more epitopes is inaccessible/unavailable for binding by the CAR extracellular domain when the cell-cell junction is not disrupted, such as when the cell-cell junction is between healthy, non-cancerous cells or between cells within organized tissue (see FIG. 1, left panel). In another embodiment, the one or more epitopes is accessible/available for binding by the CAR extracellular domain when the cell-cell junction is disrupted, such as when the cell-cell junction is between cancer or tumour cells or the cell-cell junction is between a healthy, non-cancerous cell and a cancer cell, or between cells within disorganized tissue (see FIG. 1, right panel). In a further embodiment, the one or more epitopes is accessible/available for binding by the CAR extracellular domain only when the cell-cell junction is disrupted, such as only when the cell-cell junction is between cancer or tumour cells or the cell-cell junction is between a healthy, non-cancerous cell and a cancer or tumour cell, or between cells within disorganized tissue.

In a further embodiment, the one or more epitopes is inaccessible/unavailable for binding by the CAR extracellular domain when the cell-cell junction is not compromised, such as when the cell-cell junction is between healthy, non-cancerous cells, or between cells within organized tissue. In another embodiment, the one or more epitopes is accessible/available for binding by the CAR extracellular domain when the cell-cell junction is compromised, such as when the cell-cell junction is between cancer or tumour cells, the cell-cell junction is between a healthy, non-cancerous cell and a cancer cell, or the cell-cell junction is between cells within disorganized tissue. In a further embodiment, the one or more epitopes is accessible/available for binding by the CAR extracellular domain only when the cell-cell junction is compromised, such as only when the cell-cell junction is between cancer or tumour cells, the cell-cell junction is between a healthy, non-cancerous cell and a cancer cell, or the cell-cell junction is between cells within disorganized tissue.

Further examples of epitopes which are only accessible and/or available for binding in the context of cancer include those present in cellular components (e.g., cell junction proteins) which are mislocalised in cancer cells compared to in healthy, non-cancerous cells. Such mislocalisation may be the result of overexpression or upregulation of the cellular component, mutations, changes to the post-translational modifications of a protein, changes to cellular polarisation and/or tissue disorganisation. Thus, in some embodiments, the one or more epitopes is inaccessible/unavailable for binding by the CAR extracellular domain when the cell-cell junction comprises a cell junction protein containing the target epitope which is not mislocalized, such as when the cell-cell junction is between healthy, non-cancerous cells or between cells within organized tissue. In further embodiments, the one or more epitopes is accessible/available for binding by the CAR extracellular domain when the cell-cell junction comprises a cell junction protein containing the target epitope which is mislocalised, such as when the cell-cell junction is between cancer or tumour cells, the cell-cell junction is between a healthy, non-cancerous cell and a cancer or tumour cell, or the cell-cell junction is between cells within disorganized tissue. In other embodiments, the one or more epitopes is accessible/available for binding by the CAR extracellular domain only when the cell-cell junction comprises a cell junction protein containing the target epitope which is mislocalized, such as only when the cell-cell junction is between cancer or tumour cells, the cell-cell junction is between a healthy, non-cancerous cell and a cancer cell, or the cell-cell junction is between cells within disorganized tissue. In a yet further embodiment, the one or more epitopes is accessible/available for binding by the CAR extracellular domain only when the cell-cell junction comprises a cell junction protein containing the target epitope which is mislocalized outside of the cell-cell junction, such as only when the cell-cell junction is between cancer cells, the cell-cell junction is between a healthy cell and a cancer cell, or when the cell-cell junction is otherwise compromised or disrupted, such as between cells within disorganized tissue.

Thus, as will be appreciated, the terms “accessible” and “available” are used interchangeably herein and may refer to the spatial and/or steric accessibility/availability of the epitope, or to the expression of the epitope, on cancer cells compared to healthy, non-cancerous cells. Similarly, the terms “inaccessible” and “unavailable” are also used interchangeably herein.

In a particular embodiment, the one or more epitopes is present in a cell junction protein located within a tight junction.

Tight junctions (also known as occluding junctions or zonulae occludentes) are multiprotein complexes between epithelial cells whose general function is to prevent the leakage of transported solutes and water across the epithelial barrier and to seal the paracellular pathway. They may also provide a leaky pathway by forming selective channels for small molecules such as cations, anions or water and whether an epithelial barrier is classified as ‘tight’ or ‘leaky’ depends on the ability of the tight junctions between the cells to prevent the movement of solutes and water. A non-limiting example of a ‘tight’ epithelial barrier is the blood-brain barrier and a non-limiting example of a ‘leaky’ epithelial barrier is in the kidney proximal tubule. Therefore, not only do tight junctions function to hold cells together in order to form an epithelial barrier, they also prevent/control the passage of molecules and ions through the space between the membranes of adjacent cells, as well as maintain the polarity of cells by preventing lateral diffusion of cell membrane components between their apical and lateral/basal surfaces.

Tight junctions are composed of a branching network of sealing strands with each strand acting independently from the others. Each strand is formed from a row of transmembrane proteins embedded in the plasma membranes of the epithelial cells, with extracellular domains joining one another directly. There are at least 40 different proteins found in tight junctions and they comprise both transmembrane and cytoplasmic proteins. The three major transmembrane proteins found in tight junctions are occludin, claudins, and junction adhesion molecule (JAM) proteins. These associate with different peripheral membrane proteins such as ZO-1 located on the intracellular side of plasma membrane which anchor the strands to the actin component of the cytoskeleton. In this way, tight junctions join together the cytoskeletons of adjacent cells.

Occludin is a NADH oxidase that influences certain aspects of cell metabolism such as glucose uptake, ATP production and gene expression. It is also important for the function of tight junctions in which it has been shown to interact with Tight junction protein 1, Tight junction protein 2 and YES1, and, although not required for the assembly of tight junctions, plays a role in the maintenance of barrier properties. The mutation or absence of occludin increases epithelial leakiness and loss of or abnormal expression of occludin has been shown to cause increased invasion, reduced adhesion and significantly reduced tight junction function in breast cancer tissues.

Claudins are small (20-27 kDa) transmembrane proteins which are found in many organisms. Claudins span the cellular membrane four times (i.e., have four transmembrane domains), with the N- and C-termini both located in the cytoplasm and two extracellular loops which show the highest degree of conservation. The first extracellular loop (ECL1) is approximately 53 amino acids in length and the second extracellular loop (ECL2) is approximately 24 amino acids in length. ECL1 controls paracellular ion selectivity and ECL2 controls homo- and heterodimerisation with adjacent claudin proteins within the tight junction. The N-terminal end is usually short (e.g., 4-10 amino acids), while the C-terminal end is longer and varies in length from, e.g., 21-63 amino acids and is necessary for the localisation of these proteins in tight junctions. It is suspected that cysteines of individual or separate claudins form disulphide bonds. All human claudins (with the exception of Claudin 12) have domains which allow them bind to PDZ domains of scaffold proteins.

Junction adhesion molecule (JAM) proteins are located in the tight junctions between high endothelial cells and are involved not only in the formation of these junctions, but also function as adhesive ligands for immune cells. They have also been implicated in endothelial cell polarity through their interactions with PAR-3 and JAM3.

In one embodiment, the cell junction protein is a member of the claudin family of proteins, e.g., is selected from any of: claudin-1, claudin-2, claudin-3, claudin-4, claudin-5, claudin-6, claudin-7, claudin-8, claudin-9, claudin-10a, claudin-10b, claudin-11, claudin-12, claudin-14, claudin-15, claudin-16, claudin-17, claudin-18, claudin-19, claudin-20, claudin-22, claudin-23 and claudin-25, or the related claudin domain containing 1, claudin domain containing 2, transmembrane protein 204 and peripheral myelin protein 22.

In yet further embodiments, the cell junction protein is claudin-3. In particular embodiments, the cell junction protein is human claudin-3 (e.g., human claudin-3 as shown in SEQ ID NO: 13).

Claudin-3 (also known as CLDN3) is encoded in humans by the CLDN3 gene and, as well as being an integral component of tight junctions, is also a low-affinity receptor for Clostridium perfringens enterotoxin. It has been shown to interact with CLDN1 and CLDNS and human claudin-3 has the following amino acid sequence:

(SEQ ID NO: 13)
MSMGLETTGTALAVLGWLGTIVCCALPMWRVSAFIGSNIITSQNIWEGLW
MNCVVQSTGQMQCKVYDSLLALPQDLQAARALIVVAILLAAFGLLVALVG
AQCTNCVQDDTAKAKITIVAGVLFLLAALLTLVPVSWSANTIIRDFYNPV
VPEAQKREMGAGLYVGWAAAALQLLGGALLCCSCPPREKKYTATKVVYSA
PRSTGPGASLGTGYDRKDYV.

The underlined residues in bold above indicate N38 and E153. In a further embodiment, the one or more epitopes is present in one or more extracellular loops of the cell junction protein. In some embodiments, said extracellular loops include extracellular loop 2 (ECL2) of human claudin-3 which comprises the following sequence:

(SEQ ID NO: 14)
WSANTIIRDFYNPVVPEAQKREM.

In some embodiments, said extracellular loops include extracellular loop 1 (ECL1) of human claudin-3 which comprises the following sequence:

(SEQ ID NO: 26)
RVSAFIGSNIITSQNIWEGLWMNCWQSTGQMQCKVYDSLLALPQDLQAA
R.

In a yet further embodiment, the one or more epitopes is present uniquely in claudin-3. The terms “unique” and “present uniquely” as used herein refer to wherein the recited feature is not found in other, closely related proteins within the same protein family. For example, wherein one or more epitopes is present uniquely in claudin-3, said one or more epitopes is not found in another claudin family protein, such as the closely related proteins claudin-4, claudin-6, claudin-5, claudin-9 or claudin-17, in particular said one or more epitopes is not found in either claudin-4, claudin-6, claudin-5 or claudin-9, such as claudin-4 which is the closest known homolog to claudin-3. Such epitopes may be small or may be a short length of amino acids, such as 4 to 10 amino acids, e.g., 4, 5, 6, 7, 8, 9 or 10 amino acids. Thus, in one embodiment, the one or more epitopes is 4 amino acids in length. An epitope present uniquely in claudin-3 can be a linear epitope or a conformational epitope. Linear epitopes are comprised of continuous residues within a protein primary sequence. For example, an epitope comprising the amino acid sequence PWP (SEQ ID NO: 15) is a linear epitope that is present in claudin-3, but which is not present in other closely related claudin family proteins, such as claudin-4 and thus may be considered an epitope present uniquely in claudin-3. Conformational epitopes are comprised of residues that are discontinuous in the primary protein sequence yet come within close proximity to form an antigenic surface on the three-dimensional structure of the protein/antigen. An epitope present uniquely in claudin-3 may also be a conformational epitope.

In some embodiments, the one or more epitopes is a conformational epitope comprised of residues present in the ECL1 and the ECL2 of claudin-3. In some embodiments, the one or more epitopes is a conformational epitope comprising at least one residue present in the ECL1 of claudin-3 and at least one residue present in the ECL2 of claudin-3. In some embodiments, the one or more epitopes is a conformational epitope comprising at least N38 present in the ECL1 of claudin-3 and at least E153 present in the ECL2 of claudin-3 when numbered according to SEQ ID NO:13. In some embodiments, the one or more epitopes comprises at least N38 and E153 of SEQ ID NO:13. In some embodiments, the one or more epitopes consists essentially of N38 and E153 of SEQ ID NO:13.

In one embodiment, there is provided an isolated claudin-3 binding protein that binds to a discontinuous epitope on human claudin-3 comprising at least N38 and E153 of SEQ ID NO:13. In one embodiment, there is provided an isolated claudin-3 binding protein that binds to a discontinuous epitope on human claudin-3 consisting essentially of N38 and E153 of SEQ ID NO:13. In one embodiment, wherein the claudin-3 binding protein is chimeric or humanized; and/or wherein the claudin-3 binding protein is selected from the group consisting of: a monoclonal antibody, a human IgG1 isotype, a Camel Ig, Ig NAR, Fab fragments, Fab′ fragments, F(ab)′2 fragments, F(ab)′3 fragments, Fv, scFv, bis-scFv, (scFv)2, minibody, diabody, triabody, tetrabody, disulfide stabilized Fv protein (dsFv), and sdAb. In yet another embodiment, there is provided a chimeric antigen receptor (CAR) comprising a polypeptide comprising: a) an extracellular domain which comprises the isolated claudin-3 binding protein that binds to a discontinuous epitope on human claudin-3 comprising at least N38 and E153 of SEQ ID NO:13; b) a transmembrane domain; and c) one or more intracellular signalling domains. In another embodiment, the extracellular domain comprises the isolated claudin-3 binding protein that binds to a discontinuous epitope on human claudin-3 consisting essentially of N38 and E153 of SEQ ID NO:13

The terms “specific binding affinity”, “specifically binds”, “specifically bound”, “specific binding” or “specifically targets” as used herein, describe binding of an antigen/epitope binding domain (or a CAR comprising the same) to an epitope which is only accessible and/or available for binding on cancer cells. In certain embodiments, a binding domain (or a CAR comprising a binding domain or a fusion protein containing a binding domain) binds to a target with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹ or 10¹² M⁻¹ “High affinity” binding domains (or single chain fusion proteins thereof) refers to those binding domains with a Ka of at least 10⁷ M⁻¹, at least 10⁸ M⁻¹, at least 10⁹ M⁻¹, at least 10¹⁰ M⁻¹, at least 10¹¹ M⁻¹ or at least 10¹² M⁻¹ or greater. A binding domain (or a CAR comprising a binding domain or a fusion protein containing a binding domain) may be considered to “specifically bind” to claudin-3 if it binds to or associates with claudin-3 with an affinity or Ka (i.e., an equilibrium association constant of a particular binding interaction with units of I/M) of, for example 10³ M⁻¹ to 10¹⁰ M⁻¹.

Alternatively, affinity may be defined as an equilibrium dissociation constant (Kd) of a particular binding interaction with units of M (e.g., 10⁻⁵ M to 10⁻¹³ M, or less). Affinities of binding domain polypeptides and CARs according to the present disclosure can be readily determined using conventional techniques, for example by competitive ELISA (enzyme linked immunosorbent assay), by binding association, displacement assays using labelled ligands, using a surface-plasmon resonance device such as the Biacore T 100 (which is available from Biacore, Inc., Piscataway, N.J.) or optical biosensor technology such as the EPIC system or EnSpire (available from Corning and Perkin Elmer respectively (see also, e.g., Scatchard, Ann NY Acad Sci. 1949; 51(4): 660 and U.S. Pat. No. 5,283, 173 or the equivalent).

However, it will be appreciated by the skilled person that a CAR comprising an extracellular domain that comprises an antigen/epitope binding protein as disclosed herein may display greater sensitivity and selectivity than an antibody comprising the same antigen/epitope binding domain. This means that while binding may not be detected directly, such as by visualisation of said binding using a labelled antibody or CAR, binding may be determined indirectly such as through cellular functional assays and measurements. For example, binding may be directly detected by visualisation of said binding using a labelled antibody or CAR, e.g., a fluorescently labelled soluble antibody, or may be indirectly detected through the function (such as activation) of cells expressing an antibody or CAR, such as through cytokine release assays (e.g., measuring IFNγ release), measuring cell killing, or by other functional measurements/techniques as described in detail in the Examples section below. Thus, in certain embodiments, binding is detected, e.g., by the activation of CAR-expressing cells, when the antibody or antigen/epitope binding fragment is comprised in a CAR and is thus expressed by a cell in a non-soluble, cellular format, but is not detected when the antibody or antigen/epitope binding fragment is comprised in a soluble, non-cellular format (e.g., a soluble antibody or antigen binding fragment thereof). It will further be appreciated by the skilled person that a CAR as described herein may have greater selectivity for a target antigen/epitope, such as claudin-3, as compared to other related proteins, such as other claudin family member proteins including claudin-4, claudin-5, claudin-6 and/or claudin-9.

In particular embodiments, the extracellular binding domain of a CAR comprises an antigen binding protein, such as an anti-claudin-3 binding protein, wherein the antigen binding protein is selected from an antibody or antigen binding fragment thereof.

In particular embodiments, the antigen binding protein, such as an anti-claudin-3 antibody or antigen binding fragment thereof, includes but is not limited to a Camel Ig (a camelid antibody (VHH)), Ig NAR, Fab fragments, Fab′ fragments, F(ab)′2 fragments, F(ab)′3 fragments, Fv, scFv, bis-scFv, (scFv)2, minibody, diabody, triabody, tetrabody, disulfide stabilized Fv protein (“dsFv”) and sdAb (also known as Nanobody).

In one embodiment, the antigen binding protein, such as an anti-claudin-3 antibody or antigen binding fragment thereof is a scFv.

In some embodiments, a CAR extracellular domain comprises a claudin-3 binding protein. An exemplary claudin-3 binding protein is an immunoglobulin variable region specific for claudin-3 that comprises at least one human framework region. A “human framework region” refers to a wild type (i.e., naturally occurring) framework region of a human immunoglobulin variable region, an altered framework region of a human immunoglobulin variable region with less than about 50% (e.g., preferably less than about 45%, 40%, 35%, 30%, 25%, 20%, 15%, 10%, 5% or 1%) of the amino acids in the region are deleted or substituted (e.g., with one or more amino acid residues of a non-human immunoglobulin framework region at corresponding positions) or an altered framework region of a non-human immunoglobulin variable region with less than about 50% (e.g., preferably less than about 45%, 40%, 35%, 30%, 25%, 20%, 15%, 10%, 5% or 1%) of the amino acids in the region deleted or substituted (e.g., at positions of exposed residues and/or with one or more amino acid residues of a human immunoglobulin framework region at corresponding positions) so that, in one embodiment, immunogenicity is reduced.

In certain embodiments, a human framework region is a wild type framework region of a human immunoglobulin variable region. In certain other embodiments, a human framework region is an altered framework region of a human immunoglobulin variable region with amino acid deletions or substitutions at one, two, three, four, five, six, seven, eight, nine, ten or more positions. In other embodiments, a human framework region is an altered framework region of a non-human immunoglobulin variable region with amino acid deletions or substitutions at one, two, three, four, five, six, seven, eight, nine, ten or more positions.

In particular embodiments, a claudin-3 binding protein comprises at least one, two, three, four, five, six, seven or eight human framework regions (FR) selected from human light chain FR1, human heavy chain FR1, human light chain FR2, human heavy chain FR2, human light chain FR3, human heavy chain FR3, human light chain FR4 and human heavy chain FR4.

Human FRs that may be present in a claudin-3-specific binding domain also include variants of the exemplary FRS provided herein in which one, two, three, four, five, six, seven, eight, nine, ten or more amino acids of the exemplary FRs have been substituted or deleted.

In certain embodiments, a claudin-3 binding protein comprises: (a) a humanized light chain variable region that comprises a human light chain FR1, a human light chain FR2, a human light chain FR3 and a human light chain FR4; and (b) a humanized heavy chain variable region that comprises a human heavy chain FR1, a human heavy chain FR2, a human heavy chain FR3 and a human heavy chain FR4.

Claudin-3 binding proteins provided herein can also comprise one, two, three, four, five, or six CDRs. Such CDRs may be non-human CDRs or altered non-human CDRs selected from CDRH1, CDRH2 and CDRH3 of a heavy chain variable region and CDRL1, CDRL2 and CDRL3 of a light chain variable region. In certain embodiments, a claudin-3 binding protein comprises a heavy chain variable region that comprises a heavy chain CDRH1, a heavy chain CDRH1 and a heavy chain CDRH3. In certain embodiments, a claudin-3 binding protein comprises a heavy chain variable region that comprises a light chain variable region that comprises a light chain CDRL1, a light chain CDRL2 and a light chain CDRL3. In certain embodiments, a claudin-3 binding protein comprises (a) a heavy chain variable region that comprises a heavy chain CDRH1, a heavy chain CDRH1 and a heavy chain CDRH3; and (b) a light chain variable region that comprises a light chain CDRL1, a light chain CDRL2 and a light chain CDRL3.

Thus, in one embodiment, a claudin-3 binding protein comprises any one or a combination of CDRs selected from CDRH1, CDRH2 and CDRH3 from SEQ ID NO: 7 and/or CDRL1, CDRL2 and CDRL3 from SEQ ID NO: 8. In a further embodiment, the claudin-3 binding protein comprises all six CDRs from SEQ ID NOs: 7 and 8.

In one embodiment, a claudin-3 binding protein comprises at least one heavy chain CDR sequence set forth in SEQ ID NOs: 1-3. In a particular embodiment, a claudin-3 binding protein comprises at least one heavy chain CDR sequence with at least 85%, 86%, 87%, 88%, 89%, 90%, 91%, 92%, 93%, 94%, 95%, 96%, 97%, 98% or 99% amino acid identity to the heavy chain CDR sequences set forth in SEQ ID NOs: 1-3. In a further embodiment, a claudin-3 binding protein comprises at least one heavy chain CDR sequence at least 90% identical to the heavy chain CDR sequences set forth in SEQ ID NOs: 1-3. In particular embodiments, the claudin-3 binding protein comprises a CDRH1 at least 90% identical to SEQ ID NO: 1, a CDRH2 at least 90% identical to SEQ ID NO: 2 and/or a CDRH3 at least 90% identical to SEQ ID NO: 3. In other embodiments, the claudin-3 binding protein comprises a CDRH1 of SEQ ID NO: 1, a CDRH2 of SEQ ID NO: 2 and/or a CDRH3 of SEQ ID NO: 3.

In one embodiment, a claudin-3 binding protein comprises at least one light chain CDR sequence set forth in SEQ ID NOs: 4-6. In a particular embodiment, a claudin-3 binding protein comprises at least one light chain CDR sequence with at least 85%, 86%, 87%, 88%, 89%, 90%, 91%, 92%, 93%, 94%, 95%, 96%, 97%, 98% or 99% amino acid identity to the light chain CDR sequences set forth in SEQ ID NOs: 4-6. In a further embodiment, a claudin-3 binding protein comprises at least one light chain CDR sequence at least 90% identical to the light chain CDR sequences set forth in SEQ ID NOs: 4-6. In particular embodiments, the claudin-3 binding protein comprises a CDRL1 at least 90% identical to SEQ ID NO: 4, a CDRL2 at least 90% identical to SEQ ID NO: 5 and/or a CDRL3 at least 90% identical to SEQ ID NO: 6. In other embodiments, the claudin-3 binding protein comprises a CDRL1 of SEQ ID NO: 4, a CDRL2 of SEQ ID NO: 5 and/or a CDRL3 of SEQ ID NO: 6.

In some embodiments, a claudin-3 binding protein comprises a CDRH1 that is at least 90% identical to SEQ ID NO: 1, a CDRH2 that is at least 90% identical to SEQ ID NO: 2, a CDRH3 that is at least 90% identical to SEQ ID NO: 3, a CDRL1 that is at least 90% identical to SEQ ID NO: 4, a CDRL2 that is at least 90% identical to SEQ ID NO: 5 and a CDRL3 that is at least 90% identical to SEQ ID NO: 6.

In a yet further embodiment, a claudin-3 binding protein comprises a CDRH1 of SEQ ID NO: 1, a CDRH2 of SEQ ID NO: 2, a CDRH3 of SEQ ID NO: 3, a CDRL1 of SEQ ID NO: 4, a CDRL2 of SEQ ID NO: 5 and a CDRL3 of SEQ ID NO: 6.

References to “VH” refer to the variable region of an immunoglobulin heavy chain, including that of an antibody, Fv, scFv, dsFv, Fab, sdAb, or other antibody fragment as disclosed herein. Illustrative examples of heavy chain variable regions that are suitable for constructing claudin-3 binding proteins contemplated herein include, but are not limited to the heavy chain variable region sequence set forth in SEQ ID NO: 7.

References to “VL” refer to the variable region of an immunoglobulin light chain, including that of an antibody, Fv, scFv, dsFv, Fab, or other antibody fragment as disclosed herein. Illustrative examples of light chain variable regions that are suitable for constructing claudin-3 binding proteins contemplated herein include, but are not limited to the light chain variable region sequence set forth in SEQ ID NO: 8.

In one embodiment, a claudin-3 binding protein comprises a VH sequence at least 75%, 85%, 86%, 87%, 88%, 89%, 90%, 91%, 92%, 93%, 94%, 95%, 96%, 97%, 98% or 99% identical to the sequence of SEQ ID NO: 7. In another embodiment, a claudin-3 binding protein comprises a VH sequence at least 90% identical to the sequence of SEQ ID NO: 7. In an alternative embodiment, a claudin-3 binding protein comprises a VH sequence of SEQ ID NO: 7.

In a further embodiment, a claudin-3 binding protein comprises a VL sequence at least 75%, 85%, 86%, 87%, 88%, 89%, 90%, 91%, 92%, 93%, 94%, 95%, 96%, 97%, 98% or 99% identical to the sequence of SEQ ID NO: 8. In another embodiment, a claudin-3 binding protein comprises a VL sequence at least 90% identical to a sequence of SEQ ID NO: 8. In an alternative embodiment, a claudin-3 binding protein comprises a VL sequence of SEQ ID NO: 8.

Thus, in one embodiment, a claudin-3 binding protein, comprises a VH sequence at least 90% identical to a sequence of SEQ ID NO: 7 and a VL sequence at least 90% identical to a sequence of SEQ ID NO: 8. In a further embodiment, a claudin-3 binding domain comprises a VH sequence of SEQ ID NO: 7 and a VL sequence of SEQ ID NO: 8.

In a particular embodiment, a claudin-3 binding protein is an sdAb comprising a VH sequence at least 90% identical to a sequence of SEQ ID NO: 7. In one embodiment, the claudin-3 binding protein is an sdAb comprising a VH sequence of SEQ ID NO: 7.

In a particular embodiment, a claudin-3 binding protein is an scFv comprising a VH sequence at least 90% identical to a sequence of SEQ ID NO: 7 and a VL sequence at least 90% identical to a sequence of SEQ ID NO: 8, and preferably comprises a VH sequence of SEQ ID NO: 7 and a VL sequence of SEQ ID NO: 8.

In some embodiments, a claudin-3 binding protein is an scFv comprising, from N-terminus to C-terminus, a VH sequence and a VL sequence, wherein the VH and VL sequences are optionally separated by a linker sequence. In a particular embodiment, a claudin-3 binding protein is an scFv comprising, from N-terminus to C-terminus, a VH sequence of SEQ ID NO: 7 and a VL sequence of SEQ ID NO: 8. In other embodiments, a claudin-3 binding protein is an scFv comprising, from N-terminus to C-terminus, a VL sequence and a VH sequence, wherein the VL and VH sequences are optionally separated by a linker sequence. In a particular embodiment, a claudin-3 binding protein is an scFv comprising, from N-terminus to C-terminus, a VL sequence of SEQ ID NO: 8 and a VH sequence of SEQ ID NO: 7.

In certain embodiments, a claudin-3 binding protein comprises a sequence that is least 85%, 86%, 87%, 88%, 89%, 90%, 91%, 92%, 93%, 94%, 95%, 96%, 97%, 98% or 99% identical to the sequence of SEQ ID NO: 11. In a further embodiment, a claudin-3 binding protein comprises a sequence at least 90% identical to SEQ ID NO: 11. In a yet further embodiment, a claudin-3 binding protein comprises the sequence of SEQ ID NO: 11.

In certain embodiments, a claudin-3 binding protein comprises a sequence that is least 85%, 86%, 87%, 88%, 89%, 90%, 91%, 92%, 93%, 94%, 95%, 96%, 97%, 98% or 99% identical to the sequence of SEQ ID NO: 18. In a further embodiment, a claudin-3 binding protein comprises a sequence at least 90% identical to SEQ ID NO: 18. In a yet further embodiment, a claudin-3 binding protein comprises the sequence of SEQ ID NO: 18.

### Linkers

In certain embodiments, the CARs comprise linker residues between the various domains, e.g., between VH and VL domains, added for appropriate spacing and conformation of the molecule. In particular embodiments the linker is a variable region linking sequence. A “variable region linking sequence” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. In particular embodiments, a linker separates one or more heavy or light chain variable domains, hinge domains, transmembrane domains, co-stimulatory domains and/or intracellular signalling domains. CARs can comprise one, two, three, four, five or more linkers. In particular embodiments, the length of a linker is about 1 to about 25 amino acids, about 5 to about 20 amino acids, about 10 to about 20 amino acids or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25 or more amino acids long.

Illustrative examples of linkers include glycine polymers (G)ₙ; glycine-serine polymers (G₁₋₅S₁₋₅)ₙ, where n is an integer of at least one, two, three, four or five (SEQ ID NO: 40); glycine-alanine polymers; alanine-serine polymers; and other flexible linkers known in the art. An exemplary linker is a glycine-serine polymer as shown in SEQ ID NO: 9.

### Spacer Domain

In particular embodiments, the extracellular domain, i.e., binding domain of the CAR is followed by one or more “spacer domains” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 domains of IgG1, IgG4 or IgD.

### Hinge Domain

The binding domain of a CAR is generally followed by one or more “hinge domains”, which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic or recombinant source. The hinge domain can include the amino acid sequence of a naturally occurring immunoglobulin hinge region or an altered immunoglobulin hinge region.

Illustrative hinge domains suitable for use in the CARs described herein include the hinge region derived from the extracellular regions of type I membrane proteins such as CD8α, and CD4, which may be wild-type hinge regions from these molecules or may be altered. In a particular embodiment, the hinge domain is derived from or comprises a CD8α hinge region.

### Transmembrane Domain

In particular embodiments, a CAR further comprises a transmembrane domain. The “transmembrane domain” (TM) is the portion of the CAR that fuses the extracellular binding portion and co-stimulatory domain/intracellular signalling domain and anchors the CAR to the plasma membrane of the immune effector cell, e.g., by traversing the cell membrane. The TM domain may be derived either from a natural, synthetic, semi-synthetic or recombinant source. The TM domain may be derived from (e.g., comprise) at least the transmembrane region(s) of alpha or beta chain of the T-cell receptor, CD3δ, CD3ε, CD3γ, CD3ζ, CD4, CDS, CD8α, CD9, CD16, CD22, CD27, CD28, CD33, CD37, CD45, CD64, CD80, CD86, CD134, CD137 (4-1BB), CD152, CD154, CD278 (ICOS) and PD1. In a particular embodiment, the TM domain is synthetic and predominantly comprises hydrophobic residues such as leucine and valine.

In one embodiment, the CARs comprise a TM domain derived from CD8α. In another embodiment, a CAR comprises a TM domain derived from CD8α and a short oligo- or polypeptide linker, preferably between 1, 2, 3, 4, 5, 6, 7, 8, 9 or 10 amino acids in length that links the TM domain and the co-stimulatory/intracellular signalling domain of the CAR. A glycine-serine based linker provides a particularly suitable linker. An exemplary TM domain derived from CD8α is shown in SEQ ID NO: 19.

### Intracellular Signalling Domains

In particular embodiments, a CAR further comprises an intracellular signalling domain. An “intracellular signalling domain” (also referred to as “intracellular effector domain” or “signalling domain”) refers to the part of a CAR that participates in transducing the message of effective binding of the extracellular domain (e.g., anti-claudin-3 CAR binding) to a target antigen (e.g., claudin-3 protein) into the interior of the immune effector cell to elicit effector cell function. The intracellular signalling domain is responsible for the activation of at least one of the normal effector functions of the immune cell in which the CAR is expressed, e.g., activation, cytokine production, proliferation and/or cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or helper activity including the secretion of a cytokine. Thus, the term “intracellular signalling domain” refers to the portion of a protein which transduces the effector function signal and that directs the cell to perform a specialized function. While usually the entire intracellular signalling domain can be employed, in many cases it is not necessary to use the entire domain. To the extent that a truncated portion of an intracellular signalling domain is used, such truncated portion may be used in place of the entire domain as long as it transduces the effector function signal.

The term intracellular signalling domain is meant to include any truncated portion of the intracellular signalling domain sufficient to transduce effector function signal.

It is known that signals generated through the T cell receptor (TCR) alone are insufficient for full activation of the T cell and that a secondary or co-stimulatory signal is also required. Thus, T cell activation can be said to be mediated by two distinct classes of signalling domains: intracellular signalling domains that initiate antigen-dependent primary activation through the TCR (e.g., a TCR/CD3 complex) and co-stimulatory signalling domains that act in an antigen-independent manner to provide a secondary or co-stimulatory signal. In some embodiments, a CAR comprises at least one “co-stimulatory signalling domain” and at least one “intracellular signalling domain.”

Intracellular signalling domains regulate primary activation of the TCR complex either in a stimulatory way or in an inhibitory way. Intracellular signalling domains that act in a stimulatory manner may contain signalling motifs which are known as immunoreceptor tyrosine-based activation motifs or ITAMs.

Illustrative examples of ITAM containing intracellular signalling domains that are suitable for use in particular embodiments of CARs described herein include those derived from FcRγ, FcRß, CD3γ, CD3ε, CD3δ, CD3ζ, CD22, CD66d, CD79a, and CD79b. In one embodiment, the one or more intracellular signalling domain is CD3ζ. An exemplary CD3ζ intracellular signalling domain is shown in SEQ ID NO: 21. In particular preferred embodiments, a CAR comprises a CD3ζ intracellular signalling domain and one or more co-stimulatory signalling domains. The intracellular signalling and co-stimulatory signalling domains may be linked in any order in tandem to the carboxyl terminus of the transmembrane domain.

### Co-Stimulatory Domains

In particular embodiments, a CAR further comprises one or more co-stimulatory signalling domains to enhance the efficacy and expansion of T cells expressing CARs. As used herein, the term “co-stimulatory signalling domain” or “co-stimulatory domain” refers to an intracellular signalling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TLR1, TLR2, TLR3, TLR4, TLR5, TLR6, TLR7, TLR8, TLR9, TLR10, TRIM and ZAP70.

In one embodiment, a CAR comprises one or more co-stimulatory signalling domains selected from the group consisting of CD28, CD134 (OX40) and CD137 (4-1BB). In a further embodiment, the one or more co-stimulatory domain is CD137 (4-1BB). An exemplary CD137 (4-1BB) co-stimulatory domain is shown in SEQ ID NO: 20.

In another embodiment, a CAR comprises a CD137 (4-1BB) co-stimulatory signalling domain and a CD3ζ intracellular signalling domain.

In certain embodiments, a CAR further comprises a leader sequence. In particular embodiments, the leader sequence is a CD8 leader sequence. An exemplary CD8 leader sequence is set forth in SEQ ID NO: 10.

Thus, in certain embodiments, the CAR contemplated herein comprises a sequence with at least 85%, 86%, 87%, 88%, 89%, 90%, 91%, 92%, 93%, 94%, 95%, 96%, 97%, 98% or 99% amino acid identity to the sequence of SEQ ID NOs: 12, 34, 35, 36, 37, 38, or 39. In a further embodiment, the CAR comprises a sequence at least 90% identical to SEQ ID NOs: 12, 34, 35, 36, 37, 38, or 39. In a yet further embodiment, the CAR comprises the sequence of SEQ ID NOs: 12, 34, 35, 36, 37, 38, or 39.

In certain embodiments, the CAR contemplated herein comprises a sequence with at least 85%, 86%, 87%, 88%, 89%, 90%, 91%, 92%, 93%, 94%, 95%, 96%, 97%, 98% or 99% amino acid identity to the sequence of SEQ ID NO: 25. In a further embodiment, the CAR comprises a sequence at least 90% identical to SEQ ID NO: 25. In a yet further embodiment, the CAR comprises the sequence of SEQ ID NO: 25.

In certain embodiments, the CAR contemplated herein comprises a sequence with at least 85%, 86%, 87%, 88%, 89%, 90%, 91%, 92%, 93%, 94%, 95%, 96%, 97%, 98% or 99% amino acid identity to the sequence of SEQ ID NO: 27. In a further embodiment, the CAR comprises a sequence at least 90% identical to SEQ ID NO: 27. In a yet further embodiment, the CAR comprises the sequence of SEQ ID NO: 27.

In certain embodiments, the CAR contemplated herein comprises a sequence with at least 85%, 86%, 87%, 88%, 89%, 90%, 91%, 92%, 93%, 94%, 95%, 96%, 97%, 98% or 99% amino acid identity to the sequence of SEQ ID NO: 28. In a further embodiment, the CAR comprises a sequence at least 90% identical to SEQ ID NO: 28. In a yet further embodiment, the CAR comprises the sequence of SEQ ID NO: 28.

In certain embodiments, the CAR contemplated herein comprises a sequence with at least 85%, 86%, 87%, 88%, 89%, 90%, 91%, 92%, 93%, 94%, 95%, 96%, 97%, 98% or 99% amino acid identity to the sequence of SEQ ID NO: 29. In a further embodiment, the CAR comprises a sequence at least 90% identical to SEQ ID NO: 29. In a yet further embodiment, the CAR comprises the sequence of SEQ ID NO: 29.

In certain embodiments, the CAR contemplated herein comprises a sequence with at least 85%, 86%, 87%, 88%, 89%, 90%, 91%, 92%, 93%, 94%, 95%, 96%, 97%, 98% or 99% amino acid identity to the sequence of SEQ ID NO: 30. In a further embodiment, the CAR comprises a sequence at least 90% identical to SEQ ID NO: 30. In a yet further embodiment, the CAR comprises the sequence of SEQ ID NO: 30.

In a particular embodiment, provided is a chimeric antigen receptor (CAR) comprising:
a) an extracellular domain which comprises a claudin-3 binding protein according to any one of the embodiments disclosed herein;
b) a transmembrane domain derived from CD8α; and
c) an intracellular signalling domain derived from CD3.

In another particular embodiment, provided is a chimeric antigen receptor (CAR) comprising:
a) an extracellular domain which comprises a claudin-3 binding protein comprising a CDRH1 sequence of SEQ ID NO: 1; a CDRH2 sequence of SEQ ID NO: 2; a CDRH3 sequence of SEQ ID NO: 3; a CDRL1 sequence of SEQ ID NO: 4; a CDRL2 sequence of SEQ ID NO: 5; and a CDRL3 sequence of SEQ ID NO: 6;
b) a transmembrane domain derived from CD8α;
c) an intracellular signalling domain derived from CD3ζ; and
d) a co-stimulatory domain derived from CD137 (4-1BB).

In some embodiments, the claudin-3 binding protein of the extracellular domain is an sdAb. In other embodiments, the claudin-3 binding protein of the extracellular domain is an sdAb comprising a CDRH1 sequence of SEQ ID NO: 1; a CDRH2 sequence of SEQ ID NO: 2; a CDRH3 sequence of SEQ ID NO: 3. In some embodiments, the claudin-3 binding protein of the extracellular domain is an scFv. In other embodiments, the claudin-3 binding protein of the extracellular domain is an scFv comprising a CDRH1 sequence of SEQ ID NO: 1; a CDRH2 sequence of SEQ ID NO: 2; a CDRH3 sequence of SEQ ID NO: 3; a CDRL1 sequence of SEQ ID NO: 4; a CDRL2 sequence of SEQ ID NO: 5; and a CDRL3 sequence of SEQ ID NO: 6. In yet other embodiments, the claudin-3 binding protein of the extracellular domain is an scFv comprising a VH sequence of SEQ ID NO: 7 and a VL sequence of SEQ ID NO: 8.

Also provided herein is a claudin-3 binding protein or a chimeric antigen receptor (CAR) that competes for binding with a CAR comprising an extracellular domain which comprises a claudin-3 binding protein comprising a CDRH1 sequence of SEQ ID NO: 1; a CDRH2 sequence of SEQ ID NO: 2; a CDRH3 sequence of SEQ ID NO: 3; a CDRL1 sequence of SEQ ID NO: 4; a CDRL2 sequence of SEQ ID NO: 5; and a CDRL3 sequence of SEQ ID NO: 6. In certain embodiments, the CAR contemplated herein competes for binding with a CAR comprising an extracellular domain comprising a claudin-3 binding protein comprising a VH sequence of SEQ ID NO: 7 and a VL sequence of SEQ ID NO: 8. Suitably, said CAR comprises a claudin-3 binding protein that is an scFv.

### Polypeptide

Various polypeptides are contemplated herein, including, but not limited to, CAR polypeptides and fragments thereof, cells and compositions comprising the same, antibodies and vectors that express polypeptides. In preferred embodiments, a polypeptide comprising one or more CARs is provided. In particular embodiments, the CAR is a claudin-3 binding CAR comprising an amino acid sequence at least 90% identical to SEQ ID NO: 11, preferably comprising a sequence at least 90% identical to SEQ ID NOs: 12, 34, 35, 36, 37, 38, or 39.

“Polypeptide”, “polypeptide fragment”, “peptide” and “protein” are used interchangeably, unless specified to the contrary, and according to conventional meaning, i.e., as a sequence of amino acids. Polypeptides may be synthesized or recombinantly produced. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides comprise a signal (or leader) sequence at the N-terminal end of the protein, which co-translationally or posttranslationally directs transfer of the protein. Illustrative examples of suitable signal sequences useful in CARs contemplated herein include, but are not limited to the IgG1 heavy chain signal polypeptide, a CD8α signal polypeptide, or a human GM-CSF receptor alpha signal polypeptide. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acids of a CAR as contemplated herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptides include “polypeptide variants”. Polypeptide variants may differ from a naturally occurring polypeptide in one or more substitutions, deletions, additions and/or insertions. Such variants may be naturally occurring or may be synthetically generated, for example, by modifying one or more of the above polypeptide sequences.

It will be appreciated that polypeptides comprising CARs, such as a CAR comprising the amino acid sequence of SEQ ID NO: 11 or SEQ ID NOs: 12, 34, 35, 36, 37, 38, or 39, as provided herein may comprise further and/or additional polypeptide sequences or elements. Such additional elements include, but are not limited to, ablation or control elements which may be used to either control expression of the polypeptide sequence in a cell or to target a polypeptide-containing cell. Elements or polypeptide sequences which control expression may comprise an internal ribosome entry site (IRES), translation start sequences and/or cleavage sites which allow for the separation of elements of the polypeptide sequence after translation.

Thus, in one embodiment, the polypeptide contemplated herein further comprises an ablation element. As used herein, an “ablation element” refers to a polypeptide sequence and/or protein expressed on the surface of a cell and which may be used to target or detect said cell (also known as “elimination markers”). For example, the ablation element may be a polypeptide sequence of a cell surface protein which comprises an extracellular epitope or binding region for an antibody or antigen binding fragment thereof. Thus, in one embodiment, the ablation element is a cell surface protein which is targeted for antibody-dependent cellular cytotoxicity (ADCC) and/or complement-dependent cytotoxicity (CDC) using an antibody or antigen binding fragment thereof specific for the cell surface protein. By utilising such mechanisms, it will therefore be appreciated that cells expressing the polypeptide contemplated herein may be specifically labelled/detected and may be specifically selected or isolated from, for example, a mixed population of transduced and untransduced cells. Furthermore, cells expressing a polypeptide comprising an ablation element may be specifically and selectively eliminated, such as eliminated/removed from the circulation of a treated subject.

Examples of suitable ablation elements include, but are not limited to, truncated human EGFR polypeptide (huEGFRt) and CD20, which may be recognised by cetuximab and rituximab, respectively (Wang et al., Blood, 2011; 118(5): 1255-1263, Paszkiewicz et al., J Clin Invest, 2016; 126(11):4262-4272, Vogler et al., Mol Ther J Am Soc Gene Ther, 2010; 18:1330-8, Griffioen et al., Haematologica, 2009; 94:1316-20 and Philip et al., Blood, 2014; 124:1277-87). Another example of a suitable ablation element is a short polypeptide epitope tag incorporated into the extracellular domain of the CAR (a so called “E-tag”) to which anti-epitope tag CARs may then be generated (Koristka et al., Cancer Immunol Immunother CII, 2019; 68:1401-15).

Thus, in one embodiment, the ablation element is selected from the group consisting of: truncated human EGFR polypeptide (huEGFRt) and CD20. In a particular embodiment, the ablation element is CD20.

In some embodiments, the ablation element is cleaved from the CAR polypeptide sequence. Thus, in one embodiment, the polypeptide contemplated herein comprises a cleavage site, such as a P2A cleavage site.

In certain embodiments, a polypeptide comprises the sequence set forth in SEQ ID NO: 24.

### Polynucleotide

In another aspect, a polynucleotide encoding one or more CARs as described herein is provided. As used herein, the terms “polynucleotide” or “nucleic acid” refer to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides.

In various illustrative embodiments, polynucleotides include expression vectors, viral vectors, and transfer plasmids, and compositions and cells comprising the same. In various illustrative embodiments, polynucleotides encode a CAR or polypeptide contemplated herein, including, but not limited to a CAR having the sequence of SEQ ID NOs: 12, 34, 35, 36, 37, 38, or 39 or a polynucleotide sequence encoding SEQ ID NOs: 12, 34, 35, 36, 37, 38, or 39 or a polynucleotide sequence set forth in SEQ ID NOs: 16 and 17.

Thus, also provided are polynucleotides encoding the antigen binding proteins disclosed herein, including polynucleotides comprising a sequence at least 75%, 85%, 86%, 87%, 88%, 89%, 90%, 91%, 92%, 93%, 94%, 95%, 96%, 97%, 98% or 99% identical to SEQ ID NOs: 16 and/or 17. In one embodiment, the polynucleotide comprises a sequence of SEQ ID NOs: 16 and/or 17.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

### Vectors

In another aspect, the present invention provides vectors which comprise a polynucleotide encoding one or more CARs and/or polypeptides as described herein.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes and viral vectors. Useful viral vectors include, e.g., replication defective retroviruses and lentiviruses.

In particular embodiments, the vectors are expression vectors. Expression vectors may be used to produce CARs and polypeptides contemplated herein. In addition, expression vectors may include additional components which allow for the production of viral vectors, which in turn comprise a polynucleotide contemplated herein. Viral vectors may be used for delivery of the polynucleotides contemplated herein to a subject or a subject's cells. Examples of expression vectors include, but are not limited to, plasmids, autonomously replicating sequences and transposable elements. Additional exemplary vectors include, without limitation, plasmids, phagemids, cosmids, transposons, artificial chromosomes such as yeast artificial chromosome (YAC), bacterial artificial chromosome (BAC), or PI-derived artificial chromosome (PAC), bacteriophages such as lambda phage or MI 3 phage, and animal viruses.

Additional examples of expression vectors are pClneo vectors (Promega) for expression in mammalian cells; pLenti4/V5-DESTTM pLenti6/V5-DESTTM and pLenti6.2/V5-GW/lacZ (Invitrogen)) for lentivirus-mediated gene transfer and expression in mammalian cells. In particular embodiments, the coding sequences of the CARs and polypeptides disclosed herein can be ligated into such expression vectors for the expression of the CARS and/or polypeptides in mammalian cells.

In particular embodiments, the expression vectors provided herein are BACs which comprise a polynucleotide as described herein. In particular embodiments, the BACs additionally comprise one or more polynucleotides encoding for proteins necessary to allow the production of a viral vector when expressed in a producer or packaging cell line. By way of example, PCT applications WO2017/089307 and WO2017/089308 describe expression vectors used to produce retroviral vectors, in particular lentiviral vectors. In a particular embodiment, the expression vectors described in WO2017/089307 and WO2017/089308, comprising a polynucleotide as described herein are provided.

The “control elements” or “regulatory sequences” present in an expression vector are those non-translated regions of the vector-origin of replication, selection cassettes, promoters, enhancers, translation initiation signals (Shine Dalgarno sequence or Kozak sequence), introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

### Vectors for Delivery

Also provided are vectors for delivery of the polynucleotides described herein to a subject and/or subject's cells. Examples of such vectors include, but are not limited to, plasmids, autonomously replicating sequences, transposable elements, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and viral vectors.

Examples of categories of animal viruses useful as viral vectors include, without limitation, retrovirus (including lentivirus), adenovirus, adeno-associated virus (AAV), herpesvirus (e.g., herpes simplex virus), poxvirus, baculovirus, papillomavirus, and papovavirus (e.g., SV40). These vectors are referred to herein as “viral vectors”.

As the skilled person will appreciate, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer.

Retroviruses are a common tool for gene delivery (Miller, 2000, Nature. 357: 455-460). In particular embodiments, a retrovirus is used to deliver a polynucleotide encoding a CAR as described herein to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus”. The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukaemia virus (M-MuLV), Moloney murine sarcoma virus (MoMSV), Harvey murine sarcoma virus (HaMuSV), murine mammary tumour virus (MuMTV), gibbon ape leukaemia virus (GaLV), feline leukaemia virus (FLV), spumavirus, Friend murine leukaemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Retroviral vectors and more particularly lentiviral vectors may be used in practicing particular embodiments. Accordingly, the term “retrovirus” or “retroviral vector” as used herein is meant to include “lentivirus” and “lentiviral vectors” respectively.

Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs”. The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and US. The U3 region contains the enhancer and promoter elements. The U5 region is the sequence between the primer binding site and the R region and contains the polyadenylation sequence. The R (repeat) region is flanked by the U3 and U5 regions. The LTR comprises U3, R, and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., J Virol. 1995; 69(4): 2101-9. Several retroviral vectors use the minimal packaging signal (also referred to as the psi [W] sequence) needed for encapsidation of the viral genome. Thus, as used herein, the terms “packaging sequence”, “packaging signal”, “psi” and the symbol “W” are used in reference to the non-coding sequence required for encapsidation of retroviral RNA strands during viral particle formation.

In various embodiments, vectors comprise modified 5′ LTR and/or 3′ LTRs. Either or both of the LTRs may comprise one or more modifications including, but not limited to, one or more deletions, insertions or substitutions. Modifications of the 3′ LTR are often made to improve the safety of lentiviral or retroviral systems by rendering viruses replication defective. As used herein, the term “replication-defective” refers to virus that is not capable of complete, effective replication such that infective virions are not produced (e.g., replication-defective lentiviral progeny). The term “replication-competent” refers to wildtype virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukaemia virus (MoMLV), Rous sarcoma virus (RSV) and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

According to certain specific embodiments, most or all of the viral vector backbone sequences are derived from a lentivirus, e.g., HIV-I. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. Moreover, a variety of lentiviral vectors are known in the art, see Naldini et a!, (Science. 1996; 272(5259): 263-7; Proc Natl Acad Sci USA. 1996; 93(21): 11382-8; Curr Opin Biotechnol. 1998; 9(5): 457-63); Zufferey al., Nat Biotechnol. 1997; 15(9): 871-5; Dull et al., J Virol. 1998; 72(11): 8463-71; U.S. Pat. Nos. 6,013,516; and 5,994, 136, many of which may be adapted to produce a viral vector or transfer plasmid.

In various embodiments, vectors comprise a promoter operably linked to a polynucleotide encoding a CAR or polypeptide as described herein.

In particular embodiments, the vector is a non-integrating vector, including but not limited to, an episomal vector or a vector that is maintained extrachromosomally. As used herein, the term “episomal” refers to a vector that is able to replicate without integration into chromosomal DNA of a host and without gradual loss from a dividing host cell also meaning that said vector replicates extrachromosomally or episomally.

In some embodiments, a vector described herein is a viral vector.

In some embodiments, a viral vector described herein is a retroviral vector.

In some embodiments, a retroviral vector described herein is a lentiviral vector.

In some embodiments, a retroviral vector as described herein is selected from the group consisting of: human immunodeficiency virus I (HIV-I); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus.

In some embodiments, a vector comprises a nucleic acid comprising the sequence of SEQ ID NO: 17. In some embodiments, the vector is a viral vector comprising a nucleic acid sequence comprising the sequence of SEQ ID NO: 17. In some embodiments, the viral vector is a retroviral vector comprising a nucleic acid sequence comprising the sequence of SEQ ID NO: 17. In some embodiments, the retroviral vector is a lentiviral vector comprising a nucleic acid comprising the sequence of SEQ ID NO: 17. In some embodiments, the retroviral vector comprising a nucleic acid comprising the sequence of SEQ ID NO: 17, is a retroviral vector selected from the group consisting of human immunodeficiency virus I (HIV-I); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus.

### Control Elements

In particular embodiments, vectors, which include but are not limited to expression vectors and viral vectors, will include exogenous, endogenous or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked” refers to a juxtaposition wherein the components described are in a relationship permitting them to function in their intended manner. In one embodiment, the term refers to a functional linkage between a nucleic acid expression control sequence (such as a promoter and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer or promoter/enhancer that continually or continuously allows for transcription of an operably linked sequence. A constitutive expression control sequence may be a “ubiquitous” promoter, enhancer or promoter/enhancer that allows expression in a wide variety of cell and tissue types or a “cell-specific”, “cell type-specific”, “cell lineage-specific” or “tissue-specific” promoter, enhancer or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukaemia virus (MoN4LV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), ß-kinesin (ßKIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken 6-actin (CAG) promoter, a ß-actin promoter and a myeloproliferative sarcoma virus enhancer, negative control region deleted, d1587rev primer binding site substituted (MND) promoter (Challita et al., J Virol. 69(2)748-55 (1995)).

In one embodiment, a vector comprises an PGK promoter.

In a particular embodiment, it may be desirable to express a polynucleotide comprising a CAR from a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type- or tissue-specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-I promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In some embodiments, a polynucleotide or cell comprising the polynucleotide utilizes a suicide gene, including an inducible suicide gene to reduce the risk of direct toxicity and/or uncontrolled proliferation. In specific embodiments, the suicide gene is not immunogenic to the host comprising the polynucleotide or cell. A certain example of a suicide gene that may be used is caspase-9 or caspase-8 or cytosine deaminase. Caspase-9 can be activated using a specific chemical inducer of dimerization (CID).

In certain embodiments, vectors comprise gene segments that cause the immune effector cells, e.g., T cells, to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound.

Negative selectable genes are known in the art, and include, inter alia the following: the Herpes simplex virus type I thymidine kinase (HSV-I TK) gene (Wigler et al., Cell I:223, 1977) which confers ganciclovir sensitivity; the cellular hypoxanthine phosphoribosyltransferase (HPRT) gene, the cellular adenine phosphoribosyltransferase (APRT) gene, and bacterial cytosine deaminase (Mullen et al., Proc. Natl. Acad. Sci. USA, 1992; 89(33)).

In some embodiments, genetically modified immune effector cells, such as T cells, comprise a polynucleotide further comprising a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al., Mol. And Cell. Biology, 1991; 11:3374-3378. In addition, in preferred embodiments, the polynucleotides encoding the CARS are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra.

### Vector Production

In particular embodiments, a cell (e.g., an immune effector cell) is transduced with a retroviral vector, e.g., a lentiviral vector, encoding a CAR. For example, an immune effector cell is transduced with a vector encoding a CAR as described herein. These transduced cells can elicit a CAR-mediated cytotoxic response.

A “host cell” includes cells electroporated, transfected, infected or transduced in vivo, ex vivo or in vitro with a vector or a polynucleotide. Host cells may include packaging cells, producer cells and cells transduced with viral vectors. In particular embodiments, host cells transduced with viral vectors are administered to a subject in need of therapy. In certain embodiments, the term “target cell” is used interchangeably with host cell and refers to transfected, infected or transduced cells of a desired cell type. In preferred embodiments, the target cell is a T cell.

Large scale viral vector production is often necessary to achieve a suitable viral titre. Viral particles may be produced by transfecting a transfer vector into a packaging cell line that comprises viral structural and/or accessory genes, e.g., gag, POI, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. In particular embodiments, a retroviral/lentiviral transfer vector is introduced into a packaging cell line, via transfection, transduction or infection, to generate a producer cell or cell line. In particular embodiments, packaging vectors are introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

As used herein, the term “packaging cell lines” is used in reference to cell lines that do not contain a packaging signal but do stably or transiently express viral structural proteins and replication enzymes (e.g., gag, pol and env) which are necessary for the correct packaging of viral particles. Any suitable cell line can be employed to prepare packaging cells. Generally, the cells are mammalian cells. In a particular embodiment, the cells used to produce the packaging cell line are human cells. Suitable cell lines which can be used include, for example, CHO cells, BHK cells, NOCK cells, C3H IOT1/2 cells, FLY cells, Psi2 cells, BOSC 23 cells, PA317 cells, WEHI cells, COS cells, BSC 1 cells, BSC 40 cells, BMT 10 cells, VERO cells, W 138 cells, MRCS cells, A549 cells, HT1080 cells, HEK293 cells, HEK293T cells, B-50 cells, 3T3 cells, NIH3T3 cells, HepG2 cells, Saos-2 cells, Huh7 cells, HeLa cells, W 163 cells, 211 cells and 21 IA cells. In preferred embodiments, the packaging cells are HEKK293 cells or HEK293 T cells. In another preferred embodiment, the cells are HEK293T cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Producer cell line includes those cell lines described in, e.g., WO2017/089307 and WO2017/089308, which comprise all of the elements which are necessary for the production of a retroviral vector, in a single locus in the host cell genome.

Methods of preparing viral stock solutions are known in the art and are illustrated by, e.g., Y. Soneoka et al., (1995) Nucl. Acids Res. 23:628-633 and N. R. Landau et al., (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G protein (VSV-G) envelope proteins, which allows HIV to infect a wider range of cells because HIV envelope proteins (encoded by the env gene) normally target the virus to CD4⁺ presenting cells. In a preferred embodiment, lentiviral envelope proteins are pseudotyped with VSV-G. In one embodiment, packaging cells produce a recombinant retrovirus, e.g., lentivirus, pseudotyped with the VSV-G envelope glycoprotein.

In other embodiments, viral vectors may be pseudotyped with an envelope protein from either another retrovirus or an unrelated virus. The skilled person will appreciate that the viral vectors described herein may be pseudotyped with any suitable envelope protein.

The delivery of a gene(s) or other polynucleotide sequence using a retroviral or lentiviral vector by means of viral infection rather than by transfection is referred to as “transduction”. In one embodiment, retroviral vectors are transduced into a cell through infection and provirus integration. In certain embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

### Immune Effector Cell

In another aspect, provided is an immune effector cell comprising a CAR, polypeptide, polynucleotide, and/or vector as described herein. In various embodiments, cells genetically modified to express the CARs contemplated herein, for use in the treatment of cancer are provided. As used herein, the term “genetically engineered” or “genetically modified” refers to the addition of extra genetic material in the form of DNA or RNA into the total genetic material in a cell. The terms “genetically modified cells”, “modified cells” and “redirected cells” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In particular embodiments, the CARs contemplated herein are introduced and expressed in immune effector cells so as to redirect specificity of the immune effector cell to a target antigen of interest, e.g., cell junction protein located within a cell-cell junction, such as a member of the claudin family of proteins, particularly claudin-3.

An “immune effector cell” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8⁺ T cells), tumour infiltrating lymphocytes (TILS) and helper T cells (HTLs; CD4⁺ T cells). In one embodiment, immune effector cells include natural killer (NK) cells. In one embodiment, immune effector cells include natural killer T cells. In another embodiment, immune effector cells include macrophages. Immune effector cells can be autologous/autogeneic (“self” or non-autologous (“nonself”), e.g., allogeneic, syngeneic or xenogeneic).

“Autologous” as used herein, refers to cells from the same subject.

“Allogeneic” as used herein, refers to cells of the same species that differ genetically to the cell in comparison.

“Syngeneic” as used herein, refers to cells of a different subject that are genetically identical to the cell in comparison.

“Xenogeneic” as used herein, refers to cells of a different species to the cell in comparison.

In preferred embodiments, the cells, e.g., immune effector cells, are allogeneic.

Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes or activated T lymphocytes. A T cell can be a T helper (Th) cell, for example a T helper I (Th1) or a T helper 2 (Th2) cell. The T cell can be a helper T cell (HTL; CD4⁺ T cell), a cytotoxic T cell (CTL; CD8⁺ T cell), CD4⁺ CD8⁺ T cell, CD4⁻ CD8⁻ T cell or any other subset of T cells. Other illustrative populations of T cells suitable for use in particular embodiments include naive T cells and memory T cells.

In some embodiments, the immune effector cell is selected from the group consisting of: a T lymphocyte, a natural killer T lymphocyte (NKT) cell, a macrophage, and a natural killer (NK) cell.

In one embodiment, the immune effector cell is a cytotoxic T lymphocyte (CD8⁺).

As would be understood by the skilled person, other cells may also be used as immune effector cells with the CARs as described herein. In particular, immune effector cells also include NK cells, NKT cells, neutrophils and macrophages. Immune effector cells also include progenitors of effector cells wherein such progenitor cells can be induced to differentiate into an immune effector cell in vivo or in vitro. Thus, in particular embodiments, immune effector cell includes progenitors of immune effectors cells such as hematopoietic stem cells (HSCs) contained within the CD34 population of cells derived from cord blood, bone marrow or mobilized peripheral blood which upon administration in a subject differentiate into mature immune effector cells, or which can be induced in vitro to differentiate into mature immune effector cells.

As used herein, immune effector cells genetically engineered to contain, e.g., a claudin-3 specific CAR may be referred to as “antigen-specific redirected immune effector cells” or “AG-specific redirected immune effector cells”.

Methods for making or generating the immune effector cells which express the CARs described herein are provided in particular embodiments. In various embodiments, such methods comprise introducing into an immune effector cell a polynucleotide and/or vector as described herein. In one embodiment, the method comprises transfecting or transducing immune effector cells isolated from an individual such that the immune effector cells express one or more CARs contemplated herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein). Thus, in certain embodiments, the immune effector cells may be stimulated and induced to proliferate by contacting the cell with antibodies or antigen binding fragments that bind CD3 and/or antibodies or antigen binding fragments that bind to CD28; thereby generating a population of immune effector cells. In further embodiments, the method of generating immune effector cells contemplated herein comprises stimulating the immune effector cell and inducing the cell to proliferate by contacting the cell with antibodies or antigen binding fragments that bind CD3 and antibodies or antigen binding fragments that bind to CD28; thereby generating a population of immune effector cells.

In particular embodiments, prior to in vitro manipulation or genetic modification of the immune effector cells described herein, the immune effector cells are obtained from a subject. In particular embodiments, the CAR-modified immune effector cells comprise T cells.

In particular embodiments, peripheral blood mononuclear cells (PBMCs) may be directly genetically modified to express CARs using methods contemplated herein. In certain embodiments, after isolation of PBMCs, T lymphocytes are further isolated and in certain embodiments, both cytotoxic and helper T lymphocytes can be sorted into naive, memory and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the CARs contemplated herein (e.g., transduced with a viral vector comprising a nucleic acid encoding a CAR) and then are activated and expanded in vitro. In various embodiments, T cells can be activated and expanded before or after genetic modification to express a CAR.

In particular embodiments, a population of modified immune effector cells for the treatment of cancer comprises a CAR as disclosed herein. For example, a population of modified immune effector cells are prepared from peripheral blood mononuclear cells (PBMCs) obtained from a patient diagnosed with cancer (autologous donors). The PBMCs form a heterogeneous population of T lymphocytes that can be CD4⁺, CD8⁺, or CD4⁺ and CD8⁺.

The PBMCs also can include other cytotoxic lymphocytes such as NK cells or NKT cells. A vector carrying the coding sequence of a CAR described herein can be introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein.

Standard procedures are used for cryopreservation of T cells expressing the CAR protein for storage and/or preparation for use in a human subject.

In a further embodiment, a mixture of, e.g., one, two, three, four, five or more, different vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

### T Cell Manufacturing Methods

Methods of manufacturing T cells for human therapy are known in the art. In preferred embodiments, the T cells manufactured by the methods contemplated herein provide improved adoptive immunotherapy compositions. Without wishing to be bound to any particular theory, it is believed that the T cell compositions manufactured by the methods in particular embodiments contemplated herein are imbued with superior properties, including increased survival, expansion in the relative absence of differentiation, persistence in vivo and superior anti-exhaustion properties. For example, T cells modified to express an anti-claudin-3 CAR exhibit a lower binding kinetic to accessible claudin-3 and have a lower potential to exhaust in vivo in the presence of the accessible claudin-3 target. Low tendency towards exhaustion of anti-claudin-3 CAR-T cells retains their effector function, leads to a low sustained expression of inhibitory receptors and retains a transcriptional state of a functional effector or memory T cells. Exhaustion is not a desired feature of a CAR-T therapy since it prevents optimal control of infection and tumours.

In one embodiment, the T cells are modified by transducing the T cells with a viral vector comprising an anti-claudin-3 CAR contemplated herein. Anti-claudin-3 CAR-T cells show low level of basal CAR activation and interferon-gamma (IFNγ) secretion in the absence of the antigen which is a desired attribute of a CAR-T therapy. CARS propensity to antigen-independent (basal) signalling might indicate a self-aggregation leading to antigen-independent CAR activation that in turn could cause early CAR exhaustion resulting in loss of therapeutic potency (Ajina and Maher, 2018 and Long et al., 2015a). Basal activation of CAR-T cells may be determined through the levels of the activation marker CD69, the exhaustion markers PD1 and TIM3, the phosphorylation of CD3ζ intracellular signalling domain, and the ability of CAR-T cells to secret IFNγ in the absence of antigen. Humanised anti-claudin-3 CAR-T cells showed similar levels of IFNγ secretion, activation and exhaustion marker expression and levels of tonic CD3 signalling in vitro to untransduced cells and lower levels compared to CAR-T cells transduced with a positive control CAR.

In one embodiment, the T cells are modified by transducing the T cells with a viral vector comprising an anti-claudin-3 CAR contemplated herein that requires a higher target threshold to be activated rendering it a ‘safer’ CAR. It has been shown that CARs with high affinity can lead to collateral targeting of healthy tissues resulting in on/off-target, off-tumour toxicity (Johnson et al., 2015; Park et al., 2017; Watanabe et al., 2018).

### Pharmaceutical Composition

Immune effector cells described herein may be incorporated into pharmaceutical compositions for use in the treatment of the human diseases described herein. In one embodiment, the pharmaceutical composition comprises an immune effector cell optionally in combination with one or more pharmaceutically acceptable carriers and/or excipients.

Such compositions comprise a pharmaceutically acceptable carrier as known and called for by acceptable pharmaceutical practice, see e.g., Remington's Pharmaceutical Sciences, 16^{th} edition (1980) Mack Publishing Co.

Pharmaceutical compositions may be administered by injection or continuous infusion (examples include, but are not limited to, intravenous, intraperitoneal, intradermal, subcutaneous, intramuscular, intraocular and intraportal). In one embodiment, the composition is suitable for intravenous administration.

A “therapeutically effective amount” of a genetically modified therapeutic cell may vary according to factors such as the disease state, age, sex and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumour size, extent of infection or metastasis and condition of the patient (subject). It can generally be stated that a pharmaceutical composition comprising the T cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a litre or less, can be 500 ml or less, even 250 ml or 100 ml or less. Hence the density of the desired cells is typically greater than 10⁶ cells/ml, e.g., greater than 10⁶, 10⁷, 10⁸ or 10⁹ cells/ml.

The clinically relevant number of immune cells can be apportioned into multiple infusions that cumulatively equal or exceed 10⁵, 10⁶, 10⁷, 10⁸ 10⁹, 10¹⁰, 10¹¹ or 10¹² cells. In some embodiments, particularly since all the infused cells will be redirected to a particular target antigen, lower numbers of cells, in the range of 10⁶/kilogram (10⁶ to 10¹¹ per patient) may be administered. In a particular embodiment, between 1×10⁷ and 9×10⁷ CAR-T cells may be administered. CAR expressing cell compositions may be administered multiple times at dosages within these ranges. For example, CAR-expressing cell compositions may be administered every 7 days. Alternatively, the CAR-expressing cell composition may be administered as a single dose. The cells may be allogeneic, syngeneic, xenogeneic or autologous to the patient undergoing therapy. If desired, the treatment may also include administration of mitogens (e.g., PHA) or lymphokines, cytokines, and/or chemokines (e.g., IFNγ, IL-2, IL-12, TNFα, IL-18, and TNFβ, GM-CSF, IL-4, IL-13, Flt3-L, RANTES, MIP1α, etc.) as described herein to enhance induction of the immune response.

Generally, compositions comprising the cells activated and expanded as described herein may be utilised in the treatment and prevention of diseases that arise in individuals who are immunocompromised. In particular, compositions comprising the CAR-modified T cells contemplated herein are used in the treatment of cancer. In particular embodiments, CAR-modified T cells may be administered either alone, or as a pharmaceutical composition in combination with carriers, diluents, excipients and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising a CAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. In particular embodiments, compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

In another preferred embodiment, compositions comprising T cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CRYOSTOR CS10, CRYOSTOR CS5, and CRYOSTOR CS2.

In a particular embodiment, compositions comprise an effective amount of CAR expressing immune effector cells, alone or in combination with one or more therapeutic agents. Thus, the CAR expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising CAR-expressing immune effector cells disclosed herein may be administered in conjunction with any number of chemotherapeutic agents which are known in the art.

A variety of other therapeutic agents may be used in conjunction with the compositions described herein. In one embodiment, the composition comprising CAR expressing immune effector cells is administered with an anti-inflammatory agent. Anti-inflammatory agents or drugs are known in the art.

In certain embodiments, the compositions described herein are administered in conjunction with a cytokine. By “cytokine” as used herein is meant a generic term for proteins released by one cell population that act on another cell as intercellular mediators. Examples of such cytokines are known in the art.

In further embodiments, the compositions and CARs contemplated herein are administered in conjunction with other CARs or CAR-expressing cells and/or compositions. For example, anti-claudin-3 CARs or compositions may be administered with anti-cell surface associated mucin 1 (MUC1) CARS or CAR-expressing cell compositions. In one embodiment, the anti-MUC1 CAR targets aberrantly glycosylated MUC1 protein (“AG-MUC1”; e.g., TnMUC1, STnMUC1, etc.), such as that expressed by cancer cells. Alternatively, the anti-claudin-3 CARs or compositions contemplated herein may be administered with anti-New York esophageal squamous cell carcinoma 1 (NY-ESO-1) T-cell receptors (TCRs) or TCR-expressing cell compositions.

The pharmaceutical composition may be included in a kit of parts containing the CAR expressing immune effector cell together with other medicaments, optionally and/or with instructions for use. For convenience, the kit may comprise the reagents in predetermined amounts with instructions for use. The kit may also include devices used for administration of the pharmaceutical composition.

The terms “individual”, “subject” and “patient” are used herein interchangeably and refer to any animal that exhibits a symptom of a disease, disorder or condition that can be treated with the CARs, gene therapy vectors, cell-based therapeutics, and methods contemplated elsewhere herein. In preferred embodiments, a subject includes any animal that exhibits symptoms of a disease, disorder or condition related to cancer that can be treated with the CARs, gene therapy vectors, cell-based therapeutics and methods contemplated elsewhere herein. Suitable subjects (e.g., patients) include laboratory animals (such as mouse, rat, rabbit or guinea pig), farm animals, and domestic animals or pets (such as a cat or dog). Non-human primates and, preferably, human patients, are included. Typical subjects include human patients that have been diagnosed with, or are at risk of having, a cancer that expresses an accessible claudin-3 protein. Thus, in one embodiment, the subject is a mammal, such as a primate, for example a marmoset or monkey. In another embodiment, the subject is a human. In a further embodiment, the subject is a mouse.

As used herein, the term “patient” refers to a subject that has been diagnosed with a particular disease, disorder or condition that can be treated with the CARs, gene therapy vectors, cell-based therapeutics, and methods disclosed elsewhere herein.

As used herein “treatment” or “treating” includes any beneficial or desirable effect on the symptoms or pathology of a disease or pathological condition and may include even minimal reductions in one or more measurable markers of the disease or condition being treated. Treatment can involve optionally either the reduction of the disease or condition, or the delaying of the progression of the disease or condition, e.g., delaying tumour outgrowth. “Treatment” does not necessarily indicate complete eradication or cure of the disease or condition, or associated symptoms thereof.

As used herein, “prevent” and similar words such as “prevented”, “preventing” etc., indicate an approach for preventing, inhibiting or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

### Cancer

As used herein, the terms “cancer”, “neoplasm” and “tumour” are used interchangeably and in either the singular or plural form, refer to cells that have undergone a malignant transformation or undergone cellular changes that result in aberrant or unregulated growth or hyperproliferation. Such changes or malignant transformations usually make such cells pathological to the host organism, thus precancers or pre-cancerous cells that are or could become pathological and require or could benefit from intervention are also intended to be included. Primary cancer cells (that is, cells obtained from near the site of malignant transformation) can be readily distinguished from non-cancerous cells by well-established techniques, such as histological examination. For example, such histological examination may distinguish cancer cells from non-cancerous cells by identifying disrupted or compromised cell-cell junctions, such as tight junctions.

Thus, in particular embodiments, the cancer cells comprise disrupted or compromised cell-cell junctions. In further embodiments, the cancer cells comprise disrupted or compromised tight junctions. In such embodiments, cell junction proteins located within cell-cell junctions, such as tight junctions, are mislocalized and become accessible and/or available for binding by the CARs and CAR expressing cells described herein. In other embodiments, the cancer cells comprise mislocalized cell junction proteins, such as those located in cell-cell junctions, on the surface. Thus, in some embodiments, the cancer cells comprise mislocalized cell junction proteins exposed to the surface.

The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumours of any stage, including what a clinician refers to as precancer, tumours, in situ growths, as well as late stage metastatic growths.

By the terms “treating”, “treatment”, and derivatives thereof as used herein, is meant therapeutic therapy. In reference to a particular condition, treating or treatment means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; or (4) to slow the progression of the condition or one or more of the biological manifestations of the condition.

As used herein, “prevention” means the prophylactic administration of a drug, such as an agent, to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term “malignant” refers to a cancer in which a group of tumour cells display one or more of uncontrolled growth (e.g., division beyond normal limits), invasion (e.g., intrusion on and destruction of adjacent tissues), and metastasis (e.g., spread to other locations in the body via lymph or blood).

A “cancer cell” refers to an individual cell of a cancerous growth or tissue. Cancer cells include both solid cancers and liquid cancers. A “tumour” or “tumour cell” refers generally to a swelling or lesion formed by an abnormal growth of cells, which may be benign, pre-malignant, or malignant. Most cancers form tumours, but liquid cancers, e.g., leukaemia, do not necessarily form tumours. For those cancers that form tumours, the terms cancer (cell) and tumour (cell) are used interchangeably. The amount of a tumour in an individual is the “tumour burden” which can be measured as the number, volume or weight of the tumour.

In one embodiment, the target cell (e.g., a cancer cell) expresses a cell junction protein comprising an antigen or epitope which is also expressed on, and in some instances to the same level as, a healthy, non-cancerous cell. In a further embodiment, the antigen or epitope of said cell junction protein is only available and/or accessible when expressed by a cancer cell. Thus, in particular embodiments, the antigen or epitope of said cell junction protein is not available or is inaccessible (e.g., it is ‘hidden’) when expressed by healthy, non-cancerous cells.

In particular embodiments, cancer comprises, or is characterized by, mislocalization of claudin-3 outside of a tight junction and/or disruption of a tight junction such that claudin-3 is accessible for binding by a CAR as described herein.

In one embodiment, the target cell is a bone cell, osteocyte, osteoblast, adipose cell, chondrocyte, chondroblast, muscle cell, skeletal muscle cell, myoblast, myocyte, smooth muscle cell, bladder cell, bone marrow cell, central nervous system (CNS) cell, peripheral nervous system (PNS) cell, glial cell, astrocyte cell, neuron, pigment cell, epithelial cell, skin cell, endothelial cell, vascular endothelial cell, breast cell, colon cell, esophagus cell, gastrointestinal cell, stomach cell, colon cell, head cell, neck cell, gum cell, tongue cell, kidney cell, liver cell, lung cell, nasopharynx cell, ovary cell, follicular cell, cervical cell, vaginal cell, uterine cell, pancreatic cell, pancreatic parenchymal cell, pancreatic duct cell, pancreatic islet cell, prostate cell, penile cell, gonadal cell, testis cell, hematopoietic cell, lymphoid cell, or myeloid cell.

In one embodiment, the target cell expresses claudin-3 protein. In one embodiment, the target cell is a hematopoietic cell, an oesophageal cell, a lung cell, an ovarian cell, a cervix cell, a pancreatic cell, a cell of the gall bladder or bile duct, a stomach cell, a colon cell, a breast cell, a goblet cell, an enterocyte, a stem cell, an endothelial cell, an epithelial cell, or any cell that express claudin-3 protein. In a particular embodiment, the target cell is an endothelial or an epithelial cell.

Illustrative examples of cells that can be targeted by the compositions and methods contemplated in particular embodiments include, but are not limited to those of the following solid cancers: adrenal cancer, adrenocortical carcinoma, anal cancer, appendix cancer, astrocytoma, atypical teratoid/rhabdoid tumour, basal cell carcinoma, bile duct cancer, bladder cancer, bone cancer, brain/CNS cancer, breast cancer, bronchial tumours, cardiac tumours, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumour, extragonadal germ cell tumour, eye cancer, fallopian tube cancer, fibrous histiosarcoma, fibrosarcoma, gallbladder cancer, gastric cancer, gastrointestinal carcinoid tumours, gastrointestinal stromal tumour (GIST), germ cell tumours, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumour, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumours, papillary carcinoma, paraganglioma, parathyroid cancer, penile cancer, pharyngeal cancer, pheochromocytoma, pinealoma, pituitary tumour, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumour.

In another embodiment, the cell is a solid cancer cell that expresses accessible claudin-3 protein. In a yet further embodiment, the cancer is a solid cancer. Exemplary solid cancer cells that express accessible claudin-3 protein which may be prevented, treated, or ameliorated with the CARs, CAR expressing cells and compositions described herein include, but are not limited to: oesophageal cancer, lung cancer (e.g., non-small cell lung cancer (NSCLC)), ovarian cancer, cervical cancer, pancreatic cancer, cholangiocarcinoma, gastric cancer, colon cancer, colorectal cancer, bladder cancer, kidney cancer, and breast cancer (e.g., triple-negative breast cancer (TNBC)) cells.

In certain embodiments, the cancer cells are colorectal cancer, pancreatic cancer, breast cancer, ovarian cancer, or lung cancer. In some embodiments, the breast cancer is triple-negative breast cancer (TNBC). In some embodiments, the lung cancer is non-small cell lung cancer (NSCLC). Thus, in further embodiments, the cancer is selected from colorectal cancer, pancreatic cancer, triple-negative breast cancer (TNBC), ovarian cancer and non-small cell lung cancer (NSCLC).

In other embodiments, the cell is an epithelial cell. In yet other embodiments, the cancer is an epithelial cancer. Exemplary epithelial cancers include, but are not limited to solid cancers, such as those described above.

Illustrative examples of liquid cancers or haematological cancers that may be prevented, treated, or ameliorated with the CARs, CAR expressing cells and compositions contemplated herein include, but are not limited to: leukaemias, lymphomas, and multiple myeloma. Illustrative examples of cells that can be targeted by CARs and compositions contemplated herein include, but are not limited to those of the following leukaemias: acute lymphocytic leukaemia (ALL), T cell acute lymphoblastic leukaemia, acute myeloid leukaemia (AML), myeloblastic, promyelocytic, myelomonocytic, monocytic, erythroleukemia, hairy cell leukaemia (HCL), chronic lymphocytic leukaemia (CLL), and chronic myeloid leukaemia (CML), chronic myelomonocytic leukaemia (CNNL) and polycythemia vera.

### Methods of Treatment and Increasing Cytotoxicity

The CAR molecules contemplated herein are intended to be used in the compositions, cells, and methods for treating cancers described herein, thereby preventing, treating, or ameliorating at least one symptom associated with said cancers. In particular embodiments, the invention relates to improved cell therapy of cancers that express epitopes which are only accessible and/or available for binding of the CAR in said cancers, using genetically modified immune effector cells.

The improved compositions and methods of adoptive cell therapy contemplated herein, provide genetically modified immune effector cells that can readily be expanded, exhibit long term persistence in vivo, and demonstrate antigen dependent cytotoxicity to cancer cells expressing the herein described epitopes.

The terms “individual”, “subject” and “patient” are used herein interchangeably. In one embodiment, the subject is an animal. In another embodiment, the subject is a mammal, such as a primate, for example a marmoset or monkey. In another embodiment, the subject is a human.

Thus, in other aspects, provided are methods for the treatment of cancer with a CAR, polypeptide, vector, immune effector cells, and compositions described herein. The genetically modified immune effector cells contemplated herein provide improved methods of adoptive immunotherapy for use in the prevention, treatment and amelioration of cancers that express accessible claudin-3 proteins, or for preventing, treating or ameliorating at least one symptom associated with accessible claudin-3 protein expressing cancer.

In various embodiments, the genetically modified immune effector cells contemplated herein provide improved methods of adoptive immunotherapy for use in increasing the cytotoxicity to cancer cells in a subject having cancer or for use in decreasing the number of cancer cells in a subject having cancer. In some embodiments, the cancer or cancer cells express claudin-3 protein that is accessible and/or available for binding by the CARs and CAR expressing cells described herein.

In particular embodiments, the specificity of a primary immune effector cell is redirected to cells expressing accessible claudin-3 protein, e.g., cancer cells, by genetically modifying the primary immune effector cell with a CAR contemplated herein. In various embodiments, a viral vector is used to genetically modify an immune effector cell with a particular polynucleotide encoding a CAR comprising claudin-3 binding protein, e.g., an anti-claudin-3 antibody or antigen binding domain; a hinge domain; a transmembrane (TM) domain; one or more co-stimulatory domains; and one or more intracellular signalling domains.

In one embodiment, a type of cellular therapy where T cells are genetically modified to express a CAR as described herein thus providing CAR-T cells, wherein the CAR-T cell is infused to a recipient or subject in need thereof is provided. The infused cell is able to kill disease causing cells in the recipient. Unlike antibody therapies, CAR-T cells are able to replicate in vivo resulting in long-term persistence that can lead to sustained cancer therapy. Furthermore, CAR-T cell therapies contemplated herein may demonstrate increased sensitivity and selectivity compared to antibody therapies as described hereinbefore. For example, a CAR comprising an extracellular domain that comprises an antigen/epitope binding domain as described herein may display greater sensitivity and selectivity than an antibody comprising the same antigen/epitope binding domain, such that activation of CAR-expressing cells is detected (i.e., when the antibody or antigen/epitope binding domain is comprised in a CAR and is thus expressed by a cell in a non-soluble, cellular format), while no binding of the soluble antibody is detected through direct visualization methods.

In one embodiment, the CAR-T cells can undergo robust in vivo T cell expansion and can persist for an extended amount of time. In another embodiment, the CAR-T cells evolve into specific memory T cells that can be reactivated to inhibit any additional tumour formation or growth.

In some embodiments, compositions comprising immune effector cells comprising the CARs contemplated herein are used in the treatment of conditions associated with cancer cells or cancer stem cells that express accessible claudin-3 proteins.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

The terms “enhance”, “promote”, “increase” or “expand” used herein refer generally to the ability of a composition contemplated herein, e.g., a genetically modified T cell or vector encoding a CAR, to produce, elicit or cause a greater physiological response (i.e., downstream effects) compared to the response caused by a control molecule/composition or in a control condition. A measurable physiological response may include an increase in T cell expansion, activation, persistence and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30, 50, 100, 200, 500 or more times the response produced by a control composition or to a control cell lineage. For example, such increased or enhanced amount may be compared to the response seen to healthy, non-cancerous cells which express inaccessible/unavailable claudin-3 protein and/or comprise intact or uncompromised (e.g., undisrupted) cell-cell junctions. Thus, in some embodiments, such increased or enhanced response is seen to cancer cells expressing accessible claudin-3 protein and/or comprise compromised/disrupted cell-cell junctions.

The terms “decrease”, “lower”, “lessen”, “reduce” or “abate” refer generally to the ability of compositions contemplated herein to produce, elicit, or cause a lesser physiological response (i.e., downstream effects) compared to the response caused by a control molecule/composition or in a control condition. A “decreased” or “reduced” amount is typically a “statistically significant” amount, and may include a decrease that is 1.1, 1.2, 1.5, 2, 3, 4, 5, 6, 7, 8, 9, 10, 15, 20, 30, 50, 100, 200, 500 or more times the response (reference response) produced by a control composition, or the response in a particular cell lineage.

In one aspect, there is provided a CAR, polypeptide, polynucleotide, vector, immune effector cell or compositions contemplated herein, for use in therapy, such as for use in the therapy and/or treatment of cancer. In a further aspect, there is provided a CAR, polypeptide, polynucleotide, vector, immune effector cell or compositions contemplated herein, for use as a medicament, such as an anti-cancer medicament. In some embodiments, the CAR, polypeptide, polynucleotide, vector, immune effector cell or compositions contemplated herein for use in therapy is for use in a method of therapy and/or a method of treatment, such as a method of therapy of cancer and/or a method of treating cancer.

In one aspect, there is provided a CAR, polypeptide, polynucleotide, vector, immune effector cell or compositions contemplated herein, for use in the treatment of cancer, wherein the cancer comprises the disruption of a cell-cell junction and/or compromised cell-cell junctions. In another aspect, there is provided a method for treating a subject afflicted with cancer, said method comprising administering to the subject a therapeutically effective amount of the CAR, polypeptide, polynucleotide, vector, immune effector cell or pharmaceutical compositions contemplated herein, wherein the cancer comprises the disruption of a cell-cell junction and/or compromised cell-cell junctions. Thus, in some embodiments, a method for the treatment of cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

One of ordinary skill in the art would recognise that multiple administrations of the compositions contemplated herein may be required to affect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more. Alternatively, only one administration of the compositions contemplated herein may be required.

In one embodiment, a subject in need thereof is administered an effective amount of a composition to increase a cellular immune response to a cancer in the subject. Thus, in a further aspect, there is provided a method for increasing cytotoxicity to cancer cells comprising disrupted cell-cell junctions in a subject in need thereof, such as a subject afflicted with cancer, said method comprising administering to the subject an amount of the CAR, polypeptide, polynucleotide, vector, immune effector cell or compositions contemplated herein.

The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analysing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & sons, NY, N.Y.

In the case of T cell-mediated killing, CAR-ligand binding initiates CAR signalling to the T cell, resulting in activation of a variety of T cell signalling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of proinflammatory cytokines that can induce target cell killing directly (or indirectly via recruitment of other killer effector cells), and up regulation of death receptor ligands (e.g., FasL) on the T cell surface that induce target cell apoptosis following binding to their cognate death receptor (e.g., Fas) on the target cell. Thus, in one aspect, there is provided a method for decreasing the number of cancer cells comprising disrupted cell-cell junctions in a subject afflicted with cancer, said method comprising administering to the subject a therapeutically effective amount of the CAR, polypeptide, polynucleotide, vector, immune effector cell or compositions contemplated herein. In one embodiment, decreasing the number of cancer cells comprising disrupted cell-cell junctions comprises T cell-mediated killing.

In one embodiment, an “effective amount”, which may include a therapeutically effective amount, is sufficient to increase the cytotoxicity to cancer cells, such as cancer cells that comprise disrupted cell-cell junctions and/or compromised cell-cell junctions compared to the cytotoxicity to cancer cells that comprise disrupted cell-cell junctions and/or compromised cell-cell junction prior to the administration. In another embodiment, the “effective amount” is sufficient to decrease the number of cancer cells, such as cancer cells that comprise disrupted cell-cell junctions and/or compromised cell-cell junctions compared to the number of the cancer cells that comprise disrupted cell-cell junctions and/or compromised cell-cell junctions prior to the administration.

In certain embodiments, the methods contemplated herein further comprise administering an activator or binding agent of the ablation element. Such activators or binding agents include, but are not limited to, antibodies (e.g., clinically approved antibodies) such as those which recognise and bind huEGFRt or CD20, i.e., cetuximab or rituximab, respectively, small molecule antagonists of CD20, etc. Administration of an activator or binding agent of the ablation element may be once treatment of the subject is deemed complete, such as following complete response of the subject or cancer. It will thus be appreciated that administration of an ablation element activator or binding agent will prevent any chronic CAR-expressing T cell activity in the subject. In a further embodiment, the methods contemplated herein further comprise utilising the ablation element to target CAR-expressing cells for antibody-dependent cellular cytotoxicity (ADCC) and/or complement-dependent cytotoxicity (CDC). Thus, in certain embodiments, the methods contemplated herein further comprise elimination of the CAR-expressing cells, such as the CAR-expressing T cells. In other embodiments, it may be desirable to suppress any acute CAR-expressing T cell activity in the subject, e.g., to treat cytokine storm, such as by administering steroids.

In a further aspect, there is provided the use of a CAR, polypeptide, polynucleotide, vector, immune effector cell or composition contemplated herein for the manufacture of a medicament, such as an anti-cancer medicament. In one embodiment, the use for the manufacture of a medicament contemplated herein is the manufacture of a medicament for the treatment of cancer.

In one embodiment, a chimeric antigen receptor (CAR) comprising: a) an extracellular domain which comprises an antigen binding protein that binds at least one epitope of a cell junction protein, wherein said cell junction protein is located within a cell-cell junction and wherein said at least one epitope of the cell junction protein is only accessible for binding by said CAR extracellular domain in cancer cells; b) a transmembrane domain; and c) one or more intracellular signalling domains. In one embodiment, the CAR further comprises one or more co-stimulatory domains. In one embodiment, the CAR according to any one of embodiments disclosed herein, wherein the cell junction protein is a tight junction protein, and/or wherein the at least one epitope is inaccessible for binding by the CAR extracellular domain when the cell-cell junction is between cells within organized tissue; and/or when the cell-cell junction is not compromised; and/or wherein the at least one epitope is accessible for binding by the extracellular domain of the CAR when the cell-cell junction is between cancer cells, between a cancer cell and a non-cancerous cell, when the cell-cell junction is compromised, and/or when the cell junction protein is mislocalized outside of the cell-cell junction.

In one embodiment, the CAR according to any one of preceding embodiments, wherein the cell junction protein is a member of the claudin family of proteins; and/or wherein the at least one epitope is present in one or more extracellular loops of the cell junction protein; and/or wherein the cell junction protein is claudin-3; and/or wherein claudin-3 is exposed to the cell surface in a solid cancer which has disrupted or disorganized tight junctions; and/or wherein claudin-3 is not exposed to the cell surface and is localized in cell-cell junctions in normal or non-cancerous cells.

In one embodiment, the CAR according to any one of preceding embodiments, wherein the at least one epitope is present uniquely in claudin-3; and/or wherein the at least one epitope is 4 amino acids in length; and/or wherein the at least one epitope is discontinuous epitope.

In one embodiment, the CAR according to any one of preceding embodiments, wherein the antigen binding protein is selected from an antibody or antigen binding fragment thereof; and/or wherein the antigen binding protein is selected from the group consisting of: a monoclonal antibody, a Camel Ig, Ig NAR, Fab fragments, Fab′ fragments, F(ab)′2 fragments, F(ab)′3 fragments, Fv, scFv, bis-scFv, (scFv)2, minibody, diabody, triabody, tetrabody, disulfide stabilized Fv protein (“dsFv”) and sdAb; and/or wherein the antigen binding protein is a scFv.

In one embodiment, the CAR according to any one of preceding embodiments, wherein the antigen binding protein comprises any one or a combination of CDRs selected from CDRH1, CDRH2 and CDRH3 from SEQ ID NO: 7 and/or CDRL1, CDRL2 and CDRL3 from SEQ ID NO: 8; or the antigen binding protein comprises all six CDRs from SEQ ID NOs: 7 and 8; or the antigen binding protein comprises: a CDRH1 sequence of SEQ ID NO: 1; a CDRH2 sequence of SEQ ID NO: 2; a CDRH3 sequence of SEQ ID NO: 3; a CDRL1 sequence of SEQ ID NO: 4; a CDRL2 sequence of SEQ ID NO: 5; and a CDRL3 sequence of SEQ ID NO: 6. Consistent with these embodiments, the antigen binding protein comprises a variable heavy chain (VH) sequence at least 90% identical to the sequence of SEQ ID NO: 7, and a variable light chain (VL) sequence at least 90% identical to the sequence of SEQ ID NO: 8; or wherein the antigen binding protein comprises a variable heavy chain (VH) sequence of SEQ ID NO: 7, and a variable light chain (VL) sequence of SEQ ID NO: 8; or wherein the antigen binding protein comprises, from N-terminus to C-terminus, a VH sequence of SEQ ID NO: 7 and a VL sequence of SEQ ID NO: 8; or wherein the antigen binding protein comprises, from N-terminus to C-terminus, a VL sequence of SEQ ID NO: 8 and a VH sequence of SEQ ID NO: 7.

In one embodiment, the CAR according to any one of the preceding embodiments, wherein the transmembrane domain is derived from a polypeptide selected from the group consisting of: alpha or beta chain of the T-cell receptor, CD3δ, CD3ε, CD3γ, CD3ζ, CD4, CDS, CD8α CD9, CD16, CD22, CD27, CD28, CD33, CD37, CD45, CD64, CD80, CD86, CD134, CD137 (4-1BB), CD152, CD154, CD278 (ICOS) and PD1; or wherein the transmembrane domain is derived from CD8α. In one embodiment, the CAR according to any one of the preceding embodiments, the CAR according to any one of the preceding embodiments, wherein the one or more intracellular signalling domains is derived from an intracellular signalling molecule selected from the group consisting of: FcRγ, FcRβ, CD3γ, CD3ε, CD3δ, CD3ζ, CD22, CD66d, CD79a and CD79b; or wherein the one or more intracellular signalling domains is CD3ζ. In one embodiment, the CAR according to any one of the preceding embodiments, the CAR further comprises one or more co-stimulatory domains that is derived from a co-stimulatory molecule selected from the group consisting of: CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TLR1, TLR2, TLR3, TLR4, TLR5, TLR6, TLR7, TLR8, TLR9, TLR10, TRIM and ZAP70; or wherein the one or more co-stimulatory domains is CD137 (4-1BB).

In one embodiment, the CAR according to any one of preceding embodiments, the extracellular domain comprises an amino acid having at least 90%, 91%, 92%, 93%, 94%, 95%, 96%, 97%,98%, 99% or 100% sequence identity to SEQ ID NO: 11; or wherein the CAR comprises an amino sequence of SEQ ID NO: 11. In one embodiment, the CAR according to any one of preceding embodiments, the extracellular domain comprises an amino acid having at least 90%, 91%, 92%, 93%, 94%, 95%, 96%, 97%,98%, 99% or 100% sequence identity to SEQ ID NO: 18; or wherein the CAR comprises an amino sequence of SEQ ID NO: 18.

In one embodiment, the CAR according to any one of preceding embodiments, the CAR comprises an amino acid sequence having at least 90%, 91%, 92%, 93%, 94%, 95%, 96%, 97%,98%, 99% or 100% sequence identity to SEQ ID NOs: 12, 24, 25, 27, 28, 29, or 30; or wherein the CAR comprises an amino acid sequence of SEQ ID NOs: 12, 24, 25, 27, 28, 29, or 30; or wherein the CAR comprises an amino acid sequence having at least 90%, 91%, 92%, 93%, 94%, 95%, 96%, 97%,98%, 99% or 100% sequence identity to SEQ ID NOs: 12, 24, 25, 27, 28, 29, or 30 without CD8 leader sequence of SEQ ID NO:10. One of ordinary skill in the art would appreciate that CD8 leader sequence of SEQ ID NO:10 that is introduced in the CAR according to any one of the preceding embodiments can be modified or deleted without affecting the function of the CAR using standard techniques known in the art. Consistent with these embodiments, the CAR according to any one of preceding embodiments, the CAR comprises an amino acid sequence having at least 90%, 91%, 92%, 93%, 94%, 95%, 96%, 97%,98%, 99% or 100% sequence identity to SEQ ID NOs: 34, 35, 36, 37, 38, or 39.

In one embodiment, the CAR according to any one of preceding embodiments, the CAR comprises a) an extracellular domain which comprises a claudin-3 binding protein comprising a CDRH1 sequence of SEQ ID NO: 1; a CDRH2 sequence of SEQ ID NO: 2; a CDRH3 sequence of SEQ ID NO: 3; a CDRL1 sequence of SEQ ID NO: 4; a CDRL2 sequence of SEQ ID NO: 5; and a CDRL3 sequence of SEQ ID NO: 6; b) a transmembrane domain derived from CD8α; c) a co-stimulatory domain derived from CD137 (4-1BB); and d) an intracellular signalling domain derived from CD3ζ. In one embodiment, there is provided a CAR that competes for binding with the CAR according to any one of the preceding embodiments.

In one embodiment, there is provided a polypeptide comprising the amino acid sequence of the CAR of any one of the preceding embodiments. In yet another embodiment, wherein the polypeptide further comprises an ablation element. Consistent with these embodiments, wherein the ablation element is a cell surface protein which is targeted for antibody-dependent cellular cytotoxicity (ADCC) and/or complement-dependent cytotoxicity (CDC) using an antibody or antigen binding fragment; and/or wherein the ablation element is derived from a polypeptide selected from the group consisting of: truncated human EGFR polypeptide (huEGFRt) and CD20 or wherein the ablation element is CD20. In one embodiment, there is provided a vector comprising the polynucleotide according to any one of the preceding embodiments. In yet another embodiment, wherein the vector is a viral vector; and/or wherein the viral vector is a retroviral vector, such as a lentiviral vector; and/or wherein the retroviral vector is selected from the group consisting of: human immunodeficiency virus I (HIV-I); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus. In one embodiment, there is provided a vector producer cell comprising the polynucleotide sequence according to any one of the embodiments disclosed herein and/or the vector according to any one of the preceding embodiments.

In one embodiment, there is provided an immune effector cell comprising the CAR, the polypeptide, the polynucleotide and/or the vector according to any one of the preceding embodiments. Consistent with these embodiments, the immune effector cell is selected from the group consisting of: a T lymphocyte, a natural killer T lymphocyte (NKT) cell, a macrophage, and a natural killer (NK) cell; or wherein the immune effector cell is a cytotoxic T lymphocyte (CD8⁺). In one embodiment, there is provided a pharmaceutical composition comprising the immune effector cell according to any one of the preceding embodiments and a pharmaceutically acceptable excipient. Also provided includes a method of generating an immune effector cell comprising a CAR according to any one of the preceding embodiments, said method comprising introducing into an immune effector cell the polynucleotide and/or the vector according to any one of the preceding embodiments. Consistent with these embodiments, said method further comprising stimulating the immune effector cell and inducing the cell to proliferate by contacting the cell with an antibody or antigen binding fragment thereof that binds CD3 and an antibody or antigen binding fragment thereof that binds to CD28; thereby generating a population of immune effector cells. In one embodiment, wherein stimulating the immune effector cell is performed before introducing into the cell the vector according to any one of the preceding embodiments; and/or wherein the immune effector cell comprises a T lymphocyte.

In one embodiment, there is provided the CAR, the polypeptide, the polynucleotide, the vector, the immune effector cell, or the pharmaceutical composition according to any one of the preceding embodiments for use in the treatment of cancer. In one embodiment, there is provided a method of treating cancer in a subject in need thereof, said method comprising administering to the subject a therapeutically effective amount of the CAR, the polypeptide, the polynucleotide, the vector, the immune effector cell, or the pharmaceutical composition according to any one of the preceding embodiments. Yet in other embodiments, a method of increasing cytotoxicity to cancer cells in a subject having cancer, said method comprising administering to the subject an effective amount of the CAR, the polypeptide, the polynucleotide, the vector, the immune effector cell, or the pharmaceutical composition according to any one of the preceding embodiments is provided. In one embodiment, there is provided a method of increasing cytotoxicity to cancer cells in a subject having cancer, said method comprising administering to the subject an effective amount of the CAR, the polypeptide, the polynucleotide, the vector, the immune effector cell, or the pharmaceutical composition according any one of the preceding embodiments. In other embodiments, a method of decreasing the number of cancer cells in a subject having cancer, said method comprising administering to the subject an effective amount of the CAR, the polypeptide, the polynucleotide, the vector, the immune effector cell, or the pharmaceutical composition according any one of the preceding embodiments is provided. Consistent with these embodiments, wherein the cancer is characterized by mislocalization of claudin-3 outside of a tight junction and/or disruption of a tight junction such that claudin-3 is accessible for binding; or wherein the cancer is characterized by claudin-3 exposed to cell surface due to the disruption of a tight junction. In one embodiment, wherein the cancer is a solid cancer; or wherein the solid cancer is colorectal cancer, pancreatic cancer, breast cancer (e.g., triple-negative breast cancer (TNBC)), ovarian cancer, lung cancer (e.g., non-small cell lung cancer (NSCLC)), or prostate cancer; or wherein the cancer is an epithelial cancer.

In one embodiment, there is provided use of the CAR, the polypeptide, the polynucleotide, the vector, the immune effector cell, or the pharmaceutical composition according any one of the preceding embodiments in the manufacture of a medicament for treatment of cancer. Yet in other embodiment, the CAR, the polypeptide, the polynucleotide, the vector, the immune effector cell, or the pharmaceutical composition according any one of the preceding embodiments according to claim 48 for use in therapy is provided.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognise a variety of noncritical parameters that could be changed or modified to yield essentially similar results.

### EXAMPLES

### Example 1

### Generation of CAR-T Cells

CD4⁺ and CD8⁺ T cells from healthy human peripheral blood were isolated and subsequently transduced with lentiviral vectors encoding for either anti-claudin-3 or control CAR constructs (control CAR was anti-CD19 CAR). CD4⁺ and CD8⁺ T cells isolated from healthy donors were all successfully transduced with lentiviral vectors encoding anti-claudin-3 CARs or control anti-CD19 CAR. CAR T cells were generated from cells isolated from multiple donors and were expanded and frozen as required for subsequent in vitro and in vivo functional assays.

### Materials and Methods

Isolation of CD4⁺ and CD8⁺ T cells and Activation of T Cells

Peripheral blood monocytes (PBMCs) were isolated from whole peripheral blood and NHS Blood and Transplant (NHSBT) cones as follows using Histopaque (Sigma, catalogue number 10771) in accordance with the manufacturer's instructions. Cells were resuspended in AutoMACS running buffer and FcR blocking reagent, CD4 Microbeads and CD8 Microbeads (all Miltenyi Biotec) were added per 10⁷ cells. Cell were mixed and incubated for 15 minutes at 4° C. Then, the cells were washed, centrifuged and resuspended in cold AutoMACS running buffer per 10⁸ cells. Cell solutions were run on the AutoMACS pro-separator (Miltenyi Biotec) using the Possel_S separation protocol. Positive fractions containing the magnetically labelled CD4⁺ and CD8⁺ T cells were washed three times with PBS to ensure at least a 200-fold reduction in the amount of EDTA within the cell solution, as EDTA can impact upon T cell activation. After the final PBS wash, cell pellets were resuspended in an appropriate volume of TEXMACS media (Miltenyi Biotec) and a sample was removed for counting on a NC-250 Nucleocounter (ChemoMetec).

Cells were resuspended in TEXMACS media. TransAct T cell activation reagent (Miltenyi Biotec) as well as IL-7 and IL-15 were added to the cells to achieve a final concentration of 10 ng/mL for each cytokine. The cell solution was plated (1×10⁶ cells/mL) into a cell culture plate and the cells were subsequently incubated at 37° C. within a humidified incubator with 5% CO₂ for 24 hours.

Transduction of T cells with Lentiviral Vectors and Expansion of CAR-T Cells

Cells were transduced with lentiviral vector encoding an anti-claudin-3 CAR (906_009) with a reporter gene (LNGFR), referred to as 906_009-LNGFR, or anti-CD19 CAR vector (control)) at an MOI of 3. CAR constructs comprised a LNGFR marker to enable detection and/or enrichment of CAR-T cell expressing T-cells. The LNGFR marker system uses transiently expressed truncated human low-affinity nerve growth factor receptor (LNGFR) molecule as a surface maker to detect and/or select transfected cells. Cells were incubated at 37° C. with 5% CO₂ within a humidified incubator. Cells were maintained in TEXMACs media and IL-17 and IL-15 at a concentration of 10 ng/mL of each cytokine throughout the culture period. For some batches, cells were cultured on IL-2 instead of IL-7 & IL-15. If IL-2 was used, the same culture procedure was followed, however 100 international units (IU)/mL of IL-2 were added instead of IL-7 & IL-15. T cells were harvested 12 days after transduction and frozen in CS5 freezing media (Sigma, #C2999) at cell densities of between 1×10⁷-1×10⁸ cells/mL.

Transduction efficiency was determined by detecting expression of truncated human low-affinity nerve growth factor receptor (LNGFR; CD271) with PE conjugated anti-LNGFR antibody (Ab) using flow cytometry (MACSQuant Analyser 10). Data was analysed using FlowJo v10.1.

For certain CAR-T batches, it was required to normalise all CAR-T populations to the lowest transduction efficiency. In order to accurately normalise the CAR-T cell populations, the frequency of LNGFR⁺ cells were analysed and cells were counted. Subsequent to this, the volume of untransduced T cells required to bring the frequency of LNGFR⁺ cells down to the defined level was calculated and added into each cell population as appropriate.

Two preparations of cells transduced with anti-claudin-3 CAR vector (906_009-LNGFR) were made, one in suspension cells (referred to as “vector 1”) one in adherent cells (referred to as “vector 3”). Other than a difference in transduction efficiency between the two different cell preparations (see discussion below), no significant difference was observed between the two different cell preparation methods.

### T Cell Enrichment for Generation of a Pure Population of CAR-T Cells

For some CAR-T batches, T cells were enriched at day 12 post-induction to generate a pure population of CAR-T cells by positive selection using AutoMACs Pro-Separator Enrichment or EasySep Enrichment, as described below.

For AutoMACs Pro-Separator Enrichment, LNGFR microbeads (Miltenyi Biotec) were added per 10⁷ cells and the cells were mixed well before incubating for 15 minutes at 4° C. The cells were washed, and cell solutions were run on the AutoMACS pro-separator using the Possel_S separation protocol. Positive fractions containing the magnetically labelled LNGFR⁺ T cells were washed three times with PBS to ensure at least a 200-fold reduction in the amount of EDTA within the cell solution, as EDTA can impact upon T cell activation.

EasySep Enrichment was performed at either day 9 post-transduction or day 12 post-transduction depending on the requirements of the assay and total cell numbers in culture. Transduced T-cells with different CARs tagged with LNGFR were positively selected using the EASYSEP Human CD271 Positive Selection Kit II (STEMCELL Technologies UK Ltd) and the EASYSEP rapidshere beads (StemCell Technologies). Depending on the number of transduced T-Cells to be enriched, either the EASYPLATE magnet (StemCell Technologies) or the EASYEIGHT magnet (StemCell Techologies) was used. Freshly thawed or cultured transduced T cells were resuspended at a density between 1×10⁷ and 2×10⁷ in TEXMACS medium supplemented with EASYSEP Human FcR Blocker (25 μL/ml) and EASYSEP Human CD271 Positive Selection Cocktail (50 μL/ml) and incubated at RT for 15 min.

For higher cell density, between 1×10⁸ and 2×10⁸ cells were resuspended in TEXMACS media supplemented with EASYSEP Human FcR Blocker and EASYSEP Human CD271 Positive Selection Cocktail at the indicated concentrations. 50 μL/mL of EASYSEP rapidshere beads were added to each sample and cells were incubated at RT for 15 min. Following the incubation, samples were topped up with washing buffer (PBS containing 2% Foetal Bovine Serum (FBS) and 2 mM EDTA), moved onto the EASYPLATE or EASYEIGHT EASYSEP Magnet and incubated for 10 minutes. Supernatant was carefully removed without disturbing the positive selected cells attached to the beads. After performing 3 more washes, the cells were resuspended in TEXMACS medium and samples were removed for counting on the NC-250 nucleocounter and for post-enrichment LNGFR analysis to confirm that the enrichment was successful.

If enrichment was performed on day 9 post-transduction, enriched CAR-T cells were re-plated with TEXMACS media with IL-7 and IL-15 at a concentration of 10 ng/mL. Cells were incubated at 37° C. with 5% CO₂ within a humidified incubator for 72 hours, and frozen as described above on day 12 post-transduction.

If enrichment was performed on day 12 post-transduction, enriched cells were either used immediately in functional assays or frozen.

### Results

Expansion of T cells, Transduction Efficiency and Enrichment and Normalisation of CAR-T Cell Populations

All T cell populations were successfully expanded, with fold expansions ranging between 14- and 178-fold, dependent on the specific donor. The average fold expansion across all T cell populations was 76.

CAR-T cells transduced with anti-claudin-3 CAR vector 1 at an MOI of 3 had transduction efficiencies (based on frequency of LNGFR positive cells) of between 41-60% across multiple donors and vector batches. CAR-T cells transduced with anti-claudin-3 CAR vector 3 at an MOI of 3 achieved lower transduction efficiencies of 29-37% in the three donors used. All control CAR T cells (anti-CD19 CAR) achieved transduction efficiencies of between 48-75% across multiple donors and vector batches using an MOI of 3.

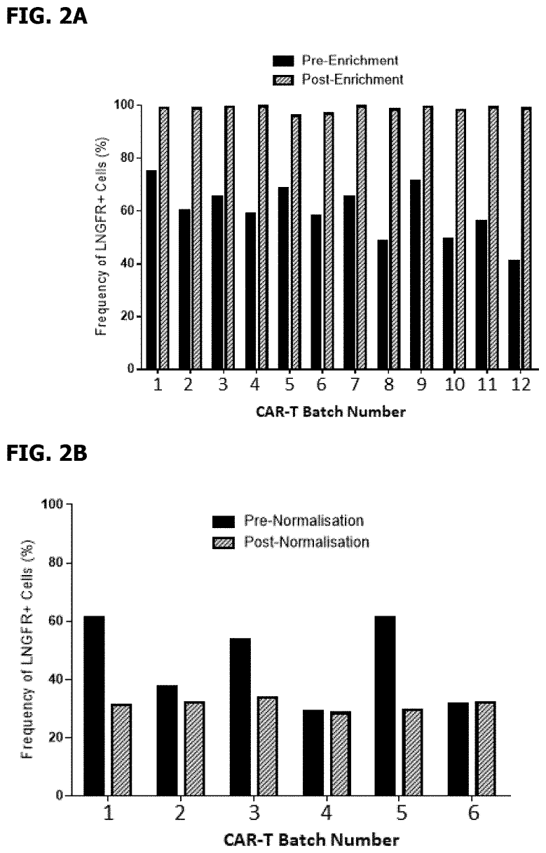

Enrichment of LNGFR⁺ CAR-T cells to provide a 100% LNGFR⁺ CAR-T cell population was successful with both AutoMACS pro-separator enrichment (data not shown) and EasySep LNGFR enrichment as shown in FIG. 2A for all CAR-T cell batches produced.

Normalisation of CAR-T cell populations to the required frequency of LNGFR expressing T cells was successful, as shown in FIG. 2B for CAR-T batches produced.

CD4 and CD8 positive selection using the AutoMACS Pro-Separator enabled a >95% CD3⁺ cell population to be transduced at day 0. This enabled vector to efficiently transduce only the desired cell types. There was minimal monocyte contamination (<5%) after CD4/CD8 positive selection, with remaining monocytes dying off over the culture period resulting in a pure CD3⁺ cell population at day 12 post-transduction.

CD4⁺ and CD8⁺ T cells isolated from healthy donors were all successfully transduced with lentiviral vectors encoding anti-claudin-3 CARs or control anti-CD19 CAR. Differences in transduction efficiency were observed between the lentiviral vectors 1 and 3. All T cell populations were successfully expanded, with some anomalous expansions observed within donors for certain CAR constructs.

Enrichment of CAR-T cells by using both AutoMACs pro-separator and EasySep LNGFR microbeads was successful and enabled the provision of 100% LNGFR⁺ CAR-T populations for subsequent functional assays. In addition to this, normalisation of CAR-T cell population to required frequency of LNGFR⁺ cells was successful.

All CAR-T cells produced were able to be used in functional assays to test anti-claudin-3 CAR vector 1. CAR-T cells produced in suspension cells were used for subsequent experiments.

### Example 2

### Effect of CAR Expression and T Cell Phenotype

The objective of this study was to evaluate the effect of tonic signalling (antigen independent signalling) for anti-claudin-3 CAR-T cells in vitro. CAR-T cells that exhibit tonic signalling lead to impaired in vitro T cell function and exhaustion and inferior in vivo efficacy. Tonic signalling is influenced by a combination of features of the CAR structure, linker or hinge, signalling domains, surface expression location and levels. Tonic signalling was assessed by measuring basal level of cytokines secreted in cell supernatants (IFNγ), differentiation of continuous T-cell phenotype by measuring activation (CD69) and exhaustion (PD-1 and TIM-3) markers, and measurement of enhanced antigen independent signalling (pCD3ζ). Responses were benchmarked versus a negative control anti-CD19 CAR and positive control (GD2-28ζ) CAR, which demonstrate low and high levels of tonic signalling, respectively.

The results demonstrate that the anti-CD19 CAR negative control and anti-claudin-3 CAR both conferred low levels of tonic signalling compared to the positive control (GD2-28ζ) CAR-T cells, indicating a low level of antigen independent activation in vitro for the claudin-3 CAR construct.

### Materials and Methods

### Generation of Negative and Positive Control CARs

The negative control anti-CD19 CAR was generated using the FMC62 ScFv with a 4-1BB-CD3ζ cytosolic signalling domain and was used herein as a control to benchmark a low level of tonic signalling response. The positive control CAR (GD2-28ζ) was generated using a 14g2a scFv with a CH₂-CH₃ IgG₁ linker and a CD28-CD3ζ transmembrane and cytosolic spanning domain. Here, the EF1a promoter was used to enhance the transduction efficiency of the CAR and should result in an increased propensity to drive a tonic signalling response. The CD28ζ transmembrane and cytosolic domain should increase the level of tonic signaling compared to 4-1BBζ cytosolic domain independent of the lentivector transduction promoter used. Furthermore, the 14g2a anti-GD2 scFv clone has a propensity to oligomerize—a feature characterized by the GD2-28ζ CAR structure resulting in intrinsic activation of CAR dependent signalling. The IgG1 CH₂-CH₃ extracellular linker used in the positive control CAR could also contribute to the level of tonic signalling observed.

### CAR T-Cell Thawing and Culture

CAR-T cells (either thawed from cryo-frozen stock or fresh cells) were resuspended in TEXMACS media and the cell density was adjusted to 2×10⁶ cells/mL in TEXMACS media with 10 ng/mL IL-7/IL-15. Resuspended cells were placed in a humidified incubator for 24 hours at 37° C. with 5% CO₂ prior to LNGFR enrichment.

### CAR-T Cell LNGFR Enrichment Post-Thawing

LNGFR expressing CAR-T wells were positively selected using the EASYSEP Human CD271 Positive Selection Kit and EASYSEP Dextran RAPIDSPHERES. The CAR-T cells were harvested and resuspended to a density of 10 to 20×10⁶ cells in TEXMACS medium supplemented with EASYSEP Human FcR Blocker and EASYSEP Human CD271 positive selection cocktail in a non-tissue culture treated 96-well plate and incubated for 15 minutes at RT. EASYSEP Dextran RAPIDSPHERES were added to the cell suspension and incubated for 15 minutes at RT. Thereafter, LNGFR expressing cells were selected using the EASYPLATE EASYSEP Magnet, resuspended in TEXMACS medium supplemented with 10 ng/mL of human IL-7/IL-15 and placed in a humidified incubator for 72 hours at 37° C. with 5% CO₂ prior to subsequent assays or cryo-preservation. Cryo-preserved LNGFR enriched cells were thawed and seeded at a density of 2.5×10⁶/well in TEXMACS media supplemented with 10 U/mL IL-2 and placed in a humidified incubator for 24 hours at 37° C. with 5% CO₂. Supernatants and cells were provided for subsequent assays.

### Lysate Generation and Protein Quantification

CAR-T and untransduced T cells were harvested from cultures and 2×10⁶ cells were resuspended in cold dPBS (with calcium and magnesium), centrifuged, and the resulting cell pellet was lysed by repeat pipetting of cold lysis buffer. The lysates were centrifuged, aliquoted, snapped frozen and stored at −80° C. for long term storage. The protein level in the lysates was quantified using the bicinchoninic acid (BCA) assay.

### Determination of IFNγ Cytokine Secretion in CAR-T Cell Supernatants

Human IFNγ meso scale discovery (MSD) plates were loaded with test samples. The plates were then sealed and incubated at room temperature on a plate shaker for 90 minutes. Plates were washed and detection antibody was added to each well. The plates were sealed and incubated at room temperature on a plate shaker for 2 hours. Following this, the plates were washed and read on the MSD Sector 600 Imager. The average and standard error of the mean for the levels of IFNγ secreted for the test CAR T cells across the 6 donors was calculated and the data plotted using GRAPHPAD PRISM (Bonferroni ONEWAY ANOVA).

### Determination of CAR-T Cell Phenotype (CD69, TIM-3, PD-1)

Untransduced T cells and CAR-T cells were thawed and counted. 2.5×10⁵ cells/well were aliquoted into a 96 well plate. The cells were then washed and the appropriate antibody mix (containing antibodies against CD3, CD8, CD69, TIM3, PD1) was added to each well. The cells were incubated for 15 minutes at room temperature, in the dark, then resuspended in media with DAPI live/dead dye at a final concentration of 1 μg/mL. The samples were analysed by flow cytometry and flow cytometric data was analysed using FLOWJO V10 software.

The expression and co-expression of activation/exhaustion markers CD69, PD-1 and TIM-3 were generated by stratifying the single, live cells by CD4⁺ and CD8⁺ populations. Once gated as either CD4⁺ or CD8⁺ cell populations, the activation/exhaustion markers were identified by single positivity only. Then subsequent Boolean gating logic was applied to characterise single, double and triple positive/negative cell populations of the three activation/exhaustion markers.

For the data analysis, the average percentage of the triple positives (PD-1, TIM-3 and CD69), double positives (CD69 and TIM-3 or CD69 and PD-1 or TIM-3 and PD-1) and single positives (PD-1 or TIM-3 or CD69) for negative control (anti-CD19 CAR), positive control (GD2-28ζ CAR) and anti-claudin-3 CAR was calculated across the six PBMC donors. The data was analysed using GraphPAD PRISM (Bonferroni ONEWAY ANOVA).

### Determination of Downstream Signaling via Phosphorylation of CAR Specific CD3ζ

Lysates were obtained from 2×10⁶ CAR-T cells and the concentrations normalized to 300 μg/mL and heated. Anti-pCD3ζ, anti-CD3ζ, GAPDH and secondary antibodies were added to the lysates prior to loading. The protein levels were assessed using the PEGGY-SUE high throughput capillary western technology. The normalized level of phosphorylated CD3ζ (pCD3ζ) was calculated based on total-CD3ζ and GAPDH loading control from a maximum of 6 donors.

Antigen independent signalling data analysis was conducted using Compass for SW software (PEGGY-SUE) producing primary metrics. Here, the Area under Peak (AuP) for respective stains was determined using the software and the responses normalized based on AuP of GAPDH (total protein load) levels. The normalized pCD3ζ levels for test CAR-T cells (positive control (GD2-28ζ) and anti-claudin-3 CAR-T cells) was divided by the normalized pCD3ζ levels detected for the negative control CAR-T cells (anti-CD19 CAR). The average and standard error of the mean for the levels of CAR specific phosphorylation (pCD3ζ) for the test CAR T cells across the 6 donors was calculated. The data was plotted using GraphPAD PRISM (Bonferroni ONEWAY ANOVA).

### Results

Basal Level IFNγ Secretion from CAR T Cells

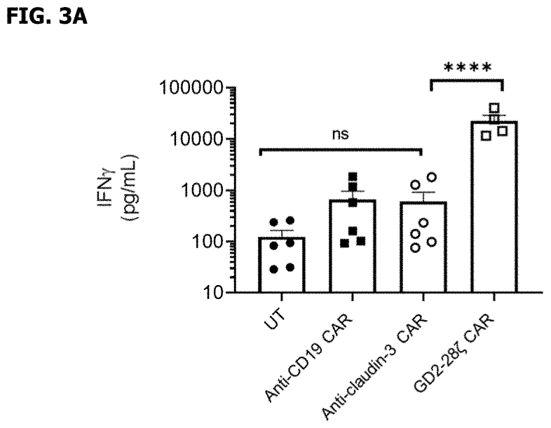

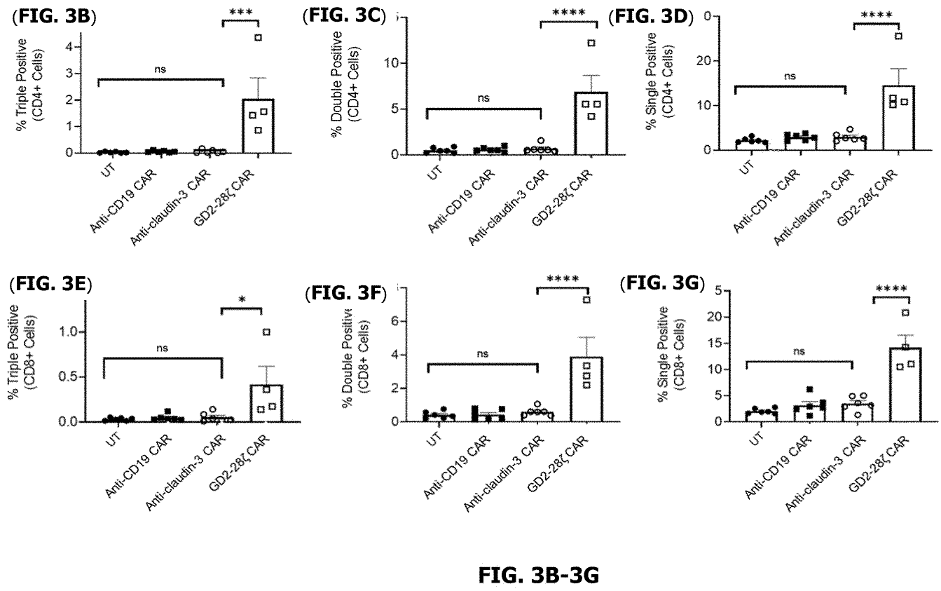

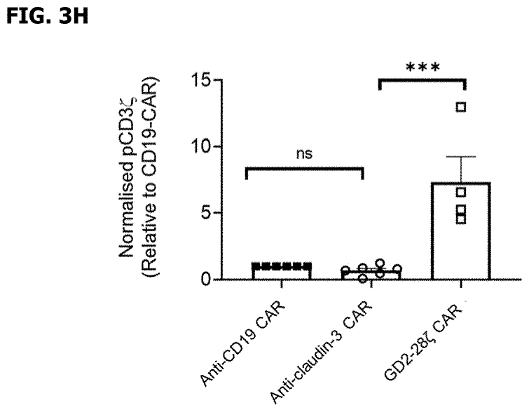

IFNγ secretion by T cells is a key measurement of T cell activation and antigen independent signalling can, in part, be assessed by the secretion of this cytokine. The data presented in FIG. 3A shows significantly less IFNγ secretion from anti-claudin-3 CAR T-cells (611.8±755.1pg/mL) compared to positive control (GD2-28ζ) CAR-T cells (22557±12903 pg/mL). No significant difference between the untransduced T cells (123.7±103.0 pg/mL), negative control (anti-CD19 CAR; 666.4±725.1 pg/mL) and anti-claudin-3 CAR-T cells was observed (FIG. 3A).

Basal T Cell Activation (CD69⁺) and Exhaustion (TIM-3⁺ and PD-1⁺) Phenotype

Differentiation of the basal continuous phenotype of T cells to show an increase in activation (CD69⁺) and exhaustion markers (TIM-3⁺ and PD-1⁺) can complement a subset of assays used to detect tonic signaling. The data presented in FIG. 3B shows significant increase in activation and exhaustion phenotype in positive control (GD2-28ζ CAR) compared to negative control (anti-CD19 CAR) and the anti-claudin-3 CAR-T cells. For the positive control (GD2-28ζ CAR), the CD4⁺ T cells (Triple positive: 2.05±1.57%, double positive: 6.89±3.61, single positive: 14.59±7.36%) displayed a higher increase in activation and exhaustion phenotype compared to the CD8⁺ T cells (Triple positive: 0.42±0.4%, double positive: 3.9±2.31%, single positive: 14.18±4.75%). For anti-claudin-3 CAR-T cells, the CD4⁺ T cells (Triple positive: 0.06±0.06%, double positive: 0.71±0.44, single positive: 2.99±0.96%) displayed a higher increase in activation and exhaustion phenotype compared to the CD8⁺ T cells (Triple positive: 0.54±0.5%, double positive: 0.61±0.25%, single positive: 3.51±1.37%) which were significantly lower (FIG. 3B).

### Phosphorylation of CAR Specific CD3ζ

From the results analysis, the normalized levels of CAR pCD3ζ for anti-claudin-3 CAR (0.68±0.39) were significantly lower compared to positive control (GD2-28ζ) CAR (7.32±3.84) and not significantly different from negative control (anti-CD19) CAR pCD3ζ (FIG. 3C).

From the results obtained from the IFNγ secretion, activation (CD69⁺) and exhaustion (TIM-3⁺ and PD-1⁺) phenotype and the CAR pCD3ζ levels, the anti-CD19 CAR and anti-claudin-3 CAR both conferred low levels of tonic signalling compared to the positive control (GD2-28ζ) CAR-T cells. The level of CAR transduction on the T cells estimated from the total-CD3ζ staining showed differences based on the promoter used. Positive control (GD2-28ζ) CAR-T cells transduced using EF1a promoter conferred a higher level of CAR specific total-CD3ζ compared to anti-CD19 CAR and anti-claudin-3 CAR expressed using the PGK promoter. Consequently, anti-CD19 CAR and anti-claudin-3 CAR also showed lower levels of phospho-CD3ζ, cytokine release and differentiation in activation and exhaustion phenotype. This reaffirms the efficiency of the vector can be a contributing factor inducing tonic signalling.

Other than the promoter, unlike the positive control (GD2-28ζ) CAR-T cells, the anti-CD19 CAR and anti-claudin-3 CAR-T cells are generated with the 4-1BBζ cytoplasmic domain without the IgG1 CH2-CH3 linker, ameliorating any tonic signalling effect. This data reaffirms that anti-claudin-3 CAR-T cells demonstrate a low level of tonic signalling and antigen independent activation which would otherwise adversely affect CAR-T cell function in vitro.

### Example 3

### Specificity of Anti-Claudin-3 CAR-T Cells to Claudin-Expressing Cell Lines

The aim of these studies was to generate claudin-3-expressing cell lines for the validation of specificity and assessment of functional activity of anti-claudin-3 CAR-T cells. Specifically, the cytotoxicity of anti-claudin-3 CAR-T cells was measured using a CYTOTOX Red assay and the confluency of target cells. In addition, the activation of CAR-T cells was assessed by measuring IFNγ release using a meso scale discovery (MSD) assay after 24 hours of co-culturing with claudin-3-expressing cells. The results suggest that anti-claudin-3 CAR-T cells kill primarily in response to hCLDN3 and there is little to no cytotoxic cross reactivity to other human Claudins.

### Materials and Methods

### Generation and Characterization of RKO-KO Cell Lines, RKO-KO Human CLDN3 GFP and Mouse CLDN3 GFP Cell Lines

RKO-KO cell lines were made by knocking out the CLDN3 gene in colorectal cancer (RKO) cell line using CRISPR-CAS editing technology.

Selected RKO-KO clones were further tested by flow cytometry using anti-CLDN3 antibodies to confirm the lack of detection of extracellular CLDN3 expression. RKO-KO Clone 26.1 was selected as primary clone as parental cell line for the further generation of overexpressing hCLDN3, mCLDN3, and other human claudins cell lines.

RKO-KO cell lines overexpressing human CLDN3 and mouse CLDN3 were generated by transducing the RKO-KO Clone 26.1 cell line with a commercial lentivirus vector (LV) containing either (i) the human CLDN3 gene expressed as a tagged protein with a C-terminal monomeric GFP tag or (ii) the mouse CLDN3 gene expressed as a tagged protein with a C-terminal monomeric GFP tag at MOI 5 and a puromycin selection marker. Flow cytometry was used to assess transduction efficiency (GFP expression).

Monoclonal cell lines were developed by selecting cells expressing High, Medium and Low levels of GFP by single-cell sorting from a heterogeneous population.

Polyclonal RKO-KO cell lines expressing human CLDN family members CLDN4, CLDNS, CLDN6, CLDN8, CLDN9 and CLDN17 cell lines were generated using similar methods as described above by transducing the RKO-KO Clone 26.1 cell line with a commercial Lentivirus encoding either the CLDN4, CLDNS, CLDN6, CLDN8, CLDN9 or CLDN17 gene expressed as a tagged protein with a C-terminal monomeric GFP tag along with a puromycin selection marker.

Characterisation of RKO-KO and RKO-KO Transduced Cell Lines by qPCR

mRNA expression of human claudin 3, 4, 5, 6, 9 and 17 and mouse CLDN3 gene was assessed relative to the housekeeping gene ACTB, in RKO-KO non-transduced cells as well as RKO-KO overexpressing CLDN3, 4, 6, 9, 17 and mCLDN3, respectively. mRNA expression was detected by real time quantitative PCR (RT-qPCR). The RT-qPCR results showed that the transduced CLDNs were overexpressed in the respective RKO-KO cell lines (data not shown).

### CAR-T Cell Thawing and Culture

Where cryo-frozen CAR-T cells were used, the cells were thawed and resuspended with TEXMACS. In some experiments CAR-T cells were enriched as described herein elsewhere.

### Coculture Setup for INCUCYTE Killing Assays

Target cells were resuspended at a density of 2×10⁵ cells/ml in cell culture media and then transferred to the respective wells of the assay plate resulting in 2×10⁴ cells per well. Assay culture plates were then transferred into a humidified INCUCYTE S3 at 36.5° C./5% CO₂ for 24 hours prior to CAR-T cell coculture.

Cell supernatants were removed from respective assay wells and replaced with fresh cell culture media containing 500 nM CYTOTOX Red reagent before the plates transferred into a humidified INCUCYTE S3 at 36.5° C./5% CO₂ prior to effector cell (CAR-T cell) addition. CAR-T cells and untransduced T cells were resuspended in cell culture medium to a density of 2×10⁵ cell/mL and added to the wells. The assay plate was placed in the humidified INCUCYTE S3 at 37° C./5% CO₂. Image acquisition was scheduled at 2-hour intervals over a 6-day time span. Image analysis was conducted to ensure specific visualisation of increase in total red area depicting target cell killing and a total red area mask generated to determine the total area (μm²/image). Normalisation was performed within each donor.

Cytokine Concentration Measurements with MSD

Normalised or enriched T cells were mixed with target cells at 1:1 E:T (effector:target cell, where “effectors” were transduced CAR-T cells) ratios and co-cultured at 37° C., 5% CO₂. After 24 hours plates were centrifuged and supernatants were collected in order to quantify cytokine secretion using a method similar to that described above in Example 2 using the appropriate detection antibodies (Sulfo-tag anti-hIFNγ, Sulfo-tag anti-hTNFa and Sulfo-tag anti-hIL2 Ab).

### Results

### Activation Response of CAR T Cells to RKO KO Expressing Claudins

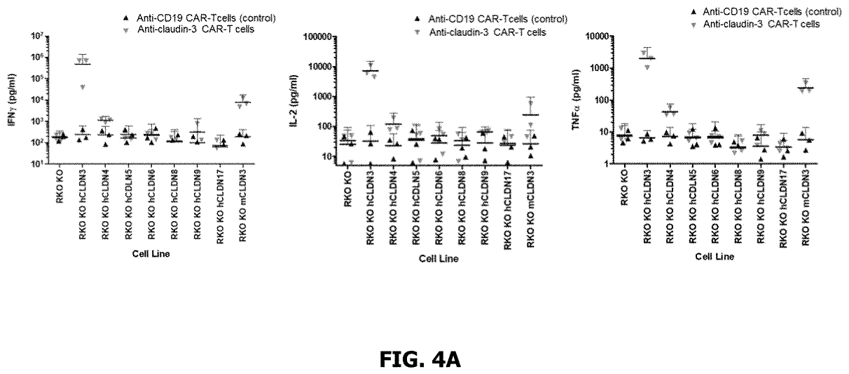

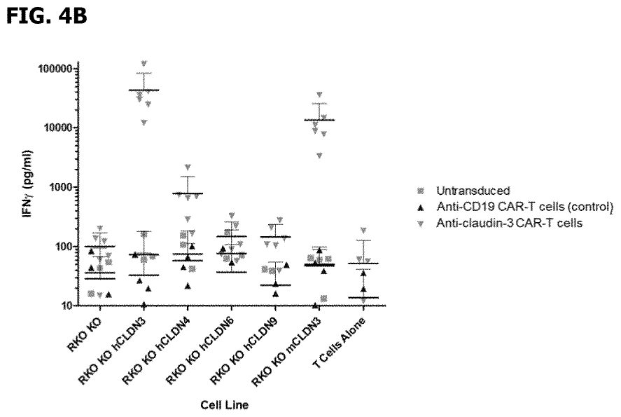

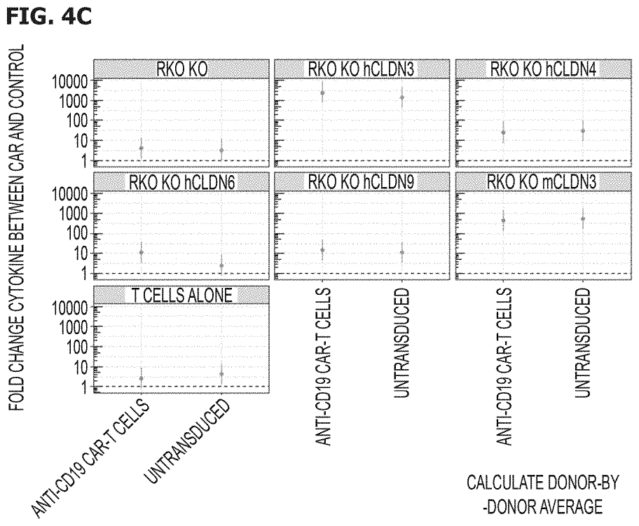

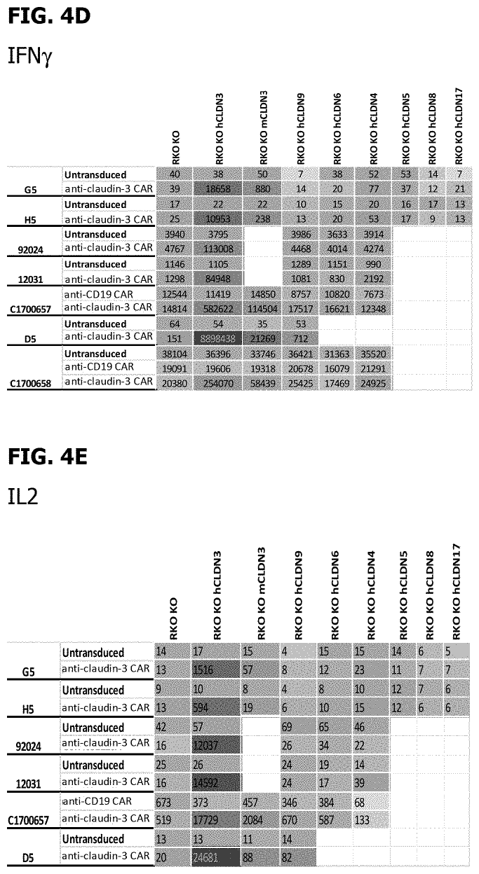

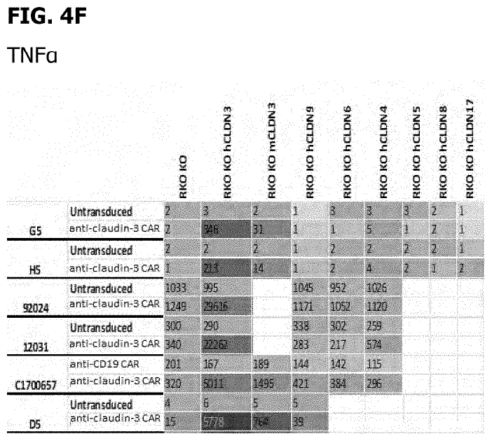

Untransduced or anti-CD19 (control) and anti-claudin-3 CAR-T cells were co-cultured with RKO KO cell lines expressing Claudin proteins closely related to Claudin 3. The supernatant from these co-cultures was then collected and the cytokines of interest were quantified. The data from these experiments are presented in FIGS. 4A-4D and Table 3.

Initially the secretion of IL-2, IFNγ and TNFα by CAR-T cells was studied in response to the full panel of cell lines. Other than the dramatic response of anti-claudin-3 CAR-T cells to hCLDN3 this data, presented in FIG. 4A, shows a small increase in secretion of all three cytokines in response to hCLDN4.

Attention was then focused on the core panel of cell lines (hCLDN4, hCLDN6 and hCLDN9) in an experiment that looked at the IFNγ response in 6 donors (presented in FIGS. 4B and 4C). Once again anti-claudin-3 CAR-T cells responded to hCLDN4 and in this experiment a response to hCLDN9 was also observed. The fold change of IFNγ secreted from anti-claudin-3 CAR-T cells vs anti-CD19 control CAR-T cells in response to hCLDN4 and hCLDN9 was 25 and 14.3 respectively. Although this response is significant, it is put into perspective when compared to a 2495 times higher response in hCLDN3 co-cultures.

The data from these two experiments is further supported by the information presented in FIG. 4D showing cytokine secretion from seven further donors. Only one of these donors showed a response to hCLDN9 but a more consistent response was seen to hCLDN4 (again this was minimal). Some of the donors presented in this figure were then used again (12031, 92024 and C1700657). In each of these cases the data supported the previous results.

Other than the strong response of anti-claudin-3 CAR-T cells to hCLDN3, the most consistent response was to mCLDN3. This could be seen not only in the form of upregulated IFNγ but also IL-2 and TNFα (FIGS. 4A, 4B and 4C). The significance of this response has also been demonstrated in FIGS. 4B and 4C and Table 3 where the IFNγ response of anti-claudin-3 CAR-T cells was 439 times higher than that of anti-CD19 control CAR-T cells.

TABLE 3
Statistical Significance of IFNg Secretion Fold Change from anti-claudin-3 CAR
T cells Compared to Control (anti-CD19 CAR-T cells). CL = confidence intervals.

| | | Estimate | | | |
|---|---|---|---|---|---|
| Target Cell Line | Control | Fold | Lower. CL | Upper. CL | p-value |
| RKO KO | UT¹ | 3.221 | 0.955 | 10.863 | 0.99389 |
| RKO KO | anti-CD19 CAR | 3.987 | 1.182 | 13.447 | 0.941671 |
| RKO KO hCLDN3 | UT | 1353.398 | 401.269 | 4564.731 | <0.00001 |
| RKO KO hCLDN3 | anti-CD19 CAR | 2495.371 | 739.853 | 8416.368 | <0.00001 |
| RKO KO hCLDN4 | UT | 29.188 | 8.654 | 98.444 | 0.000104 |
| RKO KO hCLDN4 | anti-CD19 CAR | 25.074 | 7.434 | 84.570 | 0.000311 |
| RKO KO hCLDN6 | UT | 2.588 | 0.767 | 8.728 | 0.999853 |
| RKO KO hCLDN6 | anti-CD19 CAR | 10.927 | 3.240 | 36.855 | 0.053334 |
| RKO KO hCLDN9 | UT | 11.321 | 3.357 | 38.183 | 0.0445 |
| RKO KO hCLDN9 | anti-CD19 CAR | 14.323 | 4.247 | 48.307 | 0.0121 |
| RKO KO mCLDN3 | UT | 520.965 | 154.461 | 1757.106 | <0.00001 |
| RKO KO mCLDN3 | anti-CD19 CAR | 439.364 | 130.267 | 1481.882 | <0.00001 |
| T cells alone | UT | 4.186 | 1.241 | 14.120 | 0.914967 |
| T cells alone | anti-CD19 CAR | 2.598 | 0.770 | 8.762 | 0.99984 |

¹UT = untransduced

### RKO Cell Killing by CAR-T Cells

To specifically study target cell viability when cultured with CAR-T cells a number of killing assays were performed to determine how T cell activation (measured by cytokine response) translates into cytotoxicity and subsequent target cell apoptosis.

The data collected from this series of experiments is summarised in Table 4 (reported as % Live Cells at the Assay Endpoint). No experiments led to a loss of viability of the either hCLDN4, hCLDN6 or hCLDN9 but occasionally a response to mCLDN3 was observed. This was most evident at 96 hours where RKO KO mCLDN3 reached 71% and 86% or the maximum response when cultured with anti-claudin-3 CAR-T cells from 2 donors.

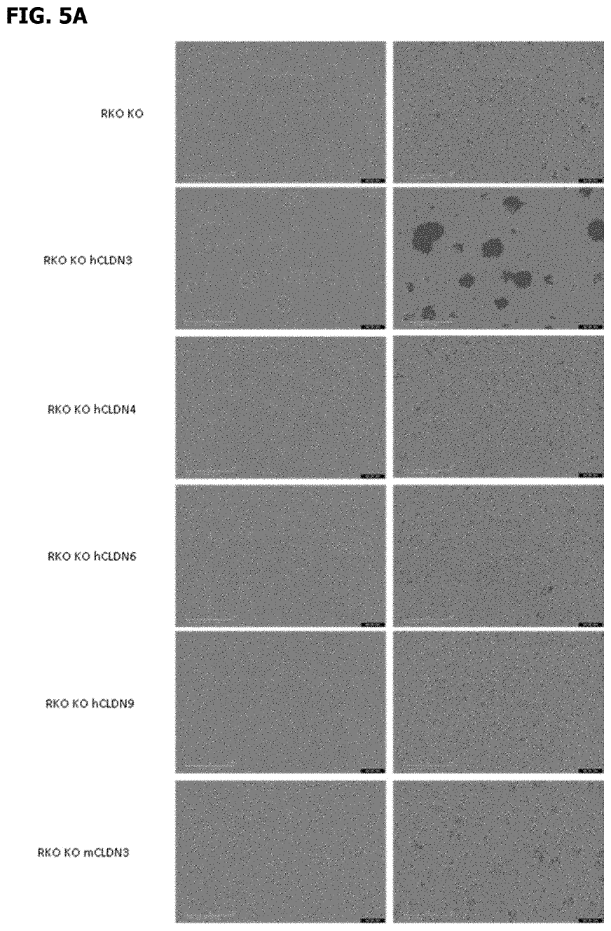

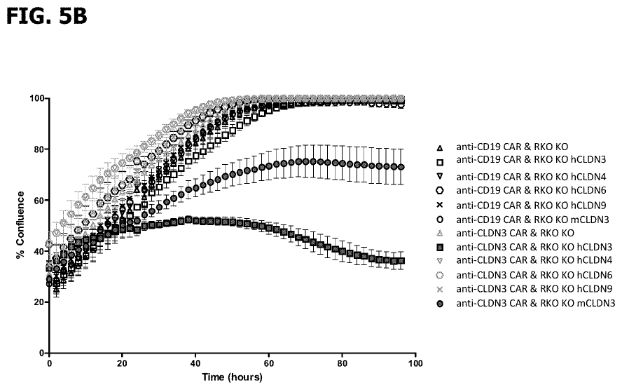

Experiments were conducted in 6 donors and the core panel of cell lines to confirm the CLDN3 specific cytotoxic response of anti-caludin-3 CAR-T cells. Two readouts were used, % Confluence (FIGS. 5A and 5B) and CYTOTOX colour development, or % Live Cells (FIGS. 6A-6C). The first of these, % confluence, indicates cell killing through the change in target cell numbers that are specific to certain co-cultures. The second read-out quantifies the red colour development that occurs when loss of viability leads to the influx of the CYTOTOX dye. Where there was an increase in cell death the CYTOTOX red response was higher converting into a low % Live Cells. Examples of the images used to collect this data are presented in FIG. 5A.

A change in the % confluency was specifically observed when anti-claudin-3 CAR-T cells were cultured with RKO KO expressing either hCLDN3 or mCLDN3 although as with IFNγ secretion the magnitude of the response to hCLDN3 was much greater. This was confirmed by the % Live Cells readout where a significant cytotoxic effect was observed when anti-claudin-3 CAR-T cells were co-cultured with RKO KO mCLDN3 or RKO KO hCLDN3 compared to the control.

TABLE 4
% Live Cells at Assay Endpoint for Polyclonal Cell Lines

| | | | Target Cell Line | | | | | |
|---|---|---|---|---|---|---|---|---|
| Endpoint | | | | RKO KO | RKO KO | RKO KO | RKO KO | RKO KO |
| (time) | Donor | Construct | RKO KO | hCLDN3 | mCLDN3 | hCLDN9 | hCLDN6 | hCLDN4 |
| 70 hrs | D5 | Anti-claudin-3 | 95.3 | 0 | 97.7 | 96.3 | | |
| | | CAR | | | | | | |
| | | Untransduced | 97.3 | 99.7 | 99 | 99.7 | | |
| 40 hrs | 92024 | Anti-claudin-3 | 97.7 | 16.3 | 89 | 96.7 | 96.3 | |
| | | CAR | | | | | | |
| | | Untransduced | 96.3 | 96 | 95.3 | 95 | 95 | |
| 96 hrs | C1700979 | Anti-claudin-3 | 98 | 29* | 71 | 92.7 | 96 | |
| | | CAR | | | | | | |
| | | Untransduced | 92.3 | 95.7* | 93 | 92.3 | 91.7 | |
| | C1700980 | Anti-claudin-3 | 101 | 57.3* | 86 | 100.7 | 100.3 | |
| | | CAR | | | | | | |
| | | Untransduced | 105 | 101.3* | 101.7 | 106 | 102 | |
| 90 hrs | 0372470 | Anti-claudin-3 | 107.7 | −6* | 94.5 | | | 94.7 |
| | | CAR | | | | | | |
| | | Anti-CD19 | 102.7 | 104* | 99.3 | | | 108.7 |
| | | control CAR | | | | | | |
| 120 hrs | 0372470 | Anti-claudin-3 | 92 | 20.3 | 81.7 | | | 90.7 |
| | | CAR | | | | | | |
| | | Anti-CD19 | 84.7 | 91.3 | 85 | | | 90.7 |
| | | control CAR | | | | | | |

*Co-culture with RKO KO hCLDN3 L14

Anti-Claudin-3 CAR-T Cells Cross-React with mCLDN3-Expressing Cells

A number of relevant experiments can be performed using mouse tissues and therefore it is useful to understand how anti-claudin-3 CAR-T cells react to mCLDN3. The experiments performed herein consistently show that anti-claudin-3 CAR-T cells are activated by mCLDN3 expression leading to secretion of IFNγ, TNFα and IL-2. Although not as high as the response to hCLDN3, the activated T cells do exhibit a significant cytotoxic response shown by loss of target cells.

Most of this data has been performed with a cell line expressing different levels of mCLDN3 expression, however in order to understand the response further a few lines expressing high or low levels of mCLDN3 were also used. In these conditions an activation response was only observed when the anti-claudin-3 CAR-T cells were cultured with RKO KO cells expressing high mCLDN3.

### Anti-Claudin-3 CAR-T Cells Do Not Kill in Response to Other Human Claudin Proteins

No activation of anti-claudin-3 CAR-T cells was observed in response to hCLND6, hCLDN5, hCLDN8 or hCLDN17 in any of the experiments presented herein. There was, however, some reactivity to hCLDN4 and hCLDN9 shown by secretion of cytokines. Although a significant activation response is described in FIG. 4A it does not compare to the response of anti-claudin-3 CAR-T cells to hCLDN3 and mCLDN3. Notably, this does not translate to any killing response and this suggests that anti-claudin-3 CAR-T cells do not significantly kill in response to any human Claudins other than hCLDN3.

The data presented herein shows that anti-claudin-3 CAR-T cells are activated, at least in part, by hCLDN4 and hCLDN9. This response is significantly less than the response to hCLDN3 and does not translate to a cytotoxic effect or cell death. The anti-claudin-3 CAR-T cells do cross react to mCLDN3 however and partial killing of the target cells has been described. This data suggests that anti-claudin-3 CAR-T cells kill primarily in response to hCLDN3 and there is little to no cytotoxic cross reactivity to other human Claudins.

### Example 4

### Cytotoxicity of Anti-Claudin-3 CAR-T Cells on Claudin-3 Positive Tumour Cell Lines

The aim of this study is to show the expression and cytotoxic potency of anti-claudin-3 CAR based on the propensity of T cells expressing this construct to specifically kill human Claudin-3 (hCLDN3) expressing target cells. These data demonstrate that anti-claudin-3 CAR-T cells were able to secrete IFNγ and kill hCLDN3-expressing cancer cell lines derived from colorectal, breast and pancreatic cancer.

### Materials and Methods

### Enrichment of LNGFR Positive CAR-T Cells by MACSQuant Tyto Sorting

Cells were washed in buffer, stained with LNGFR PE antibodies at a dilution of 1:50 for 30 minutes at 4° C., and then sorted using MACSQuant Tyto sorting according to the manufacturer's instructions. All CAR-T cells used in experiments described herein had a purity of at least 90% LNGFR positivity.

### Quantifying CAR Molecules on the T Cell Surface

1×10⁵ cells and 50 μL Bangs Labs Quantum Simply Cellular beads were resuspended in anti-LNGFR PE at a 1:20 dilution or anti-hCLDN3 PE at a 1:50 dilution and incubated for 30 minutes at 4° C. Cells and beads were washed twice and the PE signal of cells and beads were then measured on the CytoFLEX S machine.

### Co-Culture Setup for Cell Killing Assays

Co-cultures for INCUCYTE killing assays were set up as described above in Example 3, except that the INCUCYTE Zoom was used rather than INCUCYTE S3.

Co-cultures for XCELLIGENCE killing assays were set up by seeding target cells in a cell culture plate at a density of 25,000 cells/well and cultured in the cell culture incubator of the XCELLIGENCE Real-Time Cell Analysis (RTCA) instrument. Approximately 20 hours post seeding, effector cells were added at a ratio of 0.5:1 or 1:1 CAR-T cells to target cells and placed back in the cell culture incubator. The target cells used were the cancer cell lines shown in Table 5 below.

TABLE 5
Cancer cell lines:

| Target cell line | Indication |
|---|---|
| RKO-KO | Colorectal cancer |
| RKO-KO hCLDN3 | Colorectal cancer |
| RKO-KO hCLDN3 L14 (sorted for low hCLDN3 | Colorectal cancer |
| expression) | |
| RKO-KO hCLDN3 H12 (sorted for high hCLDN3 | Colorectal cancer |
| expression) | |
| HT-29-LUC | Colorectal cancer |
| COLO-205 | Colorectal cancer |
| COLO-320DM | Colorectal cancer |
| DLD-1 | Colorectal cancer |
| HCT-15 | Colorectal cancer |
| SW403 | Colorectal cancer |
| SW480 | Colorectal cancer |
| SW620 | Colorectal cancer |
| SW48 | Colorectal cancer |
| HCT116 | Colorectal cancer |
| HCT116-p53het | Colorectal cancer |
| T84 | Colorectal cancer |
| LS174-T | Colorectal cancer |
| HCC1954 | Breast cancer |
| MDA MB468 | Breast cancer |
| HCC1937 | Breast cancer |
| MT-3 | Breast cancer |
| SUM185 | Breast cancer |
| HCC38 | Breast cancer |
| AsPC-1 | Pancreatic cancer |
| Capan-1 | Pancreatic cancer |
| Capan-2 | Pancreatic cancer |
| Panc-1 | Pancreatic cancer |
| Panc02.03 | Pancreatic cancer |
| Panc08.13 | Pancreatic cancer |
| BxPC3 | Pancreatic cancer |
| HPAC | Pancreatic cancer |
| HuP-T4 | Pancreatic cancer |
| MiaPaCa2 | Pancreatic cancer |
| HPAF-II | Pancreatic cancer |

Cytokine Concentration Measurements with MSD

Target cell lines were resuspended and 2 or 2.5×10⁴ cells were then seeded into a 96-well plate. Normalised or enriched T cells were then added to the plate at 1:1 E:T (effector: target cell, where “effectors” were transduced CAR-T cells) ratios and co-cultured at 37° C., 5% CO₂ for 24-48 hours After co-culturing, the plates were centrifuged, and supernatants were collected in order to quantify cytokine secretion using the appropriate detection antibodies as described above in Example 2.

### Results

Quantification of CAR Expression and hCLDN3 Expression

Expression of CAR molecules on the T cell surface is a requirement for CAR function. LNGFR expression was measured as a surrogate for CAR expression. LNGFR and CAR molecules should be translated at a 1 to 1 ratio but due to potential differences in protein stability, quantifying LNGFR is therefore only an estimate of the CAR molecule number.

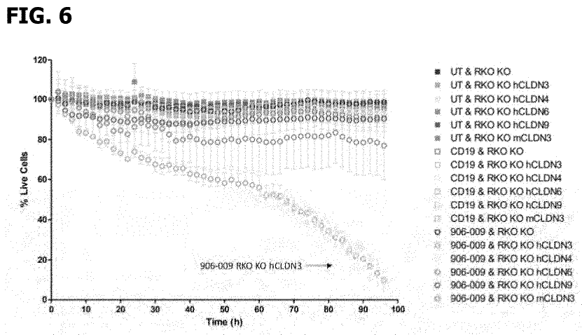

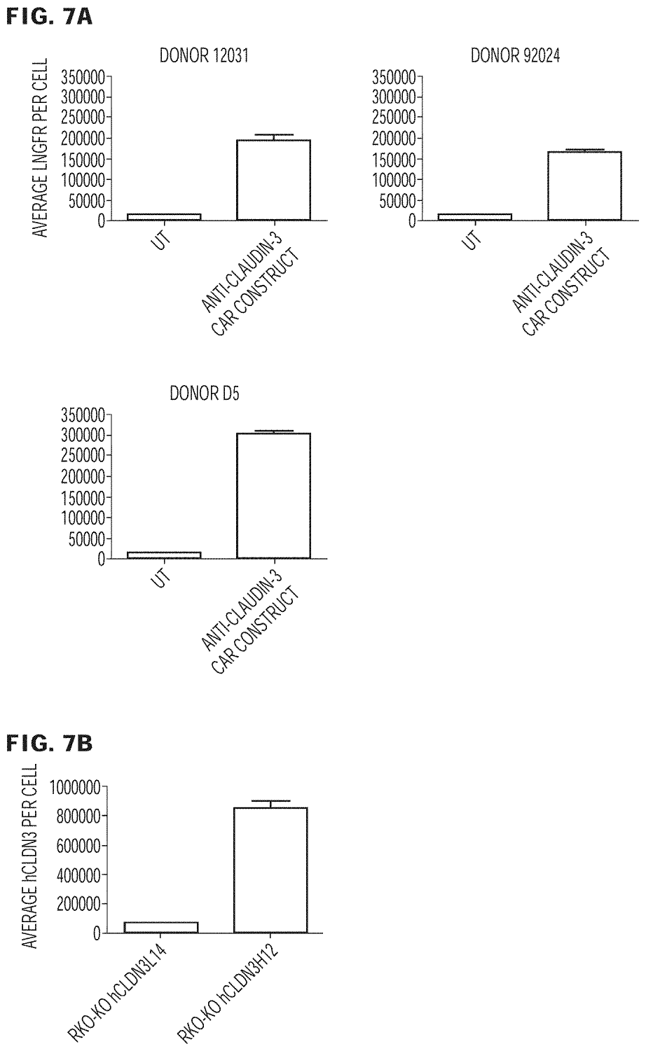

Untransduced cells (UT) only showed a very low signal for LNGFR expression; between 10,000 and 20,000 (FIG. 7A), which was considered to be the background signal.

Donors 12031 and 92024, had an average of 190,000 and 166,000 LNGFR molecules on the surface and donor D5 had 301,000 molecules on the surface. This difference was potentially due to variations in T-cell generation.

As described herein before, a hCLDN3 knock out RKO cell line (RKO-KO) was generated and used as a negative control for functional assays. Killing of RKO-KO cells expressing hCLDN3 either at varying levels (polyclonal RKO-KO hCLDN3 cell line; not single-cell sorted) or sorted for high (RKO-KO hCLDN3 H12) or low (RKO-KO hCLDN3 L14) expression is an indication of CAR potency. Differences in hCLDN3 expression was confirmed by quantifying expression with Quantum Simply Cellular beads and anti-CLDN3 PE (FIG. 7B). RKO-KO cells showed a signal below the bead level and their hCLDN3 level was therefore considered as 0. RKO-KO hCLDN3 L14 low cells had an average number of 80,000 hCLDN3 molecules on the surface whereas RKO-KO hCLDN3 H12 high cells had an average number of 800,000 hCLDN3 molecules on the surface. Thus, these two cell lines had a 10-fold difference in hCLDN3 expression.

### Cytotoxicity by CAR-T Cells

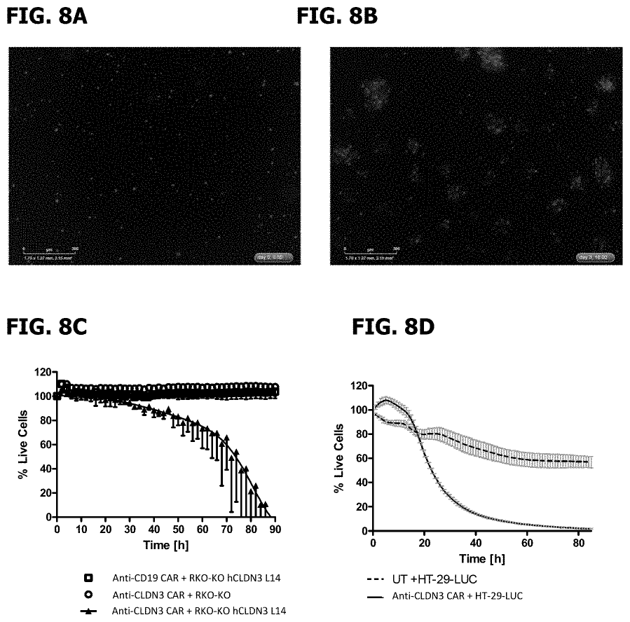

FIGS. 8A-8D show an example IncuCyte killing assay with 90 hours incubation. Example images of LNGFR enriched anti-CD19 control CAR-T cells (FIG. 8A) or anti-claudin-3 CAR-T cells (FIG. 8B) incubated with RKO-KO cells expressing hCLDN3 at a low level or RKO-KO cells are shown and corresponding killing curves show cytotoxicity with the anti-claudin-3 CAR-T cells but not the control (FIG. 8C). Signal development, measured by the area of the red dye, was visible when anti-claudin-3 CAR-T cells were incubated with the target cell line. Quantified and normalised data is shown in FIG. 8C which shows that the anti-claudin-3 CAR-T cells incubated with target-negative cells did not lead to cell lysis but anti-claudin-3 CAR-T cells incubated with hCLDN3-expressing RKO cells led to complete target cell killing while anti-CD19 control did not.

Table 6 shows the times required to reach 50% of the maximum response indicating 50% of target cell killing. These values revealed differences between assays that may be explained by differences in hCLDN3 expression but this was not consistent across experiments. While in some experiments, 50% of the maximum response was achieved after 24 to 41 hours, other experiments showed 50% of the maximum response after 51 hours up to 74 hours. Other data (not shown) for which T cells and target cells were treated equally for all 6 donors showed a reduced spread with 4 out of 6 donors between 80 and 86 hours, one donor with 68 hours and one donor did not reach 50% cytotoxicity within the 96 hours of the experiment. Differences in killing kinetics cannot be explained but importantly, all experiments led to full target cell lysis.

Similar cytotoxicity results were obtained with the xCELLigence method on the Claudin-3 expressing cell line HT-29-LUC (FIG. 8D). 100% cytotoxicity was observed at a 1:1 ratio with three donors and 100% cytotoxicity was reached after 80 hours, 30 hours and 50 hours. 100% cytotoxicity at a 1:2 ratio (Effector:Target) was achieved after 40 hours and 60 hours. Co-cultures performed in the same manner as the xCELLigence experiments were analysed for cytokine secretion by MSD (data not shown). Interferon γ (IFNγ) was detected for a donor that was frozen and thawed and a donor that was used fresh when T cells were co-cultured with HT-29-LUC but not when they were incubated without a cell line.

Secretion of IFNγ by untransduced cells or anti-CD19 control CAR-T cells in response to Claudin-3 expressing cells was at the same level as in response to cells not expressing Claudin-3. These results suggest that no non-specific cytokine secretion was observed. Secretion by anti-claudin-3 CAR-T cells in response to target cells not expressing Claudin-3 led to similar background cytokine secretion as the negative controls, indicating that anti-claudin-3 CAR does not recognise other molecules on the target cells.

Culturing anti-claudin-3 CAR-T cells with Claudin-3 expressing target cells led to IFNγ secretion in all 8 conducted experiments. The actual cytokine amount measured differed between experiments but in all cases, the specific cytokine secretion by anti-claudin-3 CAR-T cells in response to its target was at least 100-fold increased compared to anti-claudin-3 CAR-T cells in response to target-negative cells.

TABLE 6
Time to Reach 50% of Max Response (Complete Target
Cell Killing) of RKO cells Expressing CLDN3

| | | Time to 50% of max |
|---|---|---|
| Donor | CLDN3 expression | response [h]* |
| D5 | poly | 32 |
| 92024 | poly | 22 |
| 90144 | high | 38 |
| | low | 27 |
| C1700979 | high | 53 |
| | low | 54 |
| C1700980 | high | 56 |
| | low | 63 |
| 372470 | low | 74 |
| PR19t133635 | low | 68 |
| PR19K133652 | low | 81 |
| PR19T133651 | low | 84 |
| PR19K133900 | low | 80 |
| PR19C133904 | low | 86 |
| PR19W133916 | low | not reached |

*These values were determined from 3 replicates

### Titration of RKO-KO CLDN3-Expressing Target Cells

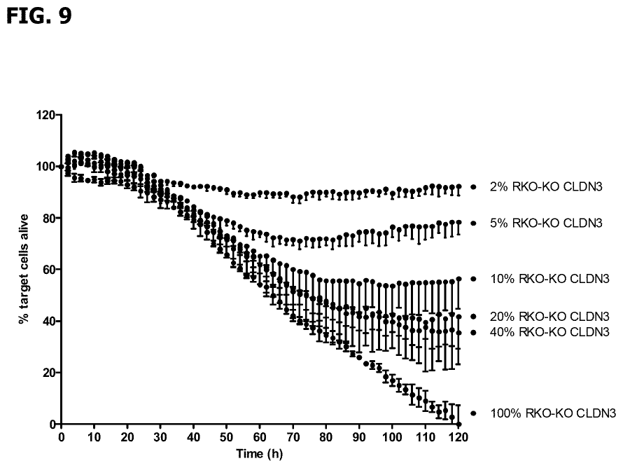

Maximum responses (complete target cell killing) differ between cell lines but can also indicate partial target cell killing. This may be due to insufficient cytotoxic effector functions or occurs when not all tumour cells express the target. To determine differences in maximum responses and to confirm that partial killing is achievable in IncuCyte assays, a titration of target cells was performed. RKO-KO and RKO-KO hCLDN3 polyclonal cells were mixed at varying ratios and incubated with anti-claudin-3 CAR-T cells. No signal was visible when only 2% of the RKO-KO cells expressed hCLDN3 but increases in the signal were visible with a higher proportion of hCLDN3 expressing cells (FIG. 9). These results indicate that partial killing can be achieved which are visible at differences in the maximum response if comparing the same target cell line.

Relationship Between hCLDN3 Expression and CAR-T Activation

Understanding how CAR-T cells respond to target density can help to predict the effect of target expression in an alternate setting. Experiments were performed to study the relationship between T cell activation and target expression.

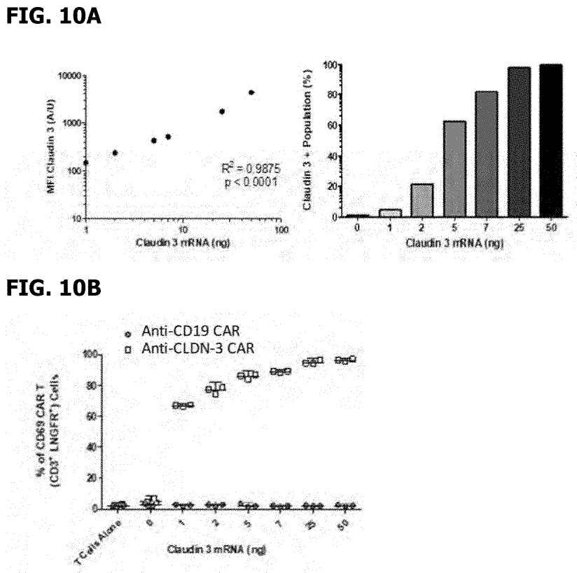

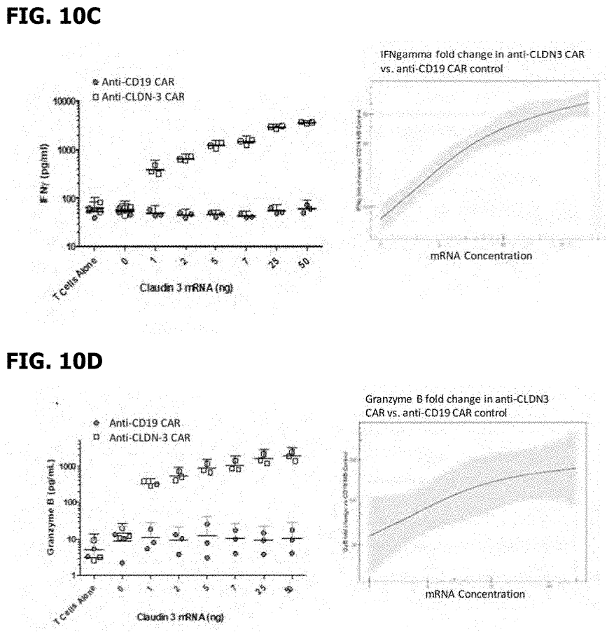

hCLDN3 expression was tightly controlled by nucleofecting a hCLDN3 Knock-out (KO) cell line (RKO-KO) with hCLDN3 mRNA (produced in vitro from linearized plasmid using the mMESSAGE mMACHINE T7 Ultra kit) in order to create a gradient of hCLDN3 expression (FIG. 10A). This allowed the study to be performed in a well-defined system independent of variable factors (such as other biomarkers) that could affect the T cell response to hCLDN3. After confirming the expression of hCLDN3, the target cells were cultured with CAR-T cells and the activation response was assessed by either CD69 expression (measured by flow cytometry as described in previous examples) or cytokine secretion (IFNγ/Granzyme B).

In each experiment activation specific to anti-claudin-3 CAR-T cells and hCLDN3 expression was observed. This hCLDN3 dependent activation presented a correlation between target expression and T secretion of IFNγ and Granzyme B (FIGS. 10C and 10D and Table 7). Although no statistical analysis was performed there was also a clear relationship between the size of the hCLDN3 positive population and CD69 expression that plateaued at 100% target expression (FIG. 10B). Overall, this shows a relationship between target expression and CAR-T activation.

In the experiments described above, RKO-KO were nucleofected with a gradient optimised that created a target gradient with decreasing hCLDN3 positive populations. In another experiment, the effect of decreasing expression of hCLDN3 within the entire cell population was studied (Table 8). In this case the data suggested that with increasing hCLDN3 expression there was decreasing T cell activation (as shown by IFNγ secretion). CD69 expression remained consistent between each expression level.

TABLE 7
Significance of Cytokine Secretion at Varying Levels of Target Expression

| Cytokine | Linear Contrast | Estimate | Lower CL | Upper CL | p-value |
|---|---|---|---|---|---|
| IFNγ | Anti-claudin3 vs anti-CD19 CAR | 2.070 | 1.508 | 2.840 | 0.0001 |
| | 2.5 ng vs 1 ng | | | | |
| | Anti-claudin3 vs anti-CD19 CAR | 3.410 | 2.423 | 4.799 | 0.0001 |
| | 5 ng vs 1 ng | | | | |
| | Anti-claudin3 vs anti-CD19 CAR | 4.812 | 3.434 | 6.744 | 0.0001 |
| | 10 ng vs 1 ng | | | | |
| | Anti-claudin3 vs anti-CD19 CAR | 6.427 | 4.746 | 8.703 | 0.0001 |
| | 25 ng vs 1 ng | | | | |
| | Anti-claudin3 vs anti-CD19 CAR | 7.519 | 5.207 | 10.859 | 0.0001 |
| | 50 ng vs 1 ng | | | | |
| Granzyme B | Anti-claudin3 vs anti-CD19 CAR | 1.890 | 0.843 | 4.237 | 0.1185 |
| | 2.5 ng vs 1 ng | | | | |
| | Anti-claudin3 vs anti-CD19 CAR | 3.029 | 0.829 | 11.059 | 0.0913 |
| | 5 ng vs 1 ng | | | | |
| | Anti-claudin3 vs anti-CD19 CAR | 4.253 | 1.007 | 17.959 | 0.0489 |
| | 10 ng vs 1 ng | | | | |
| | Anti-claudin3 vs anti-CD19 CAR | 5.495 | 1.470 | 20.539 | 0.0127 |
| | 25 ng vs 1 ng | | | | |
| | Anti-claudin3 vs anti-CD19 CAR | 6.191 | 1.551 | 24.705 | 0.0112 |
| | 50 ng vs 1 ng | | | | |

Estimates are reported as fold change in IFNγ levels between anti-claudin-3 and anti-CD19 control CAR-T cells.
CL = Confidence Intervals.

TABLE 8
Summary of Data Studying T cell Response
to a Gradient of Target Expression

| Claudin 3 mRNA | IFNγ Secretion | |
|---|---|---|
| (ng) | (pg/mL) | CD69% |
| 0 | 36 | 5.88 |
| 250 | 4115 | 74.1 |
| 500 | 3349 | 70.5 |
| 1000 | 3271 | 75.6 |
| 2000 | 2603 | 70.1 |
| 0 | 19 | 2.73 |
| 1 | 539 | 24 |
| 2 | 834 | 35.3 |
| 6 | 891 | 43.9 |
| 7 | 2324 | 47.2 |
| 50 | 8469 | 76.8 |

Screening of Cancer Cell Lines from Three Different Indications for hCLDN3 Expression and T Cell Activation

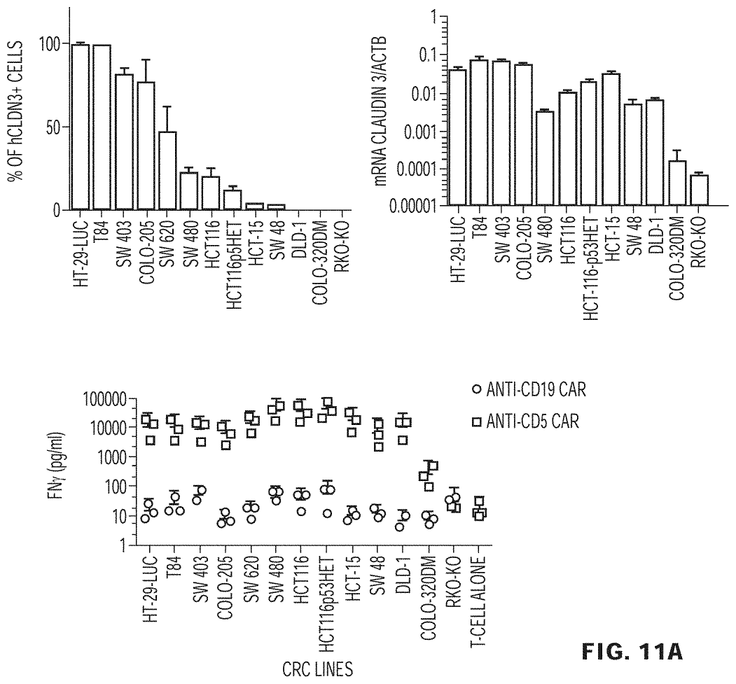

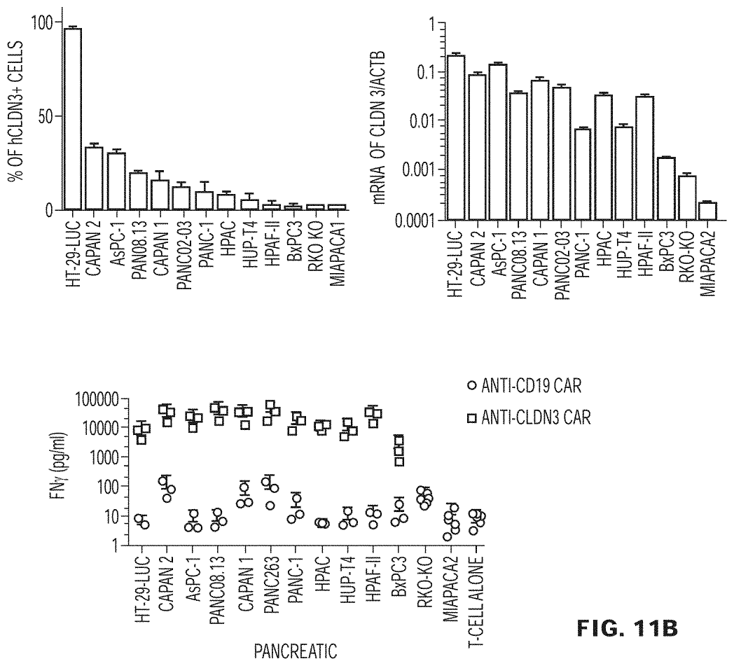

In the studies described above, the response of anti-claudin-3 CAR-T cells to only one colorectal cancer cell line expressing different levels of exogenous hCLDN3 was investigated. Here, the response of anti-claudin-3 CAR-T cells to a panel of 31 cancer cell lines from three primary indications (colorectal, pancreatic and breast cancer) expressing endogenous hCLDN3 was assessed including HT-29-LUC and RKO-KO cell lines as positive and negative control, respectively. Cell lines from colorectal (FIG. 11A), pancreatic (FIG. 11B) and breast (FIG. 11C) cancer were first screened for hCLDN3 expression by flow cytometry and real-time quantitative PCR (RT-qPCR). Different levels of hCLDN3 expression were detected on all cell lines (FIGS. 11A-11C, left) ranging from 0% to 100%. Interestingly, in partially positive cell lines, such as SW403 or SW480, a shift of the total population when cells were incubated with hCLDN3 antibody instead of a well-defined positive and negative population was seen. Expression levels of the hCLDN3 gene were quantified by RT-qPCR in the same panel of cell lines (FIGS. 11A-11C, middle) and a similar pattern was seen in the amount of CLDN3 mRNA. Small amounts of hCLDN3 mRNA could be detected even in the negative cell line control (RKO-KO) due to the CRISPR methodology. When comparing RT-qPCR and flow cytometry data, it was observed that HT-29-LUC, T84 and SW403 were the highest Claudin 3 expressers for both assays, while RKO-KO and COLO320-DM were the lowest expressers.

Anti-claudin-3 CAR-T cell activation was also studied after co-culture with the selected cancer cell lines using anti-CD19 as a negative CAR control (FIGS. 11A-11C, right). Importantly, all the cell lines expressing hCLDN3 activated anti-claudin-3 CAR-T cells, as shown by the high levels of IFNγ, whereas no response was seen from anti-claudin-3 CAR-T cells co-cultured with RKO-KO. Nevertheless, some cell lines that showed no detectable expression of hCLDN3 by flow, COLO320-DM (0.068%), DLD-1 (0.347%), HC1954 (0.55%) and BxPC3 (1.95%), were also able to activate anti-claudin-3 CAR-T cells. One possible explanation is the limit of detection of the commercial antibody used.

All of the hCLDN3 positive cell lines were able to activate anti-claudin-3 but not anti-CD19 control CAR-T cells. However, no clear correlation was seen between anti-claudin-3 CAR-T activation and hCLDN3 expression levels, either by flow cytometry or RT-qPCR.

Killing of Cancer Cell Lines from Three Indications

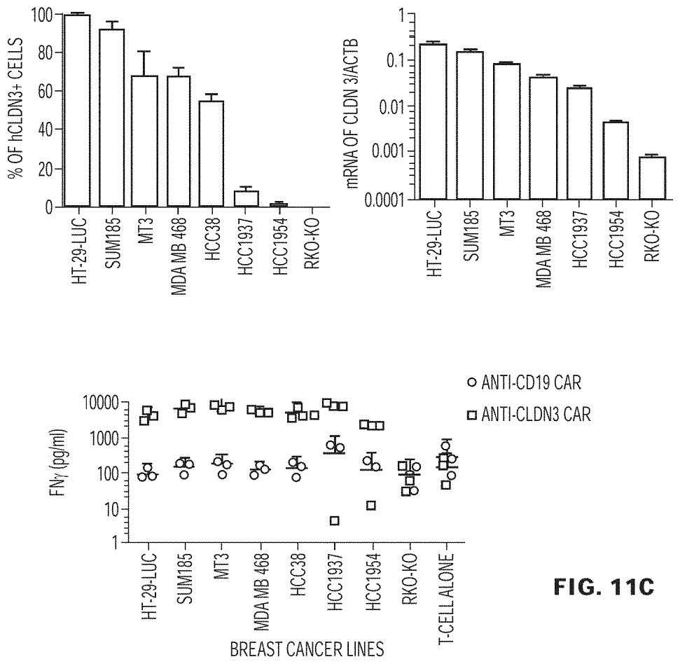

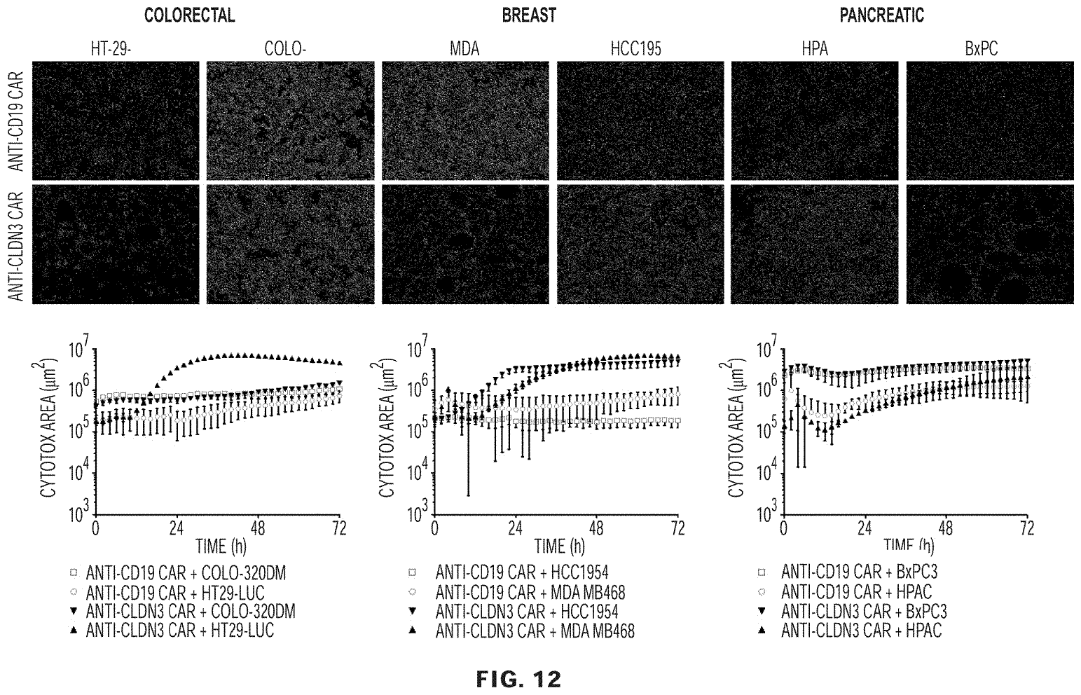

To study the functionality of anti-claudin-3 CAR-T cells against a wider range of tumours several cancer cell lines with different levels of hCLDN3 expression were chosen to perform killing assays. Table 9 shows a summary of three IncuCyte experiments conducted with three donors each. As the results were quite consistent between donors, Table 9 summarises three donors per experiment. Cell lines from the three indications were killed by anti-claudin-3 CAR-T cells showing that there is potential for this CAR to be used for several indications. HT-29-LUC complete killing was also visible (data not shown). Only one colorectal cancer line, COLO-320DM, was not killed by anti-claudin-3 CAR-T cells. Three cell lines, HCC1954, BxPC3 and HPAC, were partially killed. Partial killing was visible in the microscopy images as apoptotic cells or holes in the cell layer whereas the obtained signal was very low or absent as visible in the raw data (FIG. 12).

TABLE 9
Cytotoxicity of anti-claudin-3 CAR-T cells Towards Target
Cells Derived from Colorectal, Breast or Pancreatic Cancer

| | | Experiment | Experiment | Experiment |
|---|---|---|---|---|
| Indication | Cell line | 1 | 2 | 3 |
| Colorectal | SW403 | c | N/A | N/A |
| cancer | SW480 | c | N/A | N/A |
| | SW620 | c | N/A | N/A |
| | HT29 | c | N/A | c |
| | COLO-320DM | N/A | N/A | n |
| | DLD1_col | N/A | N/A | c |
| | HCT15 | N/A | N/A | c |
| Breast | HCC1954 | N/A | p | p |
| cancer | MDA MB468 | N/A | c | c |
| | MT-3 | N/A | c | N/A |
| | HCC1937 | N/A | N/A | c |
| Pancreatic | ASPC1 | c | N/A | N/A |
| cancer | CAPAN2 | c | N/A | N/A |
| | PANC02.03 | c | N/A | N/A |
| | BxPC3 | N/A | N/A | p |
| | HPAC | N/A | N/A | p |
| | HUPT4 | N/A | N/A | c |

n no killing
p partial killing (killing visible in images but not quantifiable)
c complete killing
N/A no data obtained

### LNGFR Molecule Quantification Indicates Robust CAR Expression

LNGFR numbers between 166,000 and 301,000 were detected. Assuming that CAR expression and LNGFR expression are comparable and considering that an average CD8⁺ T cell has 50,000 T cell receptors on the surface, hCLDN3 CAR abundance on the T cell surface is estimated to be sufficient for T cell activation.

### Anti-Claudin-3 CAR-T Cell Killing Kinetics Vary Between Experiments

One aspect for determining CAR-T cell potency is how rapidly cytotoxicity is induced. This was analysed by determining after how many hours 50% of the maximum response was achieved. These results varied between experiments which makes drawing conclusions difficult. It can, however, be stated that in all cases, full target cell killing was achieved which is an important aspect for determining CAR potency.

### Anti-Claudin-3 CAR-T Cells Specifically Kill CLDN3-Expressing Cells

Anti-claudin-3 CAR-T cells are specific for target cells expressing hCLDN3. Evidence for this stems from experiments in which RKO cells where endogenous hCLDN3 was knocked out (RKO-KO), were not killed by anti-claudin-3 CAR-T cells. In contrast, cell lines that showed hCLDN3 expression were killed by anti-claudin-3 CAR-T cells. In addition, if RKO-KO and RKO-KO overexpressing exogenous hCLDN3 cells were mixed, only partial cytotoxicity was detected, showing that even T cells are specifically activated only by target cell expressing hCLDN3. These results suggest that anti-claudin-3 CAR-T cell activity was specific for hCLDN3.

Increasing Anti-Claudin-3 CAR-T Cell Activation Correlates with Increase of Claudin 3 Expression

To investigate which patient population could be responsive to anti-claudin-3 CAR-T cell treatment, it is useful to have an understanding of the activation threshold of the CAR. As described above a controlled model was used to specifically study the relationship between target expression and T cell activation. By decreasing the expression of the target to a point where it could no longer be detected by flow cytometry, the aim was to define the target expression levels necessary to activate anti-claudin-3 CAR-T cells.

IFNγ secretion is a key measurement of the T cell activation response and CD69 is commonly used a marker of activation. Quantification of Granzyme B, an integral inducer of target cell apoptosis was also used to provide clear evidence of the relationship between target expression and CAR-T cell cytotoxicity. The upregulation of these indicators when RKO-KO were nucleofected with 1 ng of Claudin 3 mRNA leading to a 5% positive population, clearly show the sensitivity of anti-claudin-3 CAR-T cells. Even when there is low antigen availability the CAR-T cell response is efficacious. The presence of Granzyme B within the culture media suggests that target cell apoptosis occurred but without studying the target cells themselves this cannot be stated unequivocally.

Due to the detection limits of the antibody, it is difficult to state whether the expression observed is a small population of Claudin 3 expressing cells or a 100% target positive population with low expression. Any target cell apoptosis observed could not be correlated to an accurate quantification of the Claudin positive population.

Despite the observation of a clear relationship between the antigen expression and anti-claudin-3 CAR-T cell activation, a threshold of activation could not be established mainly due to the limitations of antigen detection. Importantly, however, within this artificial system there was a dose response of anti-claudin-3 CAR-T cells to Claudin 3 expression.

Anti-Claudin-3 CAR-T Cell Activation is Observed when Co-Cultured with a Panel of Cell Lines from Different Indications

In order to investigate the efficacy of anti-claudin-3 CAR-T cells with a broader panel of target cells, 31 cancer cell lines, including colorectal, pancreatic and breast cancer, were screened for Claudin 3 expression both at mRNA and protein level. Notably, all cell lines expressing hCLDN3 could activate anti-claudin-3 CAR-T cells, which expands the efficacy of this CAR to a broader panel of cancer indications.

Anti-Claudin-3 CAR-T Cells have the Potential to Kill Tumour Cells Derived from Different Indications

Activation of anti-claudin-3 CAR-T cells might not result in killing of the target cells and therefore cytotoxicity was tested for three tumour indications. Complete or partial killing was visible for most of the target cell lines tested, which was in agreement with the IFNγ release of the CAR-T cells (FIGS. 11A-11C). Only the colorectal cancer line COLO-320DM was not killed by anti-claudin-3 CAR-T cells, most likely because it showed no hCLDN3 expression even though it was able to partially activate CAR-T cells.

Anti-claudin-3 CAR is expressed on T cells at a level that suggests it can redirect T cell activity to hCLDN3-expressing tumour cells. It specifically kills target cells with exogenous expression of hCLDN3, while sparing cells where the antigen was removed via CRISPR/Cas9 technology. Anti-claudin-3 CAR-T cells were able to secrete IFNγ and kill hCLDN3-expressing cancer cell lines derived from colorectal, breast and pancreatic cancer, although an activation threshold was not able to be defined due to the limitation of the detection reagents. This suggests that the CAR is able to redirect T cells to several types of cancer. Finally, no clear correlation was seen between hCLDN3 expression in these cell lines and IFNγ release levels, probably due to the different biological characteristics of these cell lines.

### Example 5

### Repeated Antigen-Dependent Stimulation to Evaluate the Long-Term Antitumor Activity of Anti-Claudin-3 CAR-T Cells In Vitro

The aim of this study was to analyse expression, cytokine secretion and cytotoxic potency of six anti-claudin-3 CAR constructs based on the same scFv variant. In addition, the long term functionality of anti-claudin-3 CAR-T cells was assessed by repeated antigen stimulation.

### Materials and Methods

Cloning of scFvs Directed Against Claudin-3 into Three Backbones (L, S, XS) of the Miltenyi Biotec CAR Spacer Library

The plasmids encoding the scFv variants were prepared in two different orientations: heavy-light (V_{H}-V_{L}) and light-heavy (V_{L}-V_{H}). The scFvs were cloned into three backbones of the Miltenyi Biotec CAR spacer library, differing in the spacer length, long (L, hIgG4 H-CH2-CH3), short (S, hCD8) and very short spacer (XS, hIgG4 hinge) resulting in 6 different constructs:

| Spacer¹ | scFv orientation H-L | scFv orientation L-H |
|---|---|---|
| Long (L) | 906_002 | 906_007 |
| Short (S) | 906_004 | 906_009 |
| Very Short (XS) | 906_005 | 906_010 |

¹Spacer (or hinge domain): domain between the extracellular domain and the transmembrane domain.

PBMC Preparation and Isolation of Pan T cells

PBMCs were isolated from Buffy coats obtained from two healthy donors. T cells were isolated untouched using the Pan T cell human isolation kit. Isolation of Pan T cells was performed according to the manufacturer's protocol with 2×10⁸ white blood cells. The T cells were resuspended in TEXMACS medium containing IL-7 (10 ng/mL), IL-15 (10 ng/mL) and T Cell TRANSACT human (1:100) and adjusted in a concentration of 1×10⁶ cells/mL.

### Generation and Expansion of CAR-T Cells

Pan T cells were seeded in a concentration of 1×10⁶ cells/ml and 2 ml per well onto a 24-well plate (see above). One day after activation of the T cells with TRANSACT, the transduction was performed. The lentiviral vectors were added to the T cells in a multiplicity of infection (MOI) of 5. 24 to 48 hours after transduction, the supernatant of each well was removed and fresh TEXMACS containing IL-7 and IL-15 was added. Depending on the T cell density the T cell culture was split 1:2 or 1:3 every 2 to 3 days to keep the cells in a concentration between 0.5×10⁶ and 2×10⁶ cell/ml.

### Determination of Transduction Efficiency (by LNGFR) and CAR Expression

The generated CAR constructs contain LNGFR as marker gene, so the transduction efficiency was analysed via anti-LNGFR staining using anti-LNGFR-PE by flow cytometry (MACSQuant Analyzer 10) as previously described.

To analyse the CAR expression Protein L was used in order to stain the CAR via the variable light chains (kappa chain). Cells were resuspended in buffer containing Protein L-Biotin (5 μg/1000 μl) and following incubation for 45 min at 4° C. the cells were washed and resuspended in buffer containing anti-Biotin-PE. Cells were then washed and analysed by flow cytometry (MACSQuant Analyzer 10).

### Long Term Co-Culture: INCUCYTE

The target cells RKO-KO CLDN3 H1 (human Claudin-3 knock out+human Claudin-3 and GFP marker introduction via lentiviral transduction) were used for this assay. The T cells were thawed 72 hours before setting up the assay (see above) and recovered in TEXMACS with IL-7 and IL-15. On the day of the assay, T cells were resuspended well and the same conditions (same donor and expressing the same CAR construct) were pooled and taking into account the cell concentration and frequency of LNGFR positive T cells, the T cells were adjusted to their transduction efficiency and the co-culture was set up in an Effector:Target 2.5:1 for 4×10⁴ and 3×10⁴ target cells. T cell suspension was added and the co-cultures were incubated in the INCUCYTE to monitor the target cell growth via green confluence. On day 3, fresh target cell medium was added. On day 4, fresh target cells were seeded in the same concentrations as in the 1st round, into new cell culture plates roughly 4 to 5 hours before the T cells were added. The T cells from the same condition were pooled and the adjusted T cells were added in an E:T of 2.5:1 to the freshly seeded target cells. The cell culture plates were incubated in the INCUCYTE to monitor the target cell growth via green confluence. On day 7 fresh target cells were seeded in a concentration of 3×10⁴ target cells into new cell culture plates, roughly 4 to 5 hours before adding the T cells. All T cells from the same condition were pooled.

### Repeated Antigen Stimulus: Antigen Spike-In

The target cells RKO-KO CLDN3 H1 (human Claudin-3 knock out+human Claudin-3 and marker GFP) were co-cultured with T cells. T cells from Donor H5 and P were thawed 96 hours before setting up the co-culture and recovered in TEXMACS with IL-7 and IL-15.

To stain for exhaustion markers, transduction efficiency, CD4, and CD8 the following conjugates were used: LAG3 (CD223)-VioBlue, PD-1 (CD279)-PE-Vio770, TIM3 (CD366)-APC, CD8-APC-Vio770, CD4-VioGreen, LNGFR-PE, and 7-AAD. A mastermix of these conjugates was prepared. Cells were resuspended in mastermix and then incubated for 10 min at 4° C. (in the dark). Cells were resuspended in PEB (CliniMACS with 0.5% BSA) and the samples were measured at the MACSQuant Analyzer 10. The required volume of T cell suspension to reach an E:T of 2:1 based on transduced T cells was resuspended in appropriate volume of target cell medium, the T cell suspension was added to the target cells, and the co-cultures incubated into a humidified incubator (37° C. and 5% CO₂). Every 24 hours T cells were stained for exhaustion markers and fresh target cells were added to the co-culture. The last addition of fresh target cells was on day 3.

### Results

### Staining of T Cells to Analyse Transduction Efficiency (via LNGFR) and CAR Expression

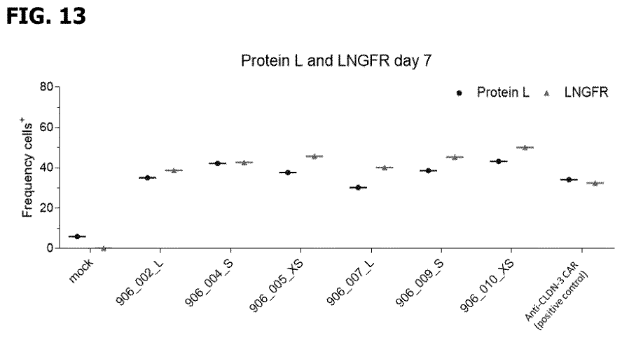

In order to analyse the transduction efficiency of CAR T cells generated from donor D5 (see above) the LNGFR marker gene expression was measured by flow cytometry on day 7. The data obtained from the staining showed that the transduction was successful and a frequency of 39% to 50% LNGFR positive T cells was achieved (see FIG. 13). Moreover, on day 15 the LNGFR expression was analysed again (see FIG. 13) in order to adjust the T cells to their transduction efficiency for functionality testing. Expression of CAR molecules on the T cell surface is a requirement for CAR function. Therefore, the CAR expression was determined via Protein L staining (see above). Data obtained showed that all generated CAR variants were expressed and frequencies of CAR positive T cell populations ranging between 35% to 43% were reached (see FIG. 13). Furthermore, CAR-T cells expressing a different scFv against claudin-3 were included as positive control.

### Luciferase Killing Assay

To evaluate cytotoxicity of anti-claudin-3 CAR-T cells a luciferase-based killing assay was performed. For co-cultures the breast cancer cell line T-47D was used. This target cell line was engineered to express both luciferase and eGFP following a transduction with a lentiviral vector encoding the two markers followed by cell. T cells expressing various anti-claudin-3 CAR or untransduced T cells were prepared and expanded and were cultivated without cytokines for 48 hours prior to the co-culture. The T cells were then added to the target cell line in 3 different effector to target (E:T) ratios adjusted according to the lowest transduction efficiency (frequency of LNGFR positive cells) namely 5:1, 1:1 and 0.2:1, and the co-cultures incubated (humidified, 37° C., 5% CO₂). Supernatant was removed 20 hours after setting up the co-culture and stored until cytokines were measured via a MACSPlex assay. D-Luciferin solution was added to the cells and 5 minutes after incubation, luminescence was read with a luminometer (VICTOR, PerkinElmer).

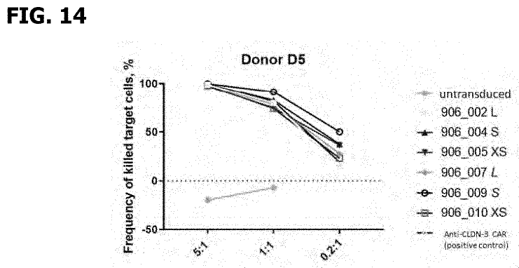

CAR T cells expressing a different scFv against claudin-3 were used as a positive control. The graph (FIG. 14) displays the frequencies of killed target cells after 20 hours of co-culture. No increase in frequency of killed target cells was visible when co-cultured with untransduced T cells. T cells expressing various anti-claudin-3 CAR constructs, co-cultured with T-47D, which were expressing human Claudin-3, showed increased frequencies of killed target cells and a cytotoxicity depending on the E:T ratio. At an E:T of 5:1, the frequency of killed target cells was 96%-99.7% and the frequency of killed target cells was found to be decreasing with lower E:T ratios. Moreover, the frequencies of killed target cells were similar between the 7 tested CAR constructs.

Determination of Cytokine Secretion into the Supernatant via MACSPlex Assay

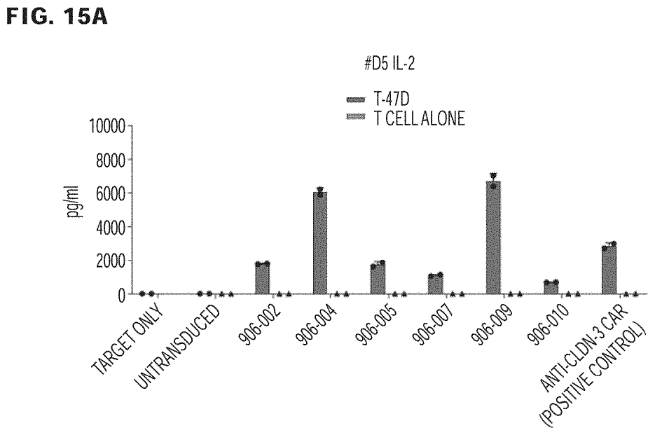

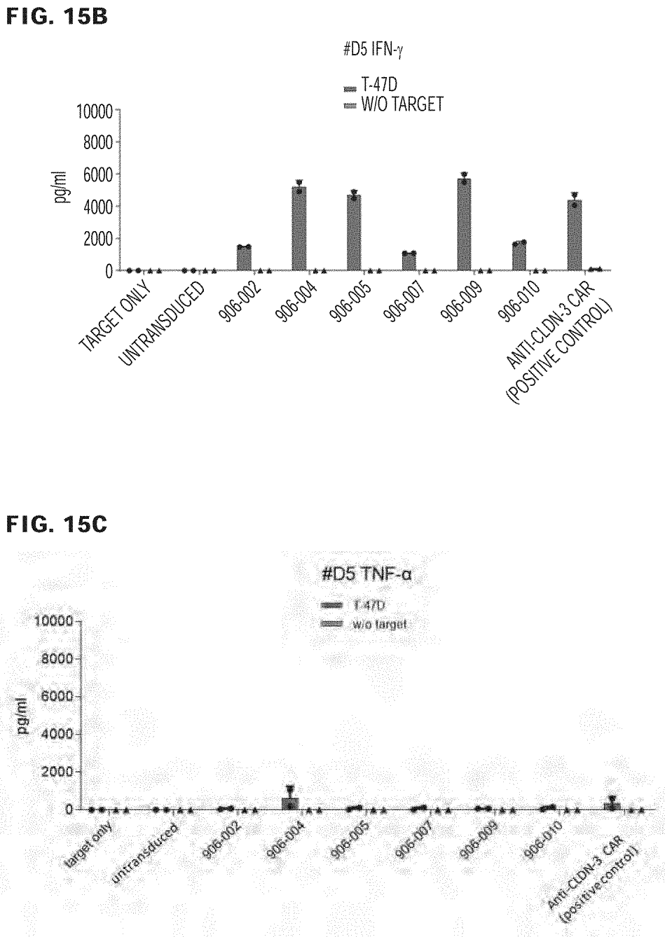

Co-culture supernatants (see above) were analysed for the concentrations of the cytokines IL-2, IFNγ and TNF-α using the MACSPlex assay. Only samples from the co-culture with an E:T ratio of 1:1 were analysed. The samples and MACSPlex assays were prepared according to the manufacturer's protocol for MACSPlex Cytokine 12 kit (human) and analysed on MACSQuant Analyzer 10. The resulting concentrations are depicted in pg/ml (FIGS. 15A-15C). The supernatants used for the analysis were not diluted. No elevated levels for IL-2, IFNγ and TNF-α were detected in the supernatant from conditions where untransduced T cells were co-cultured with the cancer cell line T-47D. Also in the samples from “target cells only” condition no cytokines could be detected. All conditions, in which CAR T cells expressing various anti-claudin-3 CARs based on the 906 variants were co-cultured with T-47D cells showed elevated levels of IL-2 and IFNγ compared to mock T cells. The highest amount of IL-2 was secreted by 906_004, 906_009 and the positive control claudin-3 CAR in presence of T-47D. These constructs including 906_005 also showed the highest IFNγ secretion. Only low levels of TNF-α were detected, whereof the highest concentrations were detected for the samples 906_009 and the positive control claudin-3 CAR.

Long Term Co-Culture of T Cells with Repeated Antigen Stimulus

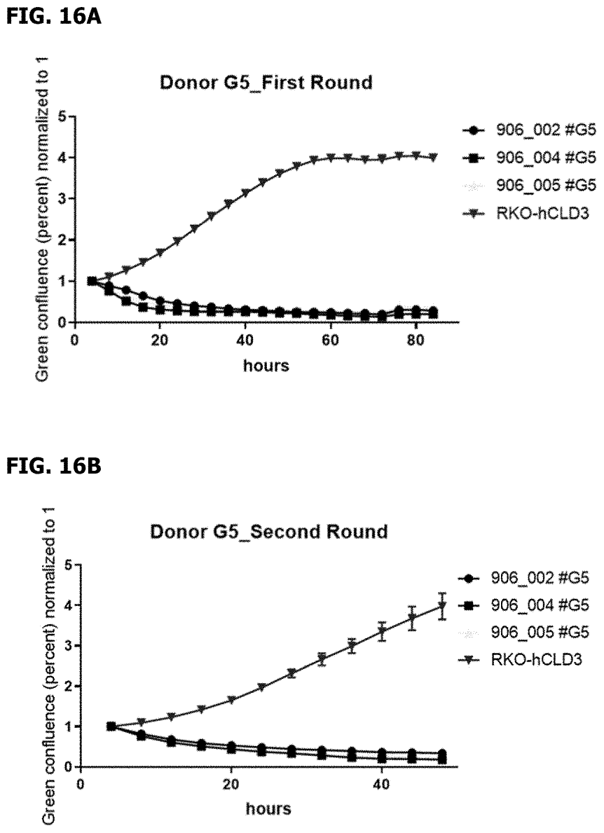

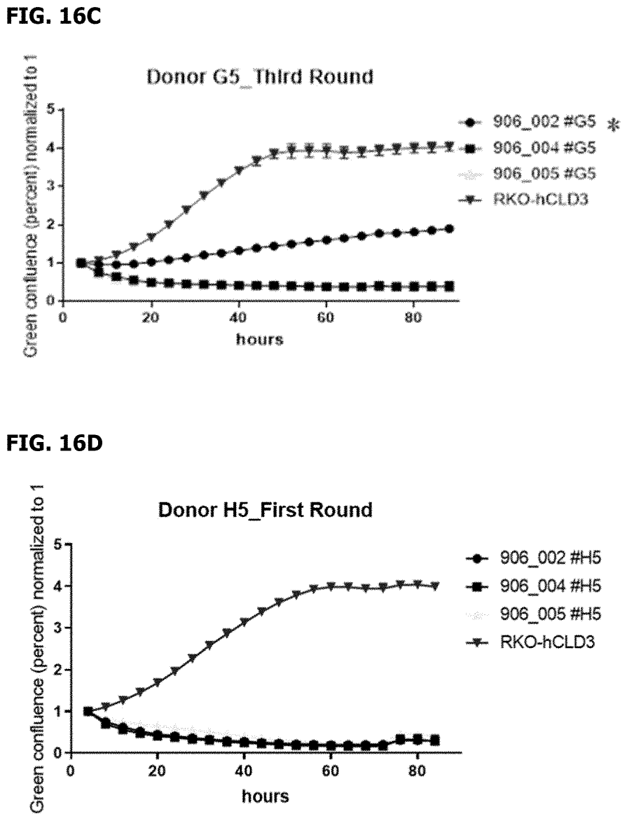

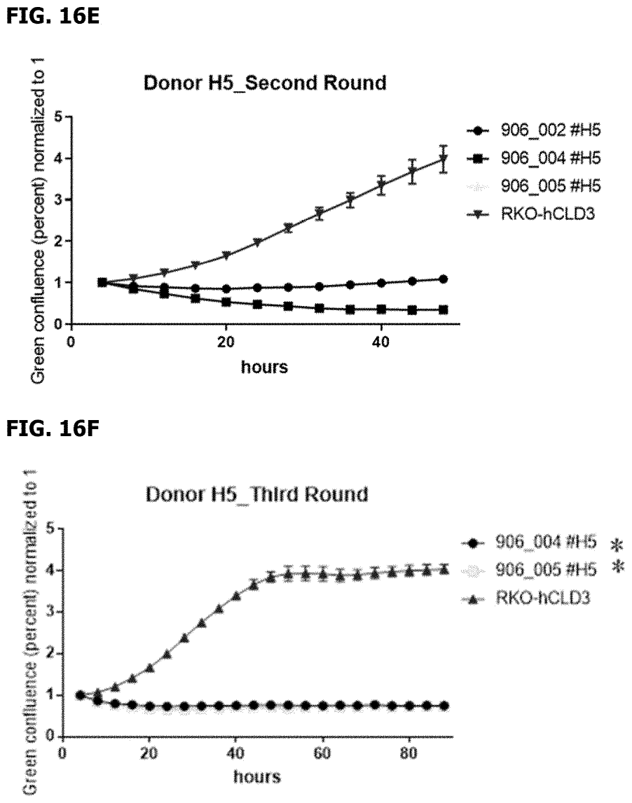

The growth of the target cells in co-culture with T cells expressing 906_002 (long spacer), 906_004 (short spacer) or 906_005 (very short spacer) CAR variants was monitored by the INCUCYTE. The results indicated for both donors (G5 and H5) that in the 1^{st} round of target cell exposure, the target cells were cleared efficiently by all three CAR constructs (FIGS. 16A and 16D). While in the controls, in which target cells RKO-KO CLDN3 H1 were cultured alone, proliferation was observed. After transferring the CAR T cells onto fresh target cells for a 2^{nd} and 3^{rd} round of target cell encounter, differences between the CAR variants became visible. T cells expressing CAR variants with a long spacer controlled target cell growth less efficiently compared to the T cells expressing anti-claudin-3 CARs with a short and very short spacer variant (FIGS. 16C and 16E). For donor H5 not enough LNGFR positive T cells were obtained after the 2^{nd} round, therefore a 3^{rd} round was not performed.

### Repeated Antigen Stimulus: Antigen Spike-In

Expression of exhaustion markers (TIM3, PD-1, LAG3) was analysed on day 0, 1, 2, 3 and 6 by flow cytometry. Only LNGFR positive T cells were included in the analysis for double (TIM3, PD-1) and triple positive (TIM3, PD-1, LAG3) T cells.

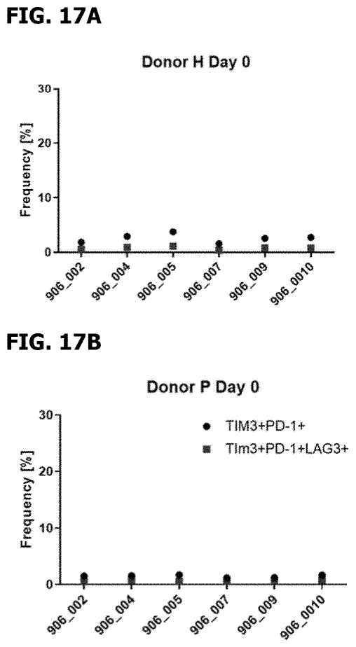

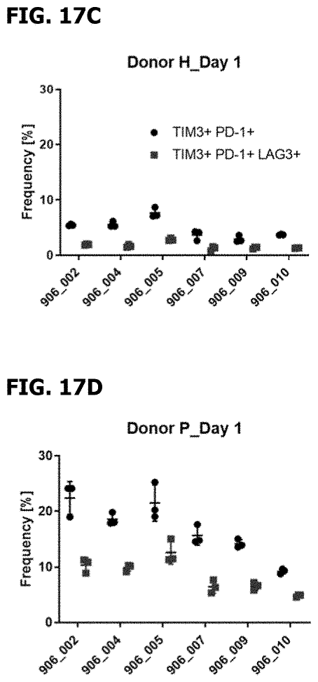

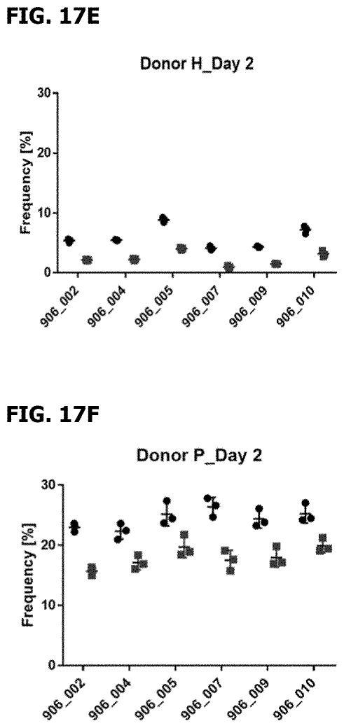

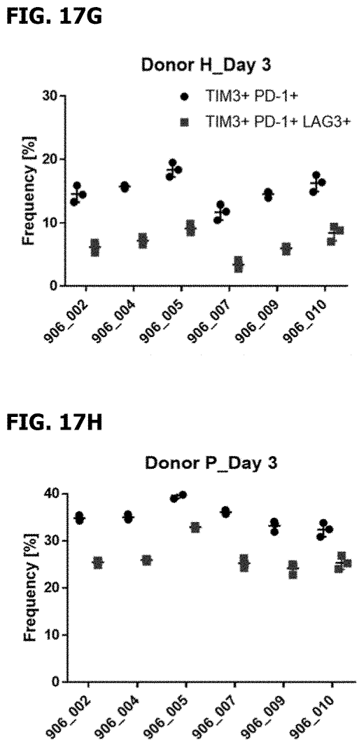

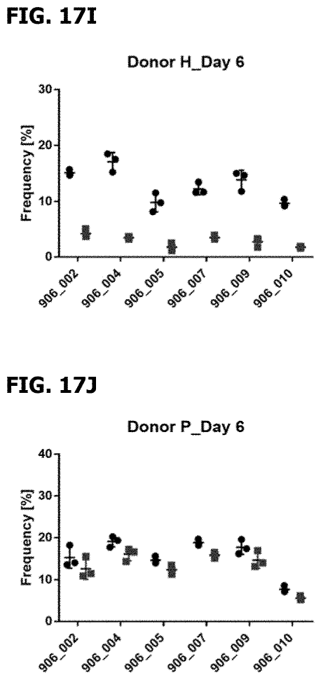

The results depicted in FIGS. 17A-17D showed that on day 0 before the first addition of target cells, the frequency of double and triple positive transduced T cells was below 5%. The frequency of double and triple positive T cells however increased with target cell encounters from day one to day three for both donors. Donor P showed a higher increase in expression of exhaustion markers compared to donor H5 (FIG. 17B). The staining on day 6, after two days (day 4 and 5) without addition of fresh target cells, indicated that the frequency of double and triple positive transduced T cells decreased, which was more pronounced for donor P than for donor H5.

T cells expressing various anti-claudin-3 CAR constructs based on the 906 scFv variant, co-cultured with T-47D, which were expressing human Claudin-3, showed increased frequencies of killed target cells depending on the E:T ratio. This was not observed when the cancer cell line was co-cultured with untransduced T cells. This indicated specific lysis of target cells expressing human Claudin-3, when co-cultured with anti-human Claudin-3 CAR-T cells. Moreover, all T cells expressing anti-claudin-3 CAR constructs, which differed in scFv orientation and spacer length (L, S and XS), showed comparable lytic capability on the tested target cells. Overall, the functionality of the anti-claudin-3 CARS with regard to lysing target cells expressing human Claudin-3 were comparable to CAR-T cells expressing the positive control claudin-3 construct.

Secreted cytokines IL-2 and IFNγ concentrations in the supernatants were increased for anti-claudin-3 CAR-T cells co-cultured with T-47D compared to untransduced T cells. This indicated specific cytokine secretion of anti-claudin-3 CAR-T cells based on the 906 variants in presence of target cells expressing human Claudin-3. Constructs 906_009 and 906_004 showed the highest concentrations of secreted cytokines, which both have a short spacer in common. For these constructs secreted cytokine levels were comparable or even higher than for CAR-T cells expressing the positive control claudin-3 construct.

In order to further evaluate the performance of T cells expressing different CAR variants based on the 906 scFv more challenging assays were performed. Therefore, a long term co-culture was performed, in which three rounds of fresh target cells were added. The CAR-T cells expressing the 906 variant with 3 different spacers (L, S and XS) performed equally well in the first round of target cell encounter. The variants expressing the short and very short spacer however were able to clear freshly added RKO-KO CLDN3 H1 cells during all three rounds of co-culture. For the CARs based on the long spacer variant, CAR-T cells from one donor (H5) cleared target cells only during the first round and CAR T cells from the second donor (G5) only during the first two rounds of target cell exposure. It is expected that orientation of the scFv would not impact these results.

In the assay, in which repeated antigen stimulus via antigen spike-in was performed, an increased expression of exhaustion markers of LNGFR positive T cells after repeated antigen encounter was apparent (FIGS. 17A-17E), however the difference between the two tested donors was more pronounced than for T cells expressing various CAR constructs based on the 906 scFv. Therefore, in this assay no conclusion could be made regarding a difference in functionality of specific CAR construct variants. Furthermore, the data on day 6 indicated that the frequency of exhaustion marker double and triple positive CAR T cells decreased, after no fresh target cells were added on day 4 and 5. It was not determined if the reduction in frequencies resulted from an actual decrease of those markers on the LNGFR positive T cells or could be ascribed to another reason e.g., to expansion of non-exhausted T cells and which might lead to reduced overall frequencies of non-exhausted T cells.

Anti-claudin-3 CARs are expressed on primary T cells at levels that suggest they can redirect T cell activity to Claudin-3-expressing tumour cells. Those anti-claudin-3 CAR-T cells were able to lyse target cells expressing Claudin-3 and secreted IL-2 and IFNγ selectively in presence of the target. Furthermore, the anti-claudin-3 CAR-T cells were able to clear Claudin-3-expressing tumour cells during several rounds of target cell exposure.

### Example 6

### Proliferative Response of Anti-Claudin-3 CAR-T Cells to CLDN3 Positive Tumour Cells

The objective of this study was to assess the ability of anti-claudin-3 CAR-T cells to proliferate in response to antigenic stimulation with Claudin-3 positive target cells.

### Materials and Methods

### Experimental Preparation(s)

Proliferation of CAR T cells was assessed by culturing effector and target cells for 72 hours.

For these experiments, T cells from 6 donors in 2 independent experiments were lentivirally transduced with 4 constructs based on the same scFv variant (906-002, 906-004, 906-007 and 906-009; see Example 5). T cells were engineered with a low-affinity nerve-growth-factor receptor (LNGFR) marker gene directly into the CAR sequence to allow for isolation of CAR positive cells by sorting with immuno-magnetic beads targeting the LNGFR marker gene expressed on the extracellular portion of the CAR molecule.

CAR-T cell proliferation was measured by the incorporation of [³H] thymidine following a 72 hour 1:1 coculture with Claudin-3 positive (RKO) and Claudin-3 negative (RKO-KO) cell lines. The thymidine incorporation assay utilizes a strategy wherein a radioactive nucleoside, 3H-thymidine, is incorporated into new strands of chromosomal DNA during mitotic cell division.

### Experimental Protocol(s)

CAR-T cell proliferation was measured by co-culturing effector cells and target cells at a 1:1 ratio. 1×10⁵ enriched CAR-T cells were co-cultured with 1×10⁵ CLDN-3 positive RKO or CLDN-3-negative (RKO huCLDN-3ko) cell lines. After 48 h, cells were pulsed with 1 μCi (37Bq) of [³H]-thymidine (PerkinElmer) and incubated for a further 21 hours to allow the T cells to incorporate the radioactivity into the newly synthesized DNA of dividing cells. Cells were harvested to a filter mat using a cell harvester (Micro 96 harvester-Skatron Instruments). In order to determine the extent of cell division that has occurred in response to [³H] thymidine incorporation, the radioactivity incorporated in DNA was measured using a Wallac 1450 MicroBeta trilux liquid scintillation and luminescence beta-counter (Perkin Elmer) and it was expressed as Counts per Minute (CPM). Data analyses were performed in GraphPad Prism, version 5.0.4. Data were expressed as mean±standard error and analyses were performed by two-tailed Student's t-test, as indicated in the figure legends. Significance of findings are defined as follows: NS, not significant; *P<0.05; **P<0.01; ***P<0.001; ****P<0.0001.

### Results

The proliferative ability of CAR-T cells that were transduced with 4 lentiviral constructs (906-002, 906-004, 906-007 and 906-009) encoding the anti-Claudin-3 scFv in two different orientations (V_{H}-V_{L} and V_{L}-V_{H}) and two spacer lengths was compared.

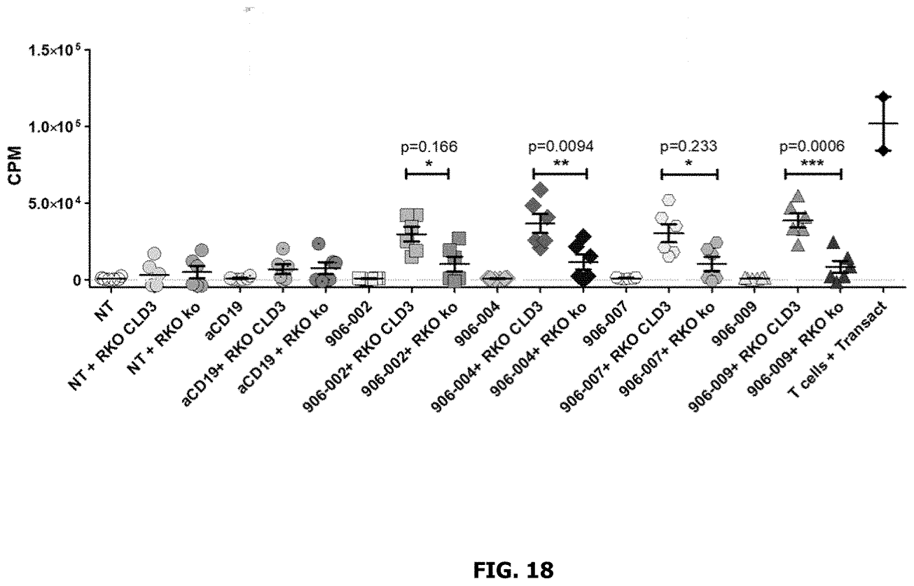

The results demonstrate that 906-009 anti-Claudin-3 CAR T cells showed significantly greater antigen-specific proliferation in vitro than T cells transduced with the other constructs 906-002, 906-004, 906-007 CAR (FIG. 18) against a Claudin-3 expressing cell line (RKO).

None of the anti-Claudin-3 CAR-T cells showed any proliferation when cultured with RKO-Claudin-3 KO cell line.

Anti-CD19 CAR-T cells showed no proliferation when cultured with Claudin-3 RKO cells.

These data predict that 906-009 anti-Claudin-3 CAR-T cells can have greater in vivo proliferation that may result in greater anti-tumour clinical activity. These findings suggest that using anti-Claudin-3 CAR-T cells as a cancer immunotherapy for colon cancer would sustain the proliferative and cytotoxic responses against the tumour antigen.

### Example 7

### In Vivo Assessment of Anti-Claudin-3 CAR-T Cells Activity in CDX NSG Model and In Vitro Assessment Against Human CRC PDX Samples

The objectives of this study were to assess the efficacy of T cells transduced with an anti-claudin-3 CAR in a mouse model in vivo and its functionality in a patient-derived Human Xenograft (PDX) model in vitro. The results demonstrate that anti-claudin-3 CAR-T cells prolonged the survival of the mice and controlled the tumour growth.

### Materials and Methods

To assess efficacy of the anti-claudin-3 CAR-T cells to kill tumour cells in vivo the CLDN3 expressing colon cancer cell line HT-29 Luc was used. Primary read-out of the study was (1) impact on tumour growth and survival of the mice. Secondary read-outs were (2) serum cytokine release to assess T cell activation, (3) distribution of T cells in the tumour and mouse tissues by histopathology and (4) CAR T detection in the blood.

Functionality of the anti-claudin-3 CAR-T cells was further assessed in Patient-derived Human Xenograft (PDX) models. At day 0 (D0) PDX cells were thawed, characterised by flow cytometry and seeded. On day 1 (D1), T cells were thawed and seeded on top of PDX cells at a 1:1 ratio. On day 2 (D2), the supernatant was collected and cytokine levels assessed via an MSD assay. The PDX and T cells were harvested and characterised for CLDN3, PD-L1, EpCAM (tumour cells) and CD69 (T cells) expression via flow cytometry.

### NSG Mice

Prior to launch of the study, the HT-29 Luc cells used for inoculation were screened for a comprehensive panel of human and murine pathogens (Charles River) and all results came back negative. In parallel, the donor blood was tested negative for Hepatitis B, C and HIV I/II.

Faeces of mice were tested during the study for additional pathogens. The faeces of mice on study as well as mice from the same supplier on another study were tested positive for astrovirus-1 and Segmented Filamentous Bacteria (SFB). Following consultation with the veterinarian it was assumed that both organisms did not bear clinical health implications. Both SFB and astrovirus-1 were reported to have implications on the development of the competent immune system, and SFB also had a role in modulation of inflammation.

### Tumour Cell Preparation and Inoculation to NSG Mice

HT-29 Luc cells were harvested and supernatant was collected for human and murine pathogen testing for confirmation of pathogen-free status of the cells. Harvested cells were counted and subsequently used for subcutaneous (s/c) inoculation of 0.5×10⁶ HT-29 Luc cells into the right flank of each NSG mouse.

### Tumour Growth, Euthanasia and Tissue Harvest

Mice were closely monitored until termination of the study. Tumour size in all mice was measured by palpation/calliper measurements and recorded three times a week to be followed by body weight recording twice a week.

Mice were culled and tissues harvested at individual end points due to end point criteria such as tumour volume. Tumours and spleens (whole tissue/organ intact) were collected in PBS on ice. One half was used for tissue processing, the other half was fixed with 10% neutral buffered formalin (NBF) for up to 48 hours for histopathological examination. Hearts, lungs, colons, kidneys, livers, ovaries, and brains were collected and directly fixed with 10% NBF. All fixed tissues were supplied to GSK TMCP UK Histology, Ware.

### T Cell Thawing, Culturing and Dosing

To ensure an even spread of tumour sizes across groups, mice were block randomised into groups of 7-8 mice according to tumour volume. When tumours were palpable (˜100 mm³), CAR-T cells were dosed via tail vein injection at a dose of 1×10⁷cells per mouse as shown in Table 10 below.

TABLE 10
Dosing of CAR-T cells in mouse studies

| Group | T Cells | Dose | Mice/group |
|---|---|---|---|
| A | No T cells (PBS) | NA | 8 |
| B | Ctrl (anti-CD19) CAR | 1 × 10⁷ | 7 |
| C | Anti-claudin-3 CAR (906-009) | 1 × 10⁷ | 8 |

### Serum Cytokine Assay and CAR-T Cell Detection in the Blood

Blood samples from all mice on study were collected prior and 7 days post T cell dosing to assess the serum cytokine release and at Day 28 post dosing to assess CAR-T cells in the blood.

Cytokines were detected in the collected mouse serum samples by MSD using the following detection antibodies: Sulfo-TAG Anti-hu IFNγ Antibody, Sulfo-TAG Anti-hu IL-1β Antibody, Sulfo-TAG Anti-hu IL-2 Antibody, Sulfo-TAG Anti-hu IL-4 Antibody, Sulfo-TAG Anti-hu IL-6 Antibody, Sulfo-TAG Anti-hu IL-8 Antibody, Sulfo-TAG Anti-hu IL-10 Antibody, Sulfo-TAG Anti-hu IL-12p70 Antibody, Sulfo-TAG Anti-hu IL-13 Antibody, and Sulfo-TAG Anti-hu TNFα Antibody.

Whole blood from each mouse still on study at Day 28 post dosing was collected and stained with the following antibodies: CD45-FITC (1/100 dilution); CD3-BUV395 (1/50 dilution); CD8-APCVio770 (1/200 dilution); CD4-PerCPVio770 (1/50 dilution); and LNGFR-PEVio770 (1/600 dilution).

### PDX Co-Cultures and Flow Cytometry

Five colorectal cancer models and one ovarian cancer model were used to establish patient-derived human xenograft (PDX) models. PDX colorectal cancer cell models (CR5052, CR5080, CR5089, CR5030, CR5087) and PDX ovarian cancer cell model (OV5287) were obtained from Crown Biosciences.

Day 0: PDX cell suspensions were thawed, counted and seeded at 50,000-100,000 cells/well.

Remaining PDX cells were characterised by flow cytometry analysis using the following antibody panel: EpCAM-BV650 (1/600 dilution); Cldn3-PE (1/10 dilution); PDL1-BV421 (1/100 dilution); CD45-FITC (1/100 dilution); LNGFR-PEVio770 (1/100 dilution); and CD69-BV786 (1/100 dilution).

Day1: T cells were thawed and added to the PDX cells at a 1: 1 CAR-T cell to PDX cell ratio. Additional wells with PDX cells alone and T cells alone were used.

Day 2: The supernatant was collected and subjected to the cytokine assay. Additionally, the co-cultured cells were harvested and assessed by flow cytometry using the same panel as used for D0.

Supernatants were collected and the cytokine assay was carried out as described above.

### Results

### Tumour Growth Retardation and Survival

The primary objective of the in vivo study was to evaluate the efficacy of anti-claudin-3 CAR-T cells in the HT-29 Luc colon cancer model in vivo.

The human T cells were phenotyped on the same day as in vivo dosing in regard to transduction efficiency, memory and exhaustion phenotype. Cells showed high viability (87-92%) and transduction efficiencies were determined as 32% for anti-CD19 CAR and 35.8% for anti-claudin-3 CAR by LNGFR staining. This was consistent with transduction efficiency and viability obtained before freezing when T cells were normalized to 30% transduction efficiency. In addition, a more complex T cell phenotyping confirmed LNGFR expression for 30% of the cells (27% for CD19 and 32.7% for anti-claudin-3 CAR) and illustrated that CD8 T cells were more abundant than CD4 T cells for both CARs. The percentage of LNGFR expression was higher for CD4 T cells than for CD8 T cells. TIM3 and PDL-1 were expressed 97% and 86-88% for CD3 T cells respectively.

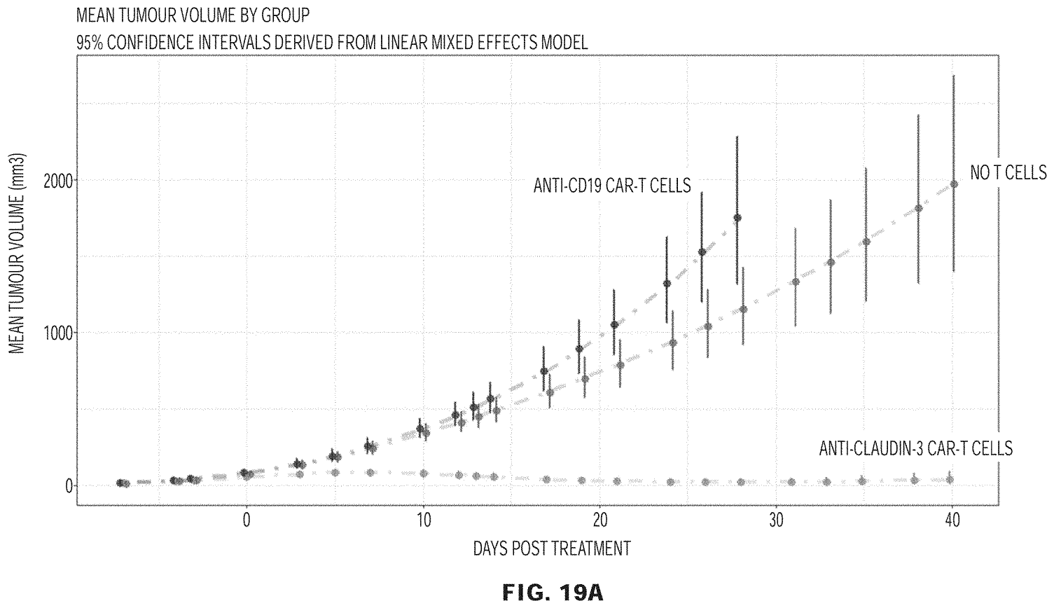

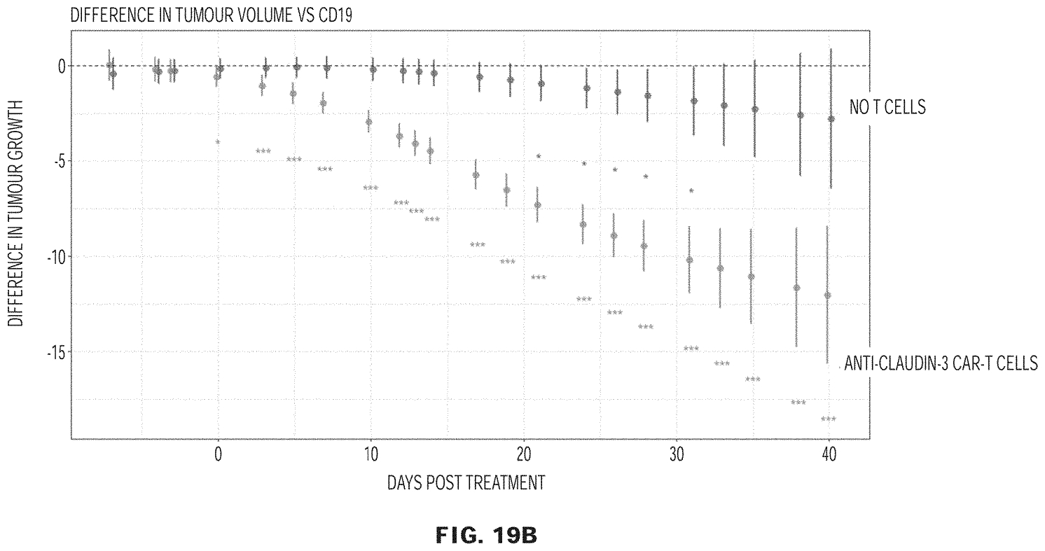

Anti-claudin-3 CAR-T cells controlled the tumour growth (FIGS. 19A-19B). Tissues from tumour inoculation sites were subjected to histological analysis ex vivo. No tumour cells could be detected thus anti-claudin-3 CAR-T cells did destroy the tumours entirely. The survival time in this study was defined as ‘time needed for a mouse's tumour to reach 1000 mm³’. The proportion of mice in each group with a tumour below 1000 mm³ is shown in FIG. 19A confirming a significant difference of survival time between anti-CD19 and anti-claudin-3 CAR-T cells.

Starting at day 30 post T cell inoculation some mice showed signs of subdued posture, squinty eyes, hair loss, poor breathing, abnormal gait, piloerection and weight loss. These mice were culled at first signs of these symptoms in accordance with animal welfare. These symptoms might have been accountable to cytokine release syndrome (CRS) associated with tumour destruction or graft-versus-host-disease (GvHD) considering the time of onset. These clinical symptoms were only observed in anti-claudin-3 CAR-T cell treated groups as all mice of the anti-CD19 CAR treated control group were sacrificed at earlier time points due to large tumour volumes

### Distribution of Anti-Claudin-3 CAR-T Cells in the Tumour and Healthy Tissues (IHC)

The tissue distribution of anti-claudin-3 CAR-T cells and potential mouse tissue damage was assessed by histopathology. The evaluation for this study indicated a widespread perivascular human T cell accumulation in murine tissues of both T cell dosed groups. As anti-claudin-3 CAR can recognise mouse CLDN3 potential toxicity effects need to be considered. A very minor increased hepatocellular and epithelial turn-over was present in animals given the anti-CD19 or anti-claudin-3 CAR-T cells. In addition, no epithelial injury in colon or lung was observed. Therefore, no histological evidence of anti-claudin-3 CAR-T cell related tissue damage or destruction of the murine endogenous target could be found.

### CAR T Cells in Peripheral Blood

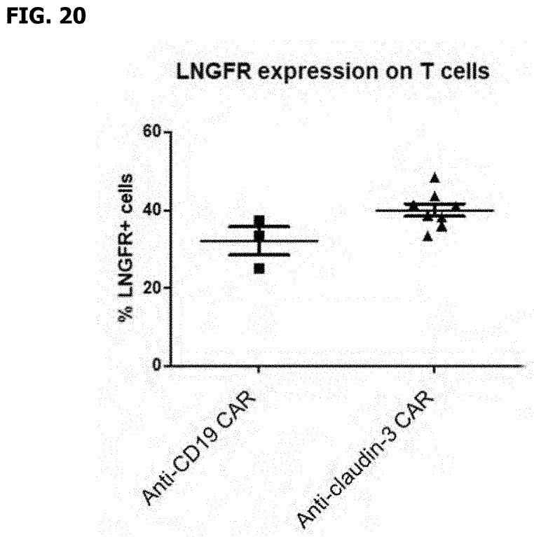

At day 28 post-dosing, flow cytometry analysis of the blood was performed for all mice still on study to identify the presence of CAR-T cells. These cells were detected with a range from 25 to 4438 CAR-T cell count/uL of whole blood. The percentage of CAR-T cells as measured by LNGFR expression on T cells was maintained at around 30-40% and was comparable to the expression tested at the day of dosing (FIG. 20). There were no major differences between the study groups. The frequencies of LNGFR expression were higher on CD4 T cells than on CD8 T cells in both anti-CD19 and anti-claudin-3 CAR groups.

### Serum Cytokine Levels

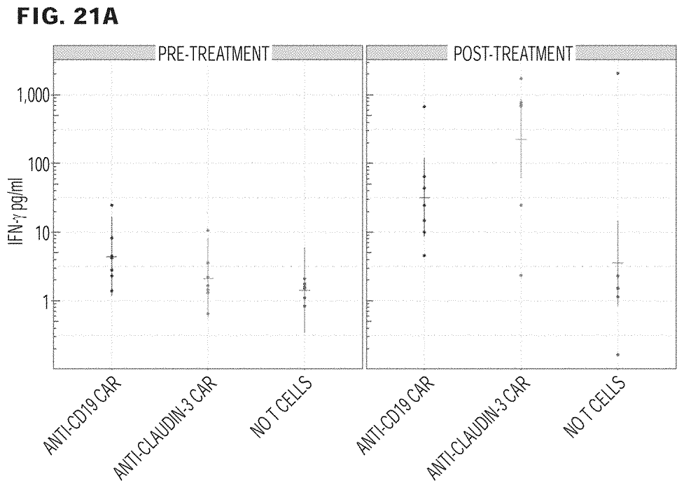

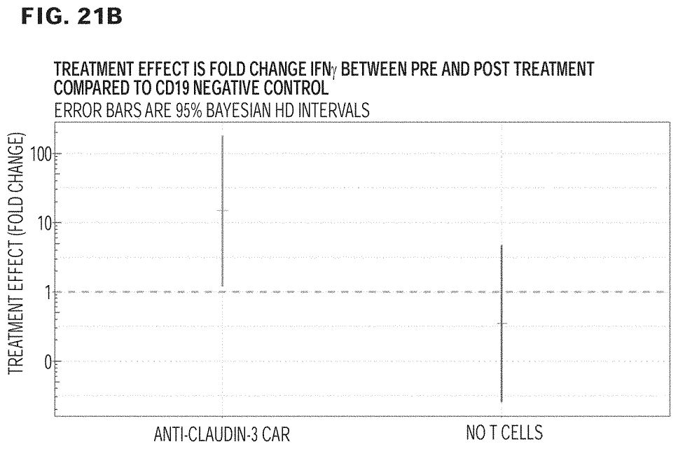

To assess cytokine levels in blood serum, samples were collected from all mice prior and at D7 post T cell dosing. Anti-claudin-3 CAR-T cell dosed mice showed increased IFNγ levels 7 days post-treatment (Median of 225 pg/mL compared to 32 pg/mL for CD19) as shown in FIGS. 21A-21B. The other tested cytokines did not show a clear trend or measurements were below the level of detection.

### Patient-Derived Xenograft (PDX) Characterisation and Co-Culture Establishment

The functionality of anti-claudin-3 CAR-T cells was tested in Patient-derived Human Xenograft (PDX) models. These models allow for the recreation of the heterogeneity of tumour cells seen in tumours in humans. Five colorectal cancer models were chosen based on CLDN3 expression, histopathological tumour characteristics and cell survival in culture in vitro. Additionally, one ovarian cancer model was chosen as a low CLDN3 expresser.

The PDX samples were then used for setting up the co-culture with anti-claudin-3 CAR vs. anti-CD19 CAR (negative control) T cells. Primary read-outs were: (1) characterisation of PDX samples after thawing (D0) via flow cytometry with the tumour marker EpCAM, PDL-1 and CLDN3 and (2) T cell activation measured by cytokine release (MSD assay on the supernatants from 24 hour co-cultures). Secondary read-outs were: (3) characterisation of co-cultured samples via flow cytometry with the tumour marker EpCAM, PDL-1 and CLDN3 and T cells markers CD45, LNGFR (indicative of CAR T cells) and CD69 (activation marker). These experiments were run with HT-29 cells as CLDN3 positive control and RKO-KO cells as CLDN3 negative control.

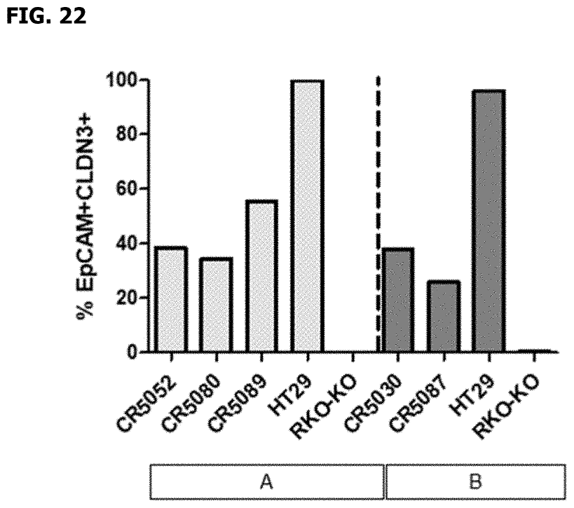

RNAseq data obtained from the PDX model supplier indicated that EpCAM would be a suitable tumour cell marker for the colorectal (CR) PDX models but not the ovarian (OV) PDX model. Percentage of EpCAM-positive cell population was ranging from 41 to 65% for the CR models but only 14 to 17% were detected in OV PDX samples. Characterisation of CR PDX samples demonstrated CLDN3 expression on EpCAM-positive tumour cells of 26 to 55% (FIG. 22). No CLDN3 could be detected in the OV model via flow cytometry (0.29%). Furthermore, no CLDN3 was detected in the RKO-KO cells (negative control) in any of the experiments as expected. The Percentage of PDL-1 expressing target cells (EpCAM+ CLDN3+ PDL-1+ population) was below 2% in PDX samples at D0 but increased after co-culture and was elevated in the anti-claudin-3 CAR-T cell co-cultures compared to anti-CD19 CAR-T cell co-cultures at D2.

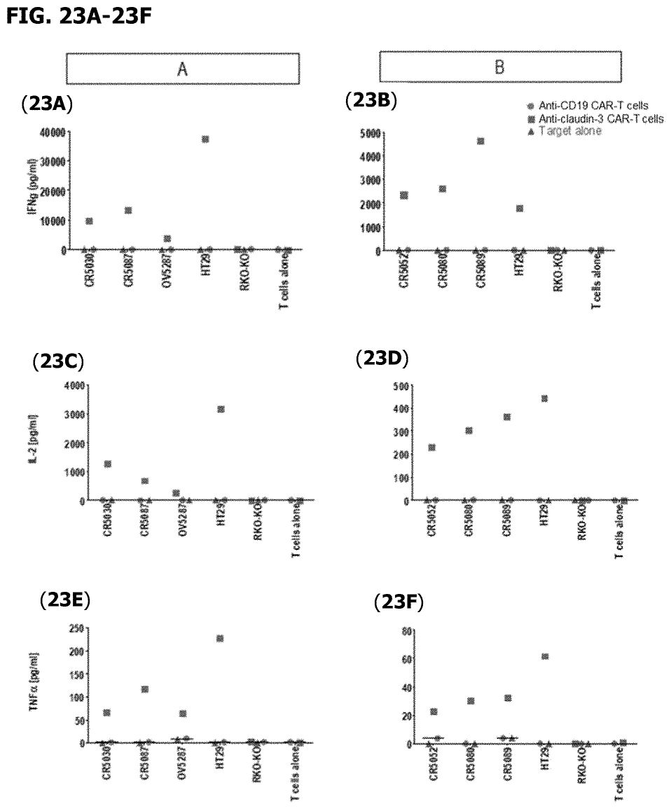

All PDX models tested within this pilot co-culture experiments (CR5030, CR5080, CR5052, CR5087, CR5089, OV5287) and the positive control (HT-29) induced anti-claudin-3 CAR-T cells cytokine release (IFNγ, IL-2 and TNF-α) while negative controls (RKO-KO, T cells alone and all co-cultures with anti-CD19 CAR T cells) did not induce T cell responses as measured in an MSD assay (FIG. 23).

Characterisation of the T cells showed elevated expression of the early T cell activation marker CD69 when comparing anti-claudin-3 CAR-T cell co-cultures to anti-CD19 CAR-T cell co-cultures (CD45+ LNGFR+ CD69+ population ranged from 69 to 82% for anti-claudin-3 CAR-T cell co-culture compared to 11 to 22% for anti-CD19 T cell co-cultures).

NSG mice with palpable HT-29 Luc tumours were inoculated with anti-claudin-3 or anti-CD19 CAR-T cells in a dose of 1×10⁷ total number of cells or PBS (no T cells). Anti-claudin-3 CAR-T cells prolonged the survival of the mice and controlled the tumour growth as confirmed by complete destruction of the tumour mass (histology). These data were supported by elevated serum levels of IFNγ at D7 post T cell dosing. Therefore, anti-claudin-3 CAR-T cells demonstrated high efficacy in terms of tumour killing in vivo.

The histopathological analysis of mouse tissues demonstrated widespread perivascular human T cell accumulation of both T cell dosed groups. No evidence of anti-claudin-3 CAR-T cell related tissue damage was observed. This supported the hypothesis that CLDN3 restricted to tight junction (TJ) in healthy tissues was not accessible for anti-claudin-3 CAR-T cells. However, when mislocalised outside the TJ CLDN3 was recognised by anti-claudin-3 CAR-T cells in the tumour.

In conclusion, anti-claudin-3 CAR-T cells proved to be an efficient anti-cancer therapy in vivo.

To evaluate efficacy of the CAR-T cells in another model, patient-derived Human Xenograft (PDX) models were used, that enable the mimicking of the heterogeneity of tumour cells as seen in humans. They were used to assess the functionality of the anti-claudin-3 CAR-T cells in vitro. Experiments with five colorectal PDX models and CLDN3 positive control cells (HT-29) showed elevated anti-claudin-3 CAR-T cells cytokine release (IFNγ, IL-2 and TNF-α) while negative controls (RKO-KO co-cultures and T cells alone and all co-cultures with anti-CD19 CAR-T cells) did not induce T cell responses. The OV model with very low CLDN3 expression by RNAseq also induced anti-claudin-3 CAR-T cell response with lower levels of IFNγ and IL-2 compared to the two CR models run in the same experiment and comparable TNF-α measurements.

Downstream PDX cell characterisation demonstrated no CLDN3 expression in the ovarian model via flow cytometry. As it was demonstrated that anti-claudin-3 CAR-T cells showed no cytotoxic cross-reactivity towards other Claudin family members (see above) and off-target binding effects were not seen in screens (see below) the ovarian cells might express low CLDN3 levels below the level of detection of flow cytometry. This is in line with previous co-culture experiments with anti-claudin-3 CAR-T cells that showed increased cytokine levels in presence of cell lines with very low CLDN3 expression. As expected no CLDN3 was detected in the RKO-KO cells (negative control) in any of the experiments. Following the co-culture, PDL-1 levels in target expressing tumour cells were elevated in the anti-claudin-3 CAR-T cell group when compared to the anti-CD19 control group. Furthermore, anti-claudin-3 CAR-T cells showed increased CD69 levels compared to anti-CD19 CAR-T cells further confirming a response to the co-culture.

### Example 8

### Inclusion of CD20 in the Anti-Claudin-3 CAR Vector Provides a Mechanism of Controlled Anti-Claudin-3 CAR-T Cell Deletion with No Change to Anti-Claudin-3 CAR-T Cell Targeted Cytotoxic Activity

The presentation of Claudin-3 comes with associated risks whereby non-tumour related aberrant Claudin-3 expression could reactivate CAR-T cells and re-direct the cytotoxic T cells to attack cancer antigens on normal cells. To improve the safety profile of the Claudin-3 targeting CAR-T cells, a pre-programmed control safety measure in the form of T cell deletion technology can be introduced within the therapeutic vector rendering the T cell product susceptible to T cell deletion.

The cell surface B cell antigen, CD20, is the target for several therapeutic antibodies, namely FDA approved Rituximab, a type I antibody which binds to the disulphide-constrained portion of the CD20 major extracellular loop and induces apoptosis via Complement Dependent Cytotoxicity (CDC) and Antibody Dependent Cellular Cytotoxicity (ADCC; Golay et al., 2013 MAbs 5:826-837).

The objective of the study was to i) evaluate CD20 as an effective CAR-T cell deletion technology, ii) evaluate whether inclusion of CD20 in the 906_009 therapeutic vector alters the cytotoxic response of 906_009 CAR-T cells to Claudin 3 expressing target cells, iii) observe any changed in Calcium flux in CAR-T cells expressing CD20 and iv) predict the immunogenicity of CD20_906_009_SO (anti-claudin-3 CAR, splice site optimized (SO) vector). The results demonstrate that inclusion of CD20 in an anti-claudin-3 CAR-T cell therapy strategy can be used as a CAR-T cell deletion technology.

### Materials and Methods

In order assess the activity of CD20, comparisons were made between CAR-T cells transduced with either claudin-3 CAR vector including the CD20 ablation element (CD20_906_009) or lacking the CD20 ablation element (906_009). Untransduced and CD20_ZSGreen (vector expressing CD20 and ZSGreen fluorescent protein) or CD20_CD19 (vector expressing CD20 and CD19) transduced

T cells were included as controls. CD20 was evaluated for targeted T cell deletion by the CDC and ADCC assays. Any changes in CAR-T cell cytotoxic activity by inclusion of CD20 upstream of 906_009 was assessed by XCELLIGENCE cytotoxic assay.

### Design and Generation of Lentiviral Transfer Vectors

Lentiviral (pG3) transfer constructs encoding CD20 cell ablation gene upstream of 906_009 CAR were designed to generate CD20_906_009. Alongside this, the anti-CD19 CAR molecule (mirroring the two architectures present in the 906_009 CAR), with upstream CD20 and short spacer CD8α hinge, was also designed, CD20_CD19_GSK. The sequences were codon optimized and further modified to remove any potential splice sites from the sequence. The resulting transgene plasmids CD20_906_009_SO and 906_009_SO have the same protein sequence as their predecessors, CD20_906_009 and 906_009 respectively.

### Enrichment of CAR T-Cells

Day 13 post transduction CAR-T cells were selected by CAR expression using Rapidspheres as described above in Example 2, except cells were resuspended in Goat anti-mouse F(Ab)2—Biotin, rather than anti-LNGFR/CD271 Ab.

### CDC and ADCC Assay

CAR-T cells and control cells were resuspended in staining solution. In particular, CD20_906_009 CAR-T cells were stained with Cell Trace Violet (CTV) and 906_009 CAR T-cells were stained with Cell Trace Far Red (CTFR) (CTFR was used to stain untransduced cells or cells expressing anti-claudin-3 CAR only, and CTV was used to stain cells expressing CD20). The CTV and CTFR stained cells were paired by donor at a 1:1 ratio.

For CDC assay, the pairs were then treated with Rituximab (MabThera) or anti-RSV Isotype control and rabbit complement (Rab) or heat inactivated rabbit complement (HI) (FIG. 25). The proportion of CTV in the total cell pool, from 13 donors, was plotted against CD20 expression cells in FIG. 26.

For ADCC assay, fresh blood was obtained from the Blood donation unit (GSK-Stevenage). PBMCs were isolated as described hereinbefore. The cells then proceeded to negative selection of NK cells using NK cell Biotin-Antibody and MicroBeads. The cells then proceeded onto magnetic separation on a LS column and the unlabelled cells were collected and added to the stained and paired T-cells. The co-cultures were incubated at 37° C. for 20 hours.

### XCELLIGENCE Cytotoxicity Assay

Co-cultures for XCELLIGENCE killing assays were set up as described above in Example 4, with target cells K562 and RKO-KO co-cultured with effector cells (CAR-T and control T cells) at a 1:1 ratio of effector to target cell. The controls present were target cell only, effector cells only and target plus 100% Lysis (0.5% Triton X).

### Calcium Flux Analysis

CAR-T cells and Untransduced controls were seeded in a cell culture plate at 5×10⁴ cells per well and incubated at 37° C., 5% CO₂ followed by addition of assay buffer containing: 1) Thapsigargin (SXFAC=18 μM, 3.6 μM FAC) and DMSO (SXFAC=0.6% v/v, 0.12% v/v FAC); and 2) Ionomycin (6XFAC=4 μM, 0.67 μM FAC). The treated cells were then analysed by FLIPR.

CD20 is Targeted by Rituximab from Both Complement Dependent Cytotoxicity (CDC) and Antibody Dependent Cellular Cytotoxicity (ADCC)

In order to confirm whether the inclusion of CD20 in the anti-claudin-3 CAR-T cells can mark the therapy cells for deletion by Rituximab, CDC and ADCC assays were performed.

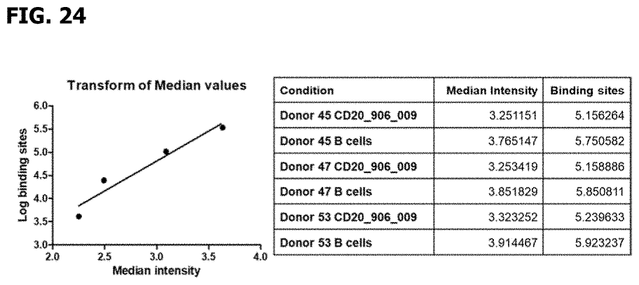

The level of CD20 expression on therapeutic anti-claudin-3 CAR-T cells was compared to the well-established Rituximab target, B cells (FIG. 24). By using beads with known human Fc binding sites and a human mAb directed against CD20 (anti-human Quantum Simply Cellular beads and anti-CD20-PE-Vio770, respectively), the number of potential CD20 binding sites were calculated using the median fluorescence intensity of CD20. The data shows that the number of CD20 binding sites on CD20_906_009 CAR-T cells ranged from 5.16 to 5.24 across 3 donors compared to donor matched B cells which ranged from 5.75 to 5.9. The CD20+ population within the CD20_906_009 CAR-T cells ranged between 35-41%. The range of CD20+ expression in CD20_906_009 CAR-T cells used throughout the CDC and ADCC data presented herein is 35-74%, therefore the cells in this assay represent the lower transduction rates which leads to the conclusion that the CD20 expression of the CD20_906_009 CAR-T cells are comparable to B cells.

A CDC assay was performed to confirm that anti-claudin-3 CAR-T cells expressing CD20 can be deleted when treated with Rituximab and complement. This data demonstrates that deletion occurs in the CTV stained cells when treated with Rabbit complement (Rab) plus Rituximab whereas the Isotype and HI treated CTV stained cells (control) are not deleted (FIGS. 25 and 26). The effect of deletion is also dependent on % CD20 expression within the CTV stained cells, whereby more cell deletion is observed as the CD20+ population increases. Further analysis compares the proportion of CTV cells (pCTV) of the Rab to HI treated condition.

As the mechanism by which Rituximab deletes CD20+ cells is not fully understood and may be by CDC and/or ADCC and because the use of Rabbit complement in place of human complement may not accurately predict CDC in humans, an ADCC assay was performed to confirm that anti-claudin-3 CAR-T cells expressing CD20 can be deleted when treated with Rituximab plus NK cells. The anti-claudin-3 CAR-T cells generated with the original CD20_906_009 and 906_009 vectors were compared to the Splice Site Optimised (SO) vectors CD20_906_009_SO and 906_009_SO. The data set for ADCC presented herein is from 3 donors.

FIG. 27 shows the pCTV ratio of NK treatment compared to media control plotted for either the Isotype or Rituximab condition. The assay for donor 62 was repeated and the data labelled 1 and 2 for the first and second experiment respectively. The results suggest that there is a decrease in cell number for both the original CD20_906_009 and the CD20_906_009_SO CAR-T cells. In donor 62, the deletion events are similar between original and SO variants for each of the independent assays however the CD20 expression differs at 54% and 76% respectively. The second assay was performed with freshly thawed cells which may have impeded the results. ADCC with donor 79 was also performed with freshly thawed cells, which, again does not demonstrate a striking result for either CD20_906_009 or CD20_906_009_SO CAR-T cells. CAR-T cell deletion in donor 87 is greater for CD20_906_009_SO than CD20_906_009 CAR-T cells which could be due to the CD20 expression being 83% and 62% respectively. To enhance the effect of ADCC, CD20_906_009 and 906_009 non-cryopreserved CAR-T cells from donor 62 and 87 were enriched by 906_009 CAR expression. A difference in the pCTV ratio is observed, likely due to the increased CD20+ population in the CTV stained condition.

### CD20 Does Not Alter 906_009 CAR T Cell Cytotoxicity of Claudin-3 Target Cells

Validation of anti-claudin-3 CAR-T cell cytotoxicity with and without CD20 was measured in real time by XCELLIGENCE assay, where cell growth is traced over time using impedance measurements. The claudin 3 expressing cell line, HT-29-Luc were targeted by 906_009 and CD20_906_009 CAR-T cells from 13 donors. Untransduced, CD20-ZSGreen T cells and CD20_CD19_GSK CAR-T cells were included for control. Cytotoxicity was measured every 30 minutes, where lack of impedance correlated with tumour cell killing. Due to over confluency of the control target cells, cytotoxicity analysis was only valid up to 24 hrs. FIG. 28 shows that the % of cells alive at 20 hrs does not differ significantly between CD20_906_009 and 906_009 CAR-T cells where both values are close to 0%, whereas the control cells are closer to 100% alive.KT50 values in FIG. 29 demonstrate that the time it takes to kill 50% of the target cells does not differ significantly between CD20_906_009 and 906_009 CAR-T cells.

FIG. 30 shows the % cells alive at 20 hours for the SO and original CAR-T cells from 4 donors. All the conditions were at or approaching 0% alive cells at 20 hours however % Cells alive at 20 hours is significantly lower in CD20_906_009 vs CD20_906_009_SO, and suggestively lower in 906_009 compared to 906_009_SO. Furthermore, the KT50 value is significantly lower in CD20_906_009 vs CD20_906_009_SO, and suggestively lower in 906_009 compared to 906_009_SO (FIG. 31).

### There is No Change in Calcium Flux in CAR-T Cells With and Without CD20

To determine whether CD20 influences CAR-T cell's calcium flux, untransduced T cells, CD20_906_009_SO and 906_009 CAR-T cells from 4 donors were first exposed to Thapsigargin which inhibits endoplasmic reticulum Ca2+-dependent ATPase, leading to increased cytosolic calcium levels, this was followed by addition of Ionomycin to stimulate calcium influx. DMSO was included for control and the treated cells were analysed by FLIPR. The DMSO condition for donor 99 906_009_SO CAR-T cells detached from the plate and therefore generated outlying negative values. The results in FIG. 32 show that there is no difference in Calcium flux between the untransduced or CAR-T cells with or without CD20.

The data presented herein supports the use of CD20 in an anti-claudin-3 CAR-T cell therapy strategy as a CAR-T cell deletion technology.

The CDC data demonstrated that anti-claudin-3 CAR-T cells expressing CD20 are marked for deletion by Rituximab. The preliminary ADCC data also suggest that deletion with Rituximab is also performed with NK cells. The performance of CAR-T cell deletion in both the CDC and ADCC assays depend on the CD20+ population which could suggest the potential for complete clearance of CD20+ CAR-T cells in these in vitro methods. Encoding CD20 upstream of the 906_009 CAR in the transgene vector did not impact the cytotoxicity of the anti-claudin-3 CAR-T cells.

CD20 is thought to act as a calcium channel in B cells however CD20 does not appear to alter the calcium flux of anti-claudin-3 CAR-T cells generated with CD20_906_009_SO compared to untransduced or 906_009 CAR-T cells.

### Example 9

### Assessing Binding of Anti-Claudin-3 CAR-T Cells to Proteins Other than the Intended Target (Off-Target Binding) Using a Plasma Membrane Protein Array

The objective of this study was to identify any off-target activities for transduced T cells. Binding of anti-claudin-3 CAR-T cells to proteins other than the intended target was assessed by a plasma membrane protein array using a set of expression vectors with a panel consisting of >5000 full-length clones covering more than 3500 different plasma membrane proteins, with many proteins represented by multiple variants. BCMA CAR-T cells were included in the study as a positive control.

### Materials and Methods

### Generating CAR-T Cells to Support Pre-, Primary and Confirmatory Screen

T cells purified from PBMCs isolated from human blood as described in Example 1 were transduced with BCMA-CAR lentiviral vector (BCMA-030), with a MOI of 2.4 or Claudin 3 CAR lentiviral vector (906-009) with a MOI of 5. Cells were incubated at 37° C. with 5% CO₂ and maintained in TEXMACS media and IL-2 at 100 IU/ml throughout the culture period. Cells were harvested 12 days after transduction and frozen in CryStor CS5 freezing media at 1×10⁸ cells/ml. Untransduced T cells were generated as a negative control. T cells were generated from one donor, 90928.

Transduction efficiency for BCMA CAR-T cells was determined by measuring binding to BCMA-AF647 using flow cytometry (MACSQuant Analyser 10). Transduction efficiency for anti-claudin-3 CAR-T cells was determined by measuring LNGFR expression using a PE conjugated anti-LNGFR Ab and flow cytometry (MACSQuant Analyser 10). Data was analysed using FlowJo v10.1.

### Plasma Membrane Protein Array

Pre-screen study: Untransduced and CAR transduced T cells (donor 90928) were added to slides of fixed untransfected HEK293 cells and HEK293 cells overexpressing BCMA, Claudin 3, known T cell interactors and control proteins to investigate the level of background staining prior to the primary screen.

Primary screen: For the primary screen, 4070 proteins encoding full-length human plasma membrane proteins were individually expressed in human HEK293 cells using reverse transfection. The cells were arrayed in duplicate across 13 microarray slides and fixed. The untransduced and CAR transduced T cells from donor 90928 were labelled with a Cell Tracer Red dye and applied to the plasma membrane protein array at a pre-optimised ratio of T cells to HEK293 cells.

Confirmatory screen: Vectors encoding the hits identified in the primary screen were spotted in duplicate and used to reverse transfect human HEK293 cells. Duplicate slides were set up. Untransduced and transduced T cells from donor 90928 (3.2×10⁷ cells per slide) were applied to the plasma membrane protein array.

Binding was assessed by imaging for fluorescence and quantitated for transduction efficiency using ImageQuant software (GE). A protein ‘hit’ was defined as duplicate spots showing a raised signal compared to background levels. This was achieved by visual inspection using the images gridded on the ImageQuant software. Hits were classified as ‘strong’, ‘medium’, ‘weak’ or ‘very weak’ depending on the intensity of the duplicate spots.

### Results

### Generating CAR-T Cells to Support Primary and Confirmatory Screen

Transduction efficiency was determined 12 days after transduction. The transduction efficiency of BCMA CAR-T cells was 63.1% and the transduction efficiency of 906-009 CAR-T cells was 50%.

### Plasma Membrane Protein Array: Pre-Screen

Donor 90928 was selected for the primary screen. The spotting pattern for HEK transduced cells is shown in FIG. 33A. Binding was observed with untransduced T cells to known T cell interactors (PVR, CD244, TNFSF4, ICOSLG, CD86) (FIG. 33B). Binding was observed with BCMA transduced T cells to BCMA transfected HEK293 cells (FIG. 33C) and with 906-009 CAR-T cells to Claudin 3 transfected HEK293 cells (FIG. 33D).

### Plasma Membrane Protein Array: Primary Screen

In total 28 hits were identified by analysing fluorescence on ImageQuant. The intensity of staining ranged from very weak to strong.

### Plasma Membrane Protein Array: Confirmatory Screen

The spotting pattern for the 28 hits is shown in FIG. 34A. Binding was observed with untransduced T cells to known T cell interactors. One specific interaction with BCMA expressing HEK cells was identified for BCMA CAR-T cells with strong intensity. One CAR-specific interaction was identified for 906-009 CAR-T cells with Claudin 3 expressing HEK cells (FIG. 34D and Table 11). Very weak intensity binding was inconsistently observed with SLC6A6 expressing HEK cells with 906-009 CAR-T cells within the confirmation screen, but not within the primary screen (data not shown).

TABLE 11
Summary of CAR - Specific Hits

| | | | | | | Confirmation | | |
|---|---|---|---|---|---|---|---|---|
| | | Protein | | Primary screen | | screen | | |
| Sample ID | Gene Id | Name | Accession | Rep 1 | Rep 2 | Rep 1 | Rep 2 | Comments |
| BCMA CAR | TNFRSF17 | TNF | BC058291 | medium | medium | strong | strong | 184aa, isoform |
| T cells | | Receptor | | | | | | 1/canonical, |
| | | Superfamily | | | | | | natural variant |
| | | Member 17/ | | | | | | 81 N-S, single- |
| | | BCMA | | | | | | pass type III |
| | | | | | | | | membrane |
| | | | | | | | | protein |
| Anti- | CLDN3 | Claudin 3 | BC016056 | weak | weak | weak | weak | 220aa, single |
| CLDN3- | | | | | | | | form/canonical, |
| LNGFR CAR | | | | | | | | multi-pass |
| T cells | | | | | | | | membrane |
| | | | | | | | | protein |

After screening the CAR-T cells for binding against human HEK293 over-expressing a library of 4070 human proteins, the untransduced T cells showed binding to many known T cell interactors. BCMA CAR-T cells, used a positive control, showed a single specific interaction with BCMA with strong intensity. 906-009 CAR-T cells demonstrated weak intensity binding to Claudin 3 expressing HEK cells.

### Example 10

### Cytokine Peak In Vivo Study

CARs are synthetic antigen receptors that reprogram T cell specificity, function and persistence. They are generally composed of ScFv or sdAbs fused to T cells activation domain—zeta chain of the CD3 complex and co-stimulatory domain—typically CD28 or 4-1BB. Engagement with the specific ligand will promote activation of CAR armoured T cells and enhance killing of target tumour cells (June and Sadelain 2018). In recent decade chimeric antigen receptor (CAR)-T therapy has become a promising field in immunotherapy showing high success in haematological tumours and demonstrating potential for treatment of solid tumours (Jackson, Rafiq, and Brentjens 2016; Fucà et al., 2020).

CLDN3 belongs to a large family of integral membrane proteins crucial for the formation of tight junctions (TJs) between epithelial cells (Itallie and Anderson 2004). Disruption of the normal tissue architecture is a hallmark of cancer, and CLDN3 altered expression has been linked to the development of various epithelial cancers including those with high unmet need such as colorectal, breast, pancreatic and ovarian carcinomas (Singh, Sharma, and Dhawan 2010). It has been reported that CLDN3 is mis localized outside of TJs in tumours but not in healthy tissues (Corsini et al., 2018), a mechanism that turns CLDN3 into a CAR-T cell target for selective killing of tumour cells while sparing the normal cells where it is hidden in the tight junctions.

“SO-CD20-906_009” is a humanised CAR T specifically targeting CLDN3 antigen composed of humanised scFv along with a CD8 hinge, CD3 signalling domain and 4-1BB co-stimulatory domain. “902_007-LNGFR” is scFv CAR-T control with similar affinity to both human and mouse CLDN3. “CD20-CD19” is a non-CLDN3 CAR-T control with CD20 ablation component.

Tissue damage due to inflammation (i.e., increased cytokine release) might lead to exposure of CLDN3 on healthy tissues due to loss of tight junctions, making it accessible to CLDN3 CAR T cells posing therefore a potential safety risk. The primary objective of this study was to assess whether the potential increase in cytokine secretion induced by CLDN3 CAR T/tumour cell engagement may result in toxicity in healthy tissues due to potential disruption in tight junctions. Towards this direction, several timepoints were selected for cytokine release measurement ex vivo in sera samples from a CDX mouse model dosed with 902_007-LNGFR, SO-CD20-906_009 or CD20-CD19. The timepoints were selected in order to ensure that the cytokine secretion peak could be identified and subsequently that normal tissues could be assessed at the time of the cytokine secretion peak. An MSD multiplex assay was used for the detection of the following cytokines: IFNγ, IL-10, IL-12p70, IL-13, IL-β, IL-2, IL-4, IL-6, IL-8, TNF-α over time. Histopathological assessment of normal tissues and tumours was performed at the individual end points.

### Materials and Methods

To assess whether the increase in cytokine secretion induced by CLDN3 CAR T/tumour cell engagement may result toxicity in healthy tissues due to potential disruption in tight junctions, we used a HT-29 Luc human colorectal cancer model. HT29-Luc tumour-bearing NSG mice were dosed with CLDN3 CAR T cells (SO-CD20-906_009, 902_007-LNGFR) or non-targeting control CD19 CAR T cells (CD20-CD19) when tumours reached average tumour volume of 320 mm3. A time-course of cytokine secretion profile was performed. This was accompanied by histopathological assessment of tumours and organs (lung, liver, spleen, heart, colon, kidney, ovaries, brain, eyes, optic nerves). Specifically, there were the following study readouts: a) cytokine release in blood sera samples measured by MSD (IFNγ, IL-10, IL-12p70, IL-13, IL-1β, IL-2, IL-4, IL-6, IL-8, TNF-α) for days: 3, 4, 5, 7 and 14 post-T cell dosing and b) histopathological assessment of tumours and organs (lung, liver, spleen, heart, colon, kidney, ovaries, brain) for days: 3, 4, 7 and 14 post-T cell dosing and eyes and optical nerves for day 14 post-T cell dosing. The timepoint ‘5-days post T cell dosing’ was included for blood/serum collection only as an intermediate between early timepoints (day 3, 4), day 7 (which was historically selected in previous in vivo efficacy studies) and late timepoint (day 14).

### Sourcing of NOD SCID Gamma (NSG) Mice

96 female 8-9 week old NSG mice were acquired from Charles River, UK.

### Tumour Cell Inoculation (Study Day 0)

Prior to study start, supernatants (3×200 ul) from the HT29-Luc cells were submitted for testing for a comprehensive PCR panel of mouse/rat pathogens (Charles River) and for sterility testing. All samples were tested negative. HT29-Luc cells were upscaled in McCoy's, 10% FBS culture medium in 5% CO₂, 37° C. incubator for two weeks before inoculation into mice. Prior to inoculation, HT29-Luc cells were harvested and supernatants (3×200 μl) were collected for mouse/rat pathogen testing (Charles River) and for sterility testing for confirmation of pathogen-free status of the cells. Harvested HT29-Luc cells were counted and resuspended on ice in pre-chilled PBS: Matrigel (1:1) to a final concentration of 0.5×10⁶ cells in 100 μl per mouse. For 95 mice in total, cell needs were as following: 95×0.5×10⁶ cells (cell dose/mouse)×2 (for syringe dead volume)=9.5×10⁷ cells. Thus, a total of 10×10⁷ cells was resuspended in 20 ml of pre-chilled PBS: Matrigel (1:1). Cells were kept on ice and transferred to IVSD (8F, animal unit) for subcutaneous (s/c) inoculation of cells into the right flank of each NSG mouse.

### T Cell Dosing (Study Day 23)

On day 12-post transduction and prior to T cell freezing, supernatants (3×200 ul) from CAR T cells (SO-CD20-906_009, 902_007-LNGFR and CD20-CD19) were submitted for testing for a comprehensive PCR panel of mouse/rat pathogens (Charles River) and for sterility testing. All samples were tested negative. All cells were frozen down on day 12-post transduction and remained in frozen status (−150° C.). The number of vials needed for T cell dosing was pre-calculated and cells were thawed for T cell dosing usage on the same day, as described below.

On day of T cell dosing, CAR T cells (SO-CD20-906_009, 902_007-LNGFR and CD20-CD19) were thawed in a water bath (37° C.) and transferred to 50 mL tubes containing cold TexMACS media and pipetted up and down gently to continue the thawing process. Cold TexMACS was added to each tube to make up to a final volume of 50 mL. After centrifugation at 300×g for 10 min, RT, cell pellets were resuspended in cold TexMACS. Cells were centrifuged at 300×g for 10 min, RT and then resuspended in warm TexMACS and counted. Then, cells were centrifuged at 300×g for 10 min, RT and resuspended in pre-chilled PBS to a final concentration of 1×10⁷ cells in 100 μl per mouse. Cells were kept on ice and transferred to IVSD to animal unit (8F) for intravenous (i.v) injection of HT29-Luc tumour-bearing NSG mice. Detailed calculations were as follows:
SO-CD20-906_009: Handed over: 5.5×10⁸ cells
902_007-LNGFR: Handed over: 6.47×10⁸ cells
CD20-CD19: Handed over: 6.4×10⁸ cells

### Study Design

FIG. 39 illustrates the study design. Briefly, female NSG mice were inoculated with HT-29Luc on study day (SD) 0. On SD23, mice were dosed with CAR T cells (when tumours reached ˜320 mm3). Blood samples were collected on SD5, SD26, SD27, SD28, SD30 and SD37. Tissues and tumours were collected on SD26, SD27, SD30 and SD37.

### Robust Study Design Considerations

In agreement with the UK SRF, the study statistician and the robust study design guidelines, randomisation and blinding strategies were as follows:

Randomisation: Animals were randomisation upon arrival. Additionally, prior to T cell dosing, animals were allocated to treatment groups according to a formal randomisation plan based on tumour volume spread after consulting with the study statistician: Jack Euesden. When tumours reached average volume ˜320 mm³, animals were randomised (based on tumour measurements one day prior to T cell dosing to allow time for randomisation decision) according to tumour volume spread. Specifically, the mean log 10 tumour volume was calculated for each cage and cages were split into two blocks—‘low’ tumour volume (lower or equal to overall median) or ‘high’ (higher to overall median) tumour volume. A randomised complete block design with two treatment factors (day and treatment) was performed using JMP v14. Randomisation was performed for all 12× groups (different treatments and different endpoints/blood sampling/tumour collection). Additionally, ex vivo MSD readout was subjected to randomisation.

Blinding: Study personnel was blinded throughout the study. Specifically, cell dose and treatment were blinded to IVSD and scientists supported T cell preparation for dosing. Also, both MSD and histopathology readouts were blinded.

### Tumour Cell Inoculation (Study Day 0)

S/c tumour implantations were carried out in a class II sterile cabinet. All equipment used was sterilised prior to use. Animals were briefly anaesthetised in a chamber by isoflurane-oxygen mix and moved to face cone. Right flank was shaved then wiped with alcohol wipe. Cells were resuspended in PBS and then mixed well with Matrigel on ice (1:1 PBS/cells:Matrigel). A total volume of 100 uL of Matrigel and PBS solution with cells were injected s/c per mouse. Animals were moved to recovery area to be monitored until fully recovered before placed back in home cage and monitored.

### T Cell Dosing (Study Day 23)

When tumours reached average volume of 320 mm³, CAR T cells were dosed via tail vein injection at a dose of 1×10⁷ cells per mouse. Intravenous (i.v.) dose of therapy was carried out in a class II sterile cabinet.

Tumour Measurements, Study Plan and Individual Endpoints Tumour size in all mice was measured by calliper measurements and recorded three times per week. Bodyweight for all mice was measured starting from study day −21 (7 days upon mice delivery). Following bodyweight measurement on study day −16, all subsequent measurements were recorded with a 2-day interval. Tumour volume was calculated with Excel as indicated below:

Tumour volume=Tumour length*(Tumour Width\textcircled{ }2)*0.5

There were individual endpoints (days: 3, 4, 7 and 14 post-T cell dosing) in this study.

### Blood Collection

Blood samples from all mice were collected on study day 5 (23 days prior to T cell dosing), except mouse #43 (‘day 3’ endpoint, group: 902_007-LNGFR) due to low bodyweight. Subsequent blood withdrawal was performed on study days 26, 27, 28, 30 and 37 (day 3, 4, 5, 7, and 14 post T-cell dosing, respectively) in mice depending to cage ID based to a formal randomisation plan, as described above. Of note, blood samples were collected from all CAR T groups across all timepoints. Approximately 100 μl blood per mouse with an additional 5 μl of blood for wastage per sample was collected. For the serum samples, whole blood was collected into Serum Microtainer tubes and allowed to clot for a minimum of 30 minutes at room temperature (RT). Once clotted the blood was centrifuged at 14840×g for 3 minutes and serum transferred into Micronics tubes. Serum was frozen at −80° C. until used in the MSD assay. According to the licence, the maximum blood volume to be taken per mouse within 28 rolling days is 10% of mice body weight.

### Serum Cytokine Assay MSD

An MSD assay using the 10 plex plates was carried out as per manufactures instructions:

Reagent and sample preparation: Frozen mouse serum samples, commercial mouse sera and V-PLEX diluents were thawed, and equilibrated RT. Assay calibrator was reconstituted according to manufacturer's instruction. All the reagent and antibodies were kept on ice when not in use during the experiment.

Calibrator and sample dilutions: Calibrator 1 supplied by the MSD kit was resuspended in 1 mL Diluent 2, inverted 3×, equilibrated at RT for 15 min and then vortexed briefly using short pulses. For the serial dilution to generate cytokine standards calibration curve: the highest calibrator 1 concentration was made by transferring 300 μl of reconstituted Calibrator 1 solution into a fresh Eppendorf tube. Then, the next calibrator dilution was made by transferring 100 μl of the highest calibrator to 300 ul of Diluent 2, and mixed well by vortexing. This 4-fold serial dilution was performed for 5 additional times to generate 7 calibrators. The 8^{th} vial was filled with Diluent 2 only. An additional calibration set (serum standards) commercially available mouse serum was included to confirm recovery: calibrator dilutions were prepared in Diluent 2 with 20% commercially available mouse sera, as described above (4-fold serial dilution). Final vial (8^{th} vial) contained 20% mouse serum in Diluent 2 only. For the sample dilution: all mouse serum samples were prepared by adding 25 ul of the serum to 100 ul of Diluent 2 (1 in 5 dilution) and mixed properly. For detection antibody solution preparation: MSD provided each detection antibody separately as a 50× stock solution. The working solution was 1×. Then, the detection antibody solution was detected immediately prior to use: combined 60 μl of each antibody (10 in total) and addes to 2.40 mL of Diluent 3. For read buffer preparation: MSD provided read buffer T as a 4× stock solution. The working solution was 2×. For 1 plate, combine 10 mL of read buffer T (4×) with 10 mL of deionized water.

Assay protocol: The plates were washed 3× with 150 μl/well of wash buffer. 50 μl of calibrators or prepared serum samples were added per well according to the plate layout. Next, the plates were incubated at RT with shaking at 750 rpm for 2 hours. Following incubation, the plates were washed 3× with 150 μl/well of wash buffer and then the detection antibody solution (25 μl/well) was added and the plates were incubated at RT with shaking at 750 rpm for 2 hours. Following incubation, the plates were washed 3× with 150 μl/well of wash buffer. Next, 150 ul of 2× read buffer T were added to each well and then the plates were read on the MSD Sector 600 Imager immediately.

### Tumour Growth

Tumour volume data (mm³) was plotted on GraphPad Prism. One-way ANOVA with Tukey's multiple comparison test was used to compare CAR T groups for individual timepoints. Two-way ANOVA with Bonferroni multiple comparison was used to compare CAR T groups over time for the ‘day 14 post—T cell dosing’ endpoint.

### Serum Cytokine Assay

The MSD raw data was analysed by the study statistician using a linear mixed model implemented in the lme4 package within R version 3.6.1. Each cytokine was modelled separately. The full dataset can be seen in eLNB: N74766-9. Cytokine release was transformed to log 10 to ensure homoskedasticity. Fixed effects were used for CAR T group and time (and their interaction). Time was modelled using a natural spline with 4 degrees of freedom (determined by AIC). Random intercepts were used for plate and mouse, with a random slope for each mouse. Linear contrasts were used to compare marginal means between constructs/time points, and multiple imputation with 1,000 iterations was used to handle values below the lower limit of quantification, with an appropriate degree of freedom correction (Barnard and Rubin 1999).

### Results

Female NSG mice were inoculated with the colorectal cancer cell line HT-29Luc (0.5×10⁶ cells/mouse) on SD0. When tumours reached ˜320 mm³ (SD23), mice were dosed with CAR T cells (SO-CD20-906_009, 902_007-LNGFR or CD20-CD19); (1×10⁷ cells/mouse). Sera samples were obtained from all mice on SD5 (23 days prior to T cell dosing), except mouse #43 (‘day 3’ endpoint, group: 902_007-LNGFR) due to low bodyweight. Next, sera samples were collected from the corresponding cages (based on the randomisation plan) on the following days post-T cell dosing: 3, 4, 5, 7 and 14 (SD26, SD27, SD28, SD30 and SD37, respectively). Timepoints: 3, 4, 7 and 14 were endpoints; blood withdrawal for subsequent serum isolation was employed prior to euthanasia. Mice from endpoint ‘day 14’ were also used for serum collection on ‘day 5’ timepoint. For all individual endpoints, tumours and tissues (lung, liver, spleen, heart, colon, kidney, ovaries, brain) were collected and submitted for histopathological assessment. For ‘day 14’ endpoint specifically, eyes and optic nerves were collected, as well.

Cytokine Release Over Time in CDX Mouse Model Dosed with SO-CD20-906_009, 902 007-LNGFR or CD20-CD19

Sera samples from all mice across all timepoints were run in 3 subsequent rounds using an MSD 10-plex assay following a formal randomisation plan. To avoid round-to-round variability and to adhere to robust study design principles, randomised plate layouts for MSD assay included samples from all timepoints. Some key observations for IFNγ, IL-2 and TNF-α secretion (FIG. 35) are summarised below:

IFNγ:
902_007-LNGFR differed significantly from baseline compared to CD20-CD19 control group at day 3, day 4, day 5 and day 7 timepoints (p<0.0001 for all) (FIG. 35A-FIG. 35B).
SO-CD20-906_009 differed significantly from baseline compared to CD20-CD19 control group at day 3, day 4, day 5 and day 7 timepoints (p<0.0001 for all) (FIG. 35A-FIG. 35B).
There was a statistically significant drop at day 14 for 902_007-LNGFR group compared to day 3, day 4, day 5 and day 7 timepoints (p<0.0001 for all, except day 3 vs. day 14: p=0.0079) (FIG. 35A-FIG. 35B).
There was a statistically significant drop at day 14 for SO-CD20-906_009 group compared to day 3, day 4, day 5 and day 7 timepoints (p<0.0001 for all), (FIG. 35A-FIG. 35B).
902_007-LNGFR and SO-CD20-906_009 groups seemed to have different kinetics; there is a trend for an earlier increase in SO-CD20-906_009 group from day 3 (FIG. 35A).
A secretion ‘peak’ could be observed for both 902_007-LNGFR and SO-CD20-906_009 groups (FIG. 35B) on day 7.

IL-2:
902_007-LNGFR differed significantly from baseline compared to CD20-CD19 control group at day 4 (p=0.0331) and day 5 timepoints (p=0.0098) (FIG. 35C-FIG. 35D).
SO-CD20-906_009 differed significantly from baseline compared to CD20-CD19 control group at day 4 (p=0.0022) and day 5 (p=0.0031) timepoints (FIG. 35C-FIG. 35D).
There was not a statistically significant drop at day 14 for 902_007-LNGFR compared to day 4 (p=0.582) and day 5 (p=0.8862) timepoints (FIG. 35C-FIG. 35D).
There was not a statistically significant drop at day 14 for SO-CD20-906_009 compared to day 4 (p=0.0963) and day 5 (p=0.0518) timepoints (FIG. 35C-FIG. 35D).

TNF-α
902_007-LNGFR differed significantly from baseline compared to CD20-CD19 control group at day 4 (p=0.0377), day 5 (p=0.0094) and day 7 (p=0.0291) timepoints (FIG. 35E-FIG. 35F).
SO-CD20-906_009 differed significantly from baseline compared to CD20-CD19 control group at day 3 (p=0.003), day 4 (p<0.0001), day 5 (p<0.0001) and day 7 (p=0.0126) timepoints (FIG. 35E-FIG. 35F).
There was a statistically significant drop at day 14 for 902_007-LNGFR group compared to day 7 (p=0.0317) (FIG. 35E-FIG. 35F).
There was a statistically significant drop at day 14 for SO-CD20-906_009 group compared to day 3 (p=0.0011), day 4 (p<0.0001), day 5 (p<0.0001) and day 7 (p=0.0002) (FIG. 35E-FIG. 35F).

Overall, there was increased cytokine secretion in both 902_007-LNGFR and SO-CD20-906_009 groups compared to the CD20-CD19 control group for all cytokines (FIG. 36) post T-cell dosing. It is worth mentioning that there is a drop of cytokine secretion at day 14 for SO-CD20-906_009 for all cytokines (FIG. 36).

### Impact of 902_007-LNGFR and SO-CD20-906_009 on Tumour Growth in CDX Mouse Model

Prior to use in this study, both CLDN3 CAR T cells (902_007-LNGFR or SO-CD20-906_009) were QC-tested in vitro. Briefly, the functional activity of 902_007-LNGFR or SO-CD20-906_009 was assessed by cytokine (IFNγ, IL-2 and TNF-α) release measured by MSD. Specifically, 902_007-LNGFR, SO-CD20-906_009, CD20-CD19 or untransduced (“UT”) cells were co-cultured with a panel of colorectal cancer cell lines, including HT-29Luc cells, for ˜22 h. This panel was selected to include cancer cell lines expressing CLDN3 target (HT-29Luc, RKO KO human CLDN3) and the RKO KO cell line in which CLDN3 expression is low/absent. Overall, CLDN3 CAR T cells passed QC successfully prior to in vivo and it was shown that CLDN3 CAR T cells secreted IFNγ, IL-2 and TNF-α in response to CLDN3-expressing colorectal tumour cells, as expected.

Functional assessment demonstrated the cross-reactivity of 902_007-LNGFR and SO-CD20-906_009 towards mouse CLDN3. 902_007-LNGFR and SO-CD20-906_009 displayed similar levels of cytotoxicity towards human CLDN3 (shown by cell area decreasing at similar rates). SO-CD20-906_009 also secreted cytokines in response to mouse CLDN3 target cells and partially killed mouse CLDN3 cell lines (shown by reduced cell area compared to controls and a mixture of live and dead cells in images at the end of the assay). The killing response of SO-CD20-906_009 to mouse CLDN3 was significantly less than the response of 902_007-LNGFR towards mouse CLDN3. When the response of 902_007-LNGFR to mouse CLDN3 and human CLDN3 was compared, the percentage of dead cell area in mouse CLDN3 and 902_007-LNGFR co-cultures was less.

Tumour growth kinetics for groups of mice from ‘day 14’ endpoint were assessed (FIG. 37). There was a statistically significant decrease in tumour volume in mice dosed with SO-CD20-906_009 compared to mice dosed with CD20-CD19 on SD35 (12 days post-T cell dosing), (p<0.01); an effect that was prolonged until the endpoint (SD37; 14 days post-T cell dosing), (p<0.0001). Of note, the transduction efficiency (T.E.) for SO-CD20-906_009 and CD20-CD19 was normalised to 63% before dosing into mice to allow such comparisons. Overall, SO-CD20-906_009 had a potent anti-tumour effect showing a drastic tumour volume reduction.

On the other hand, there was a trend of tumour growth control by 902_007-LNGFR, starting at SD33, although there was no statistically significant difference when compared to other CAR T groups (FIG. 37). This indicated that 902_007-LNGFR actively controlled tumour growth in vivo and taken into consideration the cytokine secretion profile, it seems that 902_007-LNGFR was efficacious in this tumour model. It should be noted that 902_007-LNGFR had lower T.E. compared to the other CAR molecules. Specifically, 902_007-LNGFR cells had almost half T.E. compared to CD20-CD19 and SO-CD20-906_009. 902_007-LNGFR were not normalised before dosing into mice. Thus, no assumptions or conclusions can be drawn regarding differences in tumour growth impact between 902_007-LNGFR and other CAR T groups. Of note, the present study did not aim to compare or assess the tumour growth kinetics of CLDN3 CAR T cells-treated tumours as it was not a standard efficacy study, but had a defined endpoint instead.

Finally, it is worth mentioning that there was no statistically significant difference in tumour volume in mice dosed with CD20-CD19 versus SO-CD20-906_009 or 902_007-LNGFR at ‘day 4’ or ‘day 7’ endpoints (FIG. 38).

Toxicity Assessment in CDX Mouse Model Dosed with, 902_007-LNGFR, SO-CD20-906_009 or CD20-CD19

The toxicity of 902_007-LNGFR or SO-CD20-906_009 was investigated over 2 weeks in an NSG mouse model of HT-29 Luc human colorectal carcinoma. Selected tissues including tumours were examined microscopically. Histopathological assessment was part of an investigation aimed at determining whether high circulating levels of pro-inflammatory cytokines potentially released into the blood circulation by CLDN3 CAR T cells following robust tumour engagement can induce subsequent on-target-off-tumour toxicity potentially by disrupting tight junctions in epithelia of normal tissues leading to exposure of CLDN3.

Neither 902_007-LNGFR nor SO-CD20-906_009 caused toxicity or accumulated in murine CLDN3 expressing normal non-inflamed (no inherent or induced inflammation) tissues, namely lung, liver, spleen, heart, colon, kidney, ovaries and brain. However, both CLDN3 CAR T products ablated the human CLDN3 positive colorectal carcinoma tumours.

Mislocalisation of CLDN3 in tumour tissues makes this target accessible to CAR T cell therapy for selective tumour cell killing. On the other hand, tissue damage due to inflammation (i.e., increased cytokine release) might lead to exposure of CLDN3 on healthy tissues or/and loss of tight junctions making it accessible to CLDN3 CAR-T cells posing therefore a potential risk. The effects of pro-inflammatory cytokines, such as IFNγ and TNF-α on TJs and epithelial permeability have been described (Coyne et al., 2002; Prasad et al., 2005; Capaldo and Nusrat 2009).

Absence of any CAR T cell-related effects on normal tissues where CLDN3 is known to be localised, was reported in previous in vivo studies with NSG mice. Such a finding is encouraging but is not definitive in terms of human clinical safety. Therefore, an in vivo investigation was needed to assess whether high levels of pro-inflammatory cytokines were released into the blood circulation by CLDN3 CAR T cells following robust tumour engagement. Moreover, it was assessed whether such cytokine secretion can induce subsequent on-target-off-tumour toxicity potentially by disrupting tight junctions in epithelia of normal tissues leading to exposure of CLDN3. Previous in vivo efficacy studies using the HT-29Luc tumour model and CLDN3 CAR T cells (various constructs) assessed cytokine secretion pre-T cell dosing (baseline) and 7 days post-T cell dosing. An increase in IFNγ secretion in mice dosed with CLDN3 CAR T cells compared to mice dosed with non-targeting control CAR T cells was reported 7 days post-T cell dosing compared to baseline. Nevertheless, minimum/negligible secretion of TNF-α and IL-2 was observed in mice dosed with CLDN3 CAR T cells compared to mice dosed with non-targeting control CAR T cells 7 days post-T cell dosing compared to baseline. Although such findings provided key evidence in regard to efficacy of CLDN3 CAR T cells in vivo, cytokine secretion kinetics were unexplored. Concomitantly, histopathological assessment of normal tissues and tumours was performed at the endpoint only in the aforementioned studies. Likewise, the ‘window’ to assess potential on-target-off-tumour toxicity may have been missed due to fast tissue recovery that was completed by the endpoint of these studies. Hence, it was decided to assess toxicity in timepoints close to the cytokine secretion peak. For this, both the cytokine secretion kinetics and the potential toxicity as a result of CLDN3 CAR T/tumour cell engagement effect in the presence of elevated cytokine secretion were investigated in frequent time points.

Towards this direction, established tumours in HT-29Luc-tumour bearing NSG mice were dosed with CLDN3 CAR T cells (SO-CD20-906_009 or 902_007-LNGFR) or non-targeting control CAR T cells (CD20-CD19). Of note, T cell dose remained the same compared to previous in vivo efficacy studies. To ‘stretch’ the in vivo model in order to trigger high cytokine secretion levels, the tumour volume on T cell dosing was higher compared to the previous in vivo efficacy studies. Both CLDN3 CAR T groups showed a secretion ‘peak’ for IFNγ. Although there is no statistically significant difference between earlier timepoints (day: 3 4 or 5) and day 7, a secretion ‘peak’ is not strictly defined here. It is worth highlighting that there was a statistically significant drop in IFNγ secretion at day 14 compared to day 3, day 4, day 5 and day 7 timepoints. Additionally, day 7 can be seen as the last timepoint when IFNγ secretion levels are significantly higher before dropping at day 14. Taking this into consideration, it could be suggested that the IFNγ release reached its maximum levels systemically in vivo,'peaking' at day 7 post-CLDN3 CAR T cells dosing. Day 7 post-dosing has also been reported to be the secretion ‘peak’ for KTE-X19, an anti-CD19 CAR T cell therapy in patients with relapsed or refractory mantle-cell lymphoma (Wang et al., 2020).

Of note, IFNγ secreted levels in SO-CD20-906_009 were elevated from early timepoints (day 3) and retained such a profile until day 7. However, there was no differential impact on tumour growth among all CAR T groups 7 days post-T cell dosing. By contrast, SO-CD20-906_009 significantly decreased tumour volume in vivo 12 days after dosing. This suggests that cytokine secretion following CLDN3 CAR-T/tumour cell engagement precedes tumour growth control in vivo.

Importantly, SO-CD20-906_009 and 902_007-LNGFR showed no toxicity or accumulation in murine CLDN3-expressing normal non-inflamed (no inherent or induced inflammation) tissues, namely lung, liver, spleen, heart, colon, kidney, ovaries and brain in this study.

It is worth mentioning that the histopathology readout complemented our conclusions from tumour growth measurements by calliper and that calliper measurements may have slightly lower sensitivity and later onset compared to histopathology, because tumours do not immediately implode upon ablation. SO-CD20-906_009 controlled tumour growth efficiently and this was in accordance with the histopathology readout which showed that SO-CD20-906_009 ablated human CLDN3 positive colorectal carcinoma tumours. However, the histopathology readout allowed us to conclude that 902_007-LNGFR had the potency/capability to control tumour growth, but the study was terminated too early to be able to manifest this from calliper measurements (not primary objective of this study). It should be highlighted that 902_007-LNGFR had almost half T.E. compared to CD20-CD19 and SO-CD20-906_009 in the present in vivo study. Thus, 902_007-LNGFR need more time to impact tumour growth.

In conclusion, this study has shown that increased cytokine secretion induced by either SO-CD20-906_009 or 902_007-LNGFR/tumour cell engagement in vivo did not cause toxicity or accumulation in murine CLDN3 expressing normal non-inflamed (no inherent or induced inflammation) tissues.

### Example 11

### Ablation In Vivo Study

CARs are synthetic antigen receptors that reprogram T cell specificity, function and persistence. “SO-CD20-906_009” is humanised CAR-T cells specifically targeting CLDN3 antigen composed of humanised scFv along with a CD8 hinge, CD3 signalling domain and 4-1BB co-stimulatory domain.

It has been reported that CLDN3 is mis localized outside of tight junctions (TJs) in tumours but not in healthy tissues (Corsini et al., 2018), a mechanism that turns CLDN3 into a CAR-T cell target for selective killing of tumour cells while sparing the normal cells where it is hidden in the tight junctions. However, CLDN3 may carry a risk of on-target off-tumour toxicity. To control this potential risk, a “ablation technology” enabling for the targeted depletion of inappropriately activated CAR T cells in the long term is investigated. This is achieved by CD20 co-expression of the CAR-T cells and the application of an Anti-CD20 antibody.

The objective of the present study was to provide proof of principle for ablation of SO-CD20-906_009 (CD20-co-expressing CAR) T cells in vivo by the administration of the anti-CD20 mAb, Rituximab. CAR T cell presence following mAb administration was investigated in the blood and in tissues (spleen, bone marrow, lung and liver) by ddPCR, flow cytometry and Immunohistochemistry (IHC).

Rituximab (RITUXAN, abbreviated within this report as RTX) is a mouse-human chimeric Anti-CD20 mAb, FDA-approved for the treatment of B cell lymphoma. The mode of action (MoA) of RTX in humans, is primarily mediated by macrophages and natural killer (NK) cells (Marshall et a!, 2017). In the mouse, it is thought to be mediated by myeloids, particularly macrophages while other reports demonstrate essential impact of NK cells (review by Marshall et al., 2017 and references therein, Uchida 2004, Shiokawa et al., 2010). In this study, the NSG-SGM3 mouse line which is deficient in T, B, and NK cells were used. However, this strain retains phagocyte effector function via mouse macrophages and transgenically expresses human IL3, GM-CSF and SCF which was shown to increase the mouse macrophage presence (Nicolini et al., 2004). In addition, human PBMCs (hPBMCs) were injected to these mice which include NK cells and monocytes. This system facilitates the use of the antibody-dependent cellular phagocytosis (ADCP) as well as the antibody dependent cell-mediated cytotoxicity (ADCC) mechanisms of RTX. Direct cell death and complement-dependent cytotoxicity (CDC) via the complement system are rather neglegible for RTX MoA in the mouse setting and the latter was argued to be favorable as well as unfavorable depending on the setting (Marshall et al., 2017). The mouse model used was found to be suitable for a proof of principle but as with every mouse model there are limitations regarding the translatability to patients. Within this study the following parameters were assessed:

1) Flow cytometric analysis to assess the number of SO-CD20-906_009 CAR-T cells in the terminal blood samples.

2) ddPCR analysis to assess the presence of SO-CD20-906_009 CAR-T cells in mouse blood by measure of the HIV vector integration into the DNA over the study course (pre-, 24 hrs and 72 hrs and terminal) and in the tissues (bone marrow, liver, lung, spleen) at the terminal timepoint.

4) Immunohistochemical (IHC) and in situ hybridisation (ISH) analysis to identify CD3+ T cells for general engraftment and distribution, CD20 expression as RTX-target cells, and WPRE-04 as SO-CD20-906_009 vector RNA expression in the tissues (bone marrow, liver, lung, spleen) at the terminal time point.

5) Bioanalytical analysis to assess terminal serum RTX concentrations. This is to confirm successful RTX application and assess terminal levels as potential explanation in case of absence of SO-CD20-906_009 CAR-T cell ablation within this model. This is especially relevant as immunodeficient mouse strains have been reported to display higher mAb clearance (Oldham et al., 2020).

6) Histopathological analysis of tissues (heart, colon, kidney, brain, ovary, lung, liver, spleen) to further understand the potential toxicity of CAR-T cells towards normal healthy tissues in this novel NSG-SGM3 mouse strain at the terminal time point.

Although no specific claim is being made, this study was conducted in accordance with accepted scientific practice for this type of study.

### Materials and Methods

Mouse strain: NOD.Cg-Prkdcscid Il2rgtm1WjlTg(CMVIL3,CSF2,KITLG)1Eav/MloySzJ (abbreviation: NSG-SGM3), 10-12 week old females. Upon shipment, mice were allowed to acclimatise for 10 days.

Randomisation: Mice were weighted pre-study start and randomised into treatment groups (A-D) and terminal sampling days based on their body weight.

Sample size was based on the Statistician's recommendation of ten mice per group with two cages of five mice per cage.

TABLE 12
Treatment groups with dosing regimen and sample size.

| | | | | Sample | |
|---|---|---|---|---|---|
| Group | PBMC | T Cells | mAb/vehicle | size | Name |
| A | X | N/A | RTX | 10 | No SO-CD20-906_009 ctrl |
| B | N/A | X | Vehicle | 10 | SO-CD20-906_009 and no |
| | | | | | mAb ctrl |
| C | X | X | RTX | 10 | SO-CD20-906_009 and mAb |
| D | X | X | Isotype | 10 | SO-CD20-906_009 and |
| | | | | | Isotype mAb ctrl |

X means that respective heading applies.
N/A means it does not apply (no cells).
Vehicle indicates that no mAbs but instead vehicle was administered.

On Study Day −1: hPBMC and SO-CD20-906_009 CAR-T cells from the same donor were thawed in TexMACS medium, counted, mixed in a 1:1 proportion, washed with phosphate buffered saline (PBS) once and prepared for dosing in sterile PBS. The T cell dose was based on previous in vivo efficacy study with CLDN3 CAR-T cells, the hPBMC dose was based on in-house studies. The transduction efficiency (TE) of the SO-CD20-906_009 construct was measured prior to injection with 37.8% while 32.4% CD20+ cells were detected (N74546-5). The vector copy number (VCN) of the SO-CD20-906_009 T cell product is 0.93 copies per cell. 1×10⁷ SO-CD20-906_009 T cells or 1×10⁷ hPBMCs or 1×10⁷ T cells plus 1×10⁷ hPBMCs were injected to the mice in 200 uL PBS via the tail vein (i.v.), following the regimen in Table 12 above. The cell suspension was gently agitated throughout the procedure to prevent cells from settling out in the syringe. The remaining cells were used for flow cytometic analysis to confirm TE, CD20 expression and in order to assess the cell composition.

Study Day 0: The final antibody concentrations were prepared freshly in the morning and administered at 250 ug per mouse in 100 uL via intraperitoneal (i.p.) route following the regimen in Table 12 above. The RTX dose was based on literature (Bonifant et al., 2016 and Valton et al., 2018) and consultancy of an expert in the field. As isotype control, the Anti-Respiratory Syncytial Virus (RSV) mAb Synagis was used. This is FDA-approved for the treatment of prevention of serious lower respiratory tract disease requiring hospitalisation caused by respiratory syncytial virus (RSV) in children at high risk for RSV disease. The Synagis dose is based on the RTX dose and similar or higher doses were previously used in mouse models without any toxicity being reported (Mejias et al., 2004). As vehicle control 5% Dextrose was used.

Sampling: The sampling timepoints are based on the literature and expert recommendations (Tasian et al., 2017, Bonifant et al., 2016, Valton et al., 2018, communication with expert in the field). During the in-life phase, 65 μL blood was collected from each mouse for PCR analysis at D0 before mAb dosing (pre-mAb) and then 24 and 72 hrs post-mAb or isotype or vehicle. For the blood collection, animals were placed into sterile transfer containers and warmed in a warming cabinet at an ambient temperature of 39° C. for approximately 10 minutes prior to tail bleeding. Terminal sampling was staggered over two terminal days (7 and 8 days post-mAb respectively), to ensure feasibility and high sample quality. Each terminal sampling day, five mice per group were humanely killed and samples collected. The mice sacrificed per group per day was randomised ahead of study initiation as described above.

Each mouse was deeply anaesthetised with isoflurane and terminal blood was collected via cardiac puncture for flow cytometry, PCR and serum RTX concentration assay. The mice were euthanised by cervical dislocation followed by confirmation of death by cessation of circulation via removal of the heart. In some noted cases, the blood clumped during terminal blood collection. This resulted in no serum sample for one mouse in the SO-CD20-906_009 and no mAb ctrl group.

Afterwards, bone marrow, spleen, liver and lung were harvested for PCR and histology. Additionally, heart, colon, kidney, brain and ovaries were collected for general histolopathological assessment in the mouse strain.

Blinding: The assessment and analysis of primary and secondary read-outs was fully blinded. Primary and secondary read-outs within this study were ddPCR, flow cytometry and IHC, ISH for detection of SO-CD20-906_009 and CD20 cells. Tertiary read-outs were RTX concentration and general histopathological assessment,

Flow Cytometric Analysis

### Characterisation of SO-CD20-906_009 & the Human PBMC Composition in Inoculates Pre-Inoculation

1-2×10⁵ cells were added per well into a 96-well V bottom polypropylene plate for antibody staining. Samples were first washed by centrifuging the plate (at 300×g) for 5 minutes, discarding supernatant and resuspending in 200 μL FACS buffer. Centrifugation was repeated and supernatant discarded. Samples were then resuspended in 100 μL of Fc blocker and incubated for 10 minutes at room temperature. Samples were then washed by adding 100 μL of FACS buffer and then centrifuged for 5 minutes and supernatant discarded. Samples were then stained with 100 μL of anti-f(ab′)2-biotin and incubated for 30 minutes at 4° C. in the dark. This was followed by two washes—first adding 100 μL FACS buffer, centrifuging for 5 minutes and discarding supernatant; and then repeated with 200 μL FACS buffer. Samples were then stained with 100 μL of an antibody cocktail (containing hPBMC specific antibodies and streptavidin-APC secondary) and incubated for 30 minutes at 4° C. in the dark. Two more washes were performed as described above and then samples resuspended in 100 μL of FACS buffer containing DAPI. Following 10 minute of incubation at room temperature in the dark, samples were acquired on a BD LSRFORTESSA X-20 flow cytometer.

Preparation of compensation controls: Compensation controls were prepared using ULTRA COMP EBEADS. In brief, 1 drop of ULTRA COMP EBEADS were added to a well of a 96-well v-bottom plate and 0.5 μL of each antibody-conjugate added at stock concentration. For anti-f(ab′2)-biotin+streptavidin-APC compensation control, 0.5 μL of each reagent were added to beads. For DAPI compensation control, 100 μL of cells were plated and 0.5 μL DAPI at stock concentration was added. Following 15 minutes of incubation at room temperature in the dark, compensation controls were acquired on a BD LSRFORTESSA X-20 and a compensation matrix calculated prior to the acquisition of blood samples.

### Characterisation & Counting of SO-CD20-906_009 & Human PBMCs in Mouse Terminal Blood (Flow Cytometry)

RBC lysis: RBC lysis solution was prepared as per the manufacturer's specification. Upon receipt of Mouse whole blood, approx. 400 μL of blood per mouse was transferred from vacutainers containing EDTA into 15 mL Falcon tubes containing 10 mL RBC lysis solution. Samples were vortexed briefly and then incubated for 10 minutes at room temperature. Samples were then centrifuged (at 300×g) for 7 minutes and supernatant carefully removed. Samples were then resuspended in an additional 1-5 mL of RBC lysis solution and incubated for a further 5 minutes to lyse any remaining RBC's. Following this, 5 mL of FACS buffer (DPBS+2% FBS (HI)+0.05% Sodium Azide+2 mM EDTA) was added and then samples were centrifuged for 5 minutes. After removing supernatant, samples were resuspended in the remaining supernatant (˜100-200 μL left in tube) and then transferred into a 96-well V bottom polypropylene plate for antibody staining.

Antibody staining: In preparation for antibody staining, samples were first washed by centrifuging the plate (at 300×g) for 5 minutes, discarding supernatant and resuspending in 200 μL FACS buffer. Centrifugation was repeated and supernatant discarded. Samples were then resuspended in 100 μL of Fc blocker and incubated for 10 minutes at room temperature. Samples were then washed by adding 100 μL of FACS buffer and then centrifuged for 5 minutes and supernatant discarded. Samples were then stained with 100 μL of anti-f(ab′)2-biotin and incubated for 30 minutes at 4° C. in the dark. This was followed by two washes—first adding 100 μL FACS buffer, centrifuging for 5 minutes and discarding supernatant; and then repeated with 200 μL FACS buffer. Samples were then stained with 100 μL of an antibody cocktail (containing hPBMC specific antibodies and streptavidin-APC secondary) and incubated for 30 minutes at 4° C. in the dark. Two more washes were performed as described above and then samples resuspended in 100-200 μL of FACS buffer containing DAPI. In addition, 10 μL of COUNTBRIGHT beads were added to each sample. Following 10 minute of incubation at room temperature in the dark, samples were acquired on a BD LSRFORTESSA X-20 flow cytometer.

Preparation of compensation controls: Compensation controls were prepared using ULTRA COMP EBEADS. In brief, 1 drop of ULTRA COMP EBEADS were added to a well of a 96-well v-bottom plate and 0.5 μL of each antibody-conjugate added at stock concentration. For anti-f(ab′2)-biotin+streptavidin-APC compensation control, 0.5 μL of each reagent were added to beads. For DAPI compensation control, 100 μL of cells were plated and 0.5 μL DAPI at stock concentration was added. Following 15 minutes of incubation at room temperature in the dark, compensation controls were acquired on a BD LSRFORTESSA X-20 and a compensation matrix calculated prior to the acquisition of blood samples.

Measurement of SO-CD20-906_009 CAR-T Cell DNA in Mouse Blood and Tissues Using ddPCR Extraction of DNA from Mouse Blood

DNA was extracted from mouse blood using the QIAamp DNA Micro Kit according to manufacturer's instructions. Briefly, 35 μl of buffer ATL was added to the 65 μl blood sample to make a total volume of 100 μl, to which 10 μl Proteinase K and 100 μl buffer AL was added. Samples were mixed thoroughly by vortexing and incubated at 56° C. for 10 minutes with shaking. Samples were briefly centrifuged to collect droplets from the lid and 50 μl of ethanol was added. Samples were vortexed thoroughly and incubated at room temperature for 3 minutes. The entire lysate was transferred to the QIAamp MinElute Column the lid was closed and columns were centrifuged at 6000 g for 1 minute and flowthrough was discarded. 500 μl of buffer AW2 was added and samples were centrifuged at 6,000×g for 1 minute and flowthrough was discarded. Columns were then centrifuged for 3 minutes at 20,000×g to dry the membrane completely. QIAamp MinElute columns were then placed into a clean 1.5 ml microcentrifuge tube, 20 μl of nuclease free water was added to the membrane and incubated at RT for 10 minutes. DNA was eluted by centrifuging at 20,000×g for 1 minute. DNA concentration was measured using the Nanodrop 2000.

Extraction of DNA from Mouse Tissues

Mouse organs (liver, lung and spleen) were collected into 2 ml EPPENDORF Safe-Lock tubes, to which TE buffer and 1 stainless steel bead (5 mm diameter) was added. Bone marrow pellets were resuspended in TE buffer and transferred to a 2 ml EPPENDORF Safe-Lock tube with 1 stainless steel bead (5 mm diameter). Tubes were placed in the TISSUELYSER adapter and homogenised for 20 seconds at 15 Hz. This homogenisation step was repeated until no visible clumps remained. For bone marrow samples, two samples required an additional 20 second homogenisation. For other organs, homogenisation was repeated three times, resulting in a total homogenisation time of 80 seconds. DNA was extracted from homogenised samples using the PROMEGA MAXWELL RSC Tissue DNA kit according to manufacturer's instructions. Cartridges were loaded into the deck tray and homogenised samples were added into well 1 of the cartridge. A plunger was placed into well 8 of the cartridge and an empty elution tube containing 100 μl of elution buffer was placed in the elution tube area of the rack and the run was initiated. DNA concentration was measured using the NANODROP 2000.

ddPCR

Extracted DNA was digested using MluI to generate fragments suitable for ddPCR, which was prepared in 96 well plates. For blood samples, 500 ng of DNA was digested in a 20 μl reaction and for tissue samples 1 μg of DNA was digested in a 40 μl reaction. Reactions were prepared as described in the table below and incubated at 37° C. for 15 minutes followed by 5 minutes at 80° C. 22 μl ddPCR reactions were prepared containing 50 ng of MluI digested DNA and ddPCR supermix for probes at a final concentration of 1×. Primers were used at a final concentration of 900 nM and probes were used at a final concentration of 125 nM for CDKN1A and 250 nM for HIV. Of the 22 μl reaction, 20 μl was used for droplet generation. Samples were run in duplicate. Plates were sealed using the PX1 PCR plate sealer for 5 seconds at 180° C. and reaction mixes were vortexed briefly and centrifuged. Droplets were generated using the QX200 Auto droplet generator, which partitions samples into nanolitre-sized droplets, each of which serves as an individual reaction. Following droplet generation, plates were sealed using the PX1 PCR plate sealer for 5 seconds at 180° C. Plates were inserted into a PCR thermocycler and incubated for 10 minutes at 95° C. followed by 40 cycles of 95° C. for 30 seconds and 60° C. for 1 minute. Enzyme was inactivated at 98° C. for 10 minutes and plates were cooled to 4° C. prior to droplet reading. Droplets were read on the droplet reader and HIV (FAM) was measured in channel 1 and CDKN1A (VIC) was measured in channel 2.

### Serum Rituximab Concentration

Mouse serum samples were analysed for Rituximab using a validated Gyrolab Immunoassay method based on sample dilution (1 in 10 MRD, Maximum Recovery Diluent) and an anti-idiotypic (ID) capture and anti-human antibody detection. The lower limit of quantification (LLQ) was 0.3 ug/mL using a 1 μL aliquot of serum. The higher limit of quantification (HLQ) was 100 μg/mL. Quality Control samples (QCs) containing rituximab prepared at 3 different analyte concentrations and stored with study samples, were analysed with each batch of samples against separately prepared calibration standards. For the analysis to be acceptable, no more than one-third of the total QC results and no more than one-half of the results from each concentration level were to deviate from the nominal concentration by more than 25%. The applicable analytical runs met all predefined run acceptance criteria. In short, biotinylated anti-rituximab Rexxip A and Alexa labelled anti-human IgG in Rexxip F were diluted. Working solutions in mouse serum were prepared. Working solution were added to Stock_Solution on the 384LDV plate. Control matrix was added to control wells. Serum samples were added to Stock_Solution 384LDV plate and centrifuged for 5 minutes at 1,500×g. The calibrator and quality controls were added into PCR plate by utilising LABCYTE ECHO 525. The capture antibody, detection antibody and wash buffer were added to the PCR plate and the plate centrifuged for 5 minutes at 3,000×g. Finally, the plate was sealed and run with the GYROLAB XPAND.

### Results

The SO-CD20-906_009 CAR-T cell product used within this study has 37.8% TE in the initial assessment pre-freezing. This cell batch was subject to ADCC and CDC assays to confirm that the cells can be ablated in vitro prior to initiation of the in vivo study.

In parallel to inoculation into the mice, the inoculated cells were assessed for cell composition, TE and CD20 expression via flow cytometry (FIG. 42A-42C). This analysis showed that the majority (51%) of cells in the hPBMCs are T cells, followed by monocytes (21%) and B cells (21%) and only a small fraction of NK cells (4%). As expected, the T cells alone were confirmed with 99% CD3+ T cell purity. The hPBMC and T cell mix for the groups receiving both represents the 1:1 mixture ratio, with 74% T cells, followed by 11% monocytes and 11% B cells and only 2% NK cells. On the day of inoculation (post-thaw) a TE of 35% was detected (33+2% F(ab′)2+ for group B with T cells alone) while 38% of T cells were CD20+. In the PBMC and T cell mixture, the percentage of CD20+ F(ab′)2− cells was slightly higher in comparison to T cell inoculated due to the B cells being present as well. The hPBMCs alone had 13% CD20+ F(ab′)2—cells which account for B cells.

SO-CD20-906_009 Counts and hPBMC Composition in Mouse Terminal Blood 7 or 8 Days Post-mAb Treatment (Flow Cytometry)

SO-CD20-906_009 was detected via a f(ab′)2 antibody. On average 2,293 f(ab′)2-positive cells (95% CI: 1,484-3,544) were recovered from ˜400 μL of blood from SO-CD20-906_009 and no mAb ctrl mice on day of culling (FIG. 43A-43B, Tables 12-13). This was on average 17-fold higher than that observed in No SO-CD20-906_009 ctrl mice where on average 135 f(ab′)2-positive cells (95% CI: 87-210) were recovered—reflecting the level of background f(ab′)2 detection (anti-f(ab)2′ binding to T cells) observed in this assay. The No mAb ctrl mice and No SO-CD20-906_009 ctrl mice represented the positive and negative control groups, respectively, for blood f(ab′)2 counts in this study. In SO-CD20-906_009 and mAb treated mice, f(ab′)2 counts were significantly lower 7 and 8 days post-mAb treatment compared to in SO-CD20-906_009 and Isotype mAb ctrl mice. On average, 413 f(ab′)2-positive cells (95% CI: 267-639) were detected in blood of mAb treated mice compared to 2,527 (95% CI: 1,635-3,906) in Isotype mAb ctrl mice. Furthermore, there was no difference in f(ab′)2 counts recovered from Isotype mAb ctrl mice to that from No mAb ctrl mice, both receiving the same dose of T cells. Although f(ab′)2 counts were significantly reduced in the mAb treated mice compared to the Isotype mAb ctrl mice, f(ab′)2 counts still remained higher than that in No SO-CD20-906_009 ctrl mice (p-value of <0.001). F(ab′)2 counts were on average 3-fold higher in mAb treated mice than in No SO-CD20-906_009 ctrl (an average difference of 278 counts) in contrast to the Isotype mAb ctrl mice which were on average 18.6-fold higher (an average difference of 2,392 counts). This therefore indicates, based on absolute counts, a significant but not complete ablation of f(ab′)-positive cells in mice after 7 and 8 days post-mAb treatment.

TABLE 13
Derived counts from mouse terminal blood (Flow Cytometry):

| | | | | | |
|---|---|---|---|---|---|
| | Marginal | Std. | | Lower. | Upper. |
| Group Name | Mean | Error | df | 95% CL | 95% CL |

SO-CD20-906_009 (F(ab′)2) Counts

| | | | | | |
|---|---|---|---|---|---|
| No SO-CD20-906_009 ctrl | 135.937 | 29.049 | 32 | 87.962 | 210.077 |
| SO-CD20-906_009 and no mAb | 2293.392 | 490.089 | 32 | 1484.009 | 3544.215 |
| ctrl | | | | | |
| SO-CD20-906_009 and Isotype | 2527.756 | 540.172 | 32 | 1635.661 | 3906.403 |
| mAb ctrl | | | | | |
| SO-CD20-906_009 and mAb | 413.906 | 88.45 | 32 | 267.831 | 639.652 |

CD3+ Counts

| | | | | | |
|---|---|---|---|---|---|
| No SO-CD20-906_009ctrl | 2919.09 | 520.604 | 32 | 2029.919 | 4197.747 |
| SO-CD20-906_009and no mAb | 6730.26 | 1200.305 | 32 | 4680.186 | 9678.333 |
| ctrl | | | | | |
| SO-CD20-906_009 and Isotype | 9740.574 | 1737.178 | 32 | 6773.542 | 14007.26 |
| mAb ctrl | | | | | |
| SO-CD20-906_009 and mAb | 7932.746 | 1414.762 | 32 | 5516.388 | 11407.55 |

Proportion SO-CD20-906_009 of CD3+ cells.

| | Marginal | Std. | | asymp.Lower | asymp.Upper |
|---|---|---|---|---|---|
| Group Name | Mean | Error | df | 95% CL | 95% CL |
| No SO-CD20-906_009 ctrl | 0.049 | 0.003 | Inf | 0.043 | 0.055 |
| SO-CD20-906_009 and no mAb | 0.341 | 0.013 | Inf | 0.316 | 0.367 |
| ctrl | | | | | |
| SO-CD20-906_009 and Isotype | 0.26 | 0.011 | Inf | 0.238 | 0.282 |
| mAb ctrl | | | | | |
| SO-CD20-906_009 and mAb | 0.053 | 0.003 | Inf | 0.047 | 0.059 |

Std = standard.
df = degrees of freedom.
CL = confidence interval.

TABLE 14
Statistical analysis of derived counts from Mouse terminal blood (Flow Cytometry)

| | | | | | | | |
|---|---|---|---|---|---|---|---|
| | | | | Lower. | Upper. | | |
| contrast | estimate | Std. Error | df | 95% CL | 95% CL | t. ratio | p. value |

F(ab′)2 Counts

| | | | | | | | |
|---|---|---|---|---|---|---|---|
| SO-CD20-906_009 and | 0.163745 | 0.131249 | 32 | 0.088475 | 0.303049 | −5.98734 | 1.1E−06 |
| mAb | | | | | | | |
| Vs. | | | | | | | |
| SO-CD20-906_009 and | | | | | | | |
| Isotype mAb ctrl | | | | | | | |
| SO-CD20-906_009 and | 3.04484 | 0.131249 | 32 | 1.645201 | 5.635209 | 3.684328 | 0.000843 |
| mAb | | | | | | | |
| Vs. | | | | | | | |
| No SO-CD20-906_009 ctrl | | | | | | | |

CD3+ Counts

| | | | | | | | |
|---|---|---|---|---|---|---|---|
| SO-CD20-906_009 and | 0.814402 | 0.109537 | 32 | 0.487215 | 1.361312 | −0.81398 | 0.42167 |
| mAb | | | | | | | |
| Vs. | | | | | | | |
| SO-CD20-906_009 and | | | | | | | |
| Isotype mAb ctrl | | | | | | | |
| SO-CD20-906_009 and | 2.717541 | 0.109537 | 32 | 1.625764 | 4.542497 | 3.963755 | 0.000388 |
| mAb | | | | | | | |
| Vs. | | | | | | | |
| No SO-CD20-906_009 ctrl | | | | | | | |

Proportion F(ab′)2+ cells of CD3+ cells.

| | | | | asymp. Lower | asymp. Upper | | |
|---|---|---|---|---|---|---|---|
| contrast | odds. ratio | Std. Error | df | 95% CL | 95% CL | z. ratio | p. value |
| SO-CD20-906_009 and | 0.1582 | 0.013293 | Inf | 0.134179 | 0.186521 | −21.9449 | P < 0.0001 |
| mAb | | | | | | | |
| Vs. | | | | | | | |
| SO-CD20-906_009 and | | | | | | | |
| Isotype mAb ctrl | | | | | | | |
| SO-CD20-906_009 and | 1.087773 | 0.097247 | Inf | 0.912938 | 1.296091 | 0.941081 | 0.346663 |
| mAb | | | | | | | |
| Vs. | | | | | | | |
| No SO-CD20-906_009 ctrl | | | | | | | |

Std = standard. df = degrees of freedom. CL = confidence interval.

In the f(ab′)2 counts recovered, considerable mouse-to-mouse variability was observed. This was mirrored in the number of overall human cells recovered in mice including total T cell counts (FIG. 43C). In No SO-CD20-906_009 ctrl mice, on average, T cell counts were lower than that in the no mAb ctrl mice or Isotype mAb ctrl mice due to the inoculation regimen. Moreover, a positive correlation was observed between the number of T cell counts recovered in mice and the number of f(ab′)2 counts recovered—even in No SO-CD20-906_009 ctrl mice which did not contain SO-CD20-906_009 T cells. Because of this, f(ab′)2 counts were also considered as a proportion of T cell counts recovered within each mouse to account for this (FIG. 43D). In the No mAb ctrl mice, the proportion of T cells which were f(ab′)2-positive was on average 0.34 (95% CI: 0.32-0.37) on day of culling. This did not change compared to that observed at the time of injection (FIG. 43A). In contrast, in No SO-CD20-906_009 ctrl mice, the proportion of T cells that were f(ab′)2-positive was significantly lower at on average 0.049 (95% CI: 0.043-0.055). This reflects the background level of f(ab′)2 detection observed in this assay. Similarly, in mAb treated mice, the proportion of T cells that were f(ab′)2-positive was also low at on average 0.055 (95% CI: 0.047-0.059); and this was significantly lower than the proportion of f(ab′)2-positive cells in the Isotype mAb ctrl mice which was on average 0.26 (95% CI: 0.24-0.28) (p-value of >0.0001). However, most importantly was that the proportion of T cells that were f(ab′)2-positive in mAb treated mice was comparable to that of No SO-CD20-906_009 ctrl mice (p-value of 0.35). This therefore indicates highly efficient ablation of SO-CD20-906_009 CAR-T cells in blood of mice 7 and 8 days post-mAb treatment based on the proportion of T cells that were SO-CD20-906_009.

Although a decrease in the proportion of f(ab′)2-positive cells within T cells was observed in Isotype mAb ctrl mice compared to No mAb ctrl mice (0.26 vs. 0.34 respectively), the absolute f(ab′)2 counts were comparable. Furthermore, the proportion of f(ab′)2 observed in the Isotype mAb ctrl mice at time of culling did not change to that observed at the time of injection. The difference observed in the proportion of f(ab′)2 within T cells was due to the composition of inoculates used—containing higher amounts of untransduced T cells (contributed by hPBMCs) in the Isotype mAb ctrl mice compared to that in No mAb ctrl mice that did not contain additional hPBMCs. As mentioned, an equivalent proportion of f(ab′)2-positive cells within T cells when comparing pre-inoculate and terminal blood was maintained in the No mAb ctrl mice and the Isotype mAb ctrl mice. In addition, expression of CD20 on f(ab′)2 was also maintained as the proportion of CAR and CD20 co-expressing SO-CD20-906_009 in blood at time of culling was comparable to that of the cell inoculate (FIG. 44).

In addition to f(ab′)2 counts, the hPBMC composition in mouse blood was also evaluated at the time of culling. Of the hCD45+ cells recovered in mice, representing all hPBMCs and SO-CD20-906_009 cells, the majority were T cells at time of culling (data not shown). On average T cells made up 98.44% of all hCD45+ cells in the Isotype mAb ctrl mice and 94.69% in No SO-CD20-906_009 ctrl mice. This was significantly higher than at the time of injection—where T cells made up 74.03% and 51.3% of hCD45+ cells in the Isotype mAb ctrl mice and No SO-CD20-906_009 ctrl mice respectively (FIG. 42A-42C). Although some B, NK and monocytes were detectable in mouse blood, they were generally below the level of detection sensitivity.

In summary, SO-CD20-906_009 CAR-T cells were efficiently ablated in mouse blood 7 and 8 days post-mAb treatment as detected by flow cytometry (f(ab′)2).

Detection of SO-CD20-906_009 in Mouse Samples Using ddPCR

### Mouse Blood Over Time

Prior to the administration of mAb, there were comparable HIV copy numbers in the blood between all groups which received SO-CD20-906_009 (FIG. 45A). As expected, the No SO-CD20-906_009 group had undetectable levels of HIV copies and was excluded from analysis of HIV copies as the majority of values were zero. For all groups which received SO-CD20-906_009, there was considerable variation within each group for HIV copies measured. At the terminal timepoint of the study, mice were split across sampling dates at day 7 and day 8 post-mAb. There was no significant difference in HIV copies for these both terminal days. Throughout the study, there was a steady decrease in total HIV copies observed in all groups which received SO-CD20-906_009, which is most notable by the 72 hrs post mAb and terminal timepoints. Following mAb administration, a significant reduction in HIV copies in the mAb treated group compared to Isotype mAb group was observed by 24 hrs post-mAb administration (p<0.0001, FIG. 45B). This corresponded to an 85.11% decrease in HIV copies in the mAb treated group. The reduction in HIV copies was also observed at 72 hrs post-mAb and at the terminal timepoint, where percentage decreases of 70.44% and 61.56% were observed.

### Mouse Tissues 7 or 8 Days Post-mAb Treatment

As with the blood samples, the No SO-CD20-906_009 ctrl group was excluded from analysis of HIV copies as the majority of values were zero. At the terminal timepoint of the study, mice were split across two cull dates (days 7 and 8 post mAb). Cull date had a significant impact on HIV copies measured in tissues (F test, p=0.001), therefore this term was included in statistical analysis models. In all four tissues tested (bone marrow, liver, lung and spleen), there was a significant reduction in HIV copies for the mAb treated group compared to the Isotype mAb treated group (p<0.0001, FIG. 46A-46B). There were decreases in HIV copies in the mAb group compared to the Isotype mAb group of 95.75% in bone marrow, 88.05% in liver, 95.75% in Lung and 98.66% in spleen. It was also noted that there were significant differences in HIV copies measured in the No mAb ctrl group when compared to the isotype mAb ctrl group, which was observed in the bone marrow, liver, lung and spleen (p<0.0001).

### Histology

Strong evidence was obtained that RTX had ablated SO-CD20-906_009, particularly in the spleen, but also liver and lung, 70 to 98% (95% confidence interval 30 to 100%), 7/8 days post intraperitoneal administration. SO-CD20-906_009 did not cause any toxicity in normal non-inflamed murine tissues which helps define the potential on-target-off-tumour toxicity hazard of the drug candidate.

### Serum Rituximab Concentration

The terminal serum rituximab concentration (ug/mL) was measured for all rituximab-treated mice (Table 17). No SO-CD20-906_009 ctrl group showed a concentration range of 18.038 to 39.862 μg/mL with an average of 28.672 μg/mL. mAb group shows a range of 18.657 to 38.646 μg/mL with an average of 27.372 μg/mL.

In line with previous reports about engraftment of hPBMCs in immunocompromised mouse strains (Schultz et al., 2012), the T cells present the majority of cells in the blood after 8 or 9 days post-cell infusion (D7/8 days post-mAb) with only minor engraftment of B cells or myeloid cells (data not shown). Furthermore, T cells could be detected in the blood and tissues (FIGS. 43A-43D, 44, 45A-45B, and 46A-46B), as previously shown in other studies after 6 to 7 days post infusion (Valton et al., 2018, King et al., 2008, Bonifant et al., 2016, Tasian et al., 2014).

Absolute f(ab′)2 counts in mouse blood determined by flow cytometry indicated SO-CD20-906_009 ablation following mAb treatment. As we observed that mice with higher overall T cells in blood also had higher f(ab′)2 counts even if not inoculated with SO-CD20-906_009 cells, absolute f(ab′)2 counts did not account for total T cell engraftment. This was particularly important when comparing f(ab′)2 counts between No SO-CD20-906_009 ctrl mice and the mAb mice as No SO-CD20-906_009 ctrl mice were inoculated with fewer overall T cells compared to mAb mice. Similarly, it was also important considering the mouse-to-mouse variability in T cell counts observed in blood. Because of this, f(ab′)2 counts were evaluated relative to total T cell counts and by doing so enabled a more accurate comparison of f(ab′)2 counts in mouse blood not only between mice but more importantly between treatment groups.

Some background detection were observed of f(ab′)2 in No SO-CD20-906_009 ctrl mice by flow cytometry. This was based on a small proportion of T cells with observed anti-F(ab′)2 staining. Some background f(ab′)2 detection was anticipated based on assay development work and from known count reference control's ran on the day of each experiment that demonstrated reduced sensitivity of f(ab′)2 detection under 1,000 cells (data not shown). However, this was not limited to f(ab′)2-positive cells as the sensitivity of detection of all PBMC populations was also reduced below 1,000 counts (data not shown). As a result of this, it was less able to determine precise f(ab′)2 counts in mouse blood that fell below 1,000 by flow cytometry—which included all SO-CD20-906_009 and mAb mice, as well as No SO-CD20-906_009 ctrl mice. Despite this, it was able to show that in the blood of the mAb mice (treated with RTX), f(ab′)2 counts were not detected above background (f(ab′)2 counts in blood of No SO-CD20-906_009 ctrl mice) and that this therefore indicated a highly efficient ablation of SO-CD20-906_009 T cells in the blood of mice treated with mAb.

Additionally, we observed that in the Isotype mAb ctrl mice, as well as in the no mAb ctrl mice, SO-CD20-906_009 CAR-T cells present in terminal blood retained CAR and CD20 expression equivalent to pre-inoculation levels. This indicates that the transduced cells did maintain the expression of both, CD20 and CAR, on the cell surface in vivo.

A key endpoint of the study was to compare HIV copies in the SO-CD20-906_009 and mAb group with the SO-CD20-906_009 and Isotype mAb group in blood and tissues, therefore percentage decreases were calculated to compare the mAb and Isotype treated groups. By using ddPCR, this study has shown an efficient decrease in HIV copies of up to 85.11% in blood and 98.66% in tissues of mAb group when compared to Isotype mAb ctrl group. When comparing HIV counts in blood, there was a steady decrease in total HIV copies across all SO-CD20-906_009 engrafted groups over time. This was most notable by the 72 hrs post-mAb and terminal timepoints of the study. Therefore, the percentage difference between the two groups (mAb and Isotype mAb ctrl) reduces over time (85.11% 24 hrs post-mAb, 61.56% at the terminal timepoint). Hence, percentage change data should be interpreted alongside the total HIV copies in the mAb treated groups, which show a sustained decrease in HIV copies up until the terminal timepoint of the study.

At the terminal timepoint of the study (Day 7/8 post-mAb) bone marrow, liver, lung and spleen were harvested and all tissues which received Isotype mAb had detectable HIV copies. This confirms the presence of the SO-CD20-906_009 in the tissues and suggests SO-CD20-906_009 redistribution from the blood to the tissues, which may in part contribute to the declining HIV copies measured in blood at the later study timepoints. In all tissues studied, there was a strongly significant reduction in HIV copies in the mAb group compared to the Isotype mAb group. This further confirms the successful ablation of SO-CD20-906_009 T cells in the mAb treated group.

A point to note when interpreting this data is that it is not possible to accurately quantify SO-CD20-906_009 cell number from copies of HIV (or human reference gene CDKN1A used as a control within the study, not reported here) measured in blood or tissues. Theoretically, the cell number could be estimated from the number of copies of gene measured in the PCR reaction. However, this would assume a total recovery of all DNA during the extraction procedure and full amplification in PCR. Additionally, to compare cell count across samples, this would assume an equal extraction efficiency of DNA across all samples. Preliminary experiments testing a known number of CAR-T cells into blood and extracting found a low recovery of DNA copies compared to spiked cells and variations in the total DNA yield across samples. This was normalised across samples by loading an equal volume of DNA in all PCR reactions. Therefore, PCR should not be considered an accurate quantification method for estimating total cell numbers and instead HIV copy numbers are used for any conclusions.

To confirm successful RTX application and assess terminal levels in case of absence of SO-CD20-906_009 CAR-T cell ablation, RTX concentration was measured in the terminal serum samples. Our results confirm that all mice in the RTX-treated groups were dosed with RTX and displayed levels above 10 ug/mL remained at terminal sampling. This is especially relevant as immunodeficient mouse strains have been reported to display higher mAb clearance (Oldham et al., 2020).

In Summary, the presented study shows that SO-CD20-906_009 CAR-T cells can be ablated efficiently in the given mouse model with a single RTX dose. Ablation (within 24 hrs) in the blood was demonstrated and it was able to demonstrate ablation in the tissues which are less accessible and has lower RTX efficiency compared to blood in the clinical setting (EMA, 2005).

TABLE 15
Summary of percentage changes in blood HIV copiesbetween
mice treated with SO-CD20-906_009 and mAb and mice treated
with SO-CD20-906_009 and Isotype mAb ctrl.

| | % Change HIV | | | |
|---|---|---|---|---|
| Day | copies | lower.CL | upper.CL | p. value |
| Pre-mAb | +8.04 | −33.76 | +76.23 | 0.754 |
| 24 hrs post-mAb | −85.11 | −91.01 | −75.31 | p < 0.0001 |
| 72 hrs post-mAb | −70.44 | −81.68 | −52.31 | p < 0.0001 |
| Terminal | −61.56 | −76.18 | −37.98 | 0.000141 |

CL = confidence intervals.

TABLE 16
Summary of percentage changes in HIV copies in tissues
of mice treated with SO-CD20-906_009 and mAb and mice treated
with SO-CD20-906_009 and Isotype mAb ctrl.

| | % Change HIV | | | |
|---|---|---|---|---|
| Tissue | copies | lower.CL | upper.CL | p. value |
| Liver | −88.05 | −92.12 | −81.87 | p < 0.0001 |
| Lung | −95.75 | −97.2 | −93.56 | p < 0.0001 |
| Spleen | −98.66 | −99.12 | −97.97 | p < 0.0001 |
| Bone Marrow | −95.75 | −97.2 | −93.55 | p < 0.0001 |

CL = confidence intervals.

TABLE 17
Terminal serum rituximab concentration (ug/mL) for rituximab-
treated mice. No SO-CD20-906_009 ctrl group shows a concentration
range of 18.038 to 39.862 μg/mL with an average of 28.672 μg/mL.
SO-CD20-906_009 and mAb group shows a range of 18.657 to
38.646 μg/mL with an average of 27.372 μg/mL.

| Animal | | Terminal | Rituximab |
|---|---|---|---|
| number | Group | day | Concentration (μg/mL) |
| 9 | A | 8 | 39.862 |
| 10 | A | 8 | 30.463 |
| 21 | A | 8 | 36.700 |
| 30 | A | 8 | 24.440 |
| 40 | A | 8 | 27.967 |
| 6 | A | 9 | 26.090 |
| 7 | A | 9 | 18.038 |
| 17 | A | 9 | 22.074 |
| 19 | A | 9 | 34.109 |
| 31 | A | 9 | 26.975 |
| 14 | C | 8 | 27.426 |
| 16 | C | 8 | 31.459 |
| 20 | C | 8 | 38.646 |
| 26 | C | 8 | 20.075 |
| 39 | C | 8 | 28.513 |
| 1 | C | 9 | 18.657 |
| 4 | C | 9 | 26.348 |
| 27 | C | 9 | 30.609 |
| 33 | C | 9 | 29.513 |
| 36 | C | 9 | 22.471 |

### Example 12

### Effect in Lung Cancer

Non-small cell lung cancer has high unmet patient need that could be met by CAR-T cell therapy. The aim of this study was to investigate the potency of CLDN3 CAR-T cell towards non-small cell lung cancer (NSCLC) cell lines in vitro. “906-009_LNGFR” contain the same scFv, hinge, signalling moiety, and co-stimulatory domains as “SO-CD20-906_009” CLDN3 CAR-T cells however it does not contain the CD20 domain and contains an LNGFR tag.

First, a range of NSCLC cell lines were studied for CLDN3 expression and a panel of cell lines was selected. Subsequently, functional experiments were performed to investigate the response of 906-009_LNGFR CAR-T cells (“906-009_LNGFR”) to NSCLC cell lines. The panel of cell lines used for functional experiments was selected to cover a range of CLDN3 expression levels (mRNA and protein), both disease subtypes of interest (squamous or adenocarcinoma) and metastatic and primary pathology. Activation and cytotoxicity were used as indicators for the functional response of 906-009_LNGFR towards NSCLC cell lines and were investigated in vitro by quantifying activation factors (IFNγ and Granzyme B) and Annexin V expression respectively.

All NSCLC cell lines expressing CLDN3 activated 906-009_LNGFR, leading to increased secretion of IFNγ and Granzyme B compared to UT (“untransduced”) and CD19 MB CAR-T cells (“CD19_LNGFR”). Potent cytotoxicity was also observed in response to NSCLC expressing CLDN3. Complete killing was observed in all but two of the cell lines (NCI-H1650 and Colo320DM) which had the lowest levels of CLDN3 expression. This correlated with levels of Granzyme B; all co-cultures where complete killing was observed had granzyme B levels above 1998 pg/mL (mean of 3 donors). In NCI-H1650 and Colo320DM co-cultures, where only partial, donor specific killing was observed, much lower levels of Granzyme B were quantified.

Of the completely killed cell lines, both the lowest CLDN3 mRNA (NCI-H1651) and highest CLDN3 expressing (HT-29) cell lines (9.55 and 93.93 (FPKM), 0.004 and 0.13 relative CLDN3) were able to induce similar levels of IFNγ secretion by 906-009_LNGFR (40,534 pg/mL and 31,138 pg/mL, respectively), suggesting that low levels of CLDN3 can activate 906-009_LNGFR. The activated T cells also secreted levels of Granzyme B above CD19 and UT, indirectly pointing to 906-009_LNGFR cytotoxic activity toward NSCLC cell lines.

Overall, the data in this study shows that activation and cytotoxic response of 906-009_LNGFR would be induced by NSCLC cell lines expressing high to low levels of CLDN3. Cell lines at the limit of detection by flow cytometry also induced strong responses showing the sensitivity of the CAR. Disease subtype analysis and pathology demonstrated that 906-009_LNGFR CAR-T cells would respond to NSCLC cell lines expressing CLDN3 regardless of disease subtype (squamous or adenocarcinoma) and pathology (metastatic or primary). In summary this report provides in vitro evidence that NSCLC may be an appropriate patient population for CLDN3 CAR-T cells.

### Materials and Methods

The aim of this study was to understand whether NSCLC patients could be treated with CLDN3 CAR-T cells by confirming robust CLDN3 expression in NSCLC cell lines and a functional response by CLDN3 CAR-T cells.

Initially RNASeq data was used to select cell lines with diverse levels of CLDN3 from the three major disease subpopulations. Subsequently, CLDN3 protein and CLDN3 mRNA expression were assessed by flow cytometry and qRT-PCR respectively. Based on the data collected, a range of cell lines that expressed high to low levels of target expression were selected for use in functional experiments. To account for the diversity of the NSCLC patient population, cell lines were selected from the two most common subtypes (Adenocarcinoma and squamous cell carcinoma), both metastatic and primary pathology.

The functional response of 906-009_LNGFR CAR-T cells was assessed using two key read-outs, activation factor secretion (Granzyme B and IFNγ) and killing (confirmed by expression of annexin V). The combination of T cell activation and target cell death confirms a cytotoxic response whereas the levels of activation factors alone can be used to indicate a cytotoxic response or suggest a reduced response where concentrations are low. CLDN3 expression was also assessed to compare the response to target expression on the day of target cell plating. As CRC is the primary indication in FTiH study, a number of CRC cell lines used in previous potency assays were included in the panel as a benchmark for 906-009_LNGFR. Although no specific claim is being made, this study was conducted in accordance with accepted scientific practice for this type of study.

Cell line culture. The cell lines were thawed one to two weeks in advance of co-culture using RPMI supplemented with 10% FBS and 1% GLUTAMAX. Cells were split every 3-4 days and on the day of seeding for co-culture: cells were collected with TryplE and counted on the NUCLEOCOUNTER 202.

T cell thawing. 906-009_LNGFR, CD19 MB (CD19 CAR negative control, “CD19_LNGFR”) and UT (untransduced) T cells (production described in 2021N467314) from donors PR19K133900, PR19C133904, and PR19W133916 were thawed on day of co-culture. T cells were thawed in the hand and resuspended in 10 mL of cold TEXMACS. The cells were spun down at 300×g for 10 minutes (RT) and re-suspended in cold TEXMACS. The cell suspension was spun once more at 300×g for 20 minutes and resuspended in 5 mL of cold TEXMACS. The cells were then counted on the NUCLEOCOUNTER 202 and aliquoted for further assays.

qPCR. RNA extraction: RNA was extracted from cell line pellets using the Promega Maxwell RSC SIMPLYRNA Cells Kit and following the manufacturers' protocol. In brief, cell pellets were resuspended in 200 μl homogenisation solution containing thioglycerol. Homogenised cells were then lysed upon the addition of 200 μl lysis buffer. Lysed cells were then added to the Maxwell cartridges' well 1 and 5 μl reconstituted DNAse 1 was added to well 5. Plungers were added to well 8 and the cartridges were run on the Maxwell® RSC 48 machine. RNA was eluted in 50 μL nuclease-free water and stored at −80° C. prior to cDNA synthesis. cDNA synthesis: RNA was measured using the Nanodrop 2000. RNA was then reverse transcribed using 4 μL SUPERSCRIPT IV VILO™ Master Mix and 1 μg of RNA per sample following the manufacturers' protocol. For four samples, a No RT control was generated, which contained 1 μg RNA and 4 μl of the No RT master mix. Reactions were incubated at 25° C. for 10 minutes, then 50° C. for 10 minutes followed by 85° C. for 5 minutes using a C1000 TOUCH Thermal Cycler. RT-qPCR: RT-qPCR was performed on cDNA using TAQMAN Gene Expression Assays for human CLDN3 as well as for endogenous reference genes actin B (ACTB) and Ubiquitin C (UBC). In short, sample cDNA was pre-diluted 1/5 with nuclease-free water. A 1/5 7 point gDNA serial dilution was created. Each PCR reaction was set-up up according to the manufacturers' protocol by mixing 5 μL TAQMAN Fast Advanced Master Mix, 0.5 μL TAQMAN Gene Expression Assay, 2.5 μL nuclease-free water and 2 μL of cDNA/gDNA (as prepared above). PCR was carried out in MICROAMP Optical 384-Well Reaction Plates using QUANTSTUDIO 6 Flex Real-Time PCR System.

Initial runs had gDNA contamination so troubleshooting run using IP08 and UBC were used to determine cause of failure. Contamination of IV VILO No RT master mix was the reasons runs failed so method above repeated with fresh IV VILO cDNA synthesis kit.

Flow Cytometry. Target cell lines were analysed by flow cytometry to determine CLDN3 expression. Cell suspensions were washed twice in FACS buffer (D-PBS+2% FBS), resuspended in 40 μl Human TRUSTAIN FCX Fc blocker and incubated for 10 minutes at room temperature. Cells were then stained with 40 μl of 2× PE Claudin-3 Antibody or PE REA IgG1 isotype control antibody (working concentration of 5 μg/ml) and incubated for 30 minutes at room temperature in the dark. Cells were then washed three times in FACS buffer before being resuspended in a DAPI solution (1 μg/mL DAPI in D-PBS). Cells were analysed immediately using the CYTOFLEX flow cytometer.

Co-culture set-up for cytokine detection. Target cell lines were detached and counted the day before co-culture with T cells. Cells were washed, centrifuged at 300×g and resuspended in media at the right density for each experiment. 2.5×10⁴ cells were then seeded into a 96-well plate. The day after, freshly thawed and normalized T cells were then added to the plate at 1:1 E:T (effector: target cells, where “effectors” were transduced CAR-T cells) ratios and co-cultured at 37° C., 5% CO₂. Each co-culture condition was run in triplicate. After 24 hours plates were centrifuged, supernatants collected and stored at −80° C. to quantify cytokine secretion.

Human IFNγ Cytokine U-Plex MSD Assay. An MSD assay using 2 cytokine U-Plex plates was carried out as per manufactures instructions.

Plate Preparation. Biotinylated antibodies were coupled to linkers (IFNγ to Linker 1 and Granzyme B to Linker 10) by adding Linker:antibody at a 3:2 ratio. The mixes were vortexed and incubated at RT for 30 minutes. Stop solution was added for a linker:antibody:stop solution of 3:2:2, the mixes were vortexed and then incubated at RT for 30 minutes. A coating solution was made by combining and diluting the linker coupled antibodies 1/10 in stop solution. The coating solution was vortexed and the plate was coated by adding 50 μL to each well. The plates were sealed and incubated at RT for 1 hour while shaking.

Reagent and sample preparation. Frozen samples and Diluent 3 and 2 were thawed, and equilibrated to RT. The sample plates were then spun at 2000×g for 3 minutes. Assay calibrator 1 was reconstituted in 250 μL Diluent 2 and incubated at RT for 30 minutes and assay calibrator 23 was thawed on ice.

Calibrator and sample dilutions. For the serial dilution to generate cytokine standards calibration curve: the first standard was made by diluting Calibrator 1 and Calibrator 23 1/10 in Diluent 2. Standards 2 to 7 were then made with a 4-fold serial dilution. The samples were also diluted in Diluent 2 to fit them into the top and bottom of the standard curve.

Assay protocol. The plates were washed 3× with 150 μL/well of wash buffer (PBS+0.05% Tween) and 50 μl of calibrators or diluted supernatant samples were plated. The plates were incubated at RT with shaking at least 750 rpm for 2 hours. Following incubation, the plates were washed 3× with 150 μL/well of wash buffer and then 50 μL of the detection antibody solution was added to each well (antibodies for IFNγ and Granzyme B diluted 1/100 in Diluent 3). The plates were incubated at RT, shaking at least 750 rpm for 1 hour. Following incubation, the plates were washed 3× with 150 μL/well of wash buffer. Next, 150 ul of MSD GOLD Read Buffer were added to each well and then the plates were read on the MSD Sector 600 Imager immediately.

### INCUCYTE Based Killing Assay

Plate Coating. To each of the wells of a NUNCLON Delta Surface 96 well plate, 50 μl of 0.01% Poly-L-Ornithine was added and the plates were incubated overnight at 4° C. The following day, plates were washed three times with 150 μl PBS and air dried in the biosafety cabinet for 1 hour.

Target Cell Plating. Target cell lines were detached and counted as described above and resuspended at an appropriate concentration to seed either 15,000 or 25,000 cells per well in a volume of 50 μl of co-culture media (Phenol Free RPMI+10% FBS+1% Glutamax+1% Sodium Pyruvate+1% NEAA). Seeding densities were determined individually for each cell line. Plates were incubated overnight at 37° C. with 5% CO₂.

T Cell Plating. The following day, 50 μl of Annexin V dye for apoptosis (diluted in co-culture media to achieve a final concentration of 1:500 in a 150 μl total well volume) was added to plates containing target cells. Plates were incubated at 37° C. with 5% CO₂ while T cells were prepared. T cells, which were prepared as described above, were resuspended at an appropriate concentration to achieve a 1:1 target cell: T cell ratio in 50 μl of co-culture media. Plates were then transferred to the INCUCYTE SX5 and incubated at 37° C. with 5% CO₂ throughout the experiment.

Image Collection. Images were acquired using the IncuCyte SX5 at using the Adherent Cell-by-Cell Scan type with a 10+ magnification. Data was collected in the Phase and NIR channels. Four images per well were acquired at three hour intervals for a period of 7 days.

### Results

### CLDN3 Expression by NSCLC Cell Lines.

The expression of CLDN3 mRNA and protein by 24 NSCLC cell lines and a positive (HT-29) and negative (RKO KO) CRC control was assessed. The cell lines were cultured over a 6-week period and samples were collected for three distinct flow cytometry and qPCR experiments (FIG. 47A-47B and Table 18).

As determined by flow cytometry analysis, the majority (16) of the NSCLC cell lines were composed of homogenous CLDN3 positive population, with ranging MFI's that reflect various levels of CLDN3 on the cell surface of different cell lines (1.2 (RKO KO) to 738 (HT-29) normalised MFI). Of the cell lines remaining 8 were partially positive for CLDN3, based on a population shift in fluorescence rather than distinct populations, and one was CLDN3 negative (NCI-H1703). Increasing levels of protein mostly reflected an increase in mRNA although there were some outliers such as NCI-H1650.

Based on the expression data, a range of cell lines were selected, with the aim of showing a functional response towards NSCLC CLDN3 expressing cell lines at a range of expression levels, independent of pathology (both metastatic or primary) and from the two main disease subsets (adenocarcinoma and squamous) (Table 19). A panel of twelve cell lines with a wide range of CLDN3 expression levels (based on relative CLDN3 and % membrane bound CLDN3 population) was selected to investigate the response of 906-009_LNGFR to low levels of target expression as well as high levels. Three cell lines with partial positive populations were selected as well as an NSCLC cell line, NCI-H1703, that was presumed negative based on the low relative CLDN3 mRNA, 0% membrane bound CLDN3 population and normalised MFI similar to RKO KO (a CLDN3 KO cell line which is used as negative control) (Table 18).

Several CRC cell lines that had been characterised in this application were also included in the study. The following cell lines were selected; a CRC cell line with very low expression of CLDN3 mRNA (Colo320DM), and three additional cell lines with CDLN3 expression levels that are comparable to medium and high CLDN3 expressing NSCLC cell lines (DLD1, HCT15 and HT-29). A large body of data studying the response of CLDN3 CAR-T cells to the cell lines already exists, and these CRC cell lines were used as a benchmark in this study.

CLDN3 expression in target cells was reconfirmed on the seeding day for co-culture (the day before T cell addition). Differences across experiments were noted for CLDN3 protein expression levels (analysed as normalised MFI in FIG. 47B and Table 18). Specifically, in the initial cell line screening phase, NCI-H1650, was consistently demonstrated to contain a partially positive low CLDN3 population (35%, n=3) with normalised CLDN3 MFI greater than RKO KO (2.7 vs 1.4, n=3). On the day of target cell seeding for functional experiments NCI-H1650 expressed CLDN3 levels below the background observed in and RKO KO (1.55 vs 1.8 normalised MFI) and was only 2.28% CLDN3 positive (FIG. 54A-54J). Of note, in the experiments which showed NCI-H1650 was 35% CLDN3 positive NCI-H1650 consistently had relative CLDN3 mRNA levels below NCI-H1703 (Table 18).

TABLE 18
Expression of CLDN3 by NSCLC cell lines. CLDN3 expression was assess by qPCR
(2-^{ΔCT}) and by flow cytometry (Normalised PE MFI) over three experiments.

| | Experiment 1 | | Experiment 2 | |
|---|---|---|---|---|
| | qPCR | Normalised MFI | qPCR | Normalised MFI |
| RKO-KO | 0.003335241 | 1.204845815 | 0.000824114 | 1.556936 |
| NCI-H1703 | N/A | N/A | 0.00047003 | 1.389313 |
| NCI-H2023 | 0.003670222 | 1.276021265 | 0.001647853 | 1.392581 |
| NCI-H460 | 0.003358099 | 1.413461538 | 0.001671613 | 1.51897 |
| LU65 | 0.007209422 | 1.709439528 | 0.001503094 | 1.895317 |
| NCI-H1755 | | | 0.002563686 | 3.248598 |
| NCI-H1650 | 0.001068008 | 2.733506944 | 0.000461306 | 2.686047 |
| A549 | 0.002224594 | 1.747826087 | 0.000972491 | 2.058613 |
| NCI-H2347 | | | 0.09115699 | 16.18642 |
| NCI-H441 | 0.028635202 | 9.648679679 | 0.022278655 | 15.95519 |
| NCI-H2122 | 0.033484859 | 17.30518909 | 0.0362018 | 20.27599 |
| NCI-H522 | 0.023850311 | 25.7360179 | 0.010584087 | 56.79117 |
| NCI-H2291 | 0.120269035 | 35.70586053 | 0.072841574 | 47.56344 |
| NCI-H520 | 0.041490987 | 31.74115044 | 0.026495499 | 33.58874 |
| NCI-H1581 | 0.019139604 | 30.10164425 | 0.012905015 | 32.73684 |
| NCI-H1651 | 0.013032526 | 35.05640244 | | |
| NCI-H2106 | | | | |
| NCI-H2170 | 0.07077233 | 38.60678925 | 0.042534057 | 66.98403 |
| NCI-H661 | | | 0.023307234 | 67.62993 |
| NCI-H1838 | | | 0.045997443 | 34.20717 |
| HCC0827 | 0.264411515 | 74.20557491 | 0.192664904 | 116.28 |
| NCI-H2126 | 0.090398007 | 69.48458781 | 0.048488585 | 107.0213 |
| HCC15 | 0.033510941 | 166.397454 | 0.012848113 | 195.3033 |
| HCC2935 | 0.071200384 | 81.21613237 | 0.042042582 | 149.7204 |
| NCI-H810 | 0.107472822 | 208.4030837 | | |
| HT-29 | 0.3535203 | 738.2565056 | 0.17054177 | 747.2527 |

| | Experiment 3 | |
|---|---|---|
| | qPCR | Normalised MFI |
| RKO-KO | 0.001812296 | 1.433566434 |
| NCI-H1703 | 0.001255001 | 1.154345006 |
| NCI-H2023 | 0.002431704 | 1.57408075 |
| NCI-H460 | 0.001818073 | 1.579298831 |
| LU65 | 0.001021064 | 1.730099502 |
| NCI-H1755 | 0.002473057 | 1.982664234 |
| NCI-H1650 | 0.001372299 | 2.702734839 |
| A549 | 0.003373824 | 5.378596087 |
| NCI-H2347 | 0.106000687 | 12.44494659 |
| NCI-H441 | 0.021552347 | 15.67727931 |
| NCI-H2122 | 0.036126528 | 21.36960986 |
| NCI-H522 | 0.006209105 | 30.85344828 |
| NCI-H2291 | 0.06916814 | 34.54140571 |
| NCI-H520 | 0.024306636 | 35.20707071 |
| NCI-H1581 | 0.01281286 | 35.47157623 |
| NCI-H1651 | | |
| NCI-H2106 | 0.012820521 | 38.68181818 |
| NCI-H2170 | 0.034667675 | 43.16472303 |
| NCI-H661 | 0.068285278 | 62.53742802 |
| NCI-H1838 | 0.055718987 | 95.45022624 |
| HCC0827 | 0.102515074 | 99.54487179 |
| NCI-H2126 | 0.089747535 | 102.6476101 |
| HCC15 | 0.019220253 | 120.7115385 |
| HCC2935 | 0.040968824 | 150.8306709 |
| NCI-H810 | | |
| HT-29 | 0.225382435 | 1000 |

TABLE 19
Cell lines selected for further experiments and their use in functional assays

| | Disease | | CLDN3 | | |
|---|---|---|---|---|---|
| Cell Line | subtype | Pathology | Expression | Activation | Killing |
| NCI-H2170 | Squamous | Primary | 100% | Yes | Yes |
| NCI-H520 | Squamous | Primary | 100% | Yes | Yes |
| HCC15 | Squamous | Primary | 100% | Yes | Yes |
| NCI-H1703 | Squamous | Primary | Negative | Yes | Yes |
| NCI-H2023 | Adenocarcinoma | Metastasis | partial | Yes | No |
| NCI-H1650 | Adenocarcinoma | Metastasis | partial | Yes | Yes |
| NCI-H2347 | Adenocarcinoma | Primary | partial | Yes | No |
| NCI-H2291 | Adenocarcinoma | Metastasis | 100% | Yes | No |
| NCI-H441 | Adenocarcinoma | Metastasis | 100% | Yes | No |
| NCI-H522 | Adenocarcinoma | Primary | 100% | Yes | No |
| NCI-H1651 | Adenocarcinoma | Primary | 100% | Yes | Yes |
| HCC0827 | Adenocarcinoma | Primary | 100% | Yes | No |

TABLE 20
Estimating IFNγ fold change (906-009 LNGFR vs
CD19 LNGFR) expression at selected relative CLDN3 levels.

| Contrast | Estimate | Lower.CL | Upper.CL | p. value |
|---|---|---|---|---|
| 0.00037 | 10.37 | 4.16 | 25.86 | P < 0.001 |
| 0.001 | 227.07 | 129.2 | 399.08 | P < 0.001 |
| 0.003 | 2963.58 | 1367.58 | 6422.18 | P < 0.001 |
| 0.01 | 8076.66 | 4132.53 | 15785.1 | P < 0.001 |
| 0.03 | 14593.88 | 8859.49 | 24039.91 | P < 0.001 |
| 0.1 | 4887.35 | 2536.91 | 9415.47 | P < 0.001 |

CL = confidence intervals.

TABLE 21
Estimating Granzyme B fold change (906-009 LNGER vs CD19
LNGFR) expression at selected relative CLDN3 levels.

| contrast | estimate | lower.CL | upper.CL | p. value |
|---|---|---|---|---|
| 0.00037 | 3.63 | 2.1 | 6.25 | P < 0.001 |
| 0.001 | 36.46 | 26.05 | 51.02 | P < 0.001 |
| 0.003 | 263.98 | 166.49 | 418.54 | P < 0.001 |
| 0.01 | 622.32 | 417.42 | 927.79 | P < 0.001 |
| 0.03 | 534.92 | 397.29 | 720.23 | P < 0.001 |
| 0.1 | 446.28 | 301.92 | 659.66 | P < 0.001 |

CL = confidence intervals.

TABLE 22
Qualitative summary of target cell killing
responses in 906-009 LNGFR co-cultures

| Cell Line | Indication | Donor 1 | Donor 2 | Donor 3 |
|---|---|---|---|---|
| HT-29 | Colorectal | Yes | Yes | Yes |
| RKO-KO | Colorectal | No | No | No |
| Colo 320 | Colorectal | Partial | No | No |
| DLD1 | Colorectal | Yes | Yes | Yes |
| NCI-H1650 | NSCLC | Partial | Partial | No |
| NCI-H1703 | NSCLC | No | No | No |
| NCI-H520 | NSCLC | Yes | Yes | Yes |
| NCI-H2170 | NSCLC | Yes | Yes | Yes |
| NCI-H1651 | NSCLC | Yes | Yes | Yes |
| HCC15 | NSCLC | Yes | Yes | Yes |

TABLE 23
Summary of CLDN3 expression and functional response of 906-009 LNGFR in a range of NSCLC cell lines and colorectal control cell lines.

| | | | | | Activation: | Activation: | |
|---|---|---|---|---|---|---|---|
| | Indication | | | Relative | IFNγ | Granzyme B | |
| Cell Line | (Disease subtype) | Pathology | CLDN3 (FPKM)⁷ | CLDN3 | (pg/mL) | (pg/mL) | Killing |
| RKO KO | CRC | N/A | KO Cell Line | KO Cell Line | 49 | 10 | No |
| NCl-H1650 | NSCLC - Adenocarcinoma | Metastasis | 0.61 | 0.0004 | 2525 | 147 | Partial (some donors) |
| NCl-H1703 | NSCLC - Squamous | Primary | 0.03 | 0.0006 | 12 | 6 | No |
| COLO-320DM | CRC | N/A | 0.18 | 0.0008 | 184 | 32 | Partial (some donors) |
| NCl-H2023 | NSCLC - Squamous | Metastasis | 1.33 | 0.001 | 13251 | 2314 | N/A |
| NCl-H1651 | NSCLC - Adenocarcinoma | Primary | 9.55 | 0.004 | 40535 | 2655 | Complete |
| NCl-H522 | NSCLC - Adenocarcinoma | Primary | 5.73 | 0.005 | 29574 | 1301 | N/A |
| NCl-H520 | NSCLC - Squamous | Primary | 5.82 | 0.015 | 36074 | 1998 | Complete |
| NCl-H441 | NSCLC - Adenocarcinoma | Metastasis | 10.52 | 0.018 | 63328 | 3400 | N/A |
| DLD-1 | CRC | n/a | N/A | 0.022 | 54859 | 2019 | Complete |
| HCC15 | NSCLC - Squamous | Primary | 28.45 | 0.024 | 75245 | 3332 | Complete |
| NCl-H2291 | NSCLC - Adenocarcinoma | Metastasis | 60.76 | 0.029 | 43970 | 3079 | N/A |
| NCl-H2170 | NSCLC - Squamous | Primary | 35.46 | 0.033 | 29899 | 3026 | Complete |
| NCl-H2347 | NSCLC - Adenocarcinoma | Primary | 50.76 | 0.037 | 59954 | 3012 | N/A |
| HCT15 | CRC | N/A | 62.11 | 0.072 | 57168 | 2514 | N/A |
| HCC0827 | NSCLC - Adenocarcinoma | Primary | 10.09 | 0.099 | 65415 | 3678 | N/A |
| HT29 | CRC | N/A | 93.93 | 0.13 | 31139 | 4357 | Complete |

### Activation of 906-009 LNGFR by NSCLC Cell Lines.

To investigate the efficacy of 906-009_LNGFR towards NSCLC cell lines, activation factors IFNγ and Granzyme B were quantified after 24 hours of 906-009_LNGFR and target cells co-culture. The data from this is presented as an average of the three donors in FIG. 49A-49B.

All cell lines with relative CLDN3 mRNA expression above 0.001 dCT (up to the highest CLDN3 expressing cell line HT-29, 0.13 dCT) activated 906-009_LNGFR CAR-T cells leading to robust IFNγ and Granzyme B secretion above control levels (UT/CD19_LNGFR co-cultures). In all but two of the cell lines (one CRC and one NSCLC) there were levels of Granzyme B above 800 pg/mL. Both cell lines with lower levels of Granzyme B secreted lower levels of IFNγ as well and were shown to express CLDN3 mRNA below 0.001 and CLDN3 protein at background level.

The CRC cell lines with similar levels of CLDN3 (low, medium, and high) that had been used in previous potency assays were included in the co-culture. The levels of IFNγ and Granzyme B secreted by 906-009_LNGFR in response to the CRC cell lines was no higher than the response to the NSCLC cell lines. HT-29, a CRC cell line with the highest level of CLDN3 mRNA had similar levels of IFNγ and Granzyme B (31,139 pg/mL and 4,357 pg/mL) to the highest CLDN3 mRNA expressing NSCLC cell line HCC0827 (65,415 pg/ML IFNγ and 3,678 pg/mL Granzyme B) (Table 23).

These response of 906-009_LNGFR was specific to the target expressing cells. There was no secretion of IFNγ and Granzyme B by CD19_LNGFR above the level of UT and there was no secretion of these factors by 906-009_LNGFR in response to the CLDN3 negative cell lines NCI-H1703 and RKO KO.

### Relationship Between Expression and Response.

The data presented in FIG. 3 demonstrate that a low level of CLDN3 can induce a significant activation response by 906-009_LNGFR (quantified by IFNγ and Granzyme B secretion). To understand at what level of CLDN3 mRNA the activation response might peak and plateau the relationship between expression and response was modelled (FIG. 50A-50B). The curve was then used to estimate fold change vs CD19 MB at specific levels of CLDN3 expression (Table 20 and Table 21).

These models suggest that at low levels of CLDN3 statistically significant increases in activation factors are expected. Even at 0.00037 relative CLDN3 a significant fold change (vs CD19_LNGFR) of 10.37 for IFNγ and 3.63 for Granzyme B was estimated. The highest CLDN3 mRNA level used in the model was 0.099 (HCC0827 NSCLC) cell line. Since only a few cell lines showed very low levels of CLDN3 the statistical power estimating fold change vs CD19_LNGFR at low expression levels lower; even so it is clear that 906-009_LNGFR is reactive to low levels of CLDN3 expression by NSCLC cell lines.

Based on the curve in FIG. 50A there was a plateau in IFNγ concentration at ˜0.02 relative CLDN3 mRNA expression. At 0.03 relative CLDN3 mRNA the estimated IFNγ secretion fold change was 14594 times higher vs CD19_LNGFR showing the potent activation response to NSCLC cell lines. Based on FIG. 4B the Granzyme B response plateaued at a lower level of CLDN3 expression; at relative CLDN3 expression of around 0.005 relative CLDN3 mRNA.

### Potency of 906-009 LNGFR in Inducing Target Cell Death in NSCLC Cell Lines

This work aimed to assess the ability of 906-009_LNGFR to induce cell death in a range of NSCLC target cells. During apoptotic cell death, phosphatidylserine is externalised, which can be visualised by annexin V staining. To assess target cell death in this study, annexin V staining was quantified throughout the duration of target cell coculture with 906-009_LNGFR. Total area of annexin V staining was interpreted together with phase images to assess the presence of target cells following coculture with 906-009_LNGFR.

Complete death of target cells, induced by 906-009_LNGFR, was observed in several of the NSCLC cell lines tested, which was determined by the presence of clusters of annexin V expressing cells and no visible annexin V negative target cells (FIG. 51 for CRC cell lines and FIG. 52 for NSCLC cell lines). Where target cell death was observed in coculture with 906-009_LNGFR, this occurred within a short time frame (a maximum of 4 days post addition of CAR-T cells). In the NCI-H1703 cell line, which is negative for CLDN3 expression, there was no target cell death observed during coculture with 906-009_LNGFR.

To assess the ability of 906-009_LNGFR to induce target cell death in cell lines expressing low levels of CLDN3, the CRC cell line Colo320DM and NSCLC cell line NCI-H1650 were cultured with 906-009_LNGFR. In both cell lines, partial target cell death was observed, which was defined as an increase in annexin V staining in 906-009_LNGFR co-cultures compared to CD19_LNGFR co-cultures and a reduction in target cell number or integrity (FIG. 53A-53B). For the CRC cell line Colo320DM, killing was only observed in one donor out of three tested accompanied there was an increase in Annexin V staining and reduction in target cell number. In the NSCLC cell line NCI-H1650, annexin V staining increased in 906-009_LNGFR cocultures earlier than in CD19_LNGFR cocultures in all three donors tested, however reduced target cell integrity was observed in only two of the three donors tested.

This study has demonstrated the ability of 906-009_LNGFR CLDN3 CAR-T cells to induce target cell death against a range of CLDN3 expressing NSCLC cell lines derived from both squamous cell carcinoma and adenocarcinoma NSCLC subtypes (Table 22 and FIGS. 51, 52 and 53A-53B).

To expand the potential indications to benefit from CLDN3 CAR T cells therapy, it is important to show that there is a robust functional response to a range of cell lines originating from the disease of interest. This study aimed to show this by using 906-009_LNGFR co-cultures with NSCLC cell lines and determining if there was evidence that CLDN3 CAR-T cells could be used as a therapy for NSCLC. Therefore, in this work a panel of cell lines were selected from the squamous and adenocarcinoma subtypes with differing levels of CLDN3, and metastatic and primary pathology.

There is evidence that CLDN3 protein detection by flow cytometry is limited. Firstly, no distinct CLDN3 positive and negative populations are observed in partially positive cell lines, only shifts in fluorescence compared to negative controls (FIGS. 54A, 54C, 54E, 54G, 54I). Secondly, there is 100% killing of some cell lines that are only partly positive for CLDN3 (DLD1—36% and NCI-H1651—75%) based on the flow cytometry performed on the day of functional experiments.

The data within this study shows the potency of 906-009_LNGFR towards a range of NSCLC cell lines. Complete target cell death was observed (Annexin V expression by all remaining target cells) in all cell lines with CLDN3 FPKM above 5.82 (Table 23). In all these conditions there was also a granzyme B secretion, above 800 pg/mL. The lowest level of Granzyme B (where complete killing was apparent in an equivalent experiment) was observed in NCI-H520 co-cultures with Donor PR19W133916 906-009_LNGFR (969 pg/mL). This suggests that this level of Granzyme B is indicative of a response that would lead to apoptosis in all target cells. As such it is possible that all cell lines with Granzyme B above this level (unless intrinsically able to evade T cell killing via Granzyme B) would be killed by 906-009_LNGFR. Levels of Granzyme B above 969 pg/mL following co-culture with target cells, could be observed independent of indication, disease subtype and pathology and as such were probably related to the level of relative CLDN3.

The relationship between IFNγ/Granzyme B and CLDN3 expression was also studied, using the data collected in this study to model expected levels of activation factors at changing levels of CLDN3. Important to note is the peaking of the activation response at low levels of CLDN3 expression; (˜0.02 relative CLDN3 for IFNγ and ˜0.005 relative CLDN3 for Granzyme B). This plateau of Granzyme B secretion at lower levels of CLDN3 mRNA correlates with complete killing, further suggesting that this distinct pattern of Granzyme B secretion is indicative of killing.

The four CLDN3 positive CRC cell lines that were included in the panel of cell lines induced similar levels of IFNγ and Granzyme B secretion by 906-009_LNGFR. HT-29 (which expressed 0.12 relative CLDN3 compared to the highest CLDN3 expressing NSCLC cell line HCC0827 0.099 relative CLDN3) did not secrete higher levels of IFNγ (31,138 pg/mL compared to 65,414 pg/mL) and secreted similar levels of Granzyme B (4,357 pg/mL compared to 3,678 pg/mL), demonstrating that the highest activation levels has been reached regardless of the levels of antigen in CLDN3 positive cells. In both NSCLC and CRC cell lines with the lowest levels of CLDN3 expression (mRNA and protein), IFNγ secretion by 906-009_LNGFR was also comparatively lower. Overall, this shows that NSCLC cell lines can induce an in vitro activation response as robust as the response induced by CRC cell lines with similar levels of expression.

Upregulation of IFNγ/Granzyme B was observed in co-cultures of 906-009_LNGFR CAR-T cells with NCI-H1650, despite baseline levels of CLDN3 (0.0036 relative CLDN3 and 0.61 FPKM). Based on the expression data from the functional experiment, expression of CLDN3 protein by NCI-H1650 was no higher than baseline levels, 1.5 normalised CLDN3 expression. There was however, a clear activation response (2,525 pg/mL IFNγ and 220 pg/mL Granzyme B) and partial killing in some co-cultures. As shown in the results, NCI-H1650 has previously shown consistently higher CLDN3 protein than negative cell lines despite the low relative CLDN3, suggesting that for this cell line mRNA is not indicative of protein expression. The discrepancies between experiments may be due to decreased reliability of the flow cytometry assay when approaching the lower limit of detection of the antibody used. The data may also not be representative of CLDN3 expression at the time of co-culture as T cells plate 16 hours after target cell plating. As such it is probable that this cell line expresses a low level of CLDN3 not detected by the flow antibody used in this study that is sufficient to induce an activation response and minimal Annexin V expression.

The partial killing response of NSCLC cell line NCI-H1650 was characterised by a reduced control of target cell growth and partial apoptosis (confirmed by Annexin V expression). This correlated with decreased Granzyme B secretion (220 pg/mL) compared to completely killed cell lines (where there was 969 pg/mL Granzyme B or higher). This partial killing response was similar to Colo320DM, CRC cell line with low CLDN3 protein expression, at the limit of detection by flow cytometry, but with higher CLDN3 mRNA than NCI-H1703. Partial killing was observed within one donor (with the highest IFNγ and Granzyme B concentrations) and there was continued growth of the cell line. This shows that suggest that this reduced response of 906-009_LNGFR CAR-T cells is not indication dependent.

In summary, NSCLC cell lines induced a robust activation response (significant Granzyme B and IFNγ secretion vs CD19 was estimated at as low as 0.00037 relative CLDN3) and potent killing response (100% cell death in cell lines with relative CLDN3 above 0.0038). Activation and killing was observed at low levels of CLDN3 independent of pathology and disease subset. Where there were similar levels of CLDN3 expression in CRC and NSCLC cell lines there was a similar activation and killing response. A broad data set already exists for these CRC cell lines showing the potency of CLDN3 CAR-T cells so the similar response towards NSCLC and the benchmark further validates this data set. Target cell death was induced in NSCLC cell lines from two key NSCLC subsets (adenocarcinoma and squamous cell carcinoma), indicating that 906-009_LNGFR CAR-T cells are effective against a range of NSCLC cell lines deriving from distinct disease subtypes.

A relationship was also observed between the expression of CLDN3 and activation factor secretion that differs for IFNγ and Granzyme B. Levels of these activation factors peaked at low levels of CLDN3 expression (on the day of the experiment 0.02 dCT and 0.005 dCT relative CLDN3 respectively) showing the sensitivity of 906-009_LNGFR CAR-T cells to CLDN3 expressing cell lines in this indication. A limited activation and cytotoxic response were also observed at 0.0008 relative CLDN3 mRNA and lower where only partial killing was induced and lower levels of IFNγ and Granzyme B were detected.

Overall, this confirms that a range of NSCLC cell lines expressing high and low levels of CLDN3 can activate 906-009_LNGFR CAR-T cells leading to target cell death, suggesting that NSCLC could be an indication of interest for this therapy.

### Example 13

### CLDN3 Epitope Mapping

Within the present study, 906-009_LNGFR was used for assessment of the CLDN3 CAR epitope. “906-009_LNGFR” contain the same scFv, hinge, signalling moiety, and co-stimulatory domains as “SO-CD20-906_009” CLDN3 CAR-T cells; however, it does not contain the CD20 domain and contains an LNGFR tag. The CLDN3 binding element remains the same, and the two molecules have been demonstrated to have comparable functionality. Tool RKO target cells were generated, expressing different CLDN3 mutants with alanine replacing wild type residues. 906-009_LNGFR activation was evaluated by IFNγ release following co-culture with RKO target cells lines expressing the mutants. In addition, 906-mAb, a monoclonal antibody version comprising the scFv in 906-009_LNGFR, was used in flow cytometry to evaluate binding to the cell lines.

To identify the epitope of 906-009_LNGFR, in silico protein structural analysis was performed to predict residue surface accessibility. The data was used to generate tool RKO CLDN3 KO target cells expressing different mutated CLDN3 versions with alanine replacing the candidate wild type residues Mutations were generated across both CLDN3 extracellular loops 1 and 2 (ECL-1 and ECL-2).

The epitope was determined by measuring the binding of 906-mAb to the RKO KO target cells using flow cytometry. CAR T activation was evaluated by IFNγ secretion following co-culture of 906-009_LNGFR produced from 3 healthy donors with RKO KO target cells. If a mutation is within the epitope of 906-009_LNGFR, a reduction in binding and activation is expected, and therefore a reduction in 906-mAb and IFNγ signal compared with RKO KO CLDN3 wild-type (WT) cells.

Binding of the 906-mAb was significantly reduced with N38A and E153A mutant target cells compared with RKO cells expressing WT CLDN3. Co-culture of 906-009_LNGFR with these mutants also led to a significant decrease in IFNγ release after 24 hours compared with RKO cells expressing WT CLDN3. The data suggests that amino acids N38 and E153 are critical for the binding and activation of 906-009_LNGFR and are therefore part of the CAR binding epitope. The data also shows that the 906-009_LNGFR epitope is non-linear, spanning both extracellular loop1 (N38) and extracellular loop 2 (E153) of the CLDN3 protein (see e.g., SEQ ID NO: 13).

### Materials and Methods

Protein Structural Analysis. Protein structural analysis was carried out in order to select CLDN3 mutations for cell line generation. Protein sequences were aligned in CCG (Chemical Computing Group) MOE (Molecular Operating Environment) 2018.01 or 2019.0101, either manually, or using the automated MOE Align function. Protein crystal structures were superimposed against each other using CCG MOE 2018.01. Any non-claudin chains in the structures were deleted, and where present, multiple claudin chains were separated into discrete Tags prior to superimposition. Residue surface accessibility was calculated using the Residue Properties function in CCG MOE 2018.01 or 2019.0101. Where multiple different superimposed structures were used, the output was analysed in Microsoft Excel. An automated sequence alignment generated in CCG MOE was used to manually align the protein sequences in Excel, together with the associated surface accessibility data. Mean “ASA (A\textcircled{ }2)” [surface accessible area] and “Exposure (%)” [percentage surface accessibility vs residues within Gly-X-Gly tripeptide peptide] values were averaged across relevant structures and annotated as either exposed (>36%) or partially exposed (>9%). Protein homology modelling was performed using the CCG MOE 2019.0101 Homology Model function with default parameters.

CLDN3 mutant target cell line generation. MILLIPORE SIGMA generated a total of 15 monoclonal cell lines using targeted integration of 15 different cassettes, each encoding an EF1alpha promoter driving expression of CLDN3 WT or CLDN3 single-point amino acid mutants (herein referred to as “RKO KO target cells”) and GFP at the AAVS1 locus in RKO CLDN3 KO cells (herein referred to as “RKO KO”). Cells were stored at −150° C. before use.

Anti-CLDN 3 906-mAb generation. An anti-CLDN3 906-mAb was generated as per experiment N65028-27, and PE-conjugated externally at BIORAD.

906-009 LNGFR and UT-T cell production. T cells were produced and normalised as outlined in previous sections. In brief, CD4/CD8 T cells were isolated from human whole blood, transduced and expanded. Cells were normalised to 30% transduction efficiency before cryopreservation and sotrage at −150° C.

Culture of RKO KO target cell lines. Cells were cultured in RPMI containing 10% FBS, 1% GLUTAMAX and 1% Sodium Pyruvate. Cells were analysed using flow cytometry on the same day as co-culture set-up.

RKO KO target cell line preparation for co-culture and flow. All cells were seeded in RPMI containing 10% FBS, 1% GLUTAMAX and 1% Sodium Pyruvate. RKO KO cells (negative for CLDN3, generated in-house) were used as a negative control cell line, and RKO KO CLDN3 WT cells generated by SIGMA were used as a positive control cell line. Media was removed from T75 flasks and flasks were washed with PBS before the addition of 3 mL trypLE per flask. Cells were left for no longer than 5 minutes and flasks were tapped to dislodge cells. To deactivate TrypleE, 9 mL media was added per flask and cell counts were performed using the NUCLEOCOUNTER NC-250. The volume of cells required for plating was transferred to a falcon tube and centrifuged at 400×g for 5 mins. Supernatant was removed and cells resuspended in plating media to a final concentration of 3×10⁵ cells/mL). Cells were seeded in triplicate into 96-well flat-bottom plates at 3×10⁴ cells/well in 100 uL, and incubated at 37° C., 5% CO2 for 1 hour whilst preparing the T cells for co-culture. For flow cytometry, 1×10⁵ cells were seeded into duplicate 96-well V-bottom plates.

Thawing of T cells and co-culture with target cells. T cells (stored at −150° C.) were thawed to 37° C. and transferred dropwise into 10 mL warmed plating media. Cells were centrifuged at 400×g for 5 mins, supernatant removed and resuspended in 5 mL of plating media. Cells were counted using the NUCLEOCOUNTER NC-250. Cells were seeded on top of target cells at 9×10⁴ cells/well for a 1:1 ratio of target cell:transduced T cell. Co-culture plates were incubated for 24 h at 37° C., 5% CO₂.

Flow cytometry. RKO KO target cells were seeded for flow cytometry as described in previous sections. Wells were topped up with flow buffer (PBS+2% FBS+2 mM EDTA+0.05% sodium azide) and plates were centrifuged at 300×g for 5 minutes. Supernatant was flicked off, cells were resuspended in 150 uL flow buffer. Plate was centrifuged at 300×g for 5 minutes. Supernatant was flicked off and cells were resuspended in 50 uL IgG block at 1:100 for 10 minutes at RT. Following incubation, plate was washed twice as above. Cells were resuspended in 50 uL of diluted antibodies (all a 1 in 300 dilution) in flow buffer, or flow buffer alone (unstained control). Cells were incubated for 30 minutes at RT. After incubation, plates were washed twice with 150 uL flow buffer. Cells were then resuspended in 100 uL flow buffer containing DAPI at a 1:200 concentration as a live/dead stain. Cells were run immediately on the CYTOFLEXS. Cells were acquired on fast flow rate, with a stopping condition of 10,000 live cells (based on Total>Singlets>Live).

MSD. Co-culture plates were centrifuged at 400×g for 5 mins and supernatant was transferred to 96-well V bottom plates and stored at −80° C. until MSD analysis. IFNγ MSD assay was performed following manufacturers' instructions. Supernatants were thawed to room temperature and diluted as appropriate in Diluent 2. V-Plex MSD plates were washed three times with 150 μl of PBS+0.05% Tween (Sodexo) using a plate washer. The human IFNγ calibrator was reconstituted in 1000 μl of Diluent 2, equilibrated at RT for 15 minutes and briefly vortexed. Using diluent 2, a 1:4 dilution series was performed to prepare an 8-point calibration curve, including Diluent 2 only as the blank. Each plate was loaded with 50 μl of the calibrators and relevant samples, sealed and incubated at RT with shaking for 2 h. Plates were washed as before. Detection antibody was diluted in Diluent 3 (1:50) and 25 μl was added to each well. Plates were sealed and incubated at RT with shaking for 2 h. The plates were washed as before and 150 μl of 2× read buffer T was added to each well before reading on the MSD Sector 600 Imager.

Data Analysis: Flow Cytometry. Flow cytometry data was analysed using FLOWJO and further analysed in Microsoft excel and RStudio. Binding of the 906-mAb was determined as PE-positive cells, gated on unstained controls, whilst GFP-expression was determined as FITC-positive cells gated on the GFP-negative RKO KO control cell line. Gates were set within Total Cells>Singlets>Live>906-mAb-PE positive (FIG. 55A). Statistics for % PE-positive cells (as % of live cells) and Median Fluorescence Intensity (MFI) were calculated in FLOWJO and exported to Microsoft Excel for further analysis. Statistical analysis was performed in R version 3.6.3. Briefly, MFI was log 10 transformed and a mixed model was used with a fixed effect for cell line and a random effect for Plate, whilst % Parent was analysed on a linear scale in a mixed model with a fixed effect for cell line and a random effect for plate.

Data Analysis: MSD analysis of IFNγ release. MSD data was analysed using MSD WORKBENCH software. Standards and unknowns were assigned to relevant wells on each MSD plate. Raw signals from the calibrators were used by the software to generate standard curves using a 4-parameter logistic model (or sigmoidal dose-response) with a 1/y2 weighting function. Unknown samples were interpolated from the relative standard curve and multiplied by defined dilution factors to produce a ‘calculated concentration’ of IFNγ in pg/mL. These values were exported to Microsoft Excel for statistical analysis and data plotting for presentation. Cytokine release was log₁₀ transformed and a mixed model was used with fixed effects for CAR, cell line and their interaction. Random effects were used for Plate and Donor.

### Results

Protein Structural Analysis for selection of mutant cell line generation. In silico protein structural analysis was performed to prioritise which amino acids to mutate to Alanine. Prioritisation of amino acids was based on surface accessibility predictions to select amino acids that had the highest potential to be part of the 906-009_LNGFR binding epitope. Analysis was performed sequentially, using preliminary in vitro experiments to help inform each subsequent protein analysis (data not shown).

Analysis 1. Protein crystal structures of human CLDN4 (PDB 5B2G), mouse CLDN15 (PDB 4P79) and mouse CLDN19 (PDB 3X29) were aligned and superimposed in CCG MOE 2018.01, and mean ASA and percentage surface exposure calculated across either just the CLDN4 chains or across all structures and mapped onto an aligned CLDN3 protein sequence. The surface accessibility predictions were used to select CLDN3 residues for alanine scanning mutagenesis, initially categorised as [1] “exposed” across the CLDN4 chains, [2] additional “exposed” residues from across all structures, [3] “partially exposed” across the CLDN4 chains, and [4] additional “partially exposed” residues from across all structures. Linear peptide scanning had previously identified extracellular loop 2 (ECL2) as potentially part of the epitope, and therefore this information was used visually to further prioritise the “exposed” residues for alanine scanning mutagenesis, as listed below.
1. Spatially relevant ECL2 residues: Phe146, Tyr147, Pro149, Leu150, Pro152 and Glu153
2. Spatially relevant ECL1 loop 1 residues: Ile35, Gly36, Ser37, Asn38, Ile40 and Thr41
3. Spatially relevant ECL1 loop 3 residues: Asp67, Ser68, Leu69, Leu70, Ala71, Leu72, Pro73 and Gln74
4. ECL1 loop 2, plus additional less spatially relevant ECL1/2 residues: Ser57, Thr58, Gly59, Gln60, Met61, Gln62, Cys63, Lys64, Va165, Gln77, Ala78, Asn140, Arg144, Lys156 and Glu158

Analysis 2. The alanine scanning mutagenesis identified residue Glu153, within ECL2, as potentially part of the epitope. A homology model of human CLDN3 was generated using human CLDN4 (PDB 5B2G) as a template. Mean ASA and percent exposure were calculated for the two highly surface exposed regions of ECL1 (residues 35-42 and 57-61) and, in combination with proximity to ECL2 from the alanine scanning output, were used to prioritise these ECL1 residues for alanine scanning mutagenesis in order of Ser37, Asn38, Gly36, Ile35, Ile40, Thr41, Ser57, Thr58, Gly59, Gln60 to Met61.

Analysis 3. The additional alanine scanning mutagenesis further identified residue Asn 38 within ECL1 as potentially part of the epitope. This information was mapped onto the CLDN3 homology model in CCG MOE 2019.0101, and, in combination with the previous mutagenesis data, used to visually select further residues for additional alanine scanning mutagenesis, as shown below.
1. ECL2: Phe146, Tyr147 and Gln155
2. ECL1: Gln43, Ile45, Gln56, Leu70 and Ala71

Analysis 4. Protein sequences of human claudins 3, 4, 5, 6, 8, 9 and 17 were aligned manually in CCG MOE 2019.0101. CLDN4 exhibits a small signal in a CAR-T IFNγ assay, whereas none of the remaining listed claudins show any IFNγ signal. Residues that only differ between CLDN3 and the latter claudin family members may therefore be responsible for the observed lack of signal. Residues within the extracellular loops of CLDN3 that differed either (1) to CLDN4 or (2) to any of the other CLDN sequences (but not CLDN4) were independently mapped onto the CLDN3 homology model. Analysis of these latter residues on the alignment and structure in combination with the locations of the previous mutations that exhibit effects on binding/potency of the CAR-T, however, was not able to identify any obvious further residue positions for alanine mutagenesis.

Analysis 5. Further visual inspection of all of the alanine scanning mutagenesis data mapped onto the CLDN3 homology model utilising CCG MOE 2019.0101 was used to select three additional residues for mutagenesis: Ala154, Phe34 and Arg157. The final RKO KO CLDN3 mutant cells were generated based on this protein structural analysis, as described in previous sections.

Flow cytometry analysis of 906-mAb and anti-hCLDN3 binding. To assess the effects of CLDN3 mutations on CAR T binding, the binding of a monoclonal antibody version of the scFv composing 906-009_LNGFR binding domain, known as 906-mAb, to RKO KO target cells was assessed by flow cytometry. Cell lines for analysis included RKO KO, a CLDN3 knockout (included as a negative control), RKO KO CLDN3 mutant cells (with various single-amino acid CLDN3 mutations) and RKO KO CLDN3 WT cells (positive control). RKO KO CLDN3 mutant cell lines were selected and generated as outlined in previous sections.

The 906-mAb showed binding (as represented by PE-MFI) to the RKO KO CLDN3 WT cell line and all RKO KO CLDN3 mutant cells, with the exception of the N38A and E153A mutant cell lines, which showed significantly decreased 906-mAb-PE MFI and % 906-mAb-PE positive population compared with WT (FIGS. 55B, 55C, 55D; Tables 24, 25). As expected, 906-mAb did not bind to the RKO KO negative control cell line.

GFP expression remained similar between RKO KO CLDN3 WT and mutant cell lines (FIG. 55B), suggesting that the differences in 906-mAb binding was not an artefact of differences in total CLDN3 protein expression.

These data show that mutations in residues N38 and E153 of the CLDN3 protein cause a decreased ability for 906-mAb to bind, suggesting these amino acids are involved in the 906-009_LNGFR binding epitope.

TABLE 24
Comparison of 906-mAb binding to RKO KO CLDN3 mutant
cells compared with RKO KO CLDN3 WT cells. Estimate
is fold change MFI vs CLDN3 WT, where 1x is identical.

| Cell Line | Estimate | Lower.CL | Upper.CL | p. value |
|---|---|---|---|---|
| RKO KO | 0.01 | 0 | 0.05 | P < 0.001 |
| F34A | 0.4 | 0.11 | 1.51 | 0.28 |
| N38A | 0.01 | 0 | 0.04 | P < 0.001 |
| I39A | 0.95 | 0.25 | 3.6 | 1 |
| S42A | 3.91 | 1.04 | 14.73 | 0.043 |
| Q43A | 2.68 | 0.71 | 10.12 | 0.216 |
| I45A | 3.17 | 0.84 | 11.94 | 0.109 |
| N148A | 2.75 | 0.73 | 10.39 | 0.195 |
| P149A | 2.03 | 0.54 | 7.66 | 0.545 |
| V150A | 2.98 | 0.79 | 11.26 | 0.141 |
| P152A | 5.07 | 1.34 | 19.11 | 0.013 |
| E153A | 0.01 | 0 | 0.05 | P < 0.001 |
| Q155A | 3.4 | 0.9 | 12.83 | 0.08 |
| K156A | 0.73 | 0.19 | 2.76 | 0.971 |
| R157A | 1.31 | 0.35 | 4.93 | 0.985 |

CL = confidence intervals.

TABLE 25
Comparison of 906-mAb binding to RKO KO CLDN3 mutant cells
compared with RKO KO CLDN3 WT cells. Estimate is Change
in % Parent vs CLDN3 WT, where 0% is no change.

| Cell Line | estimate | lower.CL | upper.CL | p. value |
|---|---|---|---|---|
| RKO KO | −99.72 | −102.33 | −97.11 | P < 0.001 |
| F34A | −0.95 | −3.56 | 1.66 | 0.845 |
| N38A | −99.16 | −101.78 | −96.55 | P < 0.001 |
| I39A | 0.1 | −2.51 | 2.71 | 1 |
| S42A | 0.1 | −2.51 | 2.71 | 1 |
| Q43A | 0.1 | −2.51 | 2.71 | 1 |
| I45A | 0.05 | −2.56 | 2.66 | 1 |
| N148A | 0.1 | −2.51 | 2.71 | 1 |
| P149A | 0.1 | −2.51 | 2.71 | 1 |
| V150A | 0 | −2.61 | 2.61 | 1 |
| P152A | 0.1 | −2.51 | 2.71 | 1 |
| E153A | −94.52 | −97.14 | −91.91 | P < 0.001 |
| Q155A | 0.1 | −2.51 | 2.71 | 1 |
| K156A | 0.05 | −2.56 | 2.66 | 1 |
| R157A | 0.1 | −2.51 | 2.71 | 1 |

CL = confidence intervals.

TABLE 26
IFNγ release after co-culture of 906-009 LNGFR with
RKO KO CLDN3 mutant cells compared with RKO KO CLDN3
WT cells (normalised to untransduced T cells). Estimate
is IFNγ as % of IFNγ in CLDN3 WT, where 100% is identical.

| Cell Line | Estimate | Lower.CL | Upper.CL | p. value |
|---|---|---|---|---|
| RKO KO | 0.19 | 0.11 | 0.32 | P < 0.001 |
| F34A | 79.74 | 47.25 | 134.57 | 0.395 |
| N38A | 0.11 | 0.07 | 0.19 | P < 0.001 |
| I39A | 71.45 | 42.34 | 120.58 | 0.207 |
| S42A | 95.23 | 56.43 | 160.7 | 0.854 |
| Q43A | 72.83 | 43.04 | 122.56 | 0.23 |
| I45A | 66.09 | 39.16 | 111.53 | 0.12 |
| N148A | 104.38 | 61.85 | 176.15 | 0.872 |
| P149A | 95.53 | 56.61 | 161.21 | 0.863 |
| V150A | 91.26 | 54.08 | 154 | 0.731 |
| P152A | 61.57 | 36.48 | 103.9 | 0.069 |
| E153A | 3.56 | 2.11 | 6.01 | P < 0.001 |
| Q155A | 86.26 | 51.12 | 145.57 | 0.579 |
| K156A | 101.26 | 60 | 170.87 | 0.963 |
| R157A | 106.81 | 63.29 | 180.25 | 0.804 |

CL = confidence intervals.

IFNγ cytokine secretion following co-culture of 906-009_LNGFR with RKO target cell lines. Cytokine secretion is part of the T cell response to antigen engagement and detection of IFNγ release was used to determine the effects of CLDN3 mutations on CAR T activation. IFNγ cytokine release was measured following 24 h co-culture of 906-009_LNGFR with RKO KO target cells. As expected, co-culture of 906-009_LNGFR with RKO KO cells (CLDN3 negative) did not induce IFNγ above levels in T cell alone controls (data not shown). IFNγ secretion was significantly reduced after 906-009_LNGFR co-culture with N38A and E153A mutant cell lines only compared with WT (fold-change of 0.01, P<0.001 for both mutants). Data was normalised to IFNγ secretion in co-cultures with untransduced T cells from matching donors (FIG. 56; Table 26). Co-culture with the S42A, P152A and Q155A mutant cell lines resulted in a small but significant increase in IFNγ secretion compared with WT (p<0.05) (Table 26). However, as the % 906-mAb-PE positive cell population for these mutants did not decrease significantly compared with WT cells, and there was also no significant decrease in 906-009_LNGFR IFNγ release after co-culture with these mutant cells, it is unlikely that these observation on MFI are biologically relevant. These data suggest that mutations in residues N38 and E153 of the CLDN3 (see e.g., SEQ ID NO: 13) protein cause a decreased ability for 906-009_LNGFR to become activated by target cells.

The aim of this study was to identify the 906-009_LNGFR epitope by determining amino acid residues necessary for 906-009_LNGFR binding and activation. Tool cell lines were generated by mutating single-amino acids to Alanine across both CLDN3 extracellular loops. Flow cytometry was used to identify which of these alterations would reduce CAR binding (measured by the binding of a 906-mAb, the monoclonal antibody version of 906-009_LNGFR scFv binding domain), whilst assessment of IFNγ secretion after CAR T co-culture with mutant cell lines was to determine whether mutations reduced CAR T activation.

Of the mutations tested, mutations in CLDN3 amino acid residues N38 and E153 only caused a significant decrease in binding of 906-mAb. These mutations also exclusively caused a significant decrease in activation of 906-009_LNGFR. Taken together these data suggest that N38 and E153 residues are part of the 906-009_LNGFR (and SO-CD20-906_009) binding epitope and are critical for 906-009_LNGFR target binding and subsequent activation. These data also show that the CAR epitope is non-linear and discontinuous, spanning both ECL-1 (N38) and ECL-2 (E153) of the CLDN3 protein.

Overall, the data from this study show a decrease in both activation and binding of 906-009_LNGFR (and SO-CD20-906_009) when residues N38 and E153 are mutated, providing strong evidence that these amino acids are critical in the CLDN3 CAR epitope.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A chimeric antigen receptor comprising a polypeptide comprising:
a) an extracellular domain which comprises a claudin-3 binding domain comprising a heavy chain variable region (VH) comprising a heavy chain complementarity determining region 1 (CDRH1) sequence of SEQ ID NO: 1; a heavy chain complementarity determining region 2 (CDRH2) sequence of SEQ ID NO: 2; a heavy chain complementarity determining region 3 (CDRH3) sequence of SEQ ID NO: 3;
b) a transmembrane domain; and
c) one or more intracellular signalling domains.

2. The chimeric antigen receptor according to claim 1, wherein the extracellular domain further comprises a claudin-3 binding domain comprising a light chain variable region (VL) comprising a light chain complementarity determining region 1 (CDRL1) sequence of SEQ ID NO: 4; a light chain complementarity determining region 2 (CDRL2) sequence of SEQ ID NO: 5; a light chain complementarity determining region 3 (CDRL3) sequence of SEQ ID NO: 6.

3. The chimeric antigen receptor according to claim 2, wherein the VL is located at the N-terminus of the VH, or wherein the VH is located at the N-terminus of the VL.

4. The chimeric antigen receptor according to claim 2, wherein the VL and the VH are directly fused to each other via a peptide bond or linked to each other via a peptide linker.

5. The chimeric antigen receptor according to claim 1, wherein the antigen binding domain is selected from the group consisting of: a Camel Ig, Ig NAR, Fab fragments, Fab′ fragments, F(ab)′2 fragments, F(ab)3 fragments, Fv, single-chain variable fragment (scFv), bis-scFv, (scFv)2, minibody, diabody, triabody, tetrabody, disulfide stabilized Fv protein (dsFv), and single-domain antibody (sdAb).

6. The chimeric antigen receptor according to claim 1, wherein the VH comprises an amino acid sequence having at least 90% or 95% sequence identity to SEQ ID NO: 7, or an amino acid sequence having one, two, three, four, or five amino acid substitutions, insertions, or deletions relative to SEQ ID NO: 7, and/or wherein the VL comprises an amino acid sequence having at least 90% or 95% sequence identity to SEQ ID NO: 8, or an amino acid sequence having one, two, three, four, or five amino acid substitutions, insertions, or deletions relative to SEQ ID NO: 8.

7. The chimeric antigen receptor according to claim 1, wherein the VH comprises an amino acid sequence of SEQ ID NO: 7 and/or the VL comprises an amino acid sequence of SEQ ID NO: 8.

8. The chimeric antigen receptor according to claim 1, wherein the extracellular domain comprises an amino acid sequence having at least 90% or 95% sequence identity to SEQ ID NO: 11, or an amino acid sequence having one, two, three, four, or five amino acid substitutions, insertions, or deletions relative to SEQ ID NO: 11, or wherein the extracellular domain comprises an amino acid sequence having at least 90% or 95% sequence identity to SEQ ID NO: 18, or an amino acid sequence having one, two, three, four, or five amino acid substitutions, insertions, or deletions relative to SEQ ID NO: 18.

9. The chimeric antigen receptor according to claim 1, wherein the transmembrane domain is derived from a polypeptide selected from the group consisting of: alpha or beta chain of the T-cell receptor, CD3δ, CD3ε, CD3γ, CD3ζ, CD4, CD5, CD8α CD9, CD16, CD22, CD27, CD28, CD33, CD37, CD45, CD64, CD80, CD86, CD134, CD137 (4-1BB), CD152, CD154, CD278 (ICOS) and PD1.

10. The chimeric antigen receptor according to claim 9, wherein the transmembrane domain is derived from CD8α.

11. The chimeric antigen receptor according to claim 1, wherein the one or more intracellular signalling domains is derived from an intracellular signalling molecule selected from the group consisting of: FcRγ, FcRβ, CD3γ, CD3ε, CD3δ, CD3ζ, CD22, CD66d, CD79a and CD79b or wherein the one or more intracellular signalling domains is CD3ζ.

12. The chimeric antigen receptor according to claim 1, further comprising a co-stimulatory signalling domain.

13. The chimeric antigen receptor according to claim 12, wherein the co-stimulatory domain is derived from a co-stimulatory molecule selected from the group consisting of: CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TLR1, TLR2, TLR3, TLR4, TLR5, TLR6, TLR7, TLR8, TLR9, TLR10, TRIM and ZAP70.

14. A chimeric antigen receptor comprising:
a) an extracellular domain which comprises a claudin-3 binding protein comprising a CDRH1 sequence of SEQ ID NO: 1; a CDRH2 sequence of SEQ ID NO: 2; a CDRH3 sequence of SEQ ID NO: 3; a CDRL1 sequence of SEQ ID NO: 4; a CDRL2 sequence of SEQ ID NO: 5; and a CDRL3 sequence of SEQ ID NO: 6;
b) a transmembrane domain derived from CD8α;
c) a costimulatory domain derived from CD137 (4-1BB); and
d) an intracellular signalling domain derived from CD3ζ.

15. The chimeric antigen receptor according to claim 1, wherein the chimeric antigen receptor comprises an amino acid sequence having at least 90% or 95% sequence identity to SEQ ID NOs: 12, 34, 35, 36, 37, 38, or 39, or an amino acid sequence having one, two, three, four, or five amino acid substitutions, insertions, or deletions relative to SEQ ID NOs: 12, 34, 35, 36, 37, 38, or 39, or wherein the chimeric antigen receptor comprises an amino acid sequence of SEQ ID NOs: 12, 34, 35, 36, 37, 38, or 39.

16. A chimeric antigen receptor that competes for binding with the chimeric antigen receptor according to claim 1.

17. An engineered immune effector cell, comprising the chimeric antigen receptor according to claim 1.

18. The immune effector cell according to claim 17, wherein the immune effector cell is selected from the group consisting of: a T cell, a cytotoxic T lymphocyte, a natural killer T lymphocyte cell, a macrophage, and a natural killer cell.

19. The immune effector cell according to claim 17, further comprising an ablation element.

20. The immune effector cell according to claim 17, wherein the ablation element is derived from a polypeptide selected from the group consisting of: truncated human EGFR polypeptide and CD20, or wherein the ablation element is CD20.

21. A polynucleotide encoding the chimeric antigen receptor according to claim 1.

22. A vector comprising the polynucleotide of claim 21.

23. The vector according to claim 22, wherein the vector is selected from the group consisting of: retroviral vector, lentiviral vector, adeno-associated virus (AAV) vector, human immunodeficiency virus I (HIV-I), human immunodeficiency virus 2 (HIV-2), visna-maedi virus (VMV) virus, caprine arthritis-encephalitis virus (CAEV), equine infectious anemia virus (EIAV), feline immunodeficiency virus (FIV), bovine immune deficiency virus (BIV), and simian immunodeficiency virus.

24. A pharmaceutical composition comprising the engineered immune effector cell according to claim 17, and a pharmaceutically acceptable excipient.

25. A method of treating cancer in a patient in need thereof, said method comprising administering to the patient a therapeutically effective amount of the pharmaceutical composition according to claim 24.

26. The method according to claim 25, wherein the cancer is solid cancer, epithelial cancer, colorectal cancer, pancreatic cancer, breast cancer, triple-negative breast cancer (TNBC), ovarian cancer, lung cancer, non-small cell lung cancer (NSCLC), or prostate cancer.

27. The method according to claim 26, wherein the cancer is colorectal cancer.

28. A claudin-3 binding protein, comprising a heavy chain variable region (VH) comprising a heavy chain complementarity determining region 1 (CDRH1) sequence of SEQ ID NO: 1; a heavy chain complementarity determining region 2 (CDRH2) sequence of SEQ ID NO: 2; a heavy chain complementarity determining region 3 (CDRH3) sequence of SEQ ID NO: 3; and a light chain variable region (VL) comprising a light chain complementarity determining region 1 (CDRL1) sequence of SEQ ID NO: 4; a light chain complementarity determining region 2 (CDRL2) sequence of SEQ ID NO: 5; a light chain complementarity determining region 3 (CDRL3) sequence of SEQ ID NO: 6.

29. A polynucleotide encoding the claudin-3 binding protein according to claim 28.

30. A vector comprising the polynucleotide of claim 29.

31. A pharmaceutical composition comprising the claudin-3 binding protein according claim 28, and a pharmaceutically acceptable excipient.

32. A method of treating cancer in a patient in need thereof, said method comprising administering to the patient a therapeutically effective amount of the pharmaceutical composition according to claim 31.

33. An isolated claudin-3 binding protein that binds to a discontinuous epitope on human claudin-3 comprising at least N38 and E153 of SEQ ID NO:13.

34. A chimeric antigen receptor comprising a polypeptide comprising:
a) an extracellular domain which comprises the isolated claudin-3 binding protein according to claim 33;
b) a transmembrane domain; and
c) one or more intracellular signalling domains.

35. An engineered immune effector cell, comprising the chimeric antigen receptor according to claim 34.

36. A pharmaceutical composition comprising the isolated claudin-3 binding protein according to claim 33, and a pharmaceutically acceptable excipient.

37. A method of treating cancer in a patient in need thereof, said method comprising administering to the patient a therapeutically effective amount of the pharmaceutical composition according to claim 36.


SEQUENCE LISTING (40 sequences)

SEQ ID NO: 1 - protein, 5 aa, synthetic construct
NYWVH

SEQ ID NO: 2 - protein, 17 aa, synthetic construct
RIEPNSSGSQYNEKFKN

SEQ ID NO: 3 - protein, 8 aa, synthetic construct
GVMVPLDY

SEQ ID NO: 4 - protein, 11 aa, synthetic construct
KASQDINRYIA

SEQ ID NO: 5 - protein, 7 aa, synthetic construct
YTSTLQP

SEQ ID NO: 6 - protein, 8 aa, synthetic construct
LQYETLYS

SEQ ID NO: 7 - protein, 117 aa, synthetic construct
QVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSS

SEQ ID NO: 8 - protein, 106 aa, synthetic construct
DIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIK

SEQ ID NO: 9 - protein, 20 aa, synthetic construct
GGGGSGGGGSGGGGSGGGGS

SEQ ID NO: 10 - protein, 21 aa, synthetic construct
MALPVTALLLPLALLLHAARP

SEQ ID NO: 11 - protein, 243 aa, synthetic construct
DIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSS

SEQ ID NO: 12 - protein, 489 aa, synthetic construct
MALPVTALLLPLALLLHAARPDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 14 - protein, 23 aa, Homo sapiens
WSANTIIRDFYNPVVPEAQKREM

SEQ ID NO: 15 - protein, 4 aa, Homo sapiens
PVVP

SEQ ID NO: 16 - DNA, 729 bp, synthetic construct
  source 1..729: mol_type = other DNA
gacatccagatgacccagagccctagcagcctgagcgccagcgtgggagacagggtgaccatcacctgcaaggccagccaggacatcaacaggtacatcgcctggtaccagcagaagcccggcaaggcccccaagctgctgatccactacaccagcaccctgcagcccggcgtgccctctaggtttagcggcagcggcagcggcaccgacttcaccctgaccatcagcagcctccagcccgaggacttcgccacctactactgcctgcagtacgagaccctgtacagcttcggccagggcaccaagctggagattaagggcggaggtgggagcggcggaggaggcagcggcggaggcggtagcgggggcggaggcagccaggtgcagctcgtgcagagcggagccgaggtgaaaaagcccggaagctctgtcaaggtgagctgcaaggccagcggctacaccttcaccaactactgggtgcactgggtgaggcaggctcccggacagggcctggagtggatgggcaggatcgagcccaacagcagcggcagccagtacaacgagaagttcaagaacagggtgaccatcaccgccgacaagagcaccagcaccgcctacatggaactgagcagcctgaggagcgaggacaccgccgtgtattactgcgccaggggcgtgatggtgcccctggactactggggccagggcaccctggtgacagtgagcagc

SEQ ID NO: 17 - DNA, 2448 bp, synthetic construct
  source 1..2448: mol_type = other DNA
atgaccacccccaggaactccgtgaacggcaccttccccgccgagccaatgaagggccctatcgctatgcagagcggccccaagcccctgttcaggaggatgtcaagcctcgtgggccctacccagagcttcttcatgagggagagcaagaccctgggcgccgtgcagatcatgaacggcctcttccatatcgccctgggcggcctgctgatgatccccgctggcatttacgcccccatctgcgtgaccgtgtggtatcccctgtggggcggcatcatgtacatcattagcgggagcctgctggccgccaccgagaagaactctcggaagtgcctggtgaagggcaagatgatcatgaacagcctgagcctcttcgccgccatctccggcatgatcctgagcatcatggacatcctgaacatcaagatcagccacttcctgaagatggaaagcctcaacttcatcagggcccacaccccctacatcaacatctacaactgcgagcccgccaatcccagcgagaagaacagccccagcacccagtactgctacagcatccagagcctgttcctcggcatcctgagcgtgatgctgatcttcgccttcttccaagagctggtgatcgccggcatcgtggagaacgagtggaagaggacctgcagcaggccaaagagcaacatcgtgctgctgagcgccgaggagaagaaggagcagactatcgagatcaaggaggaggtggtgggcctgacagagaccagcagccagcccaagaacgaggaggacatcgagatcatccccatccaggaggaggaggaggaggaaaccgagaccaacttccccgagcccccccaggatcaggagtctagccccatcgagaacgacagcagccccggcagcagggccaaaaggagcggcagcggcgcaaccaacttcagcctgctgaagcaggccggagacgtggaggagaatcccggcccaatggcactgccagtcaccgctctgctgctgcccctggccctgctgctgcacgccgccaggcccgatattcagatgacccagtccccctctagcctgagcgccagcgtgggcgacagggtgaccatcacctgcaaggccagccaggacatcaacagatacatcgcctggtaccagcagaagcccggcaaggcccccaagctgctgatccactacaccagcaccctgcagcccggcgtgcctagcagatttagcggcagcggcagcggcaccgacttcaccctgactatcagcagcctgcagcccgaggacttcgccacctactactgcctgcagtacgagacactgtacagcttcggccagggcaccaagctggaaattaaaggcggaggcggcagcggcggcggcggctcaggcggcggaggcagcggcggcgggggcagccaggtgcagctggtccagagcggcgccgaggtgaaaaagcccggcagcagcgtgaaagtgtcctgcaaggccagcggctacaccttcaccaactactgggtgcactgggtccggcaggcccccggccagggactggagtggatggggaggatcgagcctaacagcagcggcagccagtacaacgagaagttcaagaacagggtgaccatcaccgccgacaagagcaccagcaccgcctacatggagctcagcagcctgcgcagcgaagacaccgccgtgtattactgcgccaggggcgtgatggtgcccctggactactggggacagggcaccctggtgaccgtgagcagcgcctctaccacaacccccgctcccaggccccccacccctgcccccaccattgcctcacaacccctgagcctgaggcccgaggcctgtaggcccgccgccggaggcgccgtgcacaccaggggcctggacttcgcctgcgacatctatatctgggcccccctggccggaacctgtggcgtgctgctcctgagcctggtgatcaccctgtactgcaagcggggcaggaagaagctgctgtacatcttcaagcagcccttcatgaggcccgtccagaccacccaggaggaggacgggtgcagctgcaggtttcccgaagaggaggaaggcggctgcgagctgagggtcaagtttagcaggagcgccgacgctcccgcctaccagcaagggcagaatcagctctacaacgagctgaacctgggcaggagggaggagtacgacgtgctggacaaaaggaggggcagggaccccgagatgggcggcaagcccagaaggaagaacccccaggagggcctgtacaacgagctgcagaaggacaaaatggccgaggcctacagcgagatcggcatgaagggcgagaggaggaggggcaagggccacgacggcctgtaccagggcctgagcaccgctaccaaggacacctacgacgccctgcacatgcaggccctgcctcccagatga

SEQ ID NO: 18 - protein, 243 aa, synthetic construct
QVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIK

SEQ ID NO: 19 - protein, 24 aa, synthetic construct
IYIWAPLAGTCGVLLLSLVITLYC

SEQ ID NO: 20 - protein, 42 aa, synthetic construct
KRGRKKLLYIFKQPFMRPVQTTQEEDGCSCRFPEEEEGGCEL

SEQ ID NO: 21 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 23 - protein, 29 aa, synthetic construct
GSRAKRSGSGATNFSLLKQAGDVEENPGP

SEQ ID NO: 24 - protein, 815 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSPGSRAKRSGSGATNFSLLKQAGDVEENPGPMALPVTALLLPLALLLHAARPDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 489 aa, synthetic construct
MALPVTALLLPLALLLHAARPQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 51 aa, Homo sapiens
RVSAFIGSNIITSQNIWEGLWMNCVVQSTGQMQCKVYDSLLALPQDLQAAR

SEQ ID NO: 27 - protein, 672 aa, synthetic construct
MALPVTALLLPLALLLHAARPDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 456 aa, synthetic construct
MALPVTALLLPLALLLHAARPDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 672 aa, synthetic construct
MALPVTALLLPLALLLHAARPQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKASESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 456 aa, synthetic construct
MALPVTALLLPLALLLHAARPQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKASESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 45 aa, synthetic construct
TTTPAPRPPTPAPTIASQPLSLRPEACRPAAGGAVHTRGLDFACD

SEQ ID NO: 33 - protein, 12 aa, synthetic construct
ESKYGPPCPPCP

SEQ ID NO: 34 - protein, 468 aa, synthetic construct
DIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 468 aa, synthetic construct
QVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 651 aa, synthetic construct
DIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 435 aa, synthetic construct
DIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKGGGGSGGGGSGGGGSGGGGSQVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSASESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 651 aa, synthetic construct
QVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKASESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 435 aa, synthetic construct
QVQLVQSGAEVKKPGSSVKVSCKASGYTFTNYWVHWVRQAPGQGLEWMGRIEPNSSGSQYNEKFKNRVTITADKSTSTAYMELSSLRSEDTAVYYCARGVMVPLDYWGQGTLVTVSSGGGGSGGGGSGGGGSGGGGSDIQMTQSPSSLSASVGDRVTITCKASQDINRYIAWYQQKPGKAPKLLIHYTSTLQPGVPSRFSGSGSGTDFTLTISSLQPEDFATYYCLQYETLYSFGQGTKLEIKASESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 50 aa, synthetic construct
  VARIANT 1..5: note = This region may encompass 1-5 residues
  VARIANT 6..10: note = This region may encompass 1-5 residues
  VARIANT 1..50: note = This sequence may encompass 1 to 5 or more repeating units of GxSx, wherein x=1-5
GGGGGSSSSSGGGGGSSSSSGGGGGSSSSSGGGGGSSSSSGGGGGSSSSS